SEQUENCE LISTING (133 sequences)

SEQ ID NO: 1 - protein, 252 aa, Artificial Sequence
  SARS COV2 presumed RBD region residues 336-518
  misc_feature: Synthetic
NLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEIL

SEQ ID NO: 2 - protein, 57 aa, Artificial Sequence
  SARS CoV2 presumed RBM region 443 to 499 of the Spike protein
  misc_feature: Synthetic
SKVGGNYNYLYRLFRKSNLKPFERDISTEIYQAGSTPCNGVEGFNCYFPLQSYGFQP

SEQ ID NO: 3 - protein, 14 aa, Artificial Sequence
  Segment A, Residues 443 to 456 of the RBM
  misc_feature: Synthetic
SKVGGNYNYLYRLF

SEQ ID NO: 4 - protein, 27 aa, Artificial Sequence
  Segment B, Residues 773 to 499 of the RBM
  misc_feature: Synthetic
YQAGSTPCNGVEGFNCYFPLQSYGFQP

SEQ ID NO: 5 - protein, 16 aa, Artificial Sequence
  anchor loop, residues 457 to 472
  misc_feature: Synthetic
RKSNLKPFERDISTEI

SEQ ID NO: 6 - protein, 1273 aa, Unknown
  Severe acute respiratory syndrome coronavirus 2
  MISC_FEATURE: Spike protein of SARS-COV2, NCBI Reference Sequence YP_009724390.1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - DNA, 3822 bp, Unknown
  Severe acute respiratory syndrome coronavirus 2
  misc_feature: Nucleic acid sequence encoding Spike protein of SARS-COV2, NC_045512.2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 8 - DNA, 72 bp, Artificial Sequence
  Oligo 1
  misc_feature: Synthetic
ttctattctcactccgctccagagctctggtctaaggtcggaggcaattacaattatttatatcgccttttc

SEQ ID NO: 9 - DNA, 72 bp, Artificial Sequence
  Oligo 2
  misc_feature: Synthetic
gaaaaggcgatataaataattgtaattgcctccgaccttagaccagagctctggagcggagtgagaatagaa

SEQ ID NO: 10 - DNA, 51 bp, Artificial Sequence
  Oligo 3
  misc_feature: Synthetic
  misc_feature (22)..(23): n is a, c, g, or t
  misc_feature (25)..(26): n is a, c, g, or t
  misc_feature (28)..(29): n is a, c, g, or t
aattatttatatcgccttttcnnknnknnkaccgaaatctaccaagcaggg

SEQ ID NO: 11 - DNA, 54 bp, Artificial Sequence
  Oligo 4
  misc_feature: Synthetic
  misc_feature (22)..(23): n is a, c, g, or t
  misc_feature (25)..(26): n is a, c, g, or t
  misc_feature (28)..(29): n is a, c, g, or t
  misc_feature (31)..(32): n is a, c, g, or t
aattatttatatcgccttttcnnknnknnknnkaccgaaatctaccaagcaggg

SEQ ID NO: 12 - DNA, 57 bp, Artificial Sequence
  Oligo 5
  misc_feature: Synthetic
  misc_feature (22)..(23): n is a, c, g, or t
  misc_feature (25)..(26): n is a, c, g, or t
  misc_feature (28)..(29): n is a, c, g, or t
  misc_feature (31)..(32): n is a, c, g, or t
  misc_feature (34)..(35): n is a, c, g, or t
aattatttatatcgccttttcnnknnknnknnknnkaccgaaatctaccaagcaggg

SEQ ID NO: 13 - DNA, 60 bp, Artificial Sequence
  Oligo 6
  misc_feature: Synthetic
  misc_feature (22)..(23): n is a, c, g, or t
  misc_feature (25)..(26): n is a, c, g, or t
  misc_feature (28)..(29): n is a, c, g, or t
  misc_feature (31)..(32): n is a, c, g, or t
  misc_feature (34)..(35): n is a, c, g, or t
  misc_feature (37)..(38): n is a, c, g, or t
aattatttatatcgccttttcnnknnknnknnknnknnkaccgaaatctaccaagcaggg

SEQ ID NO: 14 - DNA, 63 bp, Artificial Sequence
  Oligo 7
  misc_feature: Synthetic
  misc_feature (22)..(23): n is a, c, g, or t
  misc_feature (25)..(26): n is a, c, g, or t
  misc_feature (28)..(29): n is a, c, g, or t
  misc_feature (31)..(32): n is a, c, g, or t
  misc_feature (34)..(35): n is a, c, g, or t
  misc_feature (37)..(38): n is a, c, g, or t
  misc_feature (40)..(41): n is a, c, g, or t
aattatttatatcgccttttcnnknnknnknnknnknnknnkaccgaaatctaccaagcaggg

SEQ ID NO: 15 - DNA, 28 bp, Artificial Sequence
  Oligo 8
  misc_feature: Synthetic
taaacaactttcaacagtttcccagacg

SEQ ID NO: 16 - DNA, 42 bp, Artificial Sequence
  Oligo 9
  misc_feature: Synthetic
aattatttatatcgccttttcaccgaaatctaccaagcaggg

SEQ ID NO: 17 - protein, 1255 aa, Unknown
  Severe acute respiratory syndrome coronavirus
  MISC_FEATURE: SPIKE PROTEIN of Severe Acute Respiratory Syndrome coronavirus (SARS coronavirus) ShanghaiQXC1 , S1 protein, GenBank AAR86788.1,AAR86788, accession AY463059.1
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTNVSAAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKGELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 18 - protein, 1353 aa, Unknown
  Middle East respiratory syndrome coronavirus
  MISC_FEATURE: SPIKE PROTEIN of Middle East respiratory syndrome coronavirus (MERS), spike S1 protein GenBank AHX00731.1, GI612348173
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLGVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQFNSSYFKLSIPTNFSFGVTQEYIQTTIQKVTVDCKQYICNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYSPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 19 - protein, 661 aa, Artificial Sequence
  SARS CoV2 Spike-S1, S1 (M1---E661)
  misc_feature: Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYE

SEQ ID NO: 20 - DNA, 1983 bp, Artificial Sequence
  SARS CoV2 Spike-S1, Nucleic acid sequence Spike-S1
  misc_feature: Synthetic
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgag

SEQ ID NO: 21 - protein, 612 aa, Artificial Sequence
  SARS CoV2 Spike-S2, S2 (C662-T1273)
  misc_feature: Synthetic
CDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 22 - DNA, 1839 bp, Artificial Sequence
  SARS CoV2 Spike-S2
  misc_feature: Synthetic
tgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 23 - protein, 805 aa, Homo sapiens [sequence omitted]

SEQ ID NO: 24 - DNA, 2599 bp, Homo sapiens
  misc_feature: ACE2 mRNA, GenBank AB046569.1
tttttagtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataacgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaaaggcgacttcaggatccttatgtgcacaaaggtgacaatggacgacttcctgacagctcatcatgagatggggcatattcagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagatagagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaaaatatcattaaatgtcaaaactatgactctgttcagaaaaaaaaa

SEQ ID NO: 25 - protein, 5 aa, Artificial Sequence
  MERS CoV linker
  misc_feature: Synthetic
GFDEQ

SEQ ID NO: 26 - protein, 5 aa, Artificial Sequence
  MERS CoV linker
  misc_feature: Synthetic
PYNYH

SEQ ID NO: 27 - protein, 5 aa, Artificial Sequence
  MERS CoV linker
  misc_feature: Synthetic
SQAPL

SEQ ID NO: 28 - protein, 4 aa, Artificial Sequence
  MERS CoV linker
  misc_feature: Synthetic
LHLP

SEQ ID NO: 29 - protein, 4 aa, Artificial Sequence
  MERS CoV linker
  misc_feature: Synthetic
VVAT

SEQ ID NO: 30 - protein, 5 aa, Artificial Sequence
  Linker used for the reconstituted RBM
  misc_feature: Synthetic
  misc_feature (1)..(5): Xaa can be any naturally occurring amino acid
XXXXX

SEQ ID NO: 31 - protein, 4 aa, Artificial Sequence
  Linker used for the reconstituted RBM
  misc_feature: Synthetic
  misc_feature (1)..(4): Xaa can be any naturally occurring amino acid
XXXX

SEQ ID NO: 32 - protein, 4 aa, Artificial Sequence
  Linker used for the reconstituted RBM
  misc_feature: Synthetic
GDPM

SEQ ID NO: 33 - protein, 4 aa, Artificial Sequence
  Linker used for the reconstituted RBM
  misc_feature: Synthetic
GDPN

SEQ ID NO: 34 - protein, 4 aa, Artificial Sequence
  Linker used for the reconstituted RBM
  misc_feature: Synthetic
GEVD

SEQ ID NO: 35 - protein, 4 aa, Artificial Sequence
  Linker used for the reconstituted RBM
  misc_feature: Synthetic
GEPL

SEQ ID NO: 36 - DNA, 29903 bp, Unknown
  Severe acute respiratory syndrome coronavirus 2
  misc_feature: Severe acute respiratory syndrome coronavirus 2 isolate Wuhan-Hu-1, complete genome NCBI Reference Sequence NC_045512.2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 37 - protein, 13 aa, Artificial Sequence
  First generation anchor loop, residues 457 to 469
  misc_feature: Synthetic
RKSNLKPFERDIS

SEQ ID NO: 38 - protein, 1273 aa, Unknown
  Severe acute respiratory syndrome coronavirus 2
  MISC_FEATURE: Spike protein of SARS-COV2 comprising N501Y substitution
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 39 - protein, 661 aa, Artificial Sequence
  SARS CoV2 Spike-S1, S1 (M1---E661), comprising N501Y substitution
  misc_feature: Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYE

SEQ ID NO: 40 - protein, 59 aa, Artificial Sequence
  SARS CoV2 presumed first generation RBM region 443 to 501 of the Spike protein
  misc_feature: Synthetic
SKVGGNYNYLYRLFRKSNLKPFERDISTEIYQAGSTPCNGVEGFNCYFPLQSYGFQPTN

SEQ ID NO: 41 - protein, 1273 aa, Unknown
  Severe acute respiratory syndrome coronavirus 2
  MISC_FEATURE: Spike protein of SARS-COV2comprising N501Y, K417N, E484K substitutions
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 42 - protein, 4 aa, Artificial Sequence
  Linker used for the reconstituted RBM SARS CoV2
  misc_feature: Synthetic
QLEN

SEQ ID NO: 43 - protein, 4 aa, Artificial Sequence
  Linker used for the reconstituted RBM SARS CoV2
  misc_feature: Synthetic
LLRQ

SEQ ID NO: 44 - protein, 4 aa, Artificial Sequence
  Linker used for the reconstituted RBM SARS CoV2
  misc_feature: Synthetic
LLRK

SEQ ID NO: 45 - protein, 4 aa, Artificial Sequence
  Linker used for the reconstituted RBM SARS CoV2
  misc_feature: Synthetic
LMRQ

SEQ ID NO: 46 - protein, 4 aa, Artificial Sequence
  Linker used for the reconstituted RBM SARS CoV2
  misc_feature: Synthetic
ELEK

SEQ ID NO: 47 - protein, 5 aa, Artificial Sequence
  Linker used for the reconstituted RBM SARS CoV2
  misc_feature: Synthetic
LMRQY

SEQ ID NO: 48 - protein, 5 aa, Artificial Sequence
  Linker used for the reconstituted RBM SARS CoV2
  misc_feature: Synthetic
LLRQY

SEQ ID NO: 49 - protein, 5 aa, Artificial Sequence
  Linker used for the reconstituted RBM SARS CoV2
  misc_feature: Synthetic
LLRAL

SEQ ID NO: 50 - protein, 5 aa, Artificial Sequence
  Linker used for the reconstituted RBM SARS CoV2
  misc_feature: Synthetic
LLRSL

SEQ ID NO: 51 - protein, 5 aa, Artificial Sequence
  Linker used for the reconstituted RBM SARS CoV2
  misc_feature: Synthetic
QLREH

SEQ ID NO: 52 - protein, 5 aa, Artificial Sequence
  Linker used for the reconstituted RBM SARS CoV2
  misc_feature: Synthetic
QLREF

SEQ ID NO: 53 - protein, 22 aa, Artificial Sequence
  Residues 435-456
  misc_feature: Synthetic
AWNSNNLDSKVGGNYNYLYRLF

SEQ ID NO: 54 - protein, 38 aa, Artificial Sequence
  Residues 473-510
  misc_feature: Synthetic
YQAGSTPCNGVEGFNCYFPLQSYGFQPTNGVGYQPYRV

SEQ ID NO: 55 - protein, 42 aa, Artificial Sequence
  Residues 443-456- LINKER- 473-499
  misc_feature: Synthetic
  misc_feature (15)..(15): Xaa can be any naturally occurring amino acid
SKVGGNYNYLYRLFXYQAGSTPCNGVEGFNCYFPLQSYGFQP

SEQ ID NO: 56 - protein, 61 aa, Artificial Sequence
  Residues 435-456- LINKER- 473-510
  misc_feature: Synthetic
  misc_feature (23)..(23): Xaa can be any naturally occurring amino acid
AWNSNNLDSKVGGNYNYLYRLFXYQAGSTPCNGVEGFNCYFPLQSYGFQPTNGVGYQPYRV

SEQ ID NO: 57 - protein, 4 aa, Artificial Sequence
  Linker used for the reconstituted RBM
  misc_feature: Synthetic
  misc_feature (1)..(4): Xaa can be any naturally occurring amino acid
XXXX

SEQ ID NO: 58 - protein, 4 aa, Artificial Sequence
  N′ extension, start at residue 439
  misc_feature: Synthetic
NNLD

SEQ ID NO: 59 - protein, 5 aa, Artificial Sequence
  N′ extension, start at residue 438
  misc_feature: Synthetic
SNNLD

SEQ ID NO: 60 - protein, 6 aa, Artificial Sequence
  N′ extension, start at residue 437
  misc_feature: Synthetic
NSNNLD

SEQ ID NO: 61 - protein, 5 aa, Artificial Sequence
  C′ extension, end at residue 503
  misc_feature: Synthetic
PTNGV

SEQ ID NO: 62 - protein, 11 aa, Artificial Sequence
  C′ extension, end at residue 509
  misc_feature: Synthetic
PTNGVGYQPYR

SEQ ID NO: 63 - protein, 6 aa, Artificial Sequence
  C′ extension, end at residue 504
  misc_feature: Synthetic
PTNGVG

SEQ ID NO: 64 - protein, 5 aa, Artificial Sequence
  C′ extension, end at residue 504
  misc_feature: Synthetic
PTNGG

SEQ ID NO: 65 - protein, 52 aa, Artificial Sequence
  SARS CoV2 reconstituted RBM
  misc_feature: Synthetic
NNLDSKVGGNYNYLYRLFGVPYQAGSTPCNGVEGFNCYFPLQSYGFQPTNGV

SEQ ID NO: 66 - protein, 52 aa, Artificial Sequence
  SARS CoV2 reconstituted RBM with, the Y489N substitution
  misc_feature: Synthetic
NNLDSKVGGNYNYLYRLFGVPYQAGSTPCNGVEGFNCNFPLQSYGFQPTNGV

SEQ ID NO: 67 - protein, 56 aa, Artificial Sequence
  SARS CoV2 reconstituted RBM
  misc_feature: Synthetic
DSKVGGNYNYLYRLFQLENYQAGSTPCNGVEGFNCYFPLQSYGFQPTNGVGYQPYR

SEQ ID NO: 68 - protein, 56 aa, Artificial Sequence
  SARS CoV2 reconstituted RBM with the Q474R substitution
  misc_feature: Synthetic
DSKVGGNYNYLYRLFQLENYRAGSTPCNGVEGFNCYFPLQSYGFQPTNGVGYQPYR

SEQ ID NO: 69 - protein, 51 aa, Artificial Sequence
  SARS CoV2 reconstituted RBM
  misc_feature: Synthetic
DSKVGGNYNYLYRLFLLRQYQAGSTPCNGVEGFNCYFPLQSYGFQPTNGVG

SEQ ID NO: 70 - protein, 50 aa, Artificial Sequence
  SARS CoV2 reconstituted RBM
  misc_feature: Synthetic
SNNLDSKVGGNYNYLYRLFLLRQYQAGSTPCNGVEGFNCYFPLQSYGFQP

SEQ ID NO: 71 - protein, 54 aa, Artificial Sequence
  SARS CoV2 reconstituted RBM
  misc_feature: Synthetic
SNNLDSKVGGNYNYLYRLFLLRQYQAGSTPCNGVEGFNCYFPLQSYGFQPTNGG

SEQ ID NO: 72 - protein, 51 aa, Artificial Sequence
  SARS CoV2 reconstituted RBM
  misc_feature: Synthetic
SNNLDSKVGGNYNYLYRLFLLRKYQAGSTPCNGVEGFNCYFPLQSYGFQPT

SEQ ID NO: 73 - protein, 53 aa, Artificial Sequence
  SARS CoV2 reconstituted RBM
  misc_feature: Synthetic
NSNNLDSKVGGNYNYLYRLFLLRKYQAGSTPCNGVEGFNCYFPLQSYGFQPTN

SEQ ID NO: 74 - protein, 53 aa, Artificial Sequence
  SARS CoV2 reconstituted RBM with the V483E substitution
  misc_feature: Synthetic
NSNNLDSKVGGNYNYLYRLFLLRKYQAGSTPCNGEEGFNCYFPLQSYGFQPTN

SEQ ID NO: 75 - protein, 48 aa, Artificial Sequence
  SARS CoV2 reconstituted RBM
  misc_feature: Synthetic
NLDSKVGGNYNYLYRLFLMRQYQAGSTPCNGVEGFNCYFPLQSYGFQN

SEQ ID NO: 76 - protein, 51 aa, Artificial Sequence
  SARS CoV2 reconstituted RBM
  misc_feature: Synthetic
NSNNLDSKVGGNYNYLYRLFELEKYQAGSTPCNGVEGFNCYFPLQSYGFQP

SEQ ID NO: 77 - protein, 51 aa, Artificial Sequence
  SARS CoV2 reconstituted RBM with the Q474R substitution
  misc_feature: Synthetic
NSNNLDSKVGGNYNYLYRLFELEKYRAGSTPCNGVEGFNCYFPLQSYGFQP

SEQ ID NO: 78 - protein, 53 aa, Artificial Sequence
  SARS CoV2 reconstituted RBM
  misc_feature: Synthetic
NLDSKVGGNYNYLYRLFLMRQYYQAGSTPCNGVEGFNCYFPLQSYGFQPTNGV

SEQ ID NO: 79 - protein, 48 aa, Artificial Sequence
  SARS CoV2 reconstituted RBM
  misc_feature: Synthetic
DSKVGGNYNYLYRLFLLRQYYQAGSTPCNGVEGFNCYFPLQSYGFQPT

SEQ ID NO: 80 - protein, 48 aa, Artificial Sequence
  SARS CoV2 reconstituted RBM with the L492I substitution
  misc_feature: Synthetic
DSKVGGNYNYLYRLFLLRQYYQAGSTPCNGVEGFNCYFPIQSYGFQPT

SEQ ID NO: 81 - protein, 54 aa, Artificial Sequence
  SARS CoV2 reconstituted RBM
  misc_feature: Synthetic
NSNNLDSKVGGNYNYLYRLFLLRALYQAGSTPCNGVEGFNCYFPLQSYGFQPTN

SEQ ID NO: 82 - protein, 50 aa, Artificial Sequence
  SARS CoV2 reconstituted RBM
  misc_feature: Synthetic
NLDSKVGGNYNYLYRLFLLRSLYQAGSTPCNGVEGFNCYFPLQSYGFQPT

SEQ ID NO: 83 - protein, 52 aa, Artificial Sequence
  SARS CoV2 reconstituted RBM
  misc_feature: Synthetic
SNNLDSKVGGNYNYLYRLFQLREHYQAGSTPCNGVEGFNCYFPLQSYGFQPT

SEQ ID NO: 84 - protein, 52 aa, Artificial Sequence
  SARS CoV2 reconstituted RBM
  misc_feature: Synthetic
SNNLDSKVGGNYNYLYRLFQLREFYQAGSTPCNGVEGFNCYFPLQSYGFQPT

SEQ ID NO: 85 - protein, 8 aa, Artificial Sequence
  Residues A435-D442
  misc_feature: Synthetic
AWNSNNLD

SEQ ID NO: 86 - protein, 11 aa, Artificial Sequence
  Residues T500-V510
  misc_feature: Synthetic
TNGVGYQPYRV

SEQ ID NO: 87 - protein, 65 aa, Artificial Sequence
  The two helices of ACE2 (residues 21-85)
  misc_feature: Synthetic
IEEQAKTFLDKFNHEAEDLFYQSSLASWNYNTNITEENVQNMNNAGDKWSAFLKEQSTLAQMYPL

SEQ ID NO: 88 - protein, 35 aa, Artificial Sequence
  ACE2 Helix 1 segment residues 21-55
  misc_feature: Synthetic
IEEQAKTFLDKFNHEAEDLFYQSSLASWNYNTNIT

SEQ ID NO: 89 - protein, 11 aa, Artificial Sequence
  A second loop of ACE2 (residues 350-360) of ACE2
  misc_feature: Synthetic
DLGKGDFRILM

SEQ ID NO: 90 - protein, 32 aa, Artificial Sequence
  Segment B 1st generation residues T470 to N501
  misc_feature: Synthetic
TEIYQAGSTPCNGVEGFNCYFPLQSYGFQPTN

SEQ ID NO: 91 - DNA, 12 bp, Artificial Sequence
  BstX1 site
  misc_feature: Synthetic
  misc_feature (4)..(9): n is a, c, g, or t
ccannnnnntgg

SEQ ID NO: 92 - protein, 61 aa, Artificial Sequence
  The general scheme of the second-generation library
  misc_feature: Synthetic
  misc_feature (25)..(25): Xaa can be any naturally occurring amino acid
FYSHSAPELWSKVGGNYNYLYRLFXYQAGSTPCNGVEGFNCYFPLQSYGFQPDVWETVESC

SEQ ID NO: 93 - protein, 26 aa, Artificial Sequence
  Residues Y473 through Q498
  misc_feature: Synthetic
YQAGSTPCNGVEGFNCYFPLQSYGFQ

SEQ ID NO: 94 - DNA, 96 bp, Artificial Sequence
  Sense oligonucleotides for the construction of the 1st generation RBM, linker #1
  misc_feature: Synthetic
  misc_feature (73)..(74): n is a, c, g, or t
ttctattctcactccgctccagagctctggagcaaagttggtggaaactataattatctgtatcggctgtttnnktatcaagctggttctacaccg

SEQ ID NO: 95 - DNA, 99 bp, Artificial Sequence
  Sense oligonucleotides for the construction of the 1st generation RBM, linker #2
  misc_feature: synthetic
  misc_feature (73)..(74): n is a, c, g, or t
  misc_feature (76)..(77): n is a, c, g, or t
ttctattctcactccgctccagagctctggagcaaagttggtggaaactataattatctgtatcggctgtttnnknnktatcaagctggttctacaccg

SEQ ID NO: 96 - DNA, 102 bp, Artificial Sequence
  Sense oligonucleotides for the construction of the 1st generation RBM, linker #3
  misc_feature: Synthetic
  misc_feature (73)..(74): n is a, c, g, or t
  misc_feature (76)..(77): n is a, c, g, or t
  misc_feature (79)..(80): n is a, c, g, or t
ttctattctcactccgctccagagctctggagcaaagttggtggaaactataattatctgtatcggctgtttnnknnknnktatcaagctggttctacaccg

SEQ ID NO: 97 - DNA, 105 bp, Artificial Sequence
  Sense oligonucleotides for the construction of the 1st generation RBM, linker #4
  misc_feature: Synthetic
  misc_feature (73)..(74): n is a, c, g, or t
  misc_feature (76)..(77): n is a, c, g, or t
  misc_feature (79)..(80): n is a, c, g, or t
  misc_feature (82)..(83): n is a, c, g, or t
ttctattctcactccgctccagagctctggagcaaagttggtggaaactataattatctgtatcggctgtttnnknnknnknnktatcaagctggttctacaccg

SEQ ID NO: 98 - DNA, 108 bp, Artificial Sequence
  Sense oligonucleotides for the construction of the 2nd generation RBM, linker #5
  misc_feature: Synthetic
  misc_feature (73)..(74): n is a, c, g, or t
  misc_feature (76)..(77): n is a, c, g, or t
  misc_feature (79)..(80): n is a, c, g, or t
  misc_feature (82)..(83): n is a, c, g, or t
  misc_feature (85)..(86): n is a, c, g, or t
ttctattctcactccgctccagagctctggagcaaagttggtggaaactataattatctgtatcggctgtttnnknnknnknnknnktatcaagctggttctacaccg

SEQ ID NO: 99 - protein, 20 aa, Artificial Sequence
  Residues P479-Q498
  misc_feature: Synthetic
PCNGVEGFNCYFPLQSYGFQ

SEQ ID NO: 100 - DNA, 108 bp, Artificial Sequence
  3′ BstXI site
  misc_feature: Synthetic
acaactttcaacagtttcccagacgtctggttgaaaaccgtaactttgtaatggaaaataacagttaaatccctcgactccattgcacggtgtagaaccagcttgata

SEQ ID NO: 101 - protein, 56 aa, Artificial Sequence
  RBM residues S443 to Q498
  misc_feature: Synthetic
SKVGGNYNYLYRLFRKSNLKPFERDISTEIYQAGSTPCNGVEGFNCYFPLQSYGFQ

SEQ ID NO: 102 - protein, 12 aa, Artificial Sequence
  RBM residues P499-V510
  misc_feature: Synthetic
PTNGVGYQPYRV

SEQ ID NO: 103 - DNA, 48 bp, Artificial Sequence
  Sense oligonucleotides for the construction of the 3rd generation extended RBM libraries, linker #1, 442F_RBM
  misc_feature: Synthetic
ttctattctcactccgctccagagctctgggatagcaaagttggtgga

SEQ ID NO: 104 - DNA, 51 bp, Artificial Sequence
  Sense oligonucleotides for the construction of the 3rd generation extended RBM libraries, linker #2, 441F_RBM
  misc_feature: Synthetic
ttctattctcactccgctccagagctctggctggatagcaaagttggtgga

SEQ ID NO: 105 - DNA, 54 bp, Artificial Sequence
  Sense oligonucleotides for the construction of the 3rd generation extended RBM libraries, linker #3, 440F_RBM
  misc_feature: Synthetic
ttctattctcactccgctccagagctctggaacctggatagcaaagttggtgga

SEQ ID NO: 106 - DNA, 57 bp, Artificial Sequence
  Sense oligonucleotides for the construction of the 3rd generation extended RBM libraries, linker #4, 439F_RBM
  misc_feature: Synthetic
ttctattctcactccgctccagagctctggaataacctggatagcaaagttggtgga

SEQ ID NO: 107 - DNA, 60 bp, Artificial Sequence
  Sense oligonucleotides for the construction of the 3rd generation extended RBM libraries, linker #5, 438F_RBM
  misc_feature: Synthetic
ttctattctcactccgctccagagctctggagcaataacctggatagcaaagttggtgga

SEQ ID NO: 108 - DNA, 63 bp, Artificial Sequence
  Sense oligonucleotides for the construction of the 3rd generation extended RBM libraries, linker #6, 437F_RBM
  misc_feature: Synthetic
ttctattctcactccgctccagagctctggaatagcaataacctggatagcaaagttggtgga

SEQ ID NO: 109 - DNA, 66 bp, Artificial Sequence
  Sense oligonucleotides for the construction of the 3rd generation extended RBM libraries, linker #7, 436F_RBM
  misc_feature: Synthetic
ttctattctcactccgctccagagctctggtggaatagcaataacctggatagcaaagttggtgga

SEQ ID NO: 110 - DNA, 69 bp, Artificial Sequence
  Sense oligonucleotides for the construction of the 3rd generation extended RBM libraries, linker #8, 435F_RBM
  misc_feature: Synthetic
ttctattctcactccgctccagagctctgggcttggaatagcaataacctggatagcaaagttggtgga

SEQ ID NO: 111 - DNA, 48 bp, Artificial Sequence
  Anti-sense oligonucleotide #1 for the construction of the 3rd generation extended RBM libraries, 500R_RBM
  misc_feature: Synthetic
acaactttcaacagtttcccagacgtctggtgttggttgaaaaccgta

SEQ ID NO: 112 - DNA, 51 bp, Artificial Sequence
  Anti-sense oligonucleotide #2 for the construction of the 3rd generation extended RBM libraries, 501R_RBM
  misc_feature: Synthetic
acaactttcaacagtttcccagacgtctgggtttgttggttgaaaaccgta

SEQ ID NO: 113 - DNA, 54 bp, Artificial Sequence
  Anti-sense oligonucleotide #3 for the construction of the 3rd generation extended RBM libraries, 502R_RBM
  misc_feature: Synthetic
acaactttcaacagtttcccagacgtctggaccgtttgttggttgaaaaccgta

SEQ ID NO: 114 - DNA, 57 bp, Artificial Sequence
  Anti-sense oligonucleotide #4 for the construction of the 3rd generation extended RBM libraries, 503R_RBM
  misc_feature: Synthetic
acaactttcaacagtttcccagacgtctggaacaccgtttgttggttgaaaaccgta

SEQ ID NO: 115 - DNA, 60 bp, Artificial Sequence
  Anti-sense oligonucleotide #5 for the construction of the 3rd generation extended RBM libraries, 504R_RBM
  misc_feature: Synthetic
acaactttcaacagtttcccagacgtctggtccaacaccgtttgttggttgaaaaccgta

SEQ ID NO: 116 - DNA, 63 bp, Artificial Sequence
  Anti-sense oligonucleotide #6 for the construction of the 3rd generation extended RBM libraries, 505R_RBM
  misc_feature: Synthetic
acaactttcaacagtttcccagacgtctggatatccaacaccgtttgttggttgaaaaccgta

SEQ ID NO: 117 - DNA, 66 bp, Artificial Sequence
  Anti-sense oligonucleotide #7 for the construction of the 3rd generation extended RBM libraries, 506R_RBM
  misc_feature: Synthetic
acaactttcaacagtttcccagacgtctggttgatatccaacaccgtttgttggttgaaaaccgta

SEQ ID NO: 118 - DNA, 69 bp, Artificial Sequence
  Anti-sense oligonucleotide #8 for the construction of the 3rd generation extended RBM libraries, 507R_RBM
  misc_feature: Synthetic
acaactttcaacagtttcccagacgtctggtggttgatatccaacaccgtttgttggttgaaaaccgta

SEQ ID NO: 119 - DNA, 72 bp, Artificial Sequence
  Anti-sense oligonucleotide #9 for the construction of the 3rd generation extended RBM libraries, 508R_RBM
  misc_feature: Synthetic
acaactttcaacagtttcccagacgtctggatatggttgatatccaacaccgtttgttggttgaaaaccgta

SEQ ID NO: 120 - DNA, 75 bp, Artificial Sequence
  Anti-sense oligonucleotide #10 for the construction of the 3rd generation extended RBM libraries, 509R_RBM
  misc_feature: Synthetic
acaactttcaacagtttcccagacgtctggccgatatggttgatatccaacaccgtttgttggttgaaaaccgta

SEQ ID NO: 121 - DNA, 78 bp, Artificial Sequence
  Anti-sense oligonucleotide #11 for the construction of the 3rd generation extended RBM libraries, 510R_RBM
  misc_feature: Synthetic
acaactttcaacagtttcccagacgtctgggacccgatatggttgatatccaacaccgtttgttggttgaaaaccgta

SEQ ID NO: 122 - protein, 7 aa, Artificial Sequence
  Fragment B of the RBM, Y473-P479
  misc_feature: Synthetic
YQAGSDP

SEQ ID NO: 123 - DNA, 51 bp, Artificial Sequence
  Anti-sense oligonucleotide #1 for the construction of the 3rd generation extended RBM libraries with the N501Y substitution, Y501R_RBM
  misc_feature: Synthetic
acaactttcaacagtttcccagacgtctgggtatgttggttgaaaaccgta

SEQ ID NO: 124 - DNA, 54 bp, Artificial Sequence
  Anti-sense oligonucleotide #2 for the construction of the 3rd generation extended RBM libraries, with the N501Y substitution, Y502R_RBM
  misc_feature: Synthetic
acaactttcaacagtttcccagacgtctggaccgtatgttggttgaaaaccgta

SEQ ID NO: 125 - DNA, 57 bp, Artificial Sequence
  Anti-sense oligonucleotide #3 for the construction of the 3rd generation extended RBM libraries, with the N501Y substitution, Y503R_RBM
  misc_feature: Synthetic
acaactttcaacagtttcccagacgtctggaacaccgtatgttggttgaaaaccgta

SEQ ID NO: 126 - DNA, 60 bp, Artificial Sequence
  Anti-sense oligonucleotide #4 for the construction of the 3rd generation extended RBM libraries, with the N501Y substitution, Y504R_RBM
  misc_feature: Synthetic
acaactttcaacagtttcccagacgtctggtccaacaccgtatgttggttgaaaaccgta

SEQ ID NO: 127 - DNA, 63 bp, Artificial Sequence
  Anti-sense oligonucleotide #5 for the construction of the 3rd generation extended RBM libraries, with the N501Y substitution, Y505R_RBM
  misc_feature: Synthetic
acaactttcaacagtttcccagacgtctggatatccaacaccgtatgttggttgaaaaccgta

SEQ ID NO: 128 - DNA, 66 bp, Artificial Sequence
  Anti-sense oligonucleotide #6 for the construction of the 3rd generation extended RBM libraries, with the N501Y substitution, Y506R_RBM
  misc_feature: Synthetic
acaactttcaacagtttcccagacgtctggttgatatccaacaccgtatgttggttgaaaaccgta

SEQ ID NO: 129 - DNA, 69 bp, Artificial Sequence
  Anti-sense oligonucleotide #7 for the construction of the 3rd generation extended RBM libraries, with the N501Y substitution, Y507R_RBM
  misc_feature: Synthetic
acaactttcaacagtttcccagacgtctggtggttgatatccaacaccgtatgttggttgaaaaccgta

SEQ ID NO: 130 - DNA, 72 bp, Artificial Sequence
  Anti-sense oligonucleotide #8 for the construction of the 3rd generation extended RBM libraries, with the N501Y substitution, Y508R_RBM
  misc_feature: Synthetic
acaactttcaacagtttcccagacgtctggatatggttgatatccaacaccgtatgttggttgaaaaccgta

SEQ ID NO: 131 - DNA, 75 bp, Artificial Sequence
  Anti-sense oligonucleotide #9 for the construction of the 3rd generation extended RBM libraries, with the N501Y substitution, Y509R_RBM
  misc_feature: Synthetic
acaactttcaacagtttcccagacgtctggccgatatggttgatatccaacaccgtatgttggttgaaaaccgta

SEQ ID NO: 132 - DNA, 78 bp, Artificial Sequence
  Anti-sense oligonucleotide #10 for the construction of the 3rd generation extended RBM libraries, with the N501Y substitution, Y510R_RBM
  misc_feature: Synthetic
acaactttcaacagtttcccagacgtctgggacccgatatggttgatatccaacaccgtatgttggttgaaaaccgta

SEQ ID NO: 133 - protein, 81 aa, Artificial Sequence
  The general scheme of the second-generation library
  misc_feature: Synthetic
  misc_feature (33)..(33): Xaa can be any naturally occurring amino acid
FYSHSAPELWAWNSNNLDSKVGGNYNYLYRLFXYWAGSTPCNGVEGFNCYFPLQSYGFQPTNGVGYQPYRVPDVWETVESC

(19) United States
(12) Patent Application Publication
(10) Pub. No.: US 2023/0321218 A1
(43) Pub. Date: Oct. 12, 2023

(54) SARS-COV2 CORONAVIRUS RECONSTITUTED RBM AND USES THEREOF

(71) Applicant: RAMOT AT TEL-AVIV UNIVERSITY LTD., Tel Aviv (IL)

(72) Inventor: Jonathan M. GERSHONI, Tel Avivil (IL)

(21) Appl. No.: 17/995,733

(22) PCT Filed: Apr. 7, 2021
(86) PCT No.: PCT/IL2021/050402

§ 371 Date: Oct. 7, 2022

Related U.S. Application Data
(60) Provisional application No. 63/006,362, filed on Apr. 7, 2020.

(51) Int. Cl. A61K 39/215; A61P 37/04

(52) U.S. Cl. CPC A61K 39/215; A61P 37/04; A61K 2039/70

(57) ABSTRACT

The present invention relates to vaccines and therapeutic compositions targeted at coronaviruses, specifically, the Severe Acute Respiratory Syndrome coronavirus 2 (SARS CoV2). More specifically, the invention provides reconstituted Receptor Binding Motif (RBM) of a Spike protein of the SARS CoV2, Receptor Binding domains (RBDs), and spike proteins comprising the reconstituted RBMs or τ-linkers, compositions, vaccines, therapeutic and diagnostic methods and uses thereof.

The Sequence Listing in ASCII text file format of 186,958 bytes in size, created on Mar. 15, 2023, with the file name “2023-03-16SequenceListing_GERSHONI15,” filed in the U.S. Patent and Trademark Office on even date herewith, is hereby incorporated herein by reference.

### FIELD OF THE INVENTION

The present invention relates to vaccines and therapeutic compositions targeted at coronaviruses, specifically, the Severe Acute Respiratory Syndrome coronavirus 2 (SARS CoV2). More specifically, the invention provides reconstituted a Receptor Binding Motif (RBM) of a Spike protein of the SARS CoV2, compositions, vaccines, therapeutic and diagnostic methods and uses thereof.

### BACKGROUND ART

References considered to be relevant as background to the presently disclosed subject matter are listed below:
[1] M. Letko, V. Munster, Functional assessment of cell entry and receptor usage for lineage B (3-coronaviruses, including 2019-nCoV. (2020).
[2] N. T. Freund, A. Roitburd-Berman, J. Sui, W. A. Marasco, J. M. Gershoni; Reconstitution of the receptor-binding motif of the SARS coronavirus. PEDS 28, 567-575 (2015); published online EpubDec (10.1093/protein/gzv052).
[3] WO2017/046801.
[4] US 20180334480.
[5] V. S. Raj, et al; Dipeptidyl peptidase 4 is a functional receptor for the emerging human coronavirus-EMC. Nature 495, 251-254 (2013); published online EpubMar 14 (10.1038/nature12005).
[6] W. Li, et al., Angiotensin-converting enzyme 2 is a functional receptor for the SARS coronavirus. Nature 426, 450-454 (2003).
[7] Wrapp D, et al. (February 2020). “Cryo-EM structure of the 2019-nCoV spike in the prefusion conformation”. Science. 367 (6483): 1260-1263.
[8] Hoffman M, Kliene-Weber H, Krüger N, et al. (16 Apr. 2020). “SARS-CoV-2 Cell Entry Depends on ACE2 and TMPRSS2 and Is Blocked by a Clinically Proven Protease Inhibitor”. Cell. 181: 1-10.
[9] Anthony R. Fehr and Stanley Perlman (2015) Coronaviruses: An Overview of Their Replication and Pathogenesis; Methods Mol Biol.; 1282: 1-23.
[10] R. Yan et al., (2020) Structural basis for the recognition of the SARS-CoV-2 by full-length human ACE2; Science 10.1126/science.abb2762 (2020).
[11] Marcandalli et al., 2019, Cell 177, 1420-1431.
[12] Furong Qi, et al., 2020, Biochemical and Biophysical Research Communications, ISSN 0006-291X.
[13] Wang K et al. 2020, bioRxiv 2020.03.14.988345.

Acknowledgement of the above references herein is not to be inferred as meaning that these are in any way relevant to the patentability of the presently disclosed subject matter.

### BACKGROUND OF THE INVENTION

Fifty years ago, the first two human coronaviruses were reported (HCoV-229E and HCoV-OC43). Subsequently, in 2002, Severe Acute Respiratory Syndrome coronavirus (SARS-CoV) emerged. Since then another four coronaviruses have appeared in humans. HCoV-NL63 and HCoV-HKU1, isolated in 2004-2005 [9] Middle East Respiratory Syndrome coronavirus (MERS-CoV) in 2012, and a now the most recent member of this family, a coronavirus from Wuhan, China designated SARS-CoV-2 (2019-nCoV). SARS CoV and MERS CoV proved to be extremely fatal compared to the previous virus strains. The recent outbreak, SARS CoV2 appears to be a genuine global viral threat for which a prophylactic vaccinea are urgently needed along with targeted therapeutics and effective diagnostics. The production of such reagents is the subject of this invention. On Nov. 16, 2002 a 64 year old man from the Guangdong province in China died from an unknown respiratory disease. In the fullness of time he would be known as “Patient Zero” of the SARS epidemic. The emerging threat was reported after some 3 months to the WHO office in Beijing (10 Feb. 2003), while thousands of people in the province had already been infected and hundreds had died. Following the first case reported in Toronto (Feb. 23, 2003) the WHO called a Global Alert. Severe Acute Respiratory Syndrome (SARS) is caused by what had been a previously unknown coronavirus. The natural reservoir of the virus is bats; however, an intermediate vector, masked civets, was first identified. These predators populated the food markets frequently and so in a culling action, 10,000 civets were destroyed. By July 2003, 8,096 cases of SARS were reported in 37 countries with a fatality rate of 9.6%. No new cases have been reported since 2004. In 2012, another new coronavirus emerged in the Saudi peninsula, MERS-CoV (Middle East Respiratory Syndrome coronavirus). Similar to the SARS epidemic, these coronaviruses apparently transfer from bats to an intermediate vector, this time camels, and from camels to humans. The latest updates from WHO on the MERS threat are that 2,499 cases in 27 countries have been reported with 861 deaths (fatality rate of 34.4%). A serious focused-outbreak occurred in 2015 in South Korea where 186 cases were detected leading to 38 deaths. Over 16,950 close contacts were quarantined for two weeks and since then the epidemic seems to be relatively well controlled although there are still sporadic small outbreaks. Recently, in December 2019, there has been another emergence of a new coronavirus in the city of Wuhan, China. The fish market where the virus was first detected was shut down and disinfected immediately, the virus has been isolated and its genome made available quickly. Thus far, the virus continues to spread. As of April 2021 a total number of SARS CoV2 infections worldwide exceeds 130 million and more than 2,885,931 have died of COVID 19, the respiratory disease caused by this coronavirus. The new virus is highly homologous to SARS CoV of 2002. These three coronaviruses target the human respiratory track by associating with cell-surface proteins they exploit as receptors. Angiotensin converting enzyme 2 (ACE2) serves as the SARS-CoV receptor, and human dipeptidyl peptidase 4 (huDPP4) is used by the MERS-CoV [5]. The receptor for the Wuhan coronavirus SARS-CoV2 (2019-nCoV) has been shown to be ACE2 as well [1]. In an effort to understand the mechanisms of infection and provide insights for an epitope-based vaccine design, a novel methodology was developed by the present inventor for the functional reconstitution of the Receptor Binding Motif (RBM) of the viral spike that specifically binds its cognate receptor [2-4]. Although the receptors for each virus are very different, SARS-CoV, MERS-CoV and SARS-CoV2 share common structural motifs that may allow a universal strategy for the reconstitution of functional RBMs. There is a need for the reconstitution of the SARS-CoV2 RBM for the development of epitope-based vaccines and the production of new immuno-therapeutics.

### SUMMARY OF THE INVENTION

In a first aspect, the invention relates to a polypeptide comprising an amino acid sequence of at least one reconstituted Receptor Binding Motif (RBM) of a Spike protein of the Severe Acute Respiratory Syndrome coronavirus 2 (SARS CoV2) or of any fragment thereof. In some embodiments, the reconstituted RBM may comprise at least one linker and at least one fragment of the native RBM. More specifically, the native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 spike protein. Still further, the native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein. In more specific embodiments, the at least one linker/s replaces the anchor loop or any part, or amino acid residue/s thereof and any RBM fragment or amino acid residue/s thereof. In some embodiments the linker of the reconstituted RBM of the invention, or any polypeptide thereof is referred to herein as a τ-linker (tau linker). Thus, in some embodiments, the reconstituted RBM of the invention or any polypeptide thereof is referred to herein as the τ-modified (tau modified) RBM or polypeptide of the invention.

In a further aspect thereof, the invention provides an RBD of SARS CoV2 comprising the native RBD of the spike protein of SARS CoV2 or any fragments thereof and at least one linker that replaces an anchor loop or any part thereof or any amino acid residue/s thereof. More specifically, such anchor loop may comprise an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. In some embodiments, the linker is a bridging linker, specifically, a liker designated as the τ-linker. Thus, in some embodiments, the RBDs provided by the invention may be referred to herein as the i-modified RBDs of the invention. A further aspect of the invention relates to a Spike protein of SARS CoV2, comprising the native Spike protein of SARS CoV2 or any fragment thereof and at least one linker that replaces the anchor loop as specified by the invention. In some embodiments, these spike proteins of the invention may be referred to herein as the τ-modified spike proteins of the invention.

A further aspect of the invention relates to a multimeric and/or multivalent antigen displaying platform comprising at least one reconstituted RBM of a Spike protein of SARS CoV2 or of any fragment thereof, or any fusion protein or conjugate thereof, any polypeptide, RBD or Spike protein comprising the reconstituted RBM, any RBD or Spike protein comprising at least one linker that replaces an anchor loop or any part thereof or amino acid residue/s thereof, specifically, any of the τ-modified polypeptides of the invention and any combinations thereof.

A further aspect of the invention relates to a nucleic acid sequence encoding any of the reconstituted RBMs, RBD or Spike protein comprising the reconstituted RBM, any RBD or Spike protein comprising at least one linker that replaces an anchor loop or any part thereof, or amino acid residue/s thereof, and any multimeric and/or multivalent antigen displaying platform thereof.

A further aspect of the invention relates to a composition comprising an effective amount of at least one polypeptide comprising an amino acid sequence of at least one polypeptide, RBD or Spike protein comprising at least one reconstituted RBM as defined by the invention, at least one RBD or spike protein comprising at least one linker as defined by the invention, at least one multimeric and/or multivalent antigen displaying platform as defined by the invention and at least one nucleic acid sequence as defined by the invention. In some embodiments, the composition optionally further comprises at least one pharmaceutically acceptable carrier/s, adjuvant/s, excipient/s, auxiliaries, and/or diluent/s.

A further aspect of the invention relates to a SARS CoV2 vaccine comprising at least one polypeptide comprising an amino acid sequence of at least one polypeptide, RBD or Spike protein comprising at least one reconstituted RBM as defined by the invention, at least one RBD or spike protein comprising at least one linker as defined by the invention, at least one multimeric and/or multivalent antigen displaying platform as defined by the invention, at least one nucleic acid sequence as defined by the invention and at least one composition as defined by the invention. In some embodiments, the vaccine optionally further comprises at least one pharmaceutically acceptable carrier/s, adjuvant/s, excipient/s, auxiliaries, and/or diluent/s.

In yet a further aspect, the invention relates to a method for treating, preventing, inhibiting, reducing, eliminating, protecting or delaying the onset of an infection or an infectious clinical condition caused by SARS CoV2 in a subject in need thereof, by administering to the subject an effective amount of at least one polypeptide, RBD or Spike protein comprising at least one reconstituted RBM as defined by the invention, at least one RBD or spike protein comprising at least one linker as defined by the invention, at least one multimeric and/or multivalent antigen displaying platform as defined by the invention, at least one nucleic acid sequence as defined by the invention, at least one composition, and at least one vaccine as defined by the invention.

The invention further provides at least one polypeptide, RBD or Spike protein comprising at least one reconstituted RBM as defined by the invention, at least one RBD or spike protein comprising at least one linker as defined by the invention, at least one multimeric and/or multivalent antigen displaying platform as defined by the invention, at least one nucleic acid sequence as defined by the invention, at least one composition as defined by the invention, and at least one vaccine as defined by the invention, for use in a method for treating, preventing, inhibiting, reducing, eliminating, protecting or delaying the onset of an infection or an infectious clinical condition caused by SARS CoV2 in a subject in need thereof.

A further aspect of the invention relates to a method of inducing an immune response against a SARS CoV2 in a subject in need thereof. More specifically, the method comprising the step of administering to the subject an immunogenic effective amount of at least one polypeptide, RBD or Spike protein comprising at least one reconstituted RBM as defined by the invention, at least one RBD or Spike protein comprising at least one linker as defined by the invention, at least one multimeric and/or multivalent antigen displaying platform as defined by the invention, at least one nucleic acid sequence as defined by the invention, at least one composition as defined by the invention, and at least one vaccine as defined by the invention.

The invention further provides at least one polypeptide, RBD or Spike protein comprising at least one reconstituted RBM as defined by the invention, at least one RBD or Spike protein comprising at least one linker as defined by the invention, at least one multimeric and/or multivalent antigen displaying platform as defined by the invention, at least one nucleic acid sequence as defined by the invention, at least one composition as defined by the invention, and at least one vaccine as defined by the invention, for use in a method of inducing an immune response against a SARS CoV2 in a subject in need thereof.

A further aspect of the invention relates to a method for the preparation of a functional reconstituted RBM of a Spike protein of SARS CoV2, the method comprising the step of:

First (a), screening a conformer library of RBMs of the viral Spike protein with at least one binding molecule. Specifically, the library comprising plurality of combinatorial display platforms or any display vehicles, e.g., bacteriophages, or any other vehicles for any combinatorial display systems such as yeast display, ribosome-display, or peptide display, each expressing a reconstituted RBM comprising an amino acid sequence of at least one fragment of a native RBM of a Spike protein of the SARS CoV2 and at least one combinatorial linker. The second step of the method of the invention (b), involves identifying and producing reconstituted RBM peptides which bind at least one of the binding molecules.

In yet another aspect, the invention provides a method for producing SARS CoV2 vaccine comprising reconstituted RBM, the method comprising the steps of:

First in step (a), preparing reconstituted functional RBM/s of a Spike protein of SARS CoV2 by a method as defined by the invention and discussed herein above.

The next step (b), involves admixing at least one of the reconstituted functional RBM/s of a Spike protein of SARS CoV2 or any derivative or enantiomer thereof, or any fusion protein, conjugate, or polyvalent dendrimer comprising the same with at least one adjuvant/s, carrier/s, excipient/s, auxiliaries, and/or diluent/s.

A further aspect of the invention relates to a method for the preparation, affinity selection and/or isolation of neutralizing antibodies that neutralize the SARS CoV2 and compete with receptor binding, the method comprising the steps of: (a), contacting a serum or lymphocytes of at least one donor with an effective amount of at least one reconstituted RBM, or any polypeptide, RBDs and/or Spike proteins comprising the reconstituted RBM, any RBDs and/or spike proteins discussed above, any multimeric and/or multivalent antigen displaying platform any derivative, enantiomer, fusion protein, conjugate, polyvalent dendrimer thereof, as defined by the invention, associated or attached directly or indirectly to a solid support and/or a detectable moiety. (b), recovering the antibodies or at least one lymphocyte bound to the reconstituted RBM, or to any RBDs and/or Spike proteins comprising the reconstituted RBM, to any RBDs and/or spike proteins discussed above, any multimeric and/or multivalent antigen displaying platform any derivative, enantiomer, fusion protein, conjugate, polyvalent dendrimer thereof, used in step (a).

A further aspect of the invention relates to a method for the preparation of neutralizing antibodies directed at the Spike protein of SARS CoV2, the method comprising the step of: (a), contacting at least one lymphocyte or serum of an immunized non-human animal, with an effective amount of at least one reconstituted RBM, or any polypeptide, RBDs and/or Spike proteins comprising the reconstituted RBM, any RBDs and/or spike proteins discussed above, any multimeric and/or multivalent antigen displaying platform any derivative, enantiomer, fusion protein, conjugate, polyvalent dendrimer thereof, as defined by the invention, associated directly or indirectly to a solid support and/or a detectable moiety. (b), recovering the antibodies or lymphocyte/s bound to the at least one reconstituted RBM, or to any RBDs and/or Spike proteins comprising the reconstituted RBM, to any RBDs and/or spike proteins discussed above, any multimeric and/or multivalent antigen displaying platform any derivative, enantiomer, fusion protein, conjugate, polyvalent dendrimer thereof, immobilized to the solid support and/or detectable moiety, used in step (a) thereby obtaining a passive vaccine comprising neutralizing antibodies that neutralize SARS CoV2. It should be noted that the non-human animal is immunized with an effective amount of the reconstituted RBM, or any polypeptide, RBDs and/or Spike proteins comprising the reconstituted RBM, any RBDs and/or spike proteins discussed above, any multimeric and/or multivalent antigen displaying platform any derivative, enantiomer, fusion protein, conjugate, polyvalent dendrimer thereof, as defined by the invention, or any combinations thereof, any derivative, enantiomer, fusion protein, conjugate, polyvalent dendrimer thereof, or any SARS CoV2 virus comprising at least one linker that replaces the anchor loop of the Spike protein. In yet some further embodiments, the non-human animal may be immunized with at least one attenuated or killed SARS CoV2 virus or any variant or mutant thereof, and any composition or vaccine thereof.

In yet a further aspect thereof, the invention provides a therapeutic passive vaccine comprising neutralizing antibodies that neutralize the SARS CoV2. In more specific embodiments, such vaccine is prepared by the methods of the invention as described herein.

A further aspect of the invention relates to a method of screening for a compound that inhibits binding of at least one Spike protein of a SARS CoV2 virus to the cognate receptor in a target cell, the method comprising the step of: (a), contacting at least one candidate compound or a plurality of candidate compounds with an effective amount of at least one reconstituted RBM, or any polypeptide, RBDs and/or Spike proteins comprising the reconstituted RBM, any RBDs and/or spike proteins discussed above, any multimeric and/or multivalent antigen displaying platform any derivative, enantiomer, fusion protein, conjugate, polyvalent dendrimer thereof, as defined by the, associated directly or indirectly to a solid support and/or a detectable moiety.
(b) recovering the candidate compound bound to the reconstituted RBM immobilized to the solid support and/or detectable moiety, or any to any RBDs and/or Spike proteins comprising the reconstituted RBM, to any RBDs and/or spike proteins discussed above, any multimeric and/or multivalent antigen displaying platform any derivative, enantiomer, fusion protein, conjugate, polyvalent dendrimer thereof, used in step (a), thereby obtaining a compound that binds the Spike protein of a SARS CoV2 and inhibits binding of the virus to the cognate receptor.

In yet a further aspect thereof, the invention provides a therapeutic passive vaccine comprising neutralizing antibodies that neutralize the SARS CoV2. In more specific embodiments, such vaccine is prepared by the methods of the invention as described herein.

A further aspect of the invention relates to a method of screening for a compound that inhibits binding of at least one Spike protein of a SARS CoV2 virus to the cognate receptor in a target cell.

In some embodiments, the method comprising the step of:

First in step (a), contacting at least one candidate compound or a plurality of candidate compounds with an effective amount of at least one reconstituted RBM, or any polypeptide, RBDs and/or Spike proteins comprising the reconstituted RBM, any RBDs and/or spike proteins discussed above, any multimeric and/or multivalent antigen displaying platform any derivative, enantiomer, fusion protein, conjugate, polyvalent dendrimer thereof, as defined by the invention, associated directly or indirectly to a solid support and/or a detectable moiety. The next step (b) involves recovering the candidate compound bound to the reconstituted RBM immobilized to the solid support and/or detectable moiety, or any to any RBDs and/or Spike proteins comprising the reconstituted RBM, to any RBDs and/or spike proteins discussed above, any multimeric and/or multivalent antigen displaying platform any derivative, enantiomer, fusion protein, conjugate, polyvalent dendrimer thereof, used in step (a), thereby obtaining a compound that binds the Spike protein of a SARS CoV2 and inhibits binding of the virus to the cognate receptor.

A further aspect of the invention relates to a compound that inhibits binding of at least one Spike protein of a SARS CoV2 virus to the cognate receptor in a target cell. In some embodiments, the compound is prepared by any of the methods as defined by the invention.

Another aspect of the invention relates to a method for treating, inhibiting, reducing, eliminating, protecting or delaying the onset of COVID-19 in a subject in need thereof, by administering to the subject an effective amount of a therapeutic passive vaccine comprising neutralizing antibodies that neutralize the SARS CoV2, or of a compound that inhibits binding of at least one Spike protein of a SARS CoV2 virus to the cognate receptor in a target cell.

A further aspect of the invention relates to an effective amount of the therapeutic passive vaccine comprising neutralizing antibodies that neutralize the SARS CoV2, or of a compound that inhibits binding of at least one Spike protein of a SARS CoV2 virus to the cognate receptor in a target cell, for use in a method for treating, preventing, inhibiting, reducing, eliminating, protecting or delaying the onset of COVID-19 in a subject in need thereof.

The invention provides in a further aspect thereof, an antibody that specifically recognizes and binds at least one polypeptide comprising an amino acid sequence of at least one reconstituted RBM of a viral Spike protein or of any fragment thereof, any RBD or Spike protein comprising the reconstituted RBM, any RBDs and/or spike proteins discussed above, any multimeric and/or multivalent antigen displaying platform any derivative, enantiomer, fusion protein, conjugate, polyvalent dendrimer thereof, as defined by the invention.

A further aspect of the invention provide a diagnostic kit comprising at least one of: (a), at least one reconstituted RBM, or any polypeptide, RBDs and/or Spike proteins comprising the reconstituted RBM, any RBDs and/or spike proteins discussed above, any multimeric and/or multivalent antigen displaying platform any derivative, enantiomer, fusion protein, conjugate, polyvalent dendrimer thereof, as defined by the invention and any combinations thereof, associated directly or indirectly to a solid support and/or a detectable moiety. The kit of the invention may alternatively or additionally comprise (b), antibodies specific for said reconstituted RBM associated directly or indirectly to a solid support and/or a detectable moiety. Still further, in some embodiments, the kit of the invention may comprise (c) at least one reconstituted RBM binding molecule associated directly or indirectly to a solid support and/or a detectable moiety.

The invention further provides in an additional aspect thereof, an RBD of a Corona virus (CoV) comprising the native RBD of the spike protein of at least one CoV or any fragments thereof and at least one linker, wherein at least one of said linker/s replaces an anchor loop of said Spike protein or any part thereof or amino acid residue/s thereof, the anchor loop comprises between 5 to 20 amino acid residues and forms 2 to 5 hydrogen bonds to the core of the RBD. The amino acid sequence of the loop starts approximately 10 amino acid residues before the first hydrogen bond and ends approximately 10 amino acid residues after the last hydrogen bond to the core of the RBD, said core comprise about five beta strands, wherein at least one of said linker is a bridging linker.

A further aspect of the invention relates to a Spike protein of a CoV, comprising the native Spike protein of at least one CoV or any fragment thereof and at least one linker. At least one of the linker/s replaces an anchor loop of the Spike protein or any part or amino acid residue/s thereof. A further aspect of the invention relates to a multimeric and/or multivalent antigen displaying platform comprising at least one RBD or Spike protein of at least one CoV comprising at least one linker that replaces an anchor loop of said Spike protein or any part thereof or amino acid residue/s thereof, or any fusion protein or conjugate thereof.

Another aspect of the invention relates to a nucleic acid sequence encoding at least one RBD or at least one Spike protein of at least one CoV comprising at least one linker that replaces an anchor loop of said Spike protein or any part thereof or amino acid residue/s thereof, or any fusion protein thereof, multimeric and/or multivalent antigen displaying platform thereof or any combinations thereof.

In yet a further aspect, the invention relates to a composition comprising an effective amount of at least one RBD or at least one Spike protein of at least one CoV comprising at least one linker that replaces an anchor loop of said Spike protein or any part thereof or amino acid residue/s thereof, or any fusion protein, multimeric and/or multivalent antigen displaying platform thereof, any derivative, enantiomer, fusion protein, conjugate, polyvalent dendrimer thereof, any nucleic acid sequence encoding the same, and any combinations thereof. In some embodiments, the composition optionally further comprises at least one pharmaceutically acceptable carrier/s, adjuvant/s, excipient/s, auxiliaries, and/or diluent/s.

A further aspect of the invention relates to a CoV vaccine comprising at least east one RBD or at least one Spike protein of at least one CoV comprising at least one linker that replaces an anchor loop of said Spike protein or any part thereof, or amino acid residue/s thereof, or any fusion protein, multimeric and/or multivalent antigen displaying platform thereof, any derivative, enantiomer, fusion protein, conjugate, polyvalent dendrimer thereof, any nucleic acid sequence encoding the same, and any combinations thereof. In some embodiments the vaccine optionally further comprises at least one pharmaceutically acceptable carrier/s, excipient/s, adjuvant/s, auxiliaries, and/or diluent/s.

A further aspect of the invention relates to a method for treating, preventing, inhibiting, reducing, eliminating, protecting or delaying the onset of an infection or an infectious clinical condition caused by at least one CoV in a subject in need thereof. The method comprising the step of administering to the subject an effective amount of at least one RBD or at least one Spike protein of at least one CoV comprising at least one linker that replaces an anchor loop of said Spike protein or any part thereof, or amino acid residue/s thereof, or any fusion protein, multimeric and/or multivalent antigen displaying platform thereof, any derivative, enantiomer, fusion protein, conjugate, polyvalent dendrimer thereof, any nucleic acid sequence encoding the same, and any combinations thereof, or any composition or vaccine thereof.

In yet a further aspect, the invention relates to a composition comprising an effective amount of at least one RBD or at least one Spike protein of at least one CoV comprising at least one linker that replaces an anchor loop of said Spike protein or any part thereof, or amino acid residue/s thereof, or any fusion protein, multimeric and/or multivalent antigen displaying platform thereof, any derivative, enantiomer, fusion protein, conjugate, polyvalent dendrimer thereof, any nucleic acid sequence encoding the same, and any combinations thereof. In some embodiments, the composition optionally further comprises at least one pharmaceutically acceptable carrier/s, adjuvant/s, excipient/s, auxiliaries, and/or diluent/s.

A further aspect of the invention relates to a CoV vaccine comprising at least east one RBD or at least one Spike protein of at least one CoV comprising at least one linker that replaces an anchor loop of said Spike protein or any part thereof, or amino acid residue/s thereof, or any fusion protein, multimeric and/or multivalent antigen displaying platform thereof, any derivative, enantiomer, fusion protein, conjugate, polyvalent dendrimer thereof, any nucleic acid sequence encoding the same, and any combinations thereof. In some embodiments the vaccine optionally further comprises at least one pharmaceutically acceptable carrier/s, excipient/s, adjuvant/s, auxiliaries, and/or diluent/s.

A further aspect of the invention relates to a method for treating, preventing, inhibiting, reducing, eliminating, protecting or delaying the onset of an infection or an infectious clinical condition caused by at least one CoV in a subject in need thereof. The method comprising the step of administering to the subject an effective amount of at least one RBD or at least one Spike protein of at least one CoV comprising at least one linker that replaces an anchor loop of said Spike protein or any part thereof, or amino acid residue/s thereof, or any fusion protein, multimeric and/or multivalent antigen displaying platform thereof, any derivative, enantiomer, fusion protein, conjugate, polyvalent dendrimer thereof, any nucleic acid sequence encoding the same, and any combinations thereof, or any composition or vaccine thereof.

Another aspect of the invention relates to a method of inducing an immune response against at least one CoV in a subject in need thereof, the method comprising administering to the subject an immunogenic effective amount of at least one RBD or at least one Spike protein of at least one CoV comprising at least one linker that replaces an anchor loop of said Spike protein or any part thereof, or amino acid residue/s thereof, or any fusion protein, multimeric and/or multivalent antigen displaying platform thereof, any derivative, enantiomer, fusion protein, conjugate, polyvalent dendrimer thereof, any nucleic acid sequence encoding the same, and any combinations thereof, or any composition or vaccine thereof.

In yet another aspect, the invention provides a method for the preparation, affinity selection and/or isolation of neutralizing antibodies that neutralize at least one CoV and compete with receptor binding of said CoV, the method comprising the steps of:
(a) contacting a serum or lymphocytes of at least one donor with an effective amount of at least one RBD or at least one Spike protein of said CoV, comprising at least one linker that replaces an anchor loop of said Spike protein or any part thereof, or amino acid residue/s thereof, any multimeric and/or multivalent antigen displaying platform thereof, any derivative, enantiomer, fusion protein, conjugate, polyvalent dendrimer thereof, and any combinations thereof. (b), recovering the antibodies or at least one lymphocyte bound to the at least one RBD or at least one Spike protein of said CoV.

A further aspect of the invention relates to a therapeutic passive vaccine comprising neutralizing antibodies that neutralize at least one CoV. In some embodiments, the vaccine is prepared by the method according the invention.

Another aspect of the invention relates to a method for the preparation of neutralizing antibodies directed at the Spike protein of at least one CoV, the method comprising the step of: (a), contacting at least one lymphocyte or serum of at least one immunized non-human animal, with an effective amount of at least one RBD or at least one Spike protein of the CoV comprising at least one linker that replaces an anchor loop of the Spike protein or any part thereof, or amino acid residue/s thereof, any multimeric and/or multivalent antigen displaying platform thereof, any derivative, enantiomer, fusion protein, conjugate, polyvalent dendrimer thereof, or any CoV comprising at least one linker that replaces the anchor loop of the Spike protein, or any combinations thereof. (b), recovering the antibodies or lymphocytes bound to said at least one RBD or at least one Spike protein; thereby obtaining neutralizing antibodies that neutralize said CoV. The non-human animal is immunized with an effective amount of said at least one RBD or Spike protein comprising at least one linker replacing the anchor loop or any part thereof, or amino acid residue/s thereof, any multimeric and/or multivalent antigen displaying platform thereof, any nucleic acid sequence encoding the same, any combinations thereof, any derivative, enantiomer, fusion protein, conjugate, polyvalent dendrimer thereof, or any CoV comprising at least one linker that replaces the anchor loop of the Spike protein, at least one attenuated or killed CoV or any variant or mutant thereof, and any composition or vaccine thereof.

In yet a further aspect, the invention relates to a therapeutic passive vaccine comprising neutralizing antibodies that neutralize at least one CoV. The vaccine is prepared by the method according the invention. A further aspect of the invention relates to a method of screening for a compound that inhibits binding of at least one Spike protein of at least one CoV to the cognate receptor in a target cell, the method comprising the steps of: (a), contacting at least one candidate compound or a plurality of candidate compounds with an effective amount of at least one RBD or Spike protein of the CoV comprising at least one linker that replaces an anchor loop or any part thereof, or amino acid residue/s thereof, or any multimeric and/or multivalent antigen displaying platform thereof, and any combinations thereof, any derivative, enantiomer, fusion protein, conjugate, polyvalent dendrimer thereof, associated directly or indirectly to a solid support and/or a detectable moiety. (b), recovering the candidate compound bound to the at least one RBD or Spike protein immobilized to said solid support and/or a detectable moiety, thereby obtaining a compound that binds the Spike protein of said CoV and inhibits binding of the virus to said cognate receptor. A further aspect of the invention relates to a compound that inhibits binding of at least one Spike protein of at least one CoV to the cognate receptor in a target cell. In some embodiments, the compound is prepared by the method as defined by the invention.

Another aspect of the invention relates to a method for treating, inhibiting, reducing, eliminating, protecting or delaying the onset of at least one pathological condition caused by and/or associated with at least one CoV infection in a subject in need thereof by administering to the subject an effective amount of a therapeutic passive vaccine comprising neutralizing antibodies that neutralize said CoV, or of a compound that inhibits binding of at least one Spike protein of said CoV to the cognate receptor in a target cell. In some embodiments, the passive vaccine is as defined by the invention, and wherein the compound as defined by the invention.

The invention further provides an effective amount of the therapeutic passive vaccine comprising neutralizing antibodies that neutralize at least one CoV or of a compound that inhibits binding of at least one Spike protein of said CoV to the cognate receptor in a target cell, for use in a method for treating, preventing, inhibiting, reducing, eliminating, protecting or delaying the onset of at least one pathological condition caused by and/or associated with at least one CoV infection in a subject in need thereof. The invention further provides a diagnostic kit comprising at least one of: (a) at least one RBD or Spike protein of at least one CoV comprising at least one linker that replaces an anchor loop or any part thereof, or amino acid residue/s thereof, or any multimeric and/or multivalent antigen displaying platform thereof, and any combinations thereof, any derivative, enantiomer, fusion protein, conjugate, polyvalent dendrimer thereof, associated directly or indirectly to a solid support and/or a detectable moiety. (b) antibodies specific for the at least one RBD or Spike protein of at least one CoV associated directly or indirectly to a solid support and/or a detectable moiety. A further aspect of the invention relates to a diagnostic method for the detection of at least one CoV infection in a mammalian subject, the method comprising the steps of: (a), contacting at least one biological sample of the subject with at least one of (i) at least one RBD or Spike protein of at least one CoV comprising at least one linker that replaces an anchor loop or any part thereof, or amino acid residue/s thereof, associated directly or indirectly to a solid support and/or a detectable moiety; (ii) antibodies specific for the RBD or Spike protein associated directly or indirectly to a solid support and/or a detectable moiety; or (iii) any RBD or Spike protein binding molecule associated directly or indirectly to a solid support and/or a detectable moiety. (b), determining that the subject is infected with CoV if the detectable moiety is detected in the sample. In a further aspect thereof, the invention further provides an effective method for improving epitope-based vaccine that comprise at least one Spike protein of CoV, or any fragments or parts thereof. The method of the invention comprises the step of replacing the anchor loop of the Spike protein of said CoV, or any fragments or parts thereof with a linker.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1C: Receptor Binding Domains (RBDs) of three coronaviruses

FIG. 1A: Ribbon representations of the RBDs of SARS-CoV (PDBID:2GHV), and are shown.

FIG. 1B: Ribbon representations of the RBDs of MERS-CoV (PDBID:4KQZ) is shown.

FIG. 1C: Ribbon representations of the predicted structure for SARS-CoV2 (C) is shown.

All three structures contain a central core harboring 5 beta-strands (numbered). An excursion leaves the core, from beta-strand 4, forming an extended surface that contacts the virus receptor and is the target of neutralizing mAbs thus forming the RBM as has been confirmed for the three viruses. The RBM excursion is held in place via an “anchor loop” that forms hydrogen bonds with the RBD core.

FIG. 2A-2B: The model of the RBM for SARS-CoV2, used for the first generation reconstituted RBM

FIG. 2A: Structural model of the RBM of SARS-CoV2 with identification of the 4 amino acids that may be required for reconstitution of the viral RBM: the beginning and end of the RBM (residues 443 and 501) and the two residues that flank the “anchor loop” that needs to be deleted and replaced with linkers that functionally bridge the 10 Å gap (residues 456 and 470). Three hydrogen bonds that tacks to the loop to the core are marked with asterisks.

FIG. 2B: Schematic representation of the fragment from amino acid 443 to 501 of the Spike protein, as denoted by SEQ ID NO: 40. Segment A (as denoted by SEQ ID NO: 3) and segment B 1^{st} generation (as denoted by SEQ ID NO: 90) as well as the “anchor loop” (as denoted by SEQ ID NO: 37) of the proposed SARS-CoV2 RBM are given in which the conserved residues shared with the SARS-CoV are shown in grey.

FIG. 3: The SARS CoV2 RBD residues 336-518 (SEQ ID NO: 1) based on the cryo-EM structure 6m17.PDB.

FIG. 4: The SARS CoV2 RBD residues 336-518 (SEQ ID NO: 1) in which the RBM excursion—residues 443-501 (SEQ ID NO: 40) are highlighted in orange.

FIG. 5: The SARS CoV2 RBD residues 336-518 (SEQ ID NO: 1) in which the anchor loop (residues 457-469) are highlighted in blue.

FIG. 6: The SARS CoV2 RBD residues 336-518 in which three strands of ACE2 are shown illustrating how they interact with the RBM. Note the extended alpha-helix (residues E23 to L45 of the ACE2) that contacts multiple residues of the RBM.

FIG. 7A-7C: The SARS CoV2 RBD residues 336-518 in which the contact residues in the RBM are indicated as grey backbone (FIG. 7A), sticks (FIG. 7B) and spacefill (FIG. 7C).

FIG. 8: The SARS CoV2 RBD residues 336-518 in which the three strands of ACE2 are shown in green.

FIG. 9: The SARS CoV2 RBD residues 336-518 (SEQ ID NO: 1) with space filled contact residues and the strands of ACE2 however removal of the anchor loop as a first step in the RBM reconstitution.

FIG. 10: Design of the reconstituted RBM showing the contact residues, the orientation of the ACE2 segments and the position of the combinatorial linker bridging between residues 456 and 470 thus replacing the anchor loop (residues 457 to 469, SEQ ID NO: 37).

FIG. 11A-11B: Definition of the anchor loop in the RBD of SARS CoV

FIG. 11A: Schematic representation of the RBD of SARS CoV; core of RBD containing the 5 hallmark beta strands in pink, RBM features in grey containing the anchor loop in blue that forms hydrogen bonds with the core. The contributing members of each hydrogen bond are indicated as well as the beginning and end of each of the anchor loops.

FIG. 11B: Rulers indicating the distance from the first hydrogen bond to the first residues of the anchor loop as well as the distance from the last hydrogen bond to the last residues of the loop.

FIG. 12A-12B: Definition of the anchor loop in the RBD of MERS CoV

FIG. 12A: Schematic representation of the RBD of MERS CoV; core of RBD containing the 5 hallmark beta strands in pink, RBM features in grey containing the anchor loop in blue that forms hydrogen bonds with the core. The contributing members of each hydrogen bond are indicated as well as the beginning and end of each of the anchor loops.

FIG. 12B: Rulers indicating the distance from the first hydrogen bond to the first residues of the anchor loop as well as the distance from the last hydrogen bond to the last residues of the loop.

FIG. 13A-13B: Definition of the anchor loop in the RBD of SARS CoV2

FIG. 13A: Schematic representation of the RBD of SARS CoV2; core of RBD containing the 5 hallmark beta strands in pink, RBM features in grey containing the anchor loop in blue that forms hydrogen bonds with the core. The contributing members of each hydrogen bond are indicated as well as the beginning and end of each of the anchor loops.

FIG. 13B: Rulers indicating the distance from the first hydrogen bond to the first residues of the anchor loop as well as the distance from the last hydrogen bond to the last residues of the loop.

FIG. 14A-14E: Definition of SARS CoV2 RBD and RBM following co-crystallization with its receptor ACE2

FIG. 14A: The Receptor Binding Domain (RBD) of SARS CoV2 was co-crystallized with its receptor ACE2 [10]. The RBD (residues C336 to L518, SEQ ID NO: 1) contains an excursion (residues 5443-P499, colored light green, designated Receptor Binding Motif—RBM) with an anchor loop (457-472, (SEQ ID NO: 5) colored orange). The proposed combinatorial linkers connect residues F456 to Y473. For the Extended libraries residues A435-D442 (SEQ ID NO: 85, dark green) and T500-V510 (SEQ ID NO: 86, pink) are included to enable 88 different combinations of N and C terminal residues of constructs containing combinatorial linkers connecting residue 456 and residue 473 with 1-7 random residues.

FIG. 14B: Removal of the Anchor Loop—residues 457-472 (SEQ ID NO: 5).

FIG. 14C: The Extended RBM containing residues 443-499 (SEQ ID NO: 2) and N and C terminal extensions, 435-442 (SEQ ID NO: 85) and 500-510 (SEQ ID NO: 86) respectively.

FIG. 14D: The Extended RBM showing residues F456, Y473 and Y489. The three aromatic residues form a hydrophobic nucleus which stabilizes the RBM.

FIG. 14E: The Extended RBM showing the position of N501.

FIG. 15A-15C: Extended RBM in complex with ACE2

FIG. 15A: The Extended RBM complexed with segments of ACE2. The two helices of ACE2 (residues 21-85, (SEQ ID NO: 87)) are shown illustrating that the first helix (21-55, SEQ ID NO: 88, light blue) associates with the beta-strands of the RBM. A second loop (residues 350-360, SEQ ID NO: 89, blue) of ACE2 (SEQ ID NO: 23) contributes to the binding of RBM.

FIG. 15B: The interaction of residues K353 (blue) of ACE2 with N501 (pink) of the RBD.

FIG. 15C: Side view of the K353/N501 interaction.

FIG. 16A-16B: The Extended-combinatorial RBM library—3^{rd} generation FIG. 16A: shows a general scheme of the extended library, that contain residues 435-456 SEQ ID NO: 53, linked by combinatorial linkers from 1-5 residues long, to residues 473-510 (SEQ ID NO: 54). The whole sequence of residues 435-510 with a linker, is denoted by (SEQ ID NO: 56). This sequence includes the N′ and C′ extensions, specifically, residues 435 through 442 (AWNSNNLD, SEQ ID NO: 85), were added at the N′ terminus and residues 500-510 (TNGVGYQPYRV, SEQ ID NO: 86) were added at the C′ terminus. The sequence of the RBM residues 443-499 with a linker, is denoted by (SEQ ID NO: 55).

FIG. 16B: shows the various reconstituted RBMs, and their binding to the three neutralizing antibodies AO5, 441 and D11, used for screening of the library. The N′ extensions are denoted by SEQ ID NOs: 58-60, the various linkers are denoted by SEQ ID NOs:42-52, the C′ extensions are denoted by SEQ ID NOs: 61-64, and the various reconstituted RBMs are denoted by SEQ ID NOs:66-84. Various substitutions reveled in the screening are indicated in the figure.

FIG. 17: The linker motif, for the reconstituted RBMs

The figure shows a LOGO presentation of the five amino acid residues of the combinatorial linkers reveled in the screening.

FIG. 18: 3^{rd} generation screening

A representative dot blot filter from the screening of the extended NNK4 RBM library (3^{rd} generation) against mab 441. Note the positive control, RBD, and the negative control, fth-1, which is a phage devoid of insert and thus not presenting any peptides.

### DETAILED DESCRIPTION OF THE INVENTION

The present invention contends with development of specific and effective vaccines and vaccine-based therapeutic applications targeting viral pathogens, for example, corona viruses and specifically, SARS-CoV2 that may infect human as well as other mammals. The vaccines are based on exact reconstitution of the functional binding-motif of the SARS-CoV2 Spike protein (S1) and generation of recombinant peptides characterized by high affinity and specificity to SARS CoV2 receptors and neutralizing antibodies.

The present invention is based on the rationale that as the viral S1 is responsible for the host tropism and for immunogenicity in the host, it would be possible to reconstitute the S1 protein or even the functional part of the viral S1, namely the Receptor Binding Domain (RBD) or more specifically, the Receptor Binding Motif (RBM), that is capable of both of these functions, thereby targeting viral infections most effectively.

Particular advantageousness of the proposed approach is in achieving effective immunogen by focusing the immune response to the RBD neutralizing surface, i.e., RBM. It is important to stress that the mere knowledge of the RBM sequence and its components is insufficient to ensure RBM functionality. To guarantee a neutralizing immune response one must express RBM such that it assumes its highly conformational nature.

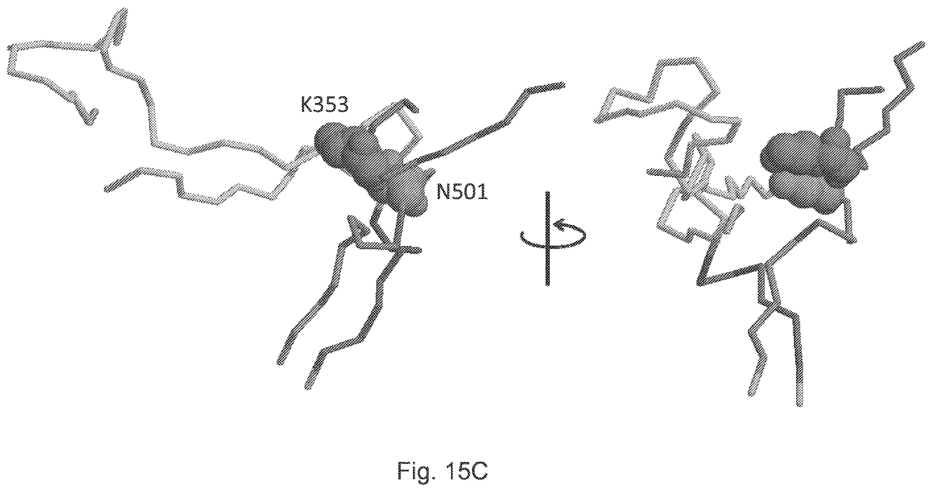

In some embodiments, the reconstituted RBM of the invention comprise a sequence of the native RBM, any partial sequences or amino acid residue/s thereof, and at least one linker. In some embodiments, the linker/s may replace at least one amino acid residues not directly involved or participate in receptor and/or neutralizing antibodies (nAb/s) binding. More specifically, residues not directly involved in binding or contact, include residues that may not serve necessarily as “contact residues”, but impact receptor and/or nAb/s binding, for example by conferring or maintaining certain conformation required for said binding. These specific residues may be replaced, substituted, excluded or removed in/from the functional reconstituted RBM polypeptides of the invention, or alternatively, in/from the entire RBD, in/from the S1 subunit, or in/from the entire Spike protein. In yet some further alternative embodiments, residues that may function as “contact residues” for the receptor and/or nAbs, may be replaced by at least one linker/s. In yet some further embodiments, at least one residue involved directly or indirectly in receptor and/or nAb/s binding, may be replaced by said linker/s. Nevertheless, at least one residue not involved in the receptor and/or nAbs interactions, may be replaced by the linker. In certain embodiments, sequences or residues that are not essential for binding and may be replaced by at least one linker, include for example, the anchor loop that tacks, attaches, connects and anchors the RBM extension to the core of the RBD via several hydrogen bonds. In some specific embodiments, for the spike protein of SARS CoV2, such anchor loop may comprise an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 spike protein. In some embodiments, the anchor loop may comprise residues 457-472 of the SARS CoV2 spike protein as denoted by the amino acid sequence of SEQ ID NO: 6, or of any homologs, mutants or variants thereof. In some embodiments, the variants or mutants may comprise any of the known spike protein variants and any combinations thereof. Of particular interest is N501Y substitution that also appears in the “British variant”, as denoted for example by SEQ ID NOs: 38 and 39, and/or the N501Y, K417N, E484K substitutions, that also appear in the “South African variant”, as denoted for example by SEQ ID NO: 41. It should be however understood that any of the following substitutions revealed for any strain, are further encompassed by the present disclosure, more specifically, the substitutions of the B1.1.7 strain (also known as the “British variant”), that include N501 Y, P681H, H69-V70 and Y144/145 deletions, A570D, D614G, P681H, T716I, S982A and D1118H. The substitutions of the B.1.1.298 strain that include H69-V70 deletion. Y453F, D614G, I692V and M1229I. The substitutions of the B.1.1.429 strain that include 8131, W152C, I452R and D614G; the substitutions of the P2 strain that include E484K, D614G, V1176F, the substitutions of the P1 strain that include L18F, T20N, P26S, D138Y, R190S, K417T, E484K, N501Y, D614G, H655Y, T1027I, V1176F. The substitutions of the B.1.351 strain (also known as 20C/501Y.V2, or the “South African variant”), that has the following variants B.1.351 V1 strain, that include the substitutions of D80A, D215G, L242/A243/L244 deletion, K417N, E484K, N501 Y, D614G, A701V; the B.1.351 V2 strain, that include the substitutions of L18F, D80A, D215G, L242/A243/L244 deletion, K417N, E484K, N501Y, D614G, A701V; and the B.1.351 V3 strain, that include the substitutions of D80A, R246I, L242/A243/L244 deletion, K417N, E484K, N501Y, D614G and A701V, Still further, as indicated in Examples 2-3, in the process of screening for the reconstituted RBMs, several substitutions that support a functional reconstituted RBM were revealed (also shown in FIG. 16). These substitutions include any one of Y489N, Q474R, V483E, Q474R, L492I. Thus, in some embodiments, a spike protein variant as referred to herein may comprise the amino acid sequence of SEQ ID NO: 6, with any substitution of at least one of Y489N, Q474R, V483E, Q474R, L492I, N501Y, K417N, E484K, or any combinations thereof. In yet some further alternative embodiments, the reconstituted RBM polypeptides of the invention may comprise at least 1, 2, 3, 4, 5, 6, 7, 8, 9, 10, 11, 1, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more residues that directly or indirectly participate in receptor and/or nAb/s binding. In more particular and non-limiting embodiments, the anchor loop comprising the amino acid sequence as denoted by SEQ ID NO: 5, and any variants, mutants and homologs thereof. In yet some further alternative embodiments, the anchor loop may comprise the amino acid sequence as denoted by SEQ ID NO: 37, and any variants, mutants and homologs thereof. The need for vaccines against emerging threats is unquestionable. This is all the more important when the viral threat has a relatively high mortality rate and is internationally spread. Furthermore, concerning the COVID-19 (disease caused by infection with SARS-COV2), the WHO considered the disease as pandemic, i.e., a disease occurring over a wide geographic area or worldwide and affecting an exceptionally high proportion of the population. Hence there is a genuine need for an effective prophylactic coronavirus vaccine, specifically, a SARS CoV2 vaccine. The present invention addresses this need, specifically, by providing reconstituted RBM molecules, as well as improved RBD and Spike proteins (either the S1 alone or the S1 and S2), that may be used as efficient vaccines, as well as for therapeutic and diagnostic applications. Thus, in a first aspect, the invention relates to a polypeptide comprising an amino acid sequence of at least one reconstituted Receptor Binding Motif (RBM) of a Spike protein of the Severe Acute Respiratory Syndrome coronavirus 2 (SARS CoV2) or of any fragment thereof. In some embodiments, the reconstituted RBM may comprise at least one linker and at least one fragment of the native RBM or of any variant or mutant thereof. More specifically, the native RBM or any variant or mutant thereof comprises an amino acid sequence starting at any one of the amino acid residues S443 (Serine 443, Ser), A435, (Alanine 435, Ala), W436 (Tryptophan 436, Trp), N437 (Asparagine 437, Asn), S438 (Serine 438, Ser), N439 (Asparagine 439, Asn), N440 (Asparagine 440, Asn), L441 (Leucine 441, Leu), D442 (Aspartic acid 442, Asp), K444 (Lysine 444, Lys), V445 (Valine 445, Val), G446 (Glycine 446, Gly), G447 (Glycine 447, Gly) or N448 (Asparagine 448, Asn), and ending at any one of the amino acid residues P499 (Proline 499, Pro), Y495 (Tyrosine 495, Tyr), G496 (Glycine 496, Gly), F497 (Phenylalanine 497, Phe), Q498 (Glutamine 498, Gln), T500 (Threonine 500, Thr), N501 (Asparagine 501, Asn), G502 (Glycine 502, Gly), V503 (Valine 503, Val), G504 (Glycine 504, Gly), Y505 (Tyrosine 505, Tyr), Q506 (Glutamine 506, Gln), P507 (Proline 507, Pro), Y508 (Tyrosine 508, Tyr), R509 (Arginine 509, Arg) or V510 (Valine 5010, Val) of the SARS CoV2 spike protein. Still further, the native RBM comprises an anchor loop comprising an amino acid sequence starting at any one of the amino acid residues F456 (Phenylalanine 456, Phe), R457 (Arginine 457, Arg), K458 (Lysine 458, Lys), R454 (Arginine 454, Arg), or L455 (Leucine 455, Leu) and ending at any one of the amino acid residues Y473 (Tyrosine 473, Tyr), I472 (Isoleucine, 472, Ile), E471 (Glutamic acid 471, Glu), T470 (Threonine 470, Thr), S469 (Serine 469, Ser), I468 (Isoleucine 468, Ile), Q474 (Glutamine 474, Gln), A475 (Alanine 475, Ala) or G476 (Glycine 476, Gly), of said SARS CoV2 Spike protein. It should be understood that the residues positions and numbering indicated herein refer to the residues of the spike protein, for example, the spike protein as denoted by SEQ ID NO: 6, or of an variants or mutants thereof. In some particular and non-limiting embodiments, the residues indicated herein refer to a variant of SEQ ID NO: 6, for example, the variant as denoted by SEQ ID NOs: 38 and 39, or the variant as denoted by SEQ ID NO: 41, or any variant of SEQ ID NO: 6 that comprises one or more of the N501Y, K417N, E484K, Y489N, Q474R, V483E, Q474R, L492I, L452R, Y453F, S13I, L18F, T20N, P26S, H69-V70 deletion, D80A, D138Y, Y144/145 deletions, W152C, R190S, D215G, L242/A243/L244 deletion, R246I, A570D, D614G, H655Y, P681H, I692V, A701V, T716I, S982A, T1027I, D1118H, V1176F, M1129I substitutions or any combinations thereof). In more specific embodiments, the at least one linker/s replaces the anchor loop or any part thereof, or amino acid residue/s thereof and any RBM fragment or amino acid residue/s thereof.

As indicated above, the polypeptide of the invention is derived from a particular region of the receptor binding domain of a spike protein, or any other peplomer, that interacts with the viral receptor on the target cell. A “peplomer”, as used herein, a glycoprotein structural unit found on viral capsid or the lipoprotein envelope of enveloped viruses. The peplomers are essential for both host specificity and viral infectivity. The term “peplomer” or “spike” is typically used to refer to a grouping of heterologous proteins on the virus surface that function together. The Spike protein comprises a receptor binding domain (RBD), that as used herein, refers to a region, segment or domain within a polypeptide studding (or covering) the envelope of a virus that is associated with or mediates the binding of the virus to a host cell, in particular to a host cell receptor. Polypeptides studding the envelope of a virus are commonly referred to as spikes or spike proteins. As noted herein before, the inventive concept behind the present invention is based on deep understanding of the unique structural/functional properties of the viral S1 protein and its components. This term herein refers to one of the functional subunits of the SARS CoV2 Spike glycoprotein, a class I viral fusion protein that forms the characteristic spikes, or peplomers, found on the viral surface that mediate virus attachment, fusion, and entry into the host cell, and thereby determines the tropism of the virus. During virus maturation, Spike glycoprotein is cleaved into two subunits: S1, which binds to receptors in the host cell, and S2, which mediates membrane fusion.

In other words, herein this term refers to SARS CoV2 protein referred to by terms Coronavirus Spike Glycoprotein, Spike Protein, Spike Glycoproteins S1, Spike glycoprotein (Gp) S1.

In yet some further embodiments, the SARS CoV2 Spike glycoprotein as used herein, refers to the SARS COV2 spike protein encoded by a nucleic acid sequence as denoted by NC_045512.2. In some specific embodiments, the SARS CoV2 spike protein encoded by a nucleic acid sequence as denoted by SEQ ID NO: 7, variants homologs, mutants and derivatives thereof. In some specific embodiments, the term SARS CoV2 Spike glycoprotein refers to the SARS CoV2 Spike glycoprotein referred to by SARS coronavirus 2 GenBank Protein Accession: YP_009724390.1. In more specific embodiments, the spike protein comprising the amino acid sequence as denoted by SEQ ID NO: 6, or any variants homologs, mutants and derivatives thereof. In some particular and non-limiting embodiments, variants and/or mutants as used herein, refer to a spike protein, for example, the spike protein comprising the amino acid sequence as denoted by SEQ ID NO: 6, with at least one of the following substitutions Y489N, Q474R, V483E, L492I, N501Y, K417N, E484K, L452R, Y453F, S13I, L18F T20N, P26S, H69-V70 deletion. D80A, D13Y, Y144/145 deletions, W152C, R190S, D215G, L242/A243/L244 deletion, R246I, A570D, D614G, H655Y, P681H, I692V, A701V, T716I, S982A, T1027I, D1118H, V1176F, M1229I or any combinations thereof. Non-limiting embodiments for such variants are denoted by SEQ ID NO: 38, 39 and 41. It should be noted that the SARS CoV2 spike protein as used herein refers to the entire spike protein that comprises the S1 and the S2 subunits. However, in some embodiments, the term SARS CoV2 refers to the SARS COV2 S1 protein that comprises residues M1 to E661 of the spike protein. In some embodiments, the S1 protein is encoded by the nucleic acid sequence as denoted by SEQ ID NO: 20, or any variants homologs, mutants and derivatives thereof. Still further, the S1 subunit of the spike protein of SARS CoV2 comprises the amino acid sequence as denoted by SEQ ID NO: 19, or any variants homologs, mutants and derivatives thereof. In this connection, the S2 subunit in accordance with some embodiments comprises residues C662 to T1273 of the SARS CoV2 Spike protein, specifically the spike protein as denoted by SEQ ID NO: 6. In yet some further embodiments, the S2 subunit of SARS CoV2 comprises an amino acid sequence as denoted by SEQ ID NO: 21, or any variants homologs, mutants and derivatives thereof. In more specific embodiments, the S2 subunit is encoded by the nucleic acid sequence as denoted by SEQ ID NO: 22, or any variants homologs, mutants and derivatives thereof. In this connection, the invention further refers to the RBD that is the receptor binding domain of the Spike protein (specifically of S1). In some specific embodiments, such RBD may comprise the amino acid sequence as denoted by SEQ ID NO: 1, or any variants homologs, mutants and derivatives thereof.

Different putative S1 sequences from SARS CoV2 isolates can be obtained from NCBI. It should be therefore appreciated that in certain embodiments, each of these S1 proteins is encompassed by the invention. Further, the present invention pertains to a specific region within the Spike Gp S1, specifically the Receptor Binding Domain (RBD), and more specifically the Receptor Binding Motif (RBM), as being responsible for the S1 bi-functional properties, namely host receptor binding and host immunity receptors. In the case of SARS CoV2, it has been demonstrated that the infection is mediated through the interaction of the viral Spike protein and its cellular receptor Angiotensin-Converting Enzyme 2 (ACE2). The SARS CoV2 RBD (amino acids C336-L518 of S protein, denoted by SEQ ID NO: 1) harbors an extended secondary structure, also referred to herein as an extended excursion that contacts the receptor, ACE2, which is RBM (amino acids 5443-P499, denoted by SEQ ID NO: 2), or any variants and mutants thereof, for example, an of the variants discussed herein, specifically, variants having at least one of the following substitutions Y489N, V483E, Q474R, L492I, N501Y, E484K, L452R, Y453F, S13I, L18F, T20N, P26S, H69-V70 deletion, D80A, D138Y, Y144/145 deletions, W152C, R190S, D215G, L242/A243/L244 deletion, R246I, A570D, D614G, H655Y, P681H, I692V, A701V, T716I, S982A, T1027I, D1118H, V1176F, M1229I or any combinations thereof. In yet some alternative embodiments, the RBM may comprise the amino acid sequence as denoted by SEQ ID NO: 40 (residues 5433-N501), or any variants thereof, as discussed herein. The term receptor binding motif (RBM) as used herein refers to a region within the RBD that is in direct contact with the host cell or the viral receptor on the host cell. In other words, the term refers to a region within a polypeptide studding (or covering) the envelope of a virus, specifically within the RBD thereof, that mediates binding between the virus and host cell though non-covalent interactions. The RBM may be identified by any method known in the art based on the interaction formed between the spike protein and a host cell. In certain embodiments, the RBM of the viral spike protein may be an extended 30, 31, 32, 33, 34, 45, 46, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, 100, 110, 120, 130, 140, 150, 160, 170, 180, 190, 200 or more amino acid excursion, or a secondary structure extension. As noted above, at least part of the RBM participates in receptor interaction. As indicated above, the invention provides reconstituted RBMs that comprise at least one linker and at least one fragment derived from a native RBM or of any variants or mutants thereof, specifically, any of the variants discussed herein (e.g., substitutions Y489N, Q474R, V483E, Q474R, L492I, N501Y, E484K, L452R, Y453F or any combinations thereof). Reconstituted RBM, as used herein refers to a recombinant or synthetically non-natural RBM, build up again from parts, reconstructed, recombined and recomposed of fragments of the native RBM, that may in certain embodiments attached or linked together by at least one linker. As noted above, the reconstituted RBM of the invention comprise at least one fragment or amino acid residue or sequence of the native or natural RBM, and at least one linker. In some embodiments, the linker is a non-native linker, synthetic linker or exogenous linker. In yet some further embodiments, the linker is not a natural part of the native RBM or of any variants and mutants thereof. Specific embodiments for the reconstituted RBMs provided by the invention are described in more detail herein after. Native protein as used herein refers to a protein in its properly folded and/or assembled form, which is operative and functional. The native state of a protein may possess all four levels of bio-molecular structure, with the secondary through quaternary structure being formed from weak interactions along the covalently-bonded backbone. In still further embodiments, this term relates to the RBM of the natural S1 protein as appropriately expressed and presented in the natural viral envelop or capsid. Therefore, in some embodiments, the linker used must differ from the replaced native sequence in at least one amino acid residue, and specifically, two, three, four, five, six, seven or more residues. In yet some further embodiments, the linker used to replace the native sequences (e.g., the loop or any fragments thereof), differs from the native replaced sequence, such that the reconstituted RBM that comprise said at least one linker cannot be considered as a natural product.

Thus, in some embodiments, the reconstituted RBMs of the invention may comprise the sequence of the native RBM or of any variants and mutants thereof, were residues, fragments or segments that are not directly involved or participate in receptor interaction or receptor contact may be replaced by at least one linker. It should be however understood that the reconstituted RBMs of the invention may comprise at least part, and preferably, most or all residues that participate in interaction with the receptor. In yet some further alternative embodiments, the linker/s of the reconstituted RBMs of the invention may replace at least one amino acid residues involved directly or indirectly in receptor and/or nAb/s binding. Nevertheless, it should be understood that in some embodiments, the reconstituted RBMs of the invention may comprise at least one or more, specifically, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 2, at least 23, at least 24, at least 25, at least 26, at least 27, at least 28, at least 29, at least 30, at least 35, at least 40, at least 45, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, or more RBM amino acid residues that are directly or indirectly involved in in receptor and/or nAb/s binding. In this connection, it should be noted that in certain embodiments, amino acid sequences or amino acid residues that are not directly or indirectly involved in interaction with various neutralizing antibodies, may be also replaced, removed, excluded or substituted by at least one linker. The reconstituted RBMs of the invention may comprise the amino acid sequence of the native RBM, or of any fragment thereof. “Fragment” with respect to polypeptide sequences means polypeptides that comprise at least 10%, at least 20%, at least 30%, at least 40%, at least 50%, at least 60%, at least 70%, at least 80%, at least 90% or at least 95% of the complete whole segment of the native RBM of said viral spike protein, specifically the SARS-CoV2 S1 protein. In some embodiments, fragments of the RBMs may comprise at least 5, at least 10, at least 15, at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more and at least 100 amino acids or more of said spike RBMs. As indicated above, the reconstituted RBM of the invention comprises at least one linker that replaces in some embodiments, the anchor loop (may be also referred to as tacking segment) of the native RBM, or any part thereof, or amino acid residue/s thereof. As such, in some embodiments the reconstituted RBM of the invention lacks at least part of the native anchor loop. In further embodiments, the reconstituted RBM of the invention may comprise more than one linker, for example, 2, 3, 5, 6, 7, 8, 9, 10 or more linkers, that replace at least part of the anchor loop, or a sequence that comprise at least part of the anchor loop. It should be further appreciated that in some particular embodiments, in addition to linker/s that replace the anchor loop, the reconstituted RBM of the invention may further comprise at least one linker that replace/s at least one amino acid residue/s located in other segments of the native RBM. In yet some further embodiments, the reconstituted RBM polypeptide of the invention may comprise at least one linker that replaces at least one amino acid residue of the RBM, or any fragments thereof not directly involved in receptor and/or nAb/s binding. Alternatively, the linker/s may replace at least one amino acid residue of the RBM directly or indirectly involved and participate in receptor and/or nAb/s binding. Still further, the reconstitute RBM polypeptides of the invention may comprise between about 10 to 100 amino acid residues, specifically, between about 20 to 75 amino acid residues. Specifically, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 45, 46, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, 100 or more amino acid residues. In more specific embodiments, the polypeptide of the invention may comprise reconstituted RBM comprising at least one linker that replace/s the anchor loop (tacking segment) or any part thereof, or at least one amino acid residue thereof. In yet further embodiments, the reconstituted RBM of the invention may further comprise additional linker/s that may replace or may be added to further residues of other RBM segments, for example, residues that are located out of the loop. As indicated above, in some particular embodiments, the reconstituted RBM of the invention may comprise at least one linker that replaces the loop tacking or anchoring segment or any part thereof and optionally at least one further linker that replaces at least one amino acid residue located in other parts of the RBM. In yet some further embodiments, the reconstituted RBM may comprise at least one linker that may replace or may be added to other parts or fragments of the native RBM. More specifically, at least one of the linker/s may replace at least one RBM fragment or amino acid residue/s not directly involved in receptor binding.

In yet some alternative embodiments, the linker comprised within the reconstituted RBM polypeptide of the invention may replace at least one RBM fragment or amino acid residue directly or indirectly involved in receptor and/or neutralizing antibodies (nAb/s) binding.

It should be appreciated however, that at least some of the reconstituted RBM polypeptides of the invention comprise at least one or more amino acid residue/s directly or indirectly involved in receptor and/or nAb/s binding. In some specific embodiments, the reconstitute RBM polypeptides of the invention may comprise at least one amino acid sequence derived from the RBM of the Coronavirus SARS CoV2 S1 protein.

Thus, the main goal of the present invention is to provide means to combat Coronaviruses (CoVs) infections, specifically SARS-CoV2 infections and to prevent spread of such infections via other animals to humans and/or to domestic animals as well as to prevent human to human infections. CoVs are common in humans and usually cause mild to moderate upper-respiratory tract illnesses. There are four main sub-groupings of coronaviruses, known as alpha, beta, gamma, and delta. The seven coronaviruses known to-date as infecting humans are: alpha coronaviruses 229E and NL63, and beta coronaviruses OC43, HKU1, SARS-CoV and SARS-CoV2, and MERS-CoV (the coronavirus that causes Middle East Respiratory Syndrome, or MERS). The SARS-CoV and SARS-CoV2 are a lineage B beta Coronavirus and the MERS-CoV is a lineage C beta Coronavirus. The present invention in particular concerns the novel coronaviruses that have emerged in humans in 2019: SARS-COV2 which is associated with high contagion and relatively high mortality rates. Currently there are a number of clinically approved vaccines that are in use worldwide. To date over 689 million shots have been given to people in more than 150 countries. That being said, however, the vaccines comprise full length Spike protein in one form or other. No vaccine modality has been approved implementing an RBD or even S1 exclusively. Using RBM of the present invention would provide a targeted immunogen, focusing the protective immune response to the most critical neutralizing surface of the virus. Furthermore, use of tau-linker modified proteins (RBD, S1 or Spike) as disclosed herein, would accentuate the RBM in their replacement of the anchor loop for at least one tau-linker (τ-linker). Moreover, the durability and length of time of protection afforded by the approved vaccines in use thus far are still unknown. There is a serious likelihood that additional boost vaccinations will be required even after initial vaccination with the already approved vaccines. Thus, the development of effective therapeutic and preventive strategies that can be readily applied to new emergent strains of SARS-COV2 is a research priority. The current proposed RBM can be used as a universal boost vaccine, regardless of the modality or source of an initial first vaccination protocol.

In the specific case of the SARS CoV2, a defined receptor-binding domain on S mediates the attachment of the virus to its cellular receptor, angiotensin-converting enzyme 2 (ACE2) (see below). Some CoVs, specifically the members of Beta CoV subgroup A, also have a shorter spike-like protein called hemagglutinin esterase (HE).

Of particular relevance to the present invention is the SARS CoV2 associated with Severe Acute Respiratory Syndrome 2, as for being the primary cause of life-threatening infectious diseases and epidemics/pandemics in humans referred to as COVID 19 (Coronavirus Disease 2019).

SARS CoV2 is a member of the subgenus Sarbecovirus (beta-CoV lineage B). Its RNA sequence is approximately 30,000 bases in length. SARS-CoV-2 is unique among known betacoronaviruses in its incorporation of a polybasic cleavage site, a characteristic known to increase pathogenicity and transmissibility in other viruses. With a sufficient number of sequenced genomes, it is possible to reconstruct a phylogenetic tree of the mutation history of a family of viruses. By 11 Feb. 2020, 81 genomes of SARS-CoV-2 had been isolated and reported by the Chinese Center for Disease Control and Prevention (CCDC) and other institutions. A phylogenetic analysis of those samples showed they were “highly related with at most seven mutations relative to a common ancestor”, implying that the first human infection occurred in November or December 2019.

On 11 Feb. 2020, the International Committee on Taxonomy of Viruses (ICTV) announced that according to existing rules that compute hierarchical relationships among coronaviruses on the basis of five conserved sequences of nucleic acids, the differences between what was then called 2019-nCoV and the virus strain from the 2003 SARS outbreak were insufficient to make it a separate viral species. Therefore, they identified 2019-nCoV (now referred to as SARS CoV2) as a strain of severe acute respiratory syndrome-related coronavirus.

Each SARS-CoV-2 virion is approximately 50 to 200 nanometres in diameter. Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the viral envelope. In some embodiments, the nucleic acid sequence of the severe acute respiratory syndrome coronavirus 2 isolate (SARS CoV2) Wuhan-Hu-1, complete genome is denoted by NCBI Reference Sequence: NC_045512.2. In yet some further embodiments, the SARS CoV2 nucleic acid sequence is as denoted by SEQ ID NO: 36. The spike protein is responsible for allowing the virus to attach to the membrane of a host cell [5]. Studies have shown that SARS-CoV-2 has a higher affinity to human ACE2 than the original SARS virus strain. An atomic-level image of the S protein has been created using cryogenic electron microscopy [7]. As noted above, the reconstituted RBMs of the invention are composed of at least one fragment of the native RBM of the spike protein of SARS CoV2, and at least one linker.

In some embodiments, the RBM of the polypeptide of the invention is of a Spike protein of the SARS CoV2 that comprises the amino acid sequence as denoted by SEQ ID NO: 6, or any variants, mutants, derivatives and homologs thereof. Although the reconstituted RBM of the invention is based on the spike protein of SARS CoV2, it should be appreciated that the invention further encompasses the use of any variant, homolog, or ortholog of the spike protein as denoted by SEQ ID NO: 6. For example, any variant, homolog, or ortholog that display between about 50%, 55%, 60%, 65%, 70%, 75%, 80%, 85%, 90%, 95%, 97%, 98%, 99%, 99.99% or 100% homology with the spike protein as denoted by SEQ ID NO: 6. In yet some further embodiments, a variant or mutant variants and/or mutants as used herein, refer to a spike protein, for example, the spike protein comprising the amino acid sequence as denoted by SEQ ID NO: 6, with at least one of the following substitutions Y489N, Q474R, V483E, L492I, N501Y, K417N, E484K, L452R, Y453F or any combinations thereof. Non-limiting embodiments for such variants are denoted by SEQ ID NO: 38, 39 and 41.

In yet some further specific embodiments, the reconstituted RBM polypeptides of the invention may comprise at least one amino acid residue derived from the RBM of the SARS-CoV2 S1 protein, as designated by SEQ ID NO: 6 and any variants thereof, specifically, those discussed above. In some embodiments, the native RBM of the SARS CoV2, comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 spike protein.

More specifically, in some embodiments, the native RBM comprises the amino acid sequence starting at residue S443 and ending at residue P499 of the spike protein of SARS CoV2 as denoted by SEQ ID NO: 6, or any homologs and variants thereof. In some specific and non-limiting embodiments, the S443 to P49 is denoted by SEQ ID NO: 2, and any variants thereof, specifically, as discussed herein. In some embodiments, the native RBM comprises the amino acid sequence starting at residue S443 and ending at residue Y495 of the spike protein of SARS CoV2 as denoted by SEQ ID NO: 6, or any homologs and variants thereof. In some embodiments, the native RBM comprises the amino acid sequence starting at residue S443 and ending at residue G496 of the spike protein of SARS CoV2 as denoted by SEQ ID NO: 6, or any homologs and variants thereof. In some embodiments, the native RBM comprises the amino acid sequence starting at residue S443 and ending at residue F497 of the spike protein of SARS CoV2 as denoted by SEQ ID NO: 6, or any homologs and variants thereof. In some embodiments, the native RBM comprises the amino acid sequence starting at residue S443 and ending at residue Q498 of the spike protein of SARS CoV2 as denoted by SEQ ID NO: 6, or any homologs and variants thereof. In some embodiments, the native RBM comprises the amino acid sequence starting at residue S443 and ending at residue T500 of the spike protein of SARS CoV2 as denoted by SEQ ID NO: 6, or any homologs and variants thereof. In some embodiments, the native RBM comprises the amino acid sequence starting at residue S443 and ending at residue N501 of the spike protein of SARS CoV2 as denoted by SEQ ID NO: 6, or any homologs and variants thereof. In some embodiments, the native RBM comprises the amino acid sequence starting at residue S443 and ending at residue G502 of the spike protein of SARS CoV2 as denoted by SEQ ID NO: 6, or any homologs and variants thereof. In some embodiments, the native RBM comprises the amino acid sequence starting at residue S443 and ending at residue V503 of the spike protein of SARS CoV2 as denoted by SEQ ID NO: 6, or any homologs and variants thereof. In some embodiments, the native RBM comprises the amino acid sequence starting at residue S443 and ending at residue G504 of the spike protein of SARS CoV2 as denoted by SEQ ID NO: 6, or any homologs and variants thereof. In some embodiments, the native RBM comprises the amino acid sequence starting at residue S443 and ending at residue Y505 of the spike protein of SARS CoV2 as denoted by SEQ ID NO: 6, or any homologs and variants thereof. In some embodiments, the native RBM comprises the amino acid sequence starting at residue S443 and ending at residue Q506 of the spike protein of SARS CoV2 as denoted by SEQ ID NO: 6, or any homologs and variants thereof. In some embodiments, the native RBM comprises the amino acid sequence starting at residue S443 and ending at residue P507 of the spike protein of SARS CoV2 as denoted by SEQ ID NO: 6, or any homologs and variants thereof. In some embodiments, the native RBM comprises the amino acid sequence starting at residue S443 and ending at residue Y508 of the spike protein of SARS CoV2 as denoted by SEQ ID NO: 6, or any homologs and variants thereof. In some embodiments, the native RBM comprises the amino acid sequence starting at residue S443 and ending at residue R509 of the spike protein of SARS CoV2 as denoted by SEQ ID NO: 6, or any homologs and variants thereof. In some embodiments, the native RBM comprises the amino acid sequence starting at residue S443 and ending at residue V510 of the spike protein of SARS CoV2 as denoted by SEQ ID NO: 6, or any homologs and variants thereof.

In some embodiments, the native RBM comprises the amino acid sequence starting at residue A435, and ending at residue P499 of the spike protein of SARS CoV2 as denoted by SEQ ID NO: 6, or any homologs and variants thereof. In some embodiments, the native RBM comprises the amino acid sequence starting at residue A435, and ending at residue Y495 of the spike protein of SARS CoV2 as denoted by SEQ ID NO: 6, or any homologs and variants thereof. In some embodiments, the native RBM comprises the amino acid sequence starting at residue A435, and ending at residue G496 of the spike protein of SARS CoV2 as denoted by SEQ ID NO: 6, or any homologs and variants thereof. In some embodiments, the native RBM comprises the amino acid sequence starting at residue A435, and ending at residue F497 of the spike protein of SARS CoV2 as denoted by SEQ ID NO: 6, or any homologs and variants thereof. In some embodiments, the native RBM comprises the amino acid sequence starting at residue A435, and ending at residue Q498 of the spike protein of SARS CoV2 as denoted by SEQ ID NO: 6, or any homologs and variants thereof. In some embodiments, the native RBM comprises the amino acid sequence starting at residue A435, and ending at residue T500 of the spike protein of SARS CoV2 as denoted by SEQ ID NO: 6, or any homologs and variants thereof. In some embodiments, the native RBM comprises the amino acid sequence starting at residue A435, and ending at residue N501 of the spike protein of SARS CoV2 as denoted by SEQ ID NO: 6, or any homologs and variants thereof. In some embodiments, the native RBM comprises the amino acid sequence starting at residue A435, and ending at residue G502 of the spike protein of SARS CoV2 as denoted by SEQ ID NO: 6, or any homologs and variants thereof. In some embodiments, the native RBM comprises the amino acid sequence starting at residue A435, and ending at residue V503 of the spike protein of SARS CoV2 as denoted by SEQ ID NO: 6, or any homologs and variants thereof. In some embodiments, the native RBM comprises the amino acid sequence starting at residue A435, and ending at residue G504 of the spike protein of SARS CoV2 as denoted by SEQ ID NO: 6, or any homologs and variants thereof. In some embodiments, the native RBM comprises the amino acid sequence starting at residue A435, and ending at residue Y505 of the spike protein of SARS CoV2 as denoted by SEQ ID NO: 6, or any homologs and variants thereof. In some embodiments, the native RBM comprises the amino acid sequence starting at residue A435, and ending at residue Q506 of the spike protein of SARS CoV2 as denoted by SEQ ID NO: 6, or any homologs and variants thereof. In some embodiments, the native RBM comprises the amino acid sequence starting at residue A435, and ending at residue P507 of the spike protein of SARS CoV2 as denoted by SEQ ID NO: 6, or any homologs and variants thereof. In some embodiments, the native RBM comprises the amino acid sequence starting at residue A435, and ending at residue Y508 of the spike protein of SARS CoV2 as denoted by SEQ ID NO: 6, or any homologs and variants thereof. In some embodiments, the native RBM comprises the amino acid sequence starting at residue A435, and ending at residue R509 of the spike protein of SARS CoV2 as denoted by SEQ ID NO: 6, or any homologs and variants thereof. In some embodiments, the native RBM comprises the amino acid sequence starting at residue A435, and ending at residue V510 of the spike protein of SARS CoV2 as denoted by SEQ ID NO: 6, or any homologs and variants thereof.

In some embodiments, the native RBM comprises the amino acid sequence starting at residue W436 and ending at residue P499 of the spike protein of SARS CoV2 as denoted by SEQ ID NO: 6, or any homologs and variants thereof. In some embodiments, the native RBM comprises the amino acid sequence starting at residue W436 and ending at residue Y495 of the spike protein of SARS CoV2 as denoted by SEQ ID NO: 6, or any homologs and variants thereof. In some embodiments, the native RBM comprises the amino acid sequence starting at residue W436 and ending at residue G496 of the spike protein of SARS CoV2 as denoted by SEQ ID NO: 6, or any homologs and variants thereof. In some embodiments, the native RBM comprises the amino acid sequence starting at residue W436 and ending at residue F497 of the spike protein of SARS CoV2 as denoted by SEQ ID NO: 6, or any homologs and variants thereof. In some embodiments, the native RBM comprises the amino acid sequence starting at residue W436 and ending at residue Q498 of the spike protein of SARS CoV2 as denoted by SEQ ID NO: 6, or any homologs and variants thereof. In some embodiments, the native RBM comprises the amino acid sequence starting at residue W436 and ending at residue T500 of the spike protein of SARS CoV2 as denoted by SEQ ID NO: 6, or any homologs and variants thereof. In some embodiments, the native RBM comprises the amino acid sequence starting at residue W436 and ending at residue N501 of the spike protein of SARS CoV2 as denoted by SEQ ID NO: 6, or any homologs and variants thereof. In some embodiments, the native RBM comprises the amino acid sequence starting at residue W436 and ending at residue G502 of the spike protein of SARS CoV2 as denoted by SEQ ID NO: 6, or any homologs and variants thereof. In some embodiments, the native RBM comprises the amino acid sequence starting at residue W436 and ending at residue V503 of the spike protein of SARS CoV2 as denoted by SEQ ID NO: 6, or any homologs and variants thereof. In some embodiments, the native RBM comprises the amino acid sequence starting at residue W436 and ending at residue G504 of the spike protein of SARS CoV2 as denoted by SEQ ID NO: 6, or any homologs and variants thereof. In some embodiments, the native RBM comprises the amino acid sequence starting at residue W436 and ending at residue Y505 of the spike protein of SARS CoV2 as denoted by SEQ ID NO: 6, or any homologs and variants thereof. In some embodiments, the native RBM comprises the amino acid sequence starting at residue W436 and ending at residue Q506 of the spike protein of SARS CoV2 as denoted by SEQ ID NO: 6, or any homologs and variants thereof. In some embodiments, the native RBM comprises the amino acid sequence starting at residue W436 and ending at residue P507 of the spike protein of SARS CoV2 as denoted by SEQ ID NO: 6, or any homologs and variants thereof. In some embodiments, the native RBM comprises the amino acid sequence starting at residue W436 and ending at residue Y508 of the spike protein of SARS CoV2 as denoted by SEQ ID NO: 6, or any homologs and variants thereof. In some embodiments, the native RBM comprises the amino acid sequence starting at residue W436 and ending at residue R509 of the spike protein of SARS CoV2 as denoted by SEQ ID NO: 6, or any homologs and variants thereof. In some embodiments, the native RBM comprises the amino acid sequence starting at residue W436 and ending at residue V510 of the spike protein of SARS CoV2 as denoted by SEQ ID NO: 6, or any homologs and variants thereof.

In some embodiments, the native RBM comprises the amino acid sequence starting at residue N437 and ending at residue P499 of the spike protein of SARS CoV2 as denoted by SEQ ID NO: 6, or any homologs and variants thereof. In some embodiments, the native RBM comprises the amino acid sequence starting at residue N437 and ending at residue Y495 of the spike protein of SARS CoV2 as denoted by SEQ ID NO: 6, or any homologs and variants thereof. In some embodiments, the native RBM comprises the amino acid sequence starting at residue N437 and ending at residue G496 of the spike protein of SARS CoV2 as denoted by SEQ ID NO: 6, or any homologs and variants thereof. In some embodiments, the native RBM comprises the amino acid sequence starting at residue N437 and ending at residue F497 of the spike protein of SARS CoV2 as denoted by SEQ ID NO: 6, or any homologs and variants thereof. In some embodiments, the native RBM comprises the amino acid sequence starting at residue N437 and ending at residue Q498 of the spike protein of SARS CoV2 as denoted by SEQ ID NO: 6, or any homologs and variants thereof. In some embodiments, the native RBM comprises the amino acid sequence starting at residue N437 and ending at residue T500 of the spike protein of SARS CoV2 as denoted by SEQ ID NO: 6, or any homologs and variants thereof. In some embodiments, the native RBM comprises the amino acid sequence starting at residue N437 and ending at residue N501 of the spike protein of SARS CoV2 as denoted by SEQ ID NO: 6, or any homologs and variants thereof. In some embodiments, the native RBM comprises the amino acid sequence starting at residue N437 and ending at residue G502 of the spike protein of SARS CoV2 as denoted by SEQ ID NO: 6, or any homologs and variants thereof. In some embodiments, the native RBM comprises the amino acid sequence starting at residue N437 and ending at residue V503 of the spike protein of SARS CoV2 as denoted by SEQ ID NO: 6, or any homologs and variants thereof. In some embodiments, the native RBM comprises the amino acid sequence starting at residue N437 and ending at residue G504 of the spike protein of SARS CoV2 as denoted by SEQ ID NO: 6, or any homologs and variants thereof. In some embodiments, the native RBM comprises the amino acid sequence starting at residue N437 and ending at residue Y505 of the spike protein of SARS CoV2 as denoted by SEQ ID NO: 6, or any homologs and variants thereof. In some embodiments, the native RBM comprises the amino acid sequence starting at residue N437 and ending at residue Q506 of the spike protein of SARS CoV2 as denoted by SEQ ID NO: 6, or any homologs and variants thereof. In some embodiments, the native RBM comprises the amino acid sequence starting at residue N437 and ending at residue P507 of the spike protein of SARS CoV2 as denoted by SEQ ID NO: 6, or any homologs and variants thereof. In some embodiments, the native RBM comprises the amino acid sequence starting at residue N437 and ending at residue Y508 of the spike protein of SARS CoV2 as denoted by SEQ ID NO: 6, or any homologs and variants thereof. In some embodiments, the native RBM comprises the amino acid sequence starting at residue N437 and ending at residue R509 of the spike protein of SARS CoV2 as denoted by SEQ ID NO: 6, or any homologs and variants thereof. In some embodiments, the native RBM comprises the amino acid sequence starting at residue N437 and ending at residue V510 of the spike protein of SARS CoV2 as denoted by SEQ ID NO: 6, or any homologs and variants thereof.

In some embodiments, the native RBM comprises the amino acid sequence starting at residue S438 and ending at residue P499 of the spike protein of SARS CoV2 as denoted by SEQ ID NO: 6, or any homologs and variants thereof. In some embodiments, the native RBM comprises the amino acid sequence starting at residue S438 and ending at residue Y495 of the spike protein of SARS CoV2 as denoted by SEQ ID NO: 6, or any homologs and variants thereof. In some embodiments, the native RBM comprises the amino acid sequence starting at residue S438 and ending at residue G496 of the spike protein of SARS CoV2 as denoted by SEQ ID NO: 6, or any homologs and variants thereof. In some embodiments, the native RBM comprises the amino acid sequence starting at residue S438 and ending at residue F497 of the spike protein of SARS CoV2 as denoted by SEQ ID NO: 6, or any homologs and variants thereof. In some embodiments, the native RBM comprises the amino acid sequence starting at residue S438 and ending at residue Q498 of the spike protein of SARS CoV2 as denoted by SEQ ID NO: 6, or any homologs and variants thereof. In some embodiments, the native RBM comprises the amino acid sequence starting at residue S438 and ending at residue T500 of the spike protein of SARS CoV2 as denoted by SEQ ID NO: 6, or any homologs and variants thereof. In some embodiments, the native RBM comprises the amino acid sequence starting at residue S438 and ending at residue N501 of the spike protein of SARS CoV2 as denoted by SEQ ID NO: 6, or any homologs and variants thereof. In some embodiments, the native RBM comprises the amino acid sequence starting at residue S438 and ending at residue G502 of the spike protein of SARS CoV2 as denoted by SEQ ID NO: 6, or any homologs and variants thereof. In some embodiments, the native RBM comprises the amino acid sequence starting at residue S438 and ending at residue V503 of the spike protein of SARS CoV2 as denoted by SEQ ID NO: 6, or any homologs and variants thereof. In some embodiments, the native RBM comprises the amino acid sequence starting at residue S438 and ending at residue G504 of the spike protein of SARS CoV2 as denoted by SEQ ID NO: 6, or any homologs and variants thereof. In some embodiments, the native RBM comprises the amino acid sequence starting at residue S438 and ending at residue Y505 of the spike protein of SARS CoV2 as denoted by SEQ ID NO: 6, or any homologs and variants thereof. In some embodiments, the native RBM comprises the amino acid sequence starting at residue S438 and ending at residue Q506 of the spike protein of SARS CoV2 as denoted by SEQ ID NO: 6, or any homologs and variants thereof. In some embodiments, the native RBM comprises the amino acid sequence starting at residue S438 and ending at residue P507 of the spike protein of SARS CoV2 as denoted by SEQ ID NO: 6, or any homologs and variants thereof. In some embodiments, the native RBM comprises the amino acid sequence starting at residue S438 and ending at residue Y508 of the spike protein of SARS CoV2 as denoted by SEQ ID NO: 6, or any homologs and variants thereof. In some embodiments, the native RBM comprises the amino acid sequence starting at residue S438 and ending at residue R509 of the spike protein of SARS CoV2 as denoted by SEQ ID NO: 6, or any homologs and variants thereof. In some embodiments, the native RBM comprises the amino acid sequence starting at residue S438 and ending at residue V510 of the spike protein of SARS CoV2 as denoted by SEQ ID NO: 6, or any homologs and variants thereof. In some embodiments, the native RBM comprises the amino acid sequence starting at residue N439 and ending at residue P499 of the spike protein of SARS CoV2 as denoted by SEQ ID NO: 6, or any homologs and variants thereof. In some embodiments, the native RBM comprises the amino acid sequence starting at residue N439 and ending at residue Y495 of the spike protein of SARS CoV2 as denoted by SEQ ID NO: 6, or any homologs and variants thereof. In some embodiments, the native RBM comprises the amino acid sequence starting at residue N439 and ending at residue G496 of the spike protein of SARS CoV2 as denoted by SEQ ID NO: 6, or any homologs and variants thereof. In some embodiments, the native RBM comprises the amino acid sequence starting at residue N439 and ending at residue F497 of the spike protein of SARS CoV2 as denoted by SEQ ID NO: 6, or any homologs and variants thereof. In some embodiments, the native RBM comprises the amino acid sequence starting at residue N439 and ending at residue Q498 of the spike protein of SARS CoV2 as denoted by SEQ ID NO: 6, or any homologs and variants thereof. In some embodiments, the native RBM comprises the amino acid sequence starting at residue N439 and ending at residue T500 of the spike protein of SARS CoV2 as denoted by SEQ ID NO: 6, or any homologs and variants thereof. In some embodiments, the native RBM comprises the amino acid sequence starting at residue N439 and ending at residue N501 of the spike protein of SARS CoV2 as denoted by SEQ ID NO: 6, or any homologs and variants thereof. In some embodiments, the native RBM comprises the amino acid sequence starting at residue N439 and ending at residue G502 of the spike protein of SARS CoV2 as denoted by SEQ ID NO: 6, or any homologs and variants thereof. In some embodiments, the native RBM comprises the amino acid sequence starting at residue N439 and ending at residue V503 of the spike protein of SARS CoV2 as denoted by SEQ ID NO: 6, or any homologs and variants thereof. In some embodiments, the native RBM comprises the amino acid sequence starting at residue N439 and ending at residue G504 of the spike protein of SARS CoV2 as denoted by SEQ ID NO: 6, or any homologs and variants thereof. In some embodiments, the native RBM comprises the amino acid sequence starting at residue N439 and ending at residue Y505 of the spike protein of SARS CoV2 as denoted by SEQ ID NO: 6, or any homologs and variants thereof. In some embodiments, the native RBM comprises the amino acid sequence starting at residue N439 and ending at residue Q506 of the spike protein of SARS CoV2 as denoted by SEQ ID NO: 6, or any homologs and variants thereof. In some embodiments, the native RBM comprises the amino acid sequence starting at residue N439 and ending at residue P507 of the spike protein of SARS CoV2 as denoted by SEQ ID NO: 6, or any homologs and variants thereof. In some embodiments, the native RBM comprises the amino acid sequence starting at residue N439 and ending at residue Y508 of the spike protein of SARS CoV2 as denoted by SEQ ID NO: 6, or any homologs and variants thereof. In some embodiments, the native RBM comprises the amino acid sequence starting at residue N439 and ending at residue R509 of the spike protein of SARS CoV2 as denoted by SEQ ID NO: 6, or any homologs and variants thereof. In some embodiments, the native RBM comprises the amino acid sequence starting at residue N439 and ending at residue V510 of the spike protein of SARS CoV2 as denoted by SEQ ID NO: 6, or any homologs and variants thereof. In some embodiments, the native RBM comprises the amino acid sequence starting at residue N440 and ending at residue P499 of the spike protein of SARS CoV2 as denoted by SEQ ID NO: 6, or any homologs and variants thereof. In some embodiments, the native RBM comprises the amino acid sequence starting at residue N440 and ending at residue Y495 of the spike protein of SARS CoV2 as denoted by SEQ ID NO: 6, or any homologs and variants thereof. In some embodiments, the native RBM comprises the amino acid sequence starting at residue N440 and ending at residue G496 of the spike protein of SARS CoV2 as denoted by SEQ ID NO: 6, or any homologs and variants thereof. In some embodiments, the native RBM comprises the amino acid sequence starting at residue N440 and ending at residue F497 of the spike protein of SARS CoV2 as denoted by SEQ ID NO: 6, or any homologs and variants thereof. In some embodiments, the native RBM comprises the amino acid sequence starting at residue N440 and ending at residue Q498 of the spike protein of SARS CoV2 as denoted by SEQ ID NO: 6, or any homologs and variants thereof. In some embodiments, the native RBM comprises the amino acid sequence starting at residue N440 and ending at residue T500 of the spike protein of SARS CoV2 as denoted by SEQ ID NO: 6, or any homologs and variants thereof. In some embodiments, the native RBM comprises the amino acid sequence starting at residue N440 and ending at residue N501 of the spike protein of SARS CoV2 as denoted by SEQ ID NO: 6, or any homologs and variants thereof. In some embodiments, the native RBM comprises the amino acid sequence starting at residue N440 and ending at residue G502 of the spike protein of SARS CoV2 as denoted by SEQ ID NO: 6, or any homologs and variants thereof. In some embodiments, the native RBM comprises the amino acid sequence starting at residue N440 and ending at residue V503 of the spike protein of SARS CoV2 as denoted by SEQ ID NO: 6, or any homologs and variants thereof.

In some embodiments, the native RBM comprises the amino acid sequence starting at residue N440 and ending at residue G504 of the spike protein of SARS CoV2 as denoted by SEQ ID NO: 6, or any homologs and variants thereof. In some embodiments, the native RBM comprises the amino acid sequence starting at residue N440 and ending at residue Y505 of the spike protein of SARS CoV2 as denoted by SEQ ID NO: 6, or any homologs and variants thereof. In some embodiments, the native RBM comprises the amino acid sequence starting at residue N440 and ending at residue Q506 of the spike protein of SARS CoV2 as denoted by SEQ ID NO: 6, or any homologs and variants thereof. In some embodiments, the native RBM comprises the amino acid sequence starting at residue N440 and ending at residue P507 of the spike protein of SARS CoV2 as denoted by SEQ ID NO: 6, or any homologs and variants thereof. In some embodiments, the native RBM comprises the amino acid sequence starting at residue N440 and ending at residue Y508 of the spike protein of SARS CoV2 as denoted by SEQ ID NO: 6, or any homologs and variants thereof. In some embodiments, the native RBM comprises the amino acid sequence starting at residue N440 and ending at residue R509 of the spike protein of SARS CoV2 as denoted by SEQ ID NO: 6, or any homologs and variants thereof. In some embodiments, the native RBM comprises the amino acid sequence starting at residue N440 and ending at residue V510 of the spike protein of SARS CoV2 as denoted by SEQ ID NO: 6, or any homologs and variants thereof. In some embodiments, the native RBM comprises the amino acid sequence starting at residue L441 and ending at residue P499 of the spike protein of SARS CoV2 as denoted by SEQ ID NO: 6, or any homologs and variants thereof. In some embodiments, the native RBM comprises the amino acid sequence starting at residue L441 and ending at residue Y495 of the spike protein of SARS CoV2 as denoted by SEQ ID NO: 6, or any homologs and variants thereof. In some embodiments, the native RBM comprises the amino acid sequence starting at residue L441 and ending at residue G496 of the spike protein of SARS CoV2 as denoted by SEQ ID NO: 6, or any homologs and variants thereof. In some embodiments, the native RBM comprises the amino acid sequence starting at residue L441 and ending at residue F497 of the spike protein of SARS CoV2 as denoted by SEQ ID NO: 6, or any homologs and variants thereof. In some embodiments, the native RBM comprises the amino acid sequence starting at residue L441 and ending at residue Q498 of the spike protein of SARS CoV2 as denoted by SEQ ID NO: 6, or any homologs and variants thereof. In some embodiments, the native RBM comprises the amino acid sequence starting at residue L441 and ending at residue T500 of the spike protein of SARS CoV2 as denoted by SEQ ID NO: 6, or any homologs and variants thereof. In some embodiments, the native RBM comprises the amino acid sequence starting at residue L441 and ending at residue N501 of the spike protein of SARS CoV2 as denoted by SEQ ID NO: 6, or any homologs and variants thereof. In some embodiments, the native RBM comprises the amino acid sequence starting at residue L441 and ending at residue G502 of the spike protein of SARS CoV2 as denoted by SEQ ID NO: 6, or any homologs and variants thereof. In some embodiments, the native RBM comprises the amino acid sequence starting at residue L441 and ending at residue V503 of the spike protein of SARS CoV2 as denoted by SEQ ID NO: 6, or any homologs and variants thereof. In some embodiments, the native RBM comprises the amino acid sequence starting at residue L441 and ending at residue G504 of the spike protein of SARS CoV2 as denoted by SEQ ID NO: 6, or any homologs and variants thereof. In some embodiments, the native RBM comprises the amino acid sequence starting at residue L441 and ending at residue Y505 of the spike protein of SARS CoV2 as denoted by SEQ ID NO: 6, or any homologs and variants thereof. In some embodiments, the native RBM comprises the amino acid sequence starting at residue L441 and ending at residue Q506 of the spike protein of SARS CoV2 as denoted by SEQ ID NO: 6, or any homologs and variants thereof. In some embodiments, the native RBM comprises the amino acid sequence starting at residue L441 and ending at residue P507 of the spike protein of SARS CoV2 as denoted by SEQ ID NO: 6, or any homologs and variants thereof. In some embodiments, the native RBM comprises the amino acid sequence starting at residue L441 and ending at residue Y508 of the spike protein of SARS CoV2 as denoted by SEQ ID NO: 6, or any homologs and variants thereof. In some embodiments, the native RBM comprises the amino acid sequence starting at residue L441 and ending at residue R509 of the spike protein of SARS CoV2 as denoted by SEQ ID NO: 6, or any homologs and variants thereof. In some embodiments, the native RBM comprises the amino acid sequence starting at residue L441 and ending at residue V510 of the spike protein of SARS CoV2 as denoted by SEQ ID NO: 6, or any homologs and variants thereof.

In some embodiments, the native RBM comprises the amino acid sequence starting at residue D442 and ending at residue P499 of the spike protein of SARS CoV2 as denoted by SEQ ID NO: 6, or any homologs and variants thereof. In some embodiments, the native RBM comprises the amino acid sequence starting at residue D442 and ending at residue Y495 of the spike protein of SARS CoV2 as denoted by SEQ ID NO: 6, or any homologs and variants thereof. In some embodiments, the native RBM comprises the amino acid sequence starting at residue D442 and ending at residue G496 of the spike protein of SARS CoV2 as denoted by SEQ ID NO: 6, or any homologs and variants thereof. In some embodiments, the native RBM comprises the amino acid sequence starting at residue D442 and ending at residue F497 of the spike protein of SARS CoV2 as denoted by SEQ ID NO: 6, or any homologs and variants thereof. In some embodiments, the native RBM comprises the amino acid sequence starting at residue D442 and ending at residue Q498 of the spike protein of SARS CoV2 as denoted by SEQ ID NO: 6, or any homologs and variants thereof. In some embodiments, the native RBM comprises the amino acid sequence starting at residue D442 and ending at residue T500 of the spike protein of SARS CoV2 as denoted by SEQ ID NO: 6, or any homologs and variants thereof. In some embodiments, the native RBM comprises the amino acid sequence starting at residue D442 and ending at residue N501 of the spike protein of SARS CoV2 as denoted by SEQ ID NO: 6, or any homologs and variants thereof. In some embodiments, the native RBM comprises the amino acid sequence starting at residue D442 and ending at residue G502 of the spike protein of SARS CoV2 as denoted by SEQ ID NO: 6, or any homologs and variants thereof. In some embodiments, the native RBM comprises the amino acid sequence starting at residue D442 and ending at residue V503 of the spike protein of SARS CoV2 as denoted by SEQ ID NO: 6, or any homologs and variants thereof. In some embodiments, the native RBM comprises the amino acid sequence starting at residue D442 and ending at residue G504 of the spike protein of SARS CoV2 as denoted by SEQ ID NO: 6, or any homologs and variants thereof. In some embodiments, the native RBM comprises the amino acid sequence starting at residue D442 and ending at residue Y505 of the spike protein of SARS CoV2 as denoted by SEQ ID NO: 6, or any homologs and variants thereof. In some embodiments, the native RBM comprises the amino acid sequence starting at residue D442 and ending at residue Q506 of the spike protein of SARS CoV2 as denoted by SEQ ID NO: 6, or any homologs and variants thereof. In some embodiments, the native RBM comprises the amino acid sequence starting at residue D442 and ending at residue P507 of the spike protein of SARS CoV2 as denoted by SEQ ID NO: 6, or any homologs and variants thereof. In some embodiments, the native RBM comprises the amino acid sequence starting at residue D442 and ending at residue Y508 of the spike protein of SARS CoV2 as denoted by SEQ ID NO: 6, or any homologs and variants thereof. In some embodiments, the native RBM comprises the amino acid sequence starting at residue D442 and ending at residue R509 of the spike protein of SARS CoV2 as denoted by SEQ ID NO: 6, or any homologs and variants thereof. In some embodiments, the native RBM comprises the amino acid sequence starting at residue D442 and ending at residue V510 of the spike protein of SARS CoV2 as denoted by SEQ ID NO: 6, or any homologs and variants thereof.

In some embodiments, the native RBM comprises the amino acid sequence starting at residue K444 and ending at residue P499 of the spike protein of SARS CoV2 as denoted by SEQ ID NO: 6, or any homologs and variants thereof. In some embodiments, the native RBM comprises the amino acid sequence starting at residue K444 and ending at residue Y495 of the spike protein of SARS CoV2 as denoted by SEQ ID NO: 6, or any homologs and variants thereof. In some embodiments, the native RBM comprises the amino acid sequence starting at residue K444 and ending at residue G496 of the spike protein of SARS CoV2 as denoted by SEQ ID NO: 6, or any homologs and variants thereof. In some embodiments, the native RBM comprises the amino acid sequence starting at residue K444 and ending at residue F497 of the spike protein of SARS CoV2 as denoted by SEQ ID NO: 6, or any homologs and variants thereof. In some embodiments, the native RBM comprises the amino acid sequence starting at residue K444 and ending at residue Q498 of the spike protein of SARS CoV2 as denoted by SEQ ID NO: 6, or any homologs and variants thereof. In some embodiments, the native RBM comprises the amino acid sequence starting at residue K444 and ending at residue T500 of the spike protein of SARS CoV2 as denoted by SEQ ID NO: 6, or any homologs and variants thereof. In some embodiments, the native RBM comprises the amino acid sequence starting at residue K444 and ending at residue N501 of the spike protein of SARS CoV2 as denoted by SEQ ID NO: 6, or any homologs and variants thereof. In some embodiments, the native RBM comprises the amino acid sequence starting at residue K444 and ending at residue G502 of the spike protein of SARS CoV2 as denoted by SEQ ID NO: 6, or any homologs and variants thereof. In some embodiments, the native RBM comprises the amino acid sequence starting at residue K444 and ending at residue V503 of the spike protein of SARS CoV2 as denoted by SEQ ID NO: 6, or any homologs and variants thereof. In some embodiments, the native RBM comprises the amino acid sequence starting at residue K444 and ending at residue G504 of the spike protein of SARS CoV2 as denoted by SEQ ID NO: 6, or any homologs and variants thereof. In some embodiments, the native RBM comprises the amino acid sequence starting at residue K444 and ending at residue Y505 of the spike protein of SARS CoV2 as denoted by SEQ ID NO: 6, or any homologs and variants thereof. In some embodiments, the native RBM comprises the amino acid sequence starting at residue K444 and ending at residue Q506 of the spike protein of SARS CoV2 as denoted by SEQ ID NO: 6, or any homologs and variants thereof. In some embodiments, the native RBM comprises the amino acid sequence starting at residue K444 and ending at residue P507 of the spike protein of SARS CoV2 as denoted by SEQ ID NO: 6, or any homologs and variants thereof. In some embodiments, the native RBM comprises the amino acid sequence starting at residue K444 and ending at residue Y508 of the spike protein of SARS CoV2 as denoted by SEQ ID NO: 6, or any homologs and variants thereof. In some embodiments, the native RBM comprises the amino acid sequence starting at residue K444 and ending at residue R509 of the spike protein of SARS CoV2 as denoted by SEQ ID NO: 6, or any homologs and variants thereof. In some embodiments, the native RBM comprises the amino acid sequence starting at residue K444 and ending at residue V510 of the spike protein of SARS CoV2 as denoted by SEQ ID NO: 6, or any homologs and variants thereof. In some embodiments, the native RBM comprises the amino acid sequence starting at residue V445 and ending at residue P499 of the spike protein of SARS CoV2 as denoted by SEQ ID NO: 6, or any homologs and variants thereof. In some embodiments, the native RBM comprises the amino acid sequence starting at residue V445 and ending at residue Y495 of the spike protein of SARS CoV2 as denoted by SEQ ID NO: 6, or any homologs and variants thereof. In some embodiments, the native RBM comprises the amino acid sequence starting at residue V445 and ending at residue G496 of the spike protein of SARS CoV2 as denoted by SEQ ID NO: 6, or any homologs and variants thereof. In some embodiments, the native RBM comprises the amino acid sequence starting at residue V445 and ending at residue F497 of the spike protein of SARS CoV2 as denoted by SEQ ID NO: 6, or any homologs and variants thereof. In some embodiments, the native RBM comprises the amino acid sequence starting at residue V445 and ending at residue Q498 of the spike protein of SARS CoV2 as denoted by SEQ ID NO: 6, or any homologs and variants thereof. In some embodiments, the native RBM comprises the amino acid sequence starting at residue V445 and ending at residue T500 of the spike protein of SARS CoV2 as denoted by SEQ ID NO: 6, or any homologs and variants thereof. In some embodiments, the native RBM comprises the amino acid sequence starting at residue V445 and ending at residue N501 of the spike protein of SARS CoV2 as denoted by SEQ ID NO: 6, or any homologs and variants thereof. In some embodiments, the native RBM comprises the amino acid sequence starting at residue V445 and ending at residue G502 of the spike protein of SARS CoV2 as denoted by SEQ ID NO: 6, or any homologs and variants thereof. In some embodiments, the native RBM comprises the amino acid sequence starting at residue V445 and ending at residue V503 of the spike protein of SARS CoV2 as denoted by SEQ ID NO: 6, or any homologs and variants thereof. In some embodiments, the native RBM comprises the amino acid sequence starting at residue V445 and ending at residue G504 of the spike protein of SARS CoV2 as denoted by SEQ ID NO: 6, or any homologs and variants thereof. In some embodiments, the native RBM comprises the amino acid sequence starting at residue V445 and ending at residue Y505 of the spike protein of SARS CoV2 as denoted by SEQ ID NO: 6, or any homologs and variants thereof. In some embodiments, the native RBM comprises the amino acid sequence starting at residue V445 and ending at residue Q506 of the spike protein of SARS CoV2 as denoted by SEQ ID NO: 6, or any homologs and variants thereof. In some embodiments, the native RBM comprises the amino acid sequence starting at residue V445 and ending at residue P507 of the spike protein of SARS CoV2 as denoted by SEQ ID NO: 6, or any homologs and variants thereof. In some embodiments, the native RBM comprises the amino acid sequence starting at residue V445 and ending at residue Y508 of the spike protein of SARS CoV2 as denoted by SEQ ID NO: 6, or any homologs and variants thereof. In some embodiments, the native RBM comprises the amino acid sequence starting at residue V445 and ending at residue R509 of the spike protein of SARS CoV2 as denoted by SEQ ID NO: 6, or any homologs and variants thereof. In some embodiments, the native RBM comprises the amino acid sequence starting at residue V445 and ending at residue V510 of the spike protein of SARS CoV2 as denoted by SEQ ID NO: 6, or any homologs and variants thereof. In some embodiments, the native RBM comprises the amino acid sequence starting at residue G446 and ending at residue P499 of the spike protein of SARS CoV2 as denoted by SEQ ID NO: 6, or any homologs and variants thereof. In some embodiments, the native RBM comprises the amino acid sequence starting at residue G446 and ending at residue Y495 of the spike protein of SARS CoV2 as denoted by SEQ ID NO: 6, or any homologs and variants thereof. In some embodiments, the native RBM comprises the amino acid sequence starting at residue G446 and ending at residue G496 of the spike protein of SARS CoV2 as denoted by SEQ ID NO: 6, or any homologs and variants thereof. In some embodiments, the native RBM comprises the amino acid sequence starting at residue G446 and ending at residue F497 of the spike protein of SARS CoV2 as denoted by SEQ ID NO: 6, or any homologs and variants thereof. In some embodiments, the native RBM comprises the amino acid sequence starting at residue G446 and ending at residue Q498 of the spike protein of SARS CoV2 as denoted by SEQ ID NO: 6, or any homologs and variants thereof. In some embodiments, the native RBM comprises the amino acid sequence starting at residue G446 and ending at residue T500 of the spike protein of SARS CoV2 as denoted by SEQ ID NO: 6, or any homologs and variants thereof. In some embodiments, the native RBM comprises the amino acid sequence starting at residue G446 and ending at residue N501 of the spike protein of SARS CoV2 as denoted by SEQ ID NO: 6, or any homologs and variants thereof. In some embodiments, the native RBM comprises the amino acid sequence starting at residue G446 and ending at residue G502 of the spike protein of SARS CoV2 as denoted by SEQ ID NO: 6, or any homologs and variants thereof. In some embodiments, the native RBM comprises the amino acid sequence starting at residue G446 and ending at residue V503 of the spike protein of SARS CoV2 as denoted by SEQ ID NO: 6, or any homologs and variants thereof. In some embodiments, the native RBM comprises the amino acid sequence starting at residue G446 and ending at residue G504 of the spike protein of SARS CoV2 as denoted by SEQ ID NO: 6, or any homologs and variants thereof. In some embodiments, the native RBM comprises the amino acid sequence starting at residue G446 and ending at residue Y505 of the spike protein of SARS CoV2 as denoted by SEQ ID NO: 6, or any homologs and variants thereof. In some embodiments, the native RBM comprises the amino acid sequence starting at residue G446 and ending at residue Q506 of the spike protein of SARS CoV2 as denoted by SEQ ID NO: 6, or any homologs and variants thereof. In some embodiments, the native RBM comprises the amino acid sequence starting at residue G446 and ending at residue P507 of the spike protein of SARS CoV2 as denoted by SEQ ID NO: 6, or any homologs and variants thereof. In some embodiments, the native RBM comprises the amino acid sequence starting at residue G446 and ending at residue Y508 of the spike protein of SARS CoV2 as denoted by SEQ ID NO: 6, or any homologs and variants thereof. In some embodiments, the native RBM comprises the amino acid sequence starting at residue G446 and ending at residue R509 of the spike protein of SARS CoV2 as denoted by SEQ ID NO: 6, or any homologs and variants thereof. In some embodiments, the native RBM comprises the amino acid sequence starting at residue G446 and ending at residue V510 of the spike protein of SARS CoV2 as denoted by SEQ ID NO: 6, or any homologs and variants thereof. In some embodiments, the native RBM comprises the amino acid sequence starting at residue G447 and ending at residue P499 of the spike protein of SARS CoV2 as denoted by SEQ ID NO: 6, or any homologs and variants thereof. In some embodiments, the native RBM comprises the amino acid sequence starting at residue G447 and ending at residue Y495 of the spike protein of SARS CoV2 as denoted by SEQ ID NO: 6, or any homologs and variants thereof. In some embodiments, the native RBM comprises the amino acid sequence starting at residue G447 and ending at residue G496 of the spike protein of SARS CoV2 as denoted by SEQ ID NO: 6, or any homologs and variants thereof. In some embodiments, the native RBM comprises the amino acid sequence starting at residue G447 and ending at residue F497 of the spike protein of SARS CoV2 as denoted by SEQ ID NO: 6, or any homologs and variants thereof. In some embodiments, the native RBM comprises the amino acid sequence starting at residue G447 and ending at residue Q498 of the spike protein of SARS CoV2 as denoted by SEQ ID NO: 6, or any homologs and variants thereof. In some embodiments, the native RBM comprises the amino acid sequence starting at residue G447 and ending at residue T500 of the spike protein of SARS CoV2 as denoted by SEQ ID NO: 6, or any homologs and variants thereof. In some embodiments, the native RBM comprises the amino acid sequence starting at residue G447 and ending at residue N501 of the spike protein of SARS CoV2 as denoted by SEQ ID NO: 6, or any homologs and variants thereof. In some embodiments, the native RBM comprises the amino acid sequence starting at residue G447 and ending at residue G502 of the spike protein of SARS CoV2 as denoted by SEQ ID NO: 6, or any homologs and variants thereof.

In some embodiments, the native RBM comprises the amino acid sequence starting at residue G447 and ending at residue V503 of the spike protein of SARS CoV2 as denoted by SEQ ID NO: 6, or any homologs and variants thereof. In some embodiments, the native RBM comprises the amino acid sequence starting at residue G447 and ending at residue G504 of the spike protein of SARS CoV2 as denoted by SEQ ID NO: 6, or any homologs and variants thereof. In some embodiments, the native RBM comprises the amino acid sequence starting at residue G447 and ending at residue Y505 of the spike protein of SARS CoV2 as denoted by SEQ ID NO: 6, or any homologs and variants thereof. In some embodiments, the native RBM comprises the amino acid sequence starting at residue G447 and ending at residue Q506 of the spike protein of SARS CoV2 as denoted by SEQ ID NO: 6, or any homologs and variants thereof. In some embodiments, the native RBM comprises the amino acid sequence starting at residue G447 and ending at residue P507 of the spike protein of SARS CoV2 as denoted by SEQ ID NO: 6, or any homologs and variants thereof. In some embodiments, the native RBM comprises the amino acid sequence starting at residue G447 and ending at residue Y508 of the spike protein of SARS CoV2 as denoted by SEQ ID NO: 6, or any homologs and variants thereof. In some embodiments, the native RBM comprises the amino acid sequence starting at residue G447 and ending at residue R509 of the spike protein of SARS CoV2 as denoted by SEQ ID NO: 6, or any homologs and variants thereof. In some embodiments, the native RBM comprises the amino acid sequence starting at residue G447 and ending at residue V510 of the spike protein of SARS CoV2 as denoted by SEQ ID NO: 6, or any homologs and variants thereof. In some embodiments, the native RBM comprises the amino acid sequence starting at residue N448 and ending at residue P499 of the spike protein of SARS CoV2 as denoted by SEQ ID NO: 6, or any homologs and variants thereof. In some embodiments, the native RBM comprises the amino acid sequence starting at residue N448 and ending at residue Y495 of the spike protein of SARS CoV2 as denoted by SEQ ID NO: 6, or any homologs and variants thereof. In some embodiments, the native RBM comprises the amino acid sequence starting at residue N448 and ending at residue G496 of the spike protein of SARS CoV2 as denoted by SEQ ID NO: 6, or any homologs and variants thereof. In some embodiments, the native RBM comprises the amino acid sequence starting at residue N448 and ending at residue F497 of the spike protein of SARS CoV2 as denoted by SEQ ID NO: 6, or any homologs and variants thereof. In some embodiments, the native RBM comprises the amino acid sequence starting at residue N448 and ending at residue Q498 of the spike protein of SARS CoV2 as denoted by SEQ ID NO: 6, or any homologs and variants thereof. In some embodiments, the native RBM comprises the amino acid sequence starting at residue N448 and ending at residue T500 of the spike protein of SARS CoV2 as denoted by SEQ ID NO: 6, or any homologs and variants thereof. In some embodiments, the native RBM comprises the amino acid sequence starting at residue N448 and ending at residue N501 of the spike protein of SARS CoV2 as denoted by SEQ ID NO: 6, or any homologs and variants thereof. In some embodiments, the native RBM comprises the amino acid sequence starting at residue N448 and ending at residue G502 of the spike protein of SARS CoV2 as denoted by SEQ ID NO: 6, or any homologs and variants thereof. In some embodiments, the native RBM comprises the amino acid sequence starting at residue N448 and ending at residue V503 of the spike protein of SARS CoV2 as denoted by SEQ ID NO: 6, or any homologs and variants thereof. In some embodiments, the native RBM comprises the amino acid sequence starting at residue N448 and ending at residue G504 of the spike protein of SARS CoV2 as denoted by SEQ ID NO: 6, or any homologs and variants thereof. In some embodiments, the native RBM comprises the amino acid sequence starting at residue N448 and ending at residue Y505 of the spike protein of SARS CoV2 as denoted by SEQ ID NO: 6, or any homologs and variants thereof. In some embodiments, the native RBM comprises the amino acid sequence starting at residue N448 and ending at residue Q506 of the spike protein of SARS CoV2 as denoted by SEQ ID NO: 6, or any homologs and variants thereof. In some embodiments, the native RBM comprises the amino acid sequence starting at residue N448 and ending at residue P507 of the spike protein of SARS CoV2 as denoted by SEQ ID NO: 6, or any homologs and variants thereof. In some embodiments, the native RBM comprises the amino acid sequence starting at residue N448 and ending at residue Y508 of the spike protein of SARS CoV2 as denoted by SEQ ID NO: 6, or any homologs and variants thereof. In some embodiments, the native RBM comprises the amino acid sequence starting at residue N448 and ending at residue R509 of the spike protein of SARS CoV2 as denoted by SEQ ID NO: 6, or any homologs and variants thereof. In some embodiments, the native RBM comprises the amino acid sequence starting at residue N448 and ending at residue V510 of the spike protein of SARS CoV2 as denoted by SEQ ID NO: 6, or any homologs and variants thereof.

In some embodiments, the reconstituted RBM of the polypeptide of the invention comprises at least one linker and at least two fragments of the native RBM. In some embodiments, these two fragments are referred to herein as fragment “A” and fragment “B”. More specifically, wherein:
(a) at least one of the at least two fragments, also referred to herein in some embodiments as fragment “A”, may comprise the amino acid sequence of any one of:
(i) residues S443 to F456; (ii) residues S443 to R457; (iii) residues S443 to K458; (iv) residues S443 to R454; (v) residues S443 to L455; (vi) residues A435 to F456; (vii) residues A435 to R457; (viii) residues A435 to K458; (ix) residues A435 to R454; (x) residues A435 to L455; (xi) residues W436 to F456; (xii) residues W436 to R457; (xiii) residues W436 to K458; (xiv) residues W436 to R454; (xv) residues W436 to L455; (xvi) residues N437 to F456; (xvii) residues N437 to R457; (xviii) residues N437 to K458; (xix) residues N437 to R454; (xx) residues N437 to L455; (xxi) residues S438 to F456; (xxii) residues S438 to R457; (xxiii) residues S438 to K458; (xxiv) residues S438 to R454; (xxv) residues S438 to L455; (xxvi) residues N439 to F456; (xxvii) residues N439 to R457; (xxviii) residues N439 to K458; (xxix) residues N439 to R454; (xxx) residues N439 to L455; (xxxi) residues N440 to F456; (xxxii) residues N440 to R457; (xxxiii) residues N440 to K458; (xxxiv) residues N440 to R454; (xxxv) residues N440 to L455; (xxxvi) residues L441 to F456; (xxxvii) residues L441 to R457; (xxxviii) residues L441 to K458; (xxxix) residues L441 to R454; (xl) residues L441 to L455; (xli) residues D442 to F456; (xlii) residues D442 to R457; (xliii) residues D442 to K458; (xliv) residues D442 to R454; (xlv) residues D442 to L455; (xlvi) residues K444 to F456; (xlvii) residues K444 to R457; (xlviii) residues K444 to K458; (xlix) residues K444 to R454; (1) residues K444 to L455; (li) residues V445 to F456; (lii) residues V445 to R457; (liii) residues V445 to K458; (liv) residues V445 to R454; (lv) residues V445 to L455; (lvi) residues G446 to F456; (lvii) residues G446 to R457; (lviii) residues G446 to K458; (lix) residues G446 to R454; (lx) residues G446 to L455; (lxi) residues G447 to F456; (lxii) residues G447 to R457; (lxiii) residues G447 to K458; (lxiv) residues G447 to R454; (lxv) residues G447 to L455; (lxvi) residues N448 to F456; (lxvii) residues N448 to R457; (lxviii) residues N448 to K458; (lxix) residues N448 to R454; (lxx) residues N448 to L455; (lxxi) the amino acid sequence of residues of the SARS CoV2 spike protein as defined in any one of (i) to (lxx) with at least one or two flanking amino acid residue/s; (lxxii) any mutant, variant, parts or fragments of the amino acid sequence of residues of the SARS CoV2 spike protein as defined in any one of (i) to (lxx); and (b) at least one of the at least two fragments, also referred to herein in some embodiments as fragment “B”, may comprises the amino acid sequence of any one of:
(i) residues Y473 to Y495; (ii) residues Y473 to G496; (iii) residues Y473 to F497; (iv) residues Y473 to Q498; (v) residues Y473 to P499; (vi) residues Y473 to T500; (vii) residues Y473 to N501; (viii) residues Y473 to G502; (ix) residues Y473 to V503; (x) residues Y473 to G504; (xi) residues Y473 to Y505; (xii) residues Y473 to Q506; (xiii) residues Y473 to P507; (xiv) residues Y473 to Y508; (xv) residues Y473 to R509; (xvi) residues Y473 to V510; (xvii) residues I472 to Y495; (xviii) residues I472 to G496; (xix) residues I472 to F497; (xx) residues I472 to Q498; (xxi) residues I472 to P499; (xxii) residues I472 to T500; (xxiii) residues I472 to N501; (xxiv) residues I472 to G502; (xxv) residues I472 to V503; (xxvi) residues I472 to G504; (xxvii) residues I472 to Y505; (xxviii) residues I472 to Q506; (xxix) residues I472 to P507; (xxx) residues I472 to Y508; (xxxi) residues I472 to R509; (xxxii) residues I472 to V510; (xxxiii) residues E471 to Y495; (xxxiv) residues E471 to G496; (xxxv) residues E471 to F497; (xxxvi) residues E471 to Q498; (xxxvii) residues E471 to P499; (xxxviii) residues E471 to T500; (xxxix) residues E471 to N501; (xl) residues E471 to G502; (xli) residues E471 to V503; (xlii) residues E471 to G504; (xliii) residues E471 to Y505; (xliv) residues E471 to Q506; (xlv) residues E471 to P507; (xlvi) residues E471 to Y508; (xlvii) residues E471 to R509; (xlviii) residues E471 to V510; (xlix) residues T470 to Y495; (1) residues T470 to G496; (li) residues T470 to F497; (lii) residues T470 to Q498; (liii) residues T470 to P499; (liv) (iv) residues T470 to T500; (lv) (v) residues T470 to N501; (lvi) residues T470 to G502; (lvii) residues T470 to V503; (lviii) residues T470 to G504; (lix) residues T470 to Y505; (lx) residues T470 to Q506; (lxi) residues T470 to P507; (lxii) residues T470 to Y508; (lxiii) residues T470 to R509; (lxiv) residues T470 to V510; (lxv) residues S469 to Y495; (lxvi) residues S469 to G496; (lxvii) residues S469 to F497; (lxviii) residues S469 to Q498; (lxix) residues S469 to P499; (lxx) residues S469 to T500; (lxxi) residues S469 to N501; (lxxii) residues S469 to G502; (lxxiii) residues S469 to V503; (lxxiv) residues S469 to G504; (lxxv) residues S469 to Y505; (lxxvi) residues S469 to Q506; (lxxvii) residues S469 to P507; (lxxviii) residues S469 to Y508; (lxxix) residues S469 to R509; (lxxx) residues S469 to V510; (lxxxi) residues I468 to Y495; (lxxxii) residues I468 to G496; (lxxxiii) residues I468 to F497; (lxxxiv) residues I468 to Q498; (lxxxv) residues I468 to P499; (lxxxvi) residues I468 to T500; (lxxxvii) residues I468 to N501; (lxxxviii) residues I468 to G502; (lxxxix) residues I468 to V503; (xc) residues I468 to G504; (xci) residues I468 to Y505; (xcii) residues I468 to Q506; (xciii) residues I468 to P507; (xciv) residues I468 to Y508; (xcv) residues I468 to R509; (xcvi) residues I468 to V510; (xcvii) residues Q474 to Y495; (xcviii) residues Q474 to G496; (xcix) residues Q474 to F497; (c) residues Q474 to Q498; (ci) residues Q474 to P499; (cii) residues Q474 to T500; (ciii) residues Q474 to N501; (civ) residues Q474 to G502; (cv) residues Q474 to V503; (cvi) residues Q474 to G504; (cvii) residues Q474 to Y505; (cviii) residues Q474 to Q506; (cix) residues Q474 to P507; (cx) residues Q474 to Y508; (cxi) residues Q474 to R509; (cxii) residues Q474 to V510; (cxiii) residues A475 to Y495; (cxiv) residues A475 to G496; (cxv) residues A475 to F497; (cxvi) residues A475 to Q498; (cxvii) residues A475 to P499; (cxviii) residues A475 to T500; (cxix) residues A475 to N501; (cxx) residues A475 to G502; (cxxi) residues A475 to V503; (cxxii) residues A475 to G504; (cxxiii) residues A475 to Y505; (cxxiv) residues A475 to Q506; (cxxv) residues A475 to P507; (cxxvi) residues A475 to Y508; (cxxvii) residues A475 to R509; (cxxviii) residues A475 to V510; (cxxix) residues G476 to Y495; (cxxx) residues G476 to G496; (cxxxi) residues G476 to F497; (cxxxii) residues G476 to Q498; (cxxxiii) residues G476 to P499; (cxxxiv) residues G476 to T500; (cxxxv) residues G476 to N501; (cxxxvi) residues G476 to G502; (cxxxvii) residues G476 to V503; (cxxxviii) residues G476 to G504; (cxxxix) residues G476 to Y505; (cxl) residues G476 to Q506; (cxli) residues G476 to P507; (cxlii) residues G476 to Y508; (cxliii) residues G476 to R509; (cxliv) residues G476 to V510; (cxlv) the amino acid sequence of residues of the SARS CoV2 Spike protein as defined in any one of (i) to (cxliv) with at least one or two flanking amino acid residue/s; (cxlvi) any variants, mutants, parts or fragments of the amino acid sequence of residues of the SARS CoV2 Spike protein as defined in any one of (i) to (cxliv). It should be understood that in some specific and non-limiting embodiments, the amino acid residues and positions disclosed herein for fragments “A” and “B”, may refer to the spike protein of SARS CoV2. In some embodiments, the spike protein may comprise the amino acid sequence as denoted by SEQ ID NO: 6, or any variants and mutants thereof. In some particular and non-limiting embodiments, variants and/or mutants as used herein, refer to a spike protein, for example, the spike protein comprising the amino acid sequence as denoted by SEQ ID NO: 6, with at least one of the following substitutions Y489N, Q474R, V483E, L492I, N501Y, K417N, E484K, L452R, Y453F, S13I, L18F, T20N, P26S, H69-V70 deletion, D80A, D138Y, Y144/145 deletions, W152C, R190S, D215G, L242/A243/L244 deletion, R246I, A570D, D614G, H655Y, P681H, I692V, A701V, T716I, S982A, T1027I, D1118H, V1176F, M1229I, or any combinations thereof. Non-limiting embodiments for such variants are denoted by SEQ ID NO: 38, 39 and 41.

In more specific embodiments, the native RBM comprises an amino acid sequence of any one of: (a) residues S443 to P499 of the SARS CoV2 Spike protein; (b) residues S443 to P499 of the SARS CoV2 Spike protein with at least one to twenty flanking amino acid residue/s; and (c) any variant, mutant, parts or fragments of the amino acid sequence of residues S443 to P499 of the SARS CoV2 Spike protein. Still further, the anchor loop of the native RBM comprises the amino acid sequence of any one of (d) residues R457 to I472 of the SARS CoV2 Spike protein; (e) residues R457 to I472 of the SARS CoV2 Spike protein with at least one or two flanking amino acid residue/s; and (f) any variant, mutant, parts or fragments of the amino acid sequence of residues R457 to I472 of the SARS CoV2 Spike protein. As indicated above, the native RBM or any variants thereof, in accordance with some embodiments of the present disclosure may comprise residues S443 to P499, optionally, with one or more flanking residues, specifically, one to twenty flanking residues that may e attached to each end of the RBM, the N′ and/or C′ termini thereof. More specifically, one, two, there, four, five, six, seven, eight, nine, ten, eleven, twelve, thirteen, fourteen, fifteen, sixteen, seventeen, eighteen, nineteen, twenty or more residues from either the N′ terminus and/or the C′ terminus of the RBM as discussed herein. In some embodiments, the RBM of the present disclosure comprises S443 to P499, as denoted by SEQ ID NO: 2, and any variants thereof, for example, any one of substitutions Y489N, Q474R, V483E, L492I, N501Y, E484K, L452R, Y453F, and any combinations thereof.

In yet some further embodiments, the reconstituted RBM of the polypeptide of the invention comprises at least one linker and at least two fragments of the native RBM. In some embodiments, the at least two fragments comprise:

A first fragment (a), also referred to herein in some embodiments as fragment “A”, that comprise the amino acid sequence of any one of:
(i) residues S443 to F456 of the SARS CoV2 Spike protein; (ii) residues S443 to F456 of the SARS CoV2 Spike protein with at least one to eight flanking amino acid residue/s; or (iii) any variants, mutants, parts or fragments of the amino acid sequence of residues S443 to F456 of the SARS CoV2 Spike protein; and

A second fragment (b), also referred to herein in some embodiments as fragment “B”, that comprise the amino acid sequence of any one of:
(i) residues Y473 to P499 of the SARS CoV2 Spike protein;
(ii) residues Y473 to P499 of the SARS CoV2 Spike protein with at least one to eleven flanking amino acid residue/s; or
(iii) any variants, mutants, parts or fragments of the amino acid sequence of residues Y473 to P499 of the SARS CoV2 Spike protein. It should be understood that in some specific and non-limiting embodiments, the amino acid residues and positions disclosed herein for fragments “A” and “B”, may refer to the spike protein of SARS CoV2. In some embodiments, the spike protein may comprise the amino acid sequence as denoted by SEQ ID NO: 6, or any variants and mutants thereof. In some particular and non-limiting embodiments, variants and/or mutants as used herein, refer to a spike protein, for example, the spike protein comprising the amino acid sequence as denoted by SEQ ID NO: 6, with at least one of the following substitutions Y489N, Q474R, V483E, Q474R, L492I, N501Y, E484K, L452R, Y453F or any combinations thereof. Non-limiting embodiments for such variants are denoted by SEQ ID NO: 38, 39 and 41. In some particular and non-limiting embodiments, fragment “A” comprises the S443 to F456 of the SARS CoV2 Spike protein, as denoted by SEQ ID NO: 3, and any variants and mutants thereof, as discussed above. In yet some further embodiments, fragment “B” comprises the Y473 to P499 of the SARS CoV2 Spike protein, as denoted by SEQ ID NO: 4, and any variants and mutants thereof, as discussed above. In some embodiments, at least one linker that is a linker that replaces the anchor loop or any part thereof, or amino acid residue/s thereof, is a bridging linker. Such bridging linker bridges and the last amino acid residue of the N′ terminal fragment (also referred to herein as the first fragment or as fragment (A)) of the reconstituted RBM, with the first amino acid residue of the C′ terminal fragment (also referred to herein as the second fragment or as fragment (B)) of the reconstituted RBM. In some embodiments the bridging linker bridges the amino acid residues that flank the replaced anchor loop or any part or amino acid residue/s thereof. In yet some further embodiments, the linker may comprise any compound bridging the two fragments of the reconstituted RBM. In some further embodiments, the linker is an inert linker. In yet some further embodiments, such linker is any inorganic or organic molecule, any small molecule, any peptide (L- as well as D-aa residues), or any combinations thereof. The at least one linker used for the reconstituted RBM of the polypeptide of the invention may be a bridging linker that bridges residue that flank the anchor loop removed by the invention. Such bridging linker, also referred to herein as a τ-linker (tau linker) may bridge any one of the amino acid residues F456, R457, K458, R454 or L455 with any one of the amino acid residues Y473, I472, E471, T470, S469, I468, Q474, A475 or G476 of the SARS CoV2 Spike protein.

In some embodiments, the bridging linker may bridge the amino acid residue F456 with Y473. In some embodiments, the bridging linker may bridge the amino acid residue F456 with I472. In some embodiments, the bridging linker may bridge the amino acid residue F456 with E471. In some embodiments, the bridging linker may bridge the amino acid residue F456 with T470. In some alternative embodiments, the bridging linker may bridge the amino acid residue F456 with S469. In some further embodiments, the bridging linker may bridge the amino acid residue F456 with I468. In some embodiments, the bridging linker may bridge the amino acid residue F456 with Q474. In some embodiments, the bridging linker may bridge the amino acid residue F456 with A475. In some embodiments, the bridging linker may bridge the amino acid residue F456 with G476. In some embodiments, the bridging linker may bridge the amino acid residue R457 with Y473. In some embodiments, the bridging linker may bridge the amino acid residue R4576 with I472. In some embodiments, the bridging linker may bridge the amino acid residue R457 with E471. Still further, in some embodiments, the bridging linker may bridge the amino acid residue R457 with T470. In some alternative embodiments, the bridging linker may bridge the amino acid residue R457 with S469. In some further embodiments, the bridging linker may bridge the amino acid residue R457 with I468. In some embodiments, the bridging linker may bridge the amino acid residue R457 with Q474. In some embodiments, the bridging linker may bridge the amino acid residue R457 with A475. In some embodiments, the bridging linker may bridge the amino acid residue R457 with G476.

In some embodiments, the bridging linker may bridge the amino acid residue K458 with Y473. In some embodiments, the bridging linker may bridge the amino acid residue K458 with I472. In some embodiments, the bridging linker may bridge the amino acid residue K458 with E471. In some embodiments, the bridging linker may bridge the amino acid residue K458 with T470. In some alternative embodiments, the bridging linker may bridge the amino acid residue K458 with S469. In some further embodiments, the bridging linker may bridge the amino acid residue K458 with I468. In some embodiments, the bridging linker may bridge the amino acid residue K458 with Q474. In some embodiments, the bridging linker may bridge the amino acid residue K458 with A475. In some embodiments, the bridging linker may bridge the amino acid residue K458 with G476. In some embodiments, the bridging linker may bridge the amino acid residue R454 with Y473. In some embodiments, the bridging linker may bridge the amino acid residue R454 with I472. In some embodiments, the bridging linker may bridge the amino acid residue R454 with E471. In some embodiments, the bridging linker may bridge the amino acid residue R454 with T470. In some alternative embodiments, the bridging linker may bridge the amino acid residue R454 with S469. In some further embodiments, the bridging linker may bridge the amino acid residue R454 with I468. In some embodiments, the bridging linker may bridge the amino acid residue R454 with Q474. In some embodiments, the bridging linker may bridge the amino acid residue R454 with A475. In some embodiments, the bridging linker may bridge the amino acid residue R454 with G476. In some embodiments, the bridging linker may bridge the amino acid residue L455 with Y473. In some embodiments, the bridging linker may bridge the amino acid residue L455 with I472. In some embodiments, the bridging linker may bridge the amino acid residue L455 with E471. In some embodiments, the bridging linker may bridge the amino acid residue L455 with T470. In some alternative embodiments, the bridging linker may bridge the amino acid residue L455 with S469. In some further embodiments, the bridging linker may bridge the amino acid residue L455 with I468. In some embodiments, the bridging linker may bridge the amino acid residue L455 with Q474. In some embodiments, the bridging linker may bridge the amino acid residue L455 with A475. In some embodiments, the bridging linker may bridge the amino acid residue L455 with G476. In some specific embodiments, the at least one linker used for the reconstituted RBM of the polypeptide of the invention may be a bridging linker that bridges residue 456 with residue 473 of the SARS CoV2 Spike protein.

The reconstituted RBM polypeptides of the invention comprise at least one linker. In some embodiments, the linker of the invention is designated herein as τ-linker or T-linker (tau-linker). The term “linker” in the context of the invention concerns an amino acid sequence of from about 1 to about 10 or more amino acid residues positioned within and/or flanking the reconstituted RBM of the invention. The linker may be positioned in the central region of the reconstituted RBM of the invention and/or in at least one of its termini, namely at the C-terminus and/or at the N-terminus thereof. The linker is covalently linked or joined to the amino acid residues in its vicinity.

For example, a linker in accordance with the invention may be of 1, 2, 3, 4, 5, 6, 7, 8, 9 or 10 or more amino acid residues long. In yet some further embodiments, the linker/s used by the invention may be a combinatorial linker screened from a combinatorial library comprising all possible linkers composed of 1, 2, 3, 4, 5, 6, 7, 8, 9, 10 or more amino acid residues that are tested and screened for functionality, i.e., to produce a functional RBM that is an RBM that is able to functionally be bound by a receptor and/or neutralizing antibody. The term linker in accordance with the present invention encompasses any amino acid residue, as dictated by the encoding NNK nucleic acid motif. In some embodiments the linker according to the present invention encompasses 1-10, 1-9, 1-8, 1-7, 1-6, 1-5, 1-4, 1-3 or 1-2 or 1 amino acid residue/s. In other embodiments the linker encompasses 1, 2, 3, 4, 5, 6, 7, 8, 9, 10 or more amino acid residues, and thus, in certain embodiments the linkers may be referred to as NNK₁, NNK₂, NNK₃, NNK₄, NNK₅, NNK₆ NNK₇, NNK₈, NNK₉ and NNK₁₀. In some specific embodiments, for SARS CoV2 reconstituted RBM polypeptide, useful linkers may include NNK₁, NNK₂, NNK₃, NNK₄, NNK₅, NNK₆, and NNK₇. As known in the art, the term “NNK” refers to a nucleic acid triad encoding an amino acid residue, where “N” denotes any nucleotide (namely a natural or a non-natural nucleotide, e.g. nucleotides based on the DNA nucleobases cytosine (C), guanine (G), adenine (A) and thymine (T)) and where “K” denotes a nucleotide based on guanine (G) or thymine (T). However, the NNN codon is also possible. The original use of NNK is to reduce the possibility of abortive termination. Thus, the UAG codon which is possible for NNK is overcome when expressing the library in a bacterial strain that contains a suppression mutation replacing UAG for a glutamine residue (such as the SupE mutation). As detailed below, the linkers as herein defined are based on nucleotide triads of the type “NNK”. The linker, when present, may have a length of n repeats which may be the same or different one from the other. In particular the linker may include one NNK (denoted as “NNK₁”), two NNK (denoted as “NNK₂”), “NNK₃”, “NNK₄” when three or four NNKs are present, respectively, etc. Specifically, the index n may have a value of between 0 to 10. In some embodiments, the index n may have a value of between 3 to 7.

As noted above, the reconstituted RBM of the polypeptide of the invention comprises at least one linker. It should be appreciated that any linkers or any combination of linkers may be used for the polypeptide of the invention. In certain and non-limiting embodiments, an amino acid linker may be used. In some embodiments, the reconstituted RBM of the polypeptide of the invention may comprise at least one linker added to or replacing at least one of the anchor loop, any part or amino acid residue/s thereof or at least one RBM fragment or amino acid residue not directly involved with receptor and/or nAb/s interaction or binding. In some particular embodiments, the reconstituted RBM of the invention may comprise additional linker/s that may be located in other segments of the RBM. In some embodiments, the at least one linker of the invention may replace any amino acid sequence or amino acid residue/of the SARS-CoV2 RBM that comprise at least part of the anchor loop as referred to herein.

It should be noted however, that in some embodiments, the reconstituted RBM of the invention may comprise at least one linker that replaces other fragments or amino acid residues of the native RBM, specifically, at least one linker that replaces fragments or amino acid residues not involved in the receptor-virus and/or nAb/s-virus interaction. These fragments or amino acid residues may be within or of out of the loop segment. Still further embodiments encompass reconstituted RBM polypeptides, wherein at least one amino acid residue/s or fragment/s of the RBM directly or indirectly involved in receptor and/or nAb/s binding is replaced with at least one linker. In more specific embodiments the reconstituted RBM of the polypeptide of the invention may comprise an amino acid linker. In some embodiments, the linkers may comprise at least 1, 2, 3, 4, 5, 6, 7, 8, 9, 10 or more amino acid residues. In yet some more particular embodiments, such linker may comprise for example, 3, 4, 5, 6, 7, 8 or more amino acid residues.

As indicated herein, the polypeptides provided by the invention comprise reconstituted RBMs comprising at least one linker, specifically, at least one τ-linker or T-linker (tau-linker). These polypeptides thereof are referred to herein in some embodiments, as the τ-modified or T-modified (tau-modified) polypeptides of the invention. Still further, in some embodiments, the τ-modified polypeptides of the invention comprise at least one τ-modified RBM.

In yet some further specific embodiments, the at least one linker used for the RBM of the polypeptide of the invention is an amino acid linker comprising 3 to 7 amino acid residues, specifically, 3, 4, 5, 6, or 7 amino acid residues.

In some embodiments, the amino acid linker in accordance with the invention is a bridging linker comprising between about 3 to about 7 consecutive amino acid residues. In yet some further embodiments, the sequence of any of said linkers of 3, 4, 5, 6, or 7 amino acid residues, does not exist in the anchor loop of the spike protein of SARS CoV2, specifically, the anchor loop as defined by the invention. In yet some further embodiments, the sequence of any of the linkers of 3, 4, 5, 6, or 7 amino acid residues, does not exist in the anchor loop of a spike protein of any beta Corona Virus. In some embodiments, the linker used for the reconstituted RBMs of the invention is a non-natural, exogenous or non-native linker, in a sense that the linker differs from the native replaced sequence (e.g., the anchor loop), in at least one amino acid residue, at least two residues, at least three residues, at least four residues, at least five residues, at least six residues, at least seven residues, at least eight residues, at least nine residues, at least ten residues or more.

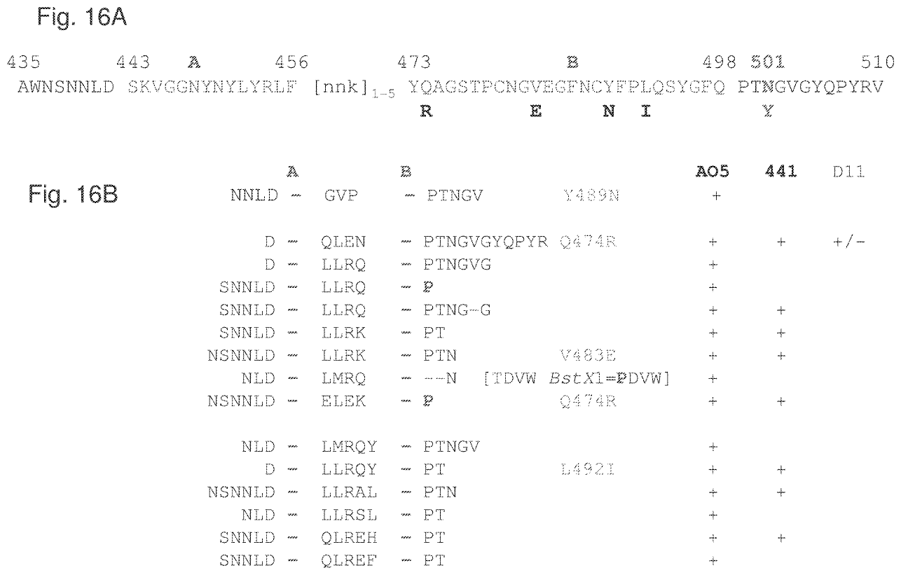

As shown by the LOGO graphical representation of the linker sequences of FIG. 17, a clear motif is apparent. Hence aliphatic methyls are preferred in the first two positions of the motif and Leucine is most prevalent. The third position is charged where arginine is most common. Positions 4 and 5 are more variable where polar residues dominate position 4 and position 5 is more hydrophobic. Thus, in some embodiments, the at least one linker used for the RBM of the polypeptide of the present disclosure comprises aliphatic methyls. In yet some further embodiments, the linkers of the present disclosure may comprise Leucine in the first two positions. Still further, in some embodiments, linkers of the present disclosure may comprise Arginine in the third position. In yet some further embodiments the likers disclosed herein may comprise Glutamine or alternatively, Glutamic acid. Still further, in some embodiments, the linker of the present invention may comprise Methionine in the second position. In the third position, the linker may comprise in some embodiments, Glutamic acid. Still further, in some embodiments, in the fourth position the linker may comprise any one of Glutamine, Lysine, Glutamic acid, Serine, Asparagine, or Alanine. In some further embodiments, the linker of the present disclosure may comprise in the fifth position Leucine, Tyrosine, Arginine, Glutamine, Histidine, Phenylalanine, and Glutamic acid. Thus, in some specific embodiments, the at least one linker used for the RBM of the polypeptide of the invention comprises the amino acid sequence of Xaa¹-Xaa²-Xaa³-Xaa₍ₙ₎ as denoted by SEQ ID NO: 57 or any fragment thereof. In some embodiments, the Xaa is any amino acid, wherein n is zero or an integer of from 1 to 4, and wherein: Xaa¹ is a Leu, Gln, Glu or Gly; Xaa² is Leu, Met or Val; and Xaa³ is Arg, Glu or Pro. In some embodiments, the linkers may be any one of an NNK₁ linker, an NNK₂ linker, an NNK₃ linker, an NNK₄ linker, an NNK₅ linker, an NNK₆ linker, an NNK₇ linker, or more. In some embodiments, the linker of the RBM of the polypeptide of the present disclosure may comprise four amino acid residues, specifically, an NNK₄ linker. In more specific embodiments, the linker of the RBM of the polypeptide of the present disclosure may comprise the amino acid sequence QLEN, as denoted by SEQ ID NO: 42.

In some other embodiments, the linker of the RBM of the polypeptide of the present disclosure may comprise the amino acid sequence LLRQ, as denoted by SEQ ID NO: 43. In some other embodiments, the linker of the RBM of the polypeptide of the present disclosure may comprise the amino acid sequence LLRK, as denoted by SEQ ID NO: 44. In some other embodiments, the linker of the RBM of the polypeptide of the present disclosure may comprise the amino acid sequence LMRQ, as denoted by SEQ ID NO: 45. In some other embodiments, the linker of the RBM of the polypeptide of the present disclosure may comprise the amino acid sequence ELEK, as denoted by SEQ ID NO: 46.

In yet some further embodiments, the linker of the RBM of the polypeptide of the present disclosure may comprise five amino acid residues, an NNK₅ linker. In some other embodiments, the linker of the RBM of the polypeptide of the present disclosure may comprise the amino acid sequence LMRQY, as denoted by SEQ ID NO: 47. In some other embodiments, the linker of the RBM of the polypeptide of the present disclosure may comprise the amino acid sequence LLRQY, as denoted by SEQ ID NO: 48. In some other embodiments, the linker of the RBM of the polypeptide of the present disclosure may comprise the amino acid sequence LLRAL, as denoted by SEQ ID NO: 49. In some other embodiments, the linker of the RBM of the polypeptide of the present disclosure may comprise the amino acid sequence LLRSL, as denoted by SEQ ID NO: 50. In some other embodiments, the linker of the RBM of the polypeptide of the present disclosure may comprise the amino acid sequence QLREH, as denoted by SEQ ID NO: 51. In some other embodiments, the linker of the RBM of the polypeptide of the present disclosure may comprise the amino acid sequence QLREF, as denoted by SEQ ID NO: 52. In yet some further embodiments, the linker of the RBM of the polypeptide of the present disclosure may comprise three amino acid residues, an NNK₃ linker. In some specific embodiments, such linker may comprise the sequence of GVP. In some specific embodiments, the polypeptides of the invention that comprise at least one reconstituted RBM as described herein, may be the Receptor Binding Domain (RBD) of the Spike protein of SARS CoV2, or any variants, mutants, parts or fragments thereof. More specifically, such RBD comprises the reconstituted RBM that comprise at least two fragments of the native RBM and at least one linker. In some specific and non-limiting embodiments such RBD may comprise residues C336 to L518 of the SARS CoV2 spike protein. In some embodiments, such spike protein may comprise the amino acid residues as denoted by SEQ ID NO: 6, or any variants or mutants thereof. In some specific and non-limiting embodiments, such variants may comprise at least one of the following substitutions Y489N, Q474R, V483E, Q474R, L492I, N501Y, K417N, E484K, or any combinations thereof. Thus, according to these embodiments, the RBD sequence disclosed herein may comprise residues C336 to L518, optionally, with one or more of the following substitutions Y489N, Q474R, V483E, Q474R, L492I, N501Y, E484K or any combinations thereof. In some embodiments, any of the RBD polypeptides may comprise the reconstituted RBM that replaces its corresponding amino acid residues. For example, such RBDs may comprise residues C336 to L518, where residues 433+/−one or two residues to 499+/−one or two residues, optionally, with one to twenty flanking residues, are replaced by any of the corresponding RBMs of the present disclosure. It should be appreciated that any of the reconstituted RBMs of the disclosure, starting at any one of S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510, of the SARS CoV2 spike protein, may replace the corresponding residues in the RBD, as disclosed herein. In yet some further embodiments, the present disclosure encompasses any RBD comprising any of the reconstituted RBMs disclosed by the present disclosure. In yet some alternative specific embodiments, the polypeptide of the invention that comprises the reconstituted RBM described herein may be the Spike protein of SARS CoV2, or any parts or fragments thereof. More specifically, the Spike protein of the invention comprises the reconstituted RBM of the invention that comprise at least two fragments of the native RBM and at least one linker. It should be noted that the spike protein provided by the invention that comprises the reconstituted RBM of the invention, encompasses in accordance with some embodiments, the S1 subunit of the spike protein. This S1 subunit comprises the reconstituted RBM of the invention as specified herein above. In yet some further embodiments, the spike protein provided herein comprises the S1 subunit and the S2 subunit. This spike protein may comprise in some embodiments the reconstituted RBM of the invention, specifically at the S1 subunit. Thus, in some embodiments, the invention provides the entire spike protein that comprises the reconstituted RBM of the invention as specified by the invention.

In some specific and non-limiting embodiments such Spike protein may comprise the amino acid residues as denoted by SEQ ID NO: 6 or any variants or mutants thereof, or alternatively, the S1 subunit of SEQ ID NO: 20, or any variants or mutants thereof. In some specific and non-limiting embodiments, such variants may comprise at least one of the following substitutions Y489N, Q474R, V483E, Q474R, L492I, N501Y, K417N, E484K, or any combinations thereof. Thus, according to these embodiments, the Spike protein or S1 subunit sequence disclosed herein may comprise the amino acid sequence of SEQ ID NO: 6 or 20, with one or more of the following substitutions Y489N, Q474R, K417N, V483E, Q474R, L492I, N501Y, E484K or any combinations thereof. In some embodiments, any of the Spike protein or S1 polypeptides may comprise the reconstituted RBM that replaces the corresponding amino acid residues within the Spike protein or S1 subunit. For example, such Spike protein or S1 subunits may comprise the amino acid sequence of SEQ ID NO: 6 or 20, where residues 433+/−one or two residues to 499+/−one or two residues, optionally, with one to twenty flanking residues, are replaced by any of the corresponding RBMs of the present disclosure. It should be appreciated that any of the reconstituted RBMs of the disclosure, starting at any one of S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510, of the SARS CoV2 spike protein, may replace the corresponding residues in the Spike protein or S1 subunit, as disclosed herein. In yet some further embodiments, the present disclosure encompasses any Spike proteins or S1 subunits comprising any of the reconstituted RBMs disclosed by the present disclosure.

As shown by the Examples, and in Example 3 and FIG. 16 in particular, screening of the combinatorial RBM libraries with several neutralizing antibodies, reveled various functional reconstituted RBMs that comprise the various linkers. In some embodiments, these reconstituted RBMs comprise residues 443 to 456 of the SARS CoV2 spike protein, referred to herein as fragment “A”, residues 773 to 499 of the SARS CoV2 spike protein, referred to herein as fragment “B”, and at least one linker that links between both fragments, and specifically, links between residue 456 to residue 473. In some embodiments, the various reconstituted RBMs comprise any of the linkers disclosed herein, for example, any of the linkers that comprise the motif of SEQ ID NO: 57. In yet some further embodiments, the various reconstituted RBMs comprise any of the linkers of SEQ ID NO: 42 to 52. Still further, as discussed herein, in some embodiments, fragment A, comprises residues 443 to 456 of the spike protein of SEQ ID. NO. 6, or any variants thereof, for example, with any substitution of at least one of Y489N, Q474R, V483E, L492I, N501Y, K417N, E484K, or any combinations thereof. In some specific embodiments, the reconstituted RBM may comprise as fragment A, the amino acid sequence as denoted by SEQ ID NO: 3, or any variants thereof. In yet some further embodiments, the reconstituted RBM may comprise as fragment B, residues 773 to 499 of SEQ ID NO: 6, or any variants thereof as discussed herein. In some embodiments, fragment B comprises the amino acid sequence as denoted by SEQ ID NO: 4, or any variants thereof (e.g., at least one of one of Y489N, Q474R, V483E, L492I, N501Y, E484K). Still further, as indicated above, in some embodiments, the reconstituted RBM may comprise fragment A extended in its N′ terminal thereof, for example, by one to eight amino acid residues. Such RBM may start at any one of residues A435, W436, N437, S438, N439, N440, L441, D442. In some specific embodiments, such reconstituted RBM may start at residue 439, adding the NNLD sequence (SEQ ID NO: 58) to fragment A. In some other specific embodiments, such reconstituted RBM may start at residue 438, adding the SNNLD sequence (SEQ ID NO: 59) to fragment A. In some further specific embodiments, such reconstituted RBM may start at residue 437, adding the NSNNLD sequence (SEQ ID NO: 60) to fragment A. In some further specific embodiments, such reconstituted RBM may start at residue 442, adding the D residue to fragment A. In some further specific embodiments, such reconstituted RBM may start at residue 440, adding the NLD sequence to fragment A.

In some specific embodiments, the reconstituted RBM may comprise fragment B, as disclosed herein (SEQ ID NO: 4, and variants thereof) extended in its C′ terminal thereof, for example, by one to eleven amino acid residues. Such RBM may end at any one of the amino acid residues T500, N501, G502, V503, G504, Y505, Q506, P507, Y508, R509 or V510. In some specific embodiments, such reconstituted RBM may end at residue 503, adding the PTNGV sequence (SEQ ID NO: 61) to fragment B. In some further embodiments, such reconstituted RBM may end at residue 509, adding the PTNGVGYQPYR sequence (SEQ ID NO: 62) to fragment B.

In some further embodiments, such reconstituted RBM may end at residue 504, adding the PTNGVG sequence (SEQ ID NO: 63) to fragment B. In some further embodiments, such reconstituted RBM may end at residue 504, adding the PTNGG sequence (SEQ ID NO: 64) to fragment B. In some further embodiments, such reconstituted RBM may end at residue 500, adding residues PT to fragment B. In some further embodiments, such reconstituted RBM may end at residue 501, adding the PTN sequence to fragment B. It should be understood that the reconstituted RBM may comprise any combination of the A fragments or any extensions and variants thereof, with any of the disclosed B fragments and any extensions and variants thereof, with any of the linkers disclosed by the invention.

Thus, in some specific and non-limiting embodiments, the reconstituted RBM may comprise residues 439 to 456, the linker GVP, and residues 473 to 503. In yet some further embodiments such reconstituted RBM may comprise the amino acid sequence NNLDSKVGGNYNYLYRLFGVPYQAGSTPCNGVEGFNCYFPLQSYGFQPTNGV, as denoted by SEQ ID NO: 65. In yet some further embodiments such reconstituted RBM may comprise, the Y489N substitution. Specifically, the amino acid sequence NNLDSKVGGNYNYLYRLFGVPYQAGSTPCNGVEGFNCNFPLQSYGFQPTNGV, as denoted by SEQ ID NO: 66. In some specific and non-limiting embodiments, the reconstituted RBM may comprise residues 442 to 456, the linker QLEN, and residues 473 to 509. In yet some further embodiments such reconstituted RBM may comprise the amino acid sequence DSKVGGNYNYLYRLFQLENYQAGSTPCNGVEGFNCYFPLQSYGFQPTNGVGYQPYR, as denoted by SEQ ID NO: 67. In yet some further embodiments such reconstituted RBM may comprise, the Q474R substitution. Specifically, the amino acid sequence DSKVGGNYNYLYRLFQLENYRAGSTPCNGVEGFNCYFPLQSYGFQPTNGVGYQPYR, as denoted by SEQ ID NO: 68. In some specific and non-limiting embodiments, the reconstituted RBM may comprise residues 442 to 456, the linker LLRQ, and residues 473 to 504. In yet some further embodiments such reconstituted RBM may comprise the amino acid sequence DSKVGGNYNYLYRLFLLRQYQAGSTPCNGVEGFNCYFPLQSYGFQPTNGVG, as denoted by SEQ ID NO: 69. In some specific and non-limiting embodiments, the reconstituted RBM may comprise residues 438 to 456, the linker LLRQ, and residues 473 to 499. In yet some further embodiments such reconstituted RBM may comprise the amino acid sequence SNNLDSKVGGNYNYLYRLFLLRQYQAGSTPCNGVEGFNCYFPLQSYGFQP, as denoted by SEQ ID NO: 70. In some specific and non-limiting embodiments, the reconstituted RBM may comprise residues 438 to 456, the linker LLRQ, and residues 473 to 504. In yet some further embodiments such reconstituted RBM may comprise the amino acid sequence SNNLDSKVGGNYNYLYRLFLLRQYQAGSTPCNGVEGFNCYFPLQSYGFQPTNGG, as denoted by SEQ ID NO: 71. In some specific and non-limiting embodiments, the reconstituted RBM may comprise residues 438 to 456, the linker LLRK, and residues 473 to 500. In yet some further embodiments such reconstituted RBM may comprise the amino acid sequence SNNLDSKVGGNYNYLYRLFLLRKYQAGSTPCNGVEGFNCYFPLQSYGFQPT, as denoted by SEQ ID NO: 72. In some specific and non-limiting embodiments, the reconstituted RBM may comprise residues 437 to 456, the linker LLRK, and residues 473 to 501. In yet some further embodiments such reconstituted RBM may comprise the amino acid sequence NSNNLDSKVGGNYNYLYRLFLLRKYQAGSTPCNGVEGFNCYFPLQSYGFQPTN, as denoted by SEQ ID NO: 73. In yet some further embodiments such reconstituted RBM may comprise, the V483E substitution. Specifically, the amino acid sequence NSNNLDSKVGGNYNYLYRLFLLRKYQAGSTPCNGEEGFNCYFPLQSYGFQPTN, as denoted by SEQ ID NO: 74. In some specific and non-limiting embodiments, the reconstituted RBM may comprise residues 440 to 456, the linker LMRQ, and residues 473 to 501. In yet some further embodiments such reconstituted RBM may comprise the amino acid sequence NLDSKVGGNYNYLYRLFLMRQYQAGSTPCNGVEGFNCYFPLQSYGFQN, as denoted by SEQ ID NO: 75. In some specific and non-limiting embodiments, the reconstituted RBM may comprise residues 437 to 456, the linker ELEK, and residues 473 to 499. In yet some further embodiments such reconstituted RBM may comprise the amino acid sequence NSNNLDSKVGGNYNYLYRLFELEKYQAGSTPCNGVEGFNCYFPLQSYGFQP, as denoted by SEQ ID NO: 76. In yet some further embodiments such reconstituted RBM may comprise, the Q474R substitution. Specifically, the amino acid sequence NSNNLDSKVGGNYNYLYRLFELEKYRAGSTPCNGVEGFNCYFPLQSYGFQP, as denoted by SEQ ID NO: 77. In some specific and non-limiting embodiments, the reconstituted RBM may comprise residues 440 to 456, the linker LMRQY, and residues 473 to 503. In yet some further embodiments such reconstituted RBM may comprise the amino acid sequence NLDSKVGGNYNYLYRLFLMRQYYQAGSTPCNGVEGFNCYFPLQSYGFQPTNGV, as denoted by SEQ ID NO: 78. In some specific and non-limiting embodiments, the reconstituted RBM may comprise residues 442 to 456, the linker LLRQY, and residues 473 to 500. In yet some further embodiments such reconstituted RBM may comprise the amino acid sequence DSKVGGNYNYLYRLFLLRQYYQAGSTPCNGVEGFNCYFPLQSYGFQPT, as denoted by SEQ ID NO: 79. In yet some further embodiments such reconstituted RBM may comprise, the L492I substitution. Specifically, the amino acid sequence DSKVGGNYNYLYRLFLLRQYYQAGSTPCNGVEGFNCYFPIQSYGFQPT, as denoted by SEQ ID NO: 80. In some specific and non-limiting embodiments, the reconstituted RBM may comprise residues 437 to 456, the linker LLRAL, and residues 473 to 501. In yet some further embodiments such reconstituted RBM may comprise the amino acid sequence NSNNLDSKVGGNYNYLYRLFLLRALYQAGSTPCNGVEGFNCYFPLQSYGFQPTN, as denoted by SEQ ID NO: 81. In some specific and non-limiting embodiments, the reconstituted RBM may comprise residues 440 to 456, the linker LLRSL, and residues 473 to 500. In yet some further embodiments such reconstituted RBM may comprise the amino acid sequence NLDSKVGGNYNYLYRLF LLRSLYQAGSTPCNGVEGFNCYFPLQSYGFQPT, as denoted by SEQ ID NO: 82. In some specific and non-limiting embodiments, the reconstituted RBM may comprise residues 438 to 456, the linker QLREH, and residues 473 to 500. In yet some further embodiments such reconstituted RBM may comprise the amino acid sequence SNNLD SKVGGNYNYLYRLFQLREHYQAGSTPCNGVEGFNCYFPLQSYGFQPT, as denoted by SEQ ID NO: 83. In some specific and non-limiting embodiments, the reconstituted RBM may comprise residues 438 to 456, the linker QLREF, and residues 473 to 500. In yet some further embodiments such reconstituted RBM may comprise the amino acid sequence SNNLD SKVGGNYNYLYRLFQLREFYQAGSTPCNGVEGFNCYFPLQSYGFQPT, as denoted by SEQ ID NO: 84. It should be understood that the various RBMs as used herein are only illustrative examples. It should be further understood that the invention further encompasses RBMs comprising the various linkers used therein, that can replace anchor loops in various positions as indicated herein. Still further, the RBMs encompassed by the invention can be composed of any combinations of the various “A” and “B” fragments as indicated above. Moreover, each of these combinations may be combined with each of the N′-terminal and/or C′-terminal extensions indicated herein, and thus, comprise various lengths, specifically, any length of 10 to 100 amino acid residues, as discussed herein before. In yet some further embodiments, the invention further pertains to any of the RBMs disclosed herein, specifically any of the RBMs that comprise the amino acid sequences of any one of SEQ ID NO: 65-84, or any variants thereof as disclosed by the invention. Still further, it should be appreciated that any of the RBMs disclosed herein are applicable for any of the aspects of the invention as disclosed herein after.

In a further aspect thereof, the invention provides an RBD of SARS CoV2 comprising the native RBD of the spike protein of SARS CoV2 or any variants mutants or fragments thereof and at least one linker. In some embodiments, at least one of the linker/s replaces an anchor loop or any part thereof, or amino acid residue/s thereof. More specifically, such anchor loop may comprise an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. It should be noted that at least one of said linker is a bridging linker. In some specific and non-limiting embodiments, the RBD of the present disclosure comprises residues 336 to 518 of the spike protein of SEQ ID NO:6, or any variants thereof, for example, the variants of SEQ ID NO: 38, 39 and 41, with the at least one linker that replaces the anchor loop as indicated above. In some specific embodiments, the RBD of the present disclosure comprises the amino acid sequence as denoted by SEQ ID NO: 1, or any variants and derivatives thereof. In some particular and non-limiting embodiments, such variants may comprise at least one of the following substitutions Y489N, V483E, Q474R, L489I, N501Y, K417N, E484K, L452R, Y453F, or any combinations thereof. In some embodiments the bridging linker bridges the amino acid residues that flank the replaced anchor loop or any part thereof, or amino acid residue/s thereof. In yet some further embodiments, the linker may comprise any compound bridging the amino acid residues that flank the replaced anchor loop or any part or amino acid residue/s thereof. In more embodiments, the linker is an inert linker. In yet some further embodiments, such linker is any inorganic or organic molecule, any small molecule, any peptide (L- as well as D-aa residues), or any combinations thereof. In yet some further embodiments, the at least one linker is an amino acid linker comprising 3 to 7 amino acid residues. It should be appreciated that any of the likers discussed above is applicable for the present aspect. More specifically, the at least one linker used for the RBD comprises the amino acid sequence of Xaa¹-Xaa²-Xaa³-Xaa₍ₙ₎ as denoted by SEQ ID NO: 57 or any fragment thereof. In some embodiments, the Xaa is any amino acid, wherein n is zero or an integer of from 1 to 4, and wherein: Xaa¹ is a Leu, Gln, Glu or Gly; Xaa² is Leu, Met or Val; and Xaa³ is Arg, Glu or Pro. In yet some further embodiments, the linkers used for the RBD may by any one of the linkers of SEQ ID NO: 42 to 52 or the NNK₃ linker GVP. It should be noted that the RBDs provided by the invention may comprise the amino acid sequence of the native RBD, or of any fragment thereof. “Fragment” with respect to polypeptide sequences means polypeptides that comprise at least 10%, at least 20%, at least 30%, at least 40%, at least 50%, at least 60%, at least 70%, at least 80%, at least 90% or at least 95% of the complete segment of the native RBD of the viral spike protein, specifically the SARS-CoV2 S1 protein. In some embodiments, fragments of the RBDs may comprise at least 5, at least 10, at least 15, at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 150 amino acids or more, at least 200 amino acids or more, at least 250 amino acids or more and at least 300 amino acids or more of the spike RBD.

A further aspect of the invention relates to a Spike protein of SARS CoV2, comprising the native Spike protein of SARS CoV2 or any variants mutants or fragment thereof and at least one linker. In some embodiments, the at least one linker/s replaces an anchor loop or any part or amino acid residue/s thereof of the spike protein. In more specific embodiments, the anchor loop comprises an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. It should be noted that in some embodiments, the at least one of such linker/s is a bridging linker. It should be noted that the spike protein as provided by the invention, encompasses in accordance with some embodiments, the S1 subunit of the spike protein and at least one linker that replaces the anchor loop as specified herein above. In some embodiments, the S1 subunit as discussed herein comprises the amino acid sequence as denoted by SEQ ID NO: 19, and any variants thereof, as discussed herein. In yet some further embodiments, the spike protein provided herein comprises the S1 subunit and the S2 subunit and at least one linker that replaces the anchor loop, specifically the anchor loop residing at the S1 subunit. Thus, in some embodiments, the invention provides the entire spike protein that comprises at least one linker that replaces the anchor loop as specified by the invention. In some embodiments, the entire spike protein comprises the amino acid sequence as denoted by SEQ ID NO:6, and any variants thereof, for example, the variants of SEQ ID NO: 38, 39 and 41, with the at least one linker that replaces the anchor loop as indicated above. In yet some further embodiments, the spike protein provided by the invention may comprise at least one linker as discussed above and at least one of the following substitutions Y489N, Q474R, V483E, L492I, N501Y, K417N, E484K, L452R, Y453F, S13I, L18F, T20N, P26S, H69-V70 deletion, D80A, D138Y, Y144/145 deletions, W152C, R190S, D215G, L242/A243/L244 deletion, R246T, A570D, D614G, H655Y, P681H, I692V, A701V, T716I, S982A, T1027I, D1118H, V1176F, M1229I, or any combinations thereof. The Spike protein of the invention may comprise the amino acid sequence of the native Spike protein, or of any fragment thereof. “Fragment” with respect to polypeptide sequences means polypeptides that comprise at least 10%, at least 20%, at least 30%, at least 40%, at least 50%, at least 60%, at least 70%, at least 80%, at least 90%, at least 95%, at least 96%, at least 97%, at least 98%, at least 99%, at least 99.9% of the viral spike protein, specifically the SARS-CoV2 S1 protein or of the viral protein that comprises S1 and S2 subunits, provided that the anchor loop or any parts of fragments thereof are replaced by the linker/s of the invention. The at least one linker used for the RBD indicated in the previous aspect above, and/or the spike protein of the invention may be a bridging linker that bridges residue that flank the anchor loop removed by the invention. Such bridging linker, also referred to herein as a τ-linker (tau linker) may bridge any one of the amino acid residues F456, R457, K458, R454 or L455 with any one of the amino acid residues Y473, I472, E471, T470, S469, I468, Q474, A475 or G476 of the SARS CoV2 Spike protein. In some embodiments the bridging linker bridges the amino acid residues that flank the replaced anchor loop or any part or amino acid residue/s thereof. In yet some further embodiments, the linker may comprise any compound bridging the amino acid residues that flank the replaced anchor loop or any part or amino acid residue/s thereof. In more embodiments, the linker is an inert linker. In yet some further embodiments, such linker is any inorganic or organic molecule, any small molecule, any peptide (L- as well as D-aa residues), or any combinations thereof. In yet some further embodiments, the at least one linker is an amino acid linker comprising 3 to 7 amino acid residues. Still further, the RBD and the Spike proteins provided by the invention comprise at least one linker, specifically, at least one τ-linker, and are therefore designated according to some embodiments of the invention as τ-modified RBDs and i-modified Spike proteins. It should be appreciated that any of the likers discussed above is applicable for the present aspect. More specifically, the at least one linker used for the spike protein comprises the amino acid sequence of Xaa¹-Xaa²-Xaa³-Xaa₍ₙ₎ as denoted by SEQ ID NO: 57 or any fragment thereof. In some embodiments, the Xaa is any amino acid, wherein n is zero or an integer of from 1 to 4, and wherein: Xaa¹ is a Leu, Gln, Glu or Gly; Xaa² is Leu, Met or Val; and Xaa³ is Arg, Glu or Pro. In yet some further embodiments, the linkers used for the spike protein may by any one of the linkers of SEQ ID NO: 42 to 52 or the NNK₃ linker GVP.

In a further aspect thereof, the invention provides at least one SARS CoV2 virus or any variant or mutant thereof, or any killed or attenuated version thereof. Such SARS CoV2 virus in accordance with some embodiments, may comprise at least one Spike protein that comprises at least one linker. In some embodiments, the SARS CoV2 virus provided by the invention may comprise at least one τ-linker, and thus may referred to herein as the τ-modified SARS CoV2 virus of the invention. At least one of the linkers replaces an anchor loop or any part thereof, or amino acid residue/s thereof. More specifically, the anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of said Spike protein. It should be and wherein the at least one linker is a bridging linker. In some embodiments the bridging linker bridges the amino acid residues that flank the replaced anchor loop or any part thereof, or amino acid residue/s thereof. In some embodiments, the linker used must differ from the replaced native sequence in at least one amino acid residue, and specifically, two, three, four, five, six, seven or more residues. In yet some further embodiments, the linker used to replace the native sequences (e.g., the loop or any fragments thereof), differs from the native replaced sequence, such that the spike protein of the SARS CoV2 virus disclosed herein comprise said at least one linker cannot be considered as a natural product. It should be appreciated that any of the linkers discussed above is applicable for the present aspect. More specifically, the at least one linker used for the spike protein comprises the amino acid sequence of Xaa¹-Xaa²-Xaa³-Xaa₍ₙ₎ as denoted by SEQ ID NO: 57 or any fragment thereof. In some embodiments, the Xaa is any amino acid, wherein n is zero or an integer of from 1 to 4, and wherein: Xaa¹ is a Leu, Gln, Glu or Gly; Xaa² is Leu, Met or Val; and Xaa³ is Arg, Glu or Pro. In yet some further embodiments, the linkers used for the spike protein may by any one of the linkers of SEQ ID NO: 42 to 52 or the NNK₃ linker GVP.

As indicated above, this aspect of the invention encompasses any variant of the SARS CoV2 virus, or any killed or attenuated version thereof. In some embodiments, the variant may comprise the spike protein as denoted by SEQ ID NO:6, and any variants thereof, for example, the variants of SEQ ID NO: 38, 39 and 41, with the at least one linker that replaces the anchor loop as indicated above. In yet some further embodiments, at least one of the spike protein of the SARS CoV2 virus, or any killed or attenuated version thereof provided by the invention, may comprise at least one linker as discussed above and at least one of the following substitutions Y489N, Q474R, V483E, L492I, N501Y, K417N, E484K, L452R, Y453F, S13I, L18F, T20N, P26S, H69-V70 deletion, D80A, D138Y, Y144/145 deletions, W152C, R190S, D215G, L242/A243/L244 deletion, R246I, A570D, D614G, H655Y, P681H, I692V, A701V, T716I, S982A, T1027I, D1118H, V1176F, M1229I, or any combinations thereof. It should be noted that the amino acid positions refer to SEQ ID NO: 6. The invention thus provides reconstituted RBMs, specifically, SARS-COV2-derived reconstituted RBMs polypeptides, or any RBDs and spike proteins (comprising S1 and/or S1+S2 subunits), that comprise at least one linker that replaces the anchor loop thereof. These RBM polypeptides or any RBDs, spike proteins, and SARS-CoV2, specifically, the τ-modified RBMs, τ-modified RBDs, τ-modified Spike proteins and τ-modified SARS-CoV2 provided by the invention, are in some embodiments, isolated and purified polypeptides. In some specific embodiments, these reconstituted RBM polypeptides or any RBDs and spike proteins of the invention may be recombinant polypeptides, that may in some embodiments be recombinantly produced. However, the invention further encompasses reconstituted RBMs or any RBDs and spike proteins disclosed by the invention that are produced synthetically.

An ‘isolated polypeptide’ is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms. By definition, isolated peptides are also non-naturally occurring, synthetic peptides. Methods for isolating or synthesizing peptides of interest with known amino acid sequences are well known in the art.

The polypeptides of the invention are therefore considered as proteinaceous material. A “proteinaceous material” is any protein, or fragment thereof, or complex containing one or more proteins formed by any means, such as covalent peptide bonds, disulfide bonds, chemical crosslinks, etc., or non-covalent associations, such as hydrogen bonding, van der Waal's contacts, electrostatic salt bridges, etc.

The reconstituted RBM polypeptides or any RBDs and spike proteins of the invention are composed of an amino acid sequence. An ‘amino acid/s’ or an ‘amino acid residue/s’ can be a natural or non-natural amino acid residue/s linked by peptide bonds or bonds different from peptide bonds. The amino acid residues can be in D-configuration or L-configuration (referred to herein as D- or L-enantiomers). An amino acid residue comprises an amino terminal part (NH₂) and a carboxy terminal part (COOH) separated by a central part (R group) comprising a carbon atom, or a chain of carbon atoms, at least one of which comprises at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature are listed in 37 C.F.R. 1.822(b)(2). Examples of non-natural amino acids are also listed in 37 C.F.R. 1.822(b)(4), other non-natural amino acid residues include, but are not limited to, modified amino acid residues, L-amino acid residues, and stereoisomers of D-amino acid residues. Naturally occurring amino acids may be further modified, e.g., hydroxyproline, γ-carboxyglutamate, and O-phosphoserine. Thus, the reconstituted RBM polypeptides of the invention may comprise natural or non-natural amino acid residues, or any combination thereof. Further, amino acids may be amino acid analogs or amino acid mimetics. Amino acid analogs refer to compounds that have the same fundamental chemical structure as naturally occurring amino acids, but modified R groups or modified peptide backbones, e.g., homoserine, norleucine, methionine sulfoxide, methionine methyl sulfonium. Amino acid mimetics refers to chemical compounds that have a structure that is different from the general chemical structure of an amino acid, but that function in a manner similar. Amino acids may be referred to herein by either their commonly known three letter symbols or by the one-letter symbols recommended by the IUPAC-IUB Biochemical Nomenclature Commission.

Further, the reconstituted RBM polypeptides or any RBDs and spike proteins of the invention may comprise ‘equivalent amino acid residues’. This term refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, equivalent amino acid residues can be regarded as conservative amino acid substitutions.

In the context of the present invention, within the meaning of the term ‘equivalent amino acid substitution’ as applied herein, is meant that in certain embodiments one amino acid may be substituted for another within the groups of amino acids indicated herein below:
(i) Amino acids having polar side chains (Asp, Glu, Lys, Arg, His, Asn, Gln, Ser, Thr, Tyr, and Cys); (ii) Amino acids having non-polar side chains (Gly, Ala, Val, Leu, Ile, Phe, Trp, Pro, and Met); (iii) Amino acids having aliphatic side chains (Gly, Ala Val, Leu, Ile); (iv) Amino acids having cyclic side chains (Phe, Tyr, Trp, His, Pro); (v) Amino acids having aromatic side chains (Phe, Tyr, Trp); (vi) Amino acids having acidic side chains (Asp, Glu); (vii) Amino acids having basic side chains (Lys, Arg, His); (viii) Amino acids having amide side chains (Asn, Gln); (ix) Amino acids having hydroxyl side chains (Ser, Thr, Tyr), or; (x) Amino acids having sulfur-containing side chains (Cys, Met); (xi) Neutral, weakly hydrophobic amino acids (Pro, Ala, Gly, Ser, Thr); (xii) Hydrophilic, acidic amino acids (Gln, Asn, Glu, Asp), and (xiii) Hydrophobic amino acids (Leu, Ile, Val).

Still further, the reconstituted RBM polypeptide of the invention, or any RBDs and spike proteins of the invention, may have secondary modifications, such as phosphorylation, acetylation, glycosylation, sulfhydryl bond formation, cleavage and the likes, as long as said modifications retain the functional properties of the original protein, specifically, the ability to interact with the viral receptor and the neutralizing antibodies. Secondary modifications are often referred to in terms of relative position to certain amino acid residues. For example, a certain sequence positioned carboxyl-terminal to a reference sequence within a polypeptide is located proximal to the carboxyl terminus of the reference sequence but is not necessarily at the carboxyl terminus of the complete polypeptide. The invention further encompasses any derivatives, enantiomers, analogues, variants or homologues of any of the reconstituted RBM polypeptides or any RBDs and spike proteins disclosed herein. More specifically, any derivatives and variants of any of the RBMs disclosed by the invention, for example, an of the RBMs of SEQ ID NO: 65 to 84, any variant or derivative of an RBD and/or spike protein that comprise any of the linkers of the invention, for example, any one of SEQ ID NO: 57, or 42 to 52, or any variant or derivative of an of the amino acid sequences as disclosed by the invention, specifically, any of the polypeptides of SEQ ID NO: 1-6, 17-19, 21, 23, 25-93, 99, 101, 102, 133. The term “derivative” or “variant” are used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof, as well as covalent modifications of a polypeptides made according to the present invention. It should be noted that the reconstituted RBM polypeptides, or any RBDs and spike proteins according to the invention can be produced either synthetically, or by recombinant DNA technology. Methods for producing polypeptides peptides are well known in the art.

In some embodiments, derivatives include, but are not limited to, polypeptides that differ in one or more amino acids in their overall sequence from the polypeptides defined herein, polypeptides that have deletions, substitutions, inversions or additions. Non-limiting examples for variants include but are not limited to variants comprising at least one of the following substitutions Y489N, Q474R, V483E, L492I, N501Y, K417N, E484K, L452R, Y453F, S13I, L18F, T20N, P26S, H69-V70 deletion, D80A, D138Y, Y144/145 deletions, W152C, R190S, D215G, L242/A243/L244 deletion, R246T, A570D, D614G, H655Y, P681H, I692V, A701V, T716I, S982A, T1027I, D1118H, V1176F, M1229I or any combinations thereof.

Further reconstituted RBM variants may be prepared and selected via Biased Random Mutagenesis, as described in Example 5 below. In some embodiments, derivatives refer to polypeptides, which differ from the polypeptides specifically defined in the present invention by insertions of amino acid residues. It should be appreciated that by the terms “insertions” or “deletions”, as used herein it is meant any addition or deletion, respectively, of amino acid residues to the polypeptides used by the invention, of between 1 to 50 amino acid residues, between 20 to 1 amino acid residues, and specifically, between 1 to 10 amino acid residues. More particularly, insertions or deletions may be of any one of 1, 2, 3, 4, 5, 6, 7, 8, 9, or 10 amino acids. It should be noted that the insertions or deletions encompassed by the invention may occur in any position of the modified peptide, as well as in any of the N′ or C′ termini thereof. It should be appreciated that in cases the deletion/s or insertion/s are in the N or C-terminus of the peptide, such derivatives may be also referred to as fragments.

The reconstituted RBM polypeptide or any RBDs and spike proteins of the invention of the invention may all be positively charged, negatively charged or neutral. In addition, they may be in the form of a dimer, a multimer or in a constrained conformation, which can be attained by internal bridges, short-range cyclization, extension or other chemical modifications.

The polypeptides of the invention can be coupled (conjugated) through any of their residues to another peptide or agent. For example, the polypeptides of the invention can be coupled through their N-terminus to a lauryl-cysteine (LC) residue and/or through their C-terminus to a cysteine (C) residue. Further, the reconstituted RBM polypeptide or any RBDs and spike proteins of the invention may be extended at the N-terminus and/or C-terminus thereof with various identical or different amino acid residues. As an example for such extension, the peptide may be extended at the N-terminus and/or C-terminus thereof with identical or different amino acid residue/s, which may be naturally occurring or synthetic amino acid residue/s. An additional example for such an extension may be provided by peptides extended both at the N-terminus and/or C-terminus thereof with a cysteine residue. Naturally, such an extension may lead to a constrained conformation due to Cys-Cys cyclization resulting from the formation of a disulfide bond. Another example may be the incorporation of an N-terminal lysyl-palmitoyl tail, the lysine serving as linker and the palmitic acid as a hydrophobic anchor. In addition, the peptides may be extended by aromatic amino acid residue/s, which may be naturally occurring or synthetic amino acid residue/s, for example, a specific aromatic amino acid residue may be tryptophan. The peptides may be extended at the N-terminus and/or C-terminus thereof with various identical or different organic moieties, which are not naturally occurring or synthetic amino acids. As an example for such extension, the reconstituted RBM polypeptide or any RBDs and spike proteins may be extended at the N-terminus and/or C-terminus thereof with an N-acetyl group. For every single peptide sequence defined by the invention and disclosed herein, this invention includes the corresponding retro-inverse sequence wherein the direction of the peptide chain has been inverted and wherein all or part of the amino acids belong to the D-series. It should be understood that the present invention includes embodiments wherein one or more of the L-amino acids is replaced with its D isomer.

In yet some further embodiments, the reconstituted RBM polypeptide or any RBDs and spike proteins of the invention may comprise at least one amino acid residue in the D-form. It should be noted that every amino acid (except glycine) can occur in two isomeric forms, because of the possibility of forming two different enantiomers (stereoisomers) around the central carbon atom. By convention, these are called L- and D-forms, analogous to left-handed and right-handed configurations. It should be appreciated that in some embodiments, the enantiomer or any derivatives of the reconstituted RBMs or any RBDs and spike proteins of the invention may exhibit at least one of enhanced activity, and superiority. In more specific embodiments, such derivatives and enantiomers may exhibit increased affinity to the nAbs or the viral receptor, enhanced stability, and increased resistance to proteolytic degradation, and possess enhanced ability to elicit production of neutralizing antibodies in a subject vaccinated by the polypeptides of the invention. The invention also encompasses any homologues of the polypeptides specifically defined by their amino acid sequence according to the invention. The term “homologues” is used to define amino acid sequences (polypeptide) which maintain a minimal homology to the amino acid sequences defined by the invention, e.g. preferably have at least about 65%, more preferably at least about 70%, at least about 75%, even more preferably at least about 80%, at least about 85%, most preferably at least about 90%, at least about 95% overall sequence homology with the entire amino acid sequence of any of the polypeptide as structurally defined above, e.g. of a specified sequence, more specifically, an amino acid sequence of the linkers and polypeptides as denoted by any one of SEQ ID NOs: 1-6, 17-19, 21, 23, 25-93, 99, 101, 102, 133, and any derivatives, enantiomers and fusion proteins thereof.

More specifically, “Homology” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- nor C-terminal extensions nor insertions or deletions shall be construed as reducing identity or homology. Methods and computer programs for the alignment are well known in the art.

In some embodiments, the present invention also encompasses polypeptides which are variants of, or analogues to, the polypeptides specifically defined in the invention by their amino acid sequence. With respect to amino acid sequences, one of skill will recognize that individual substitutions, deletions or additions to peptide, polypeptide, or protein sequence thereby altering, adding or deleting a single amino acid or a small percentage of amino acids in the encoded sequence is a “conservatively modified variant”, where the alteration results in the substitution of an amino acid with a chemically similar amino acid.

Conservative substitution tables providing functionally similar amino acids are well known in the art and disclosed herein before. Such conservatively modified variants are in addition to and do not exclude polymorphic variants, interspecies homologues, and alleles and analogous peptides of the invention. More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved.

As noted above, the peptides of the invention may be modified by omitting their N-terminal sequence. It should be appreciated that the invention further encompasses the omission of about 1, 2, 3, 4, 5, 6, 7, 8 and more amino acid residues from both, the N′ and/or the C′ termini of the peptides of the invention. Certain commonly encountered amino acids which also provide useful substitutions include, but are not limited to, β-alanine (β-Ala) and other omega-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGIy); ornithine (Orn); citrulline (Cit); t-butylalanine (t-BuA); t-butylglycine (t-BuG); N-methylisoleucine (MeIIe); phenylglycine (Phg); cyclohexylalanine (Cha); norleucine (NIe); naphthylalanine (NaI); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,4-diaminobutyric acid (Dbu); 2,4-diaminobutyric acid (Dab); p-aminophenylalanine (Phe(pNH.sub.2)); N-methyl valine (MeVal); homocysteine (hCys), homophenylalanine (hPhe) and homoserine (hSer); hydroxyproline (Hyp), homoproline (hPro), N-methylated amino acids (e.g., N-substituted glycine). Covalent modifications of the peptide are included and may be introduced by reacting targeted amino acid residues of the peptide with an organic derivatizing agent that is capable of reacting with selected side chains or terminal residues.

Cysteinyl residues most commonly are reacted with α-haloacetates (and corresponding amines) to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylprocarbonate (pH 5.5-7.0) which agent is relatively specific for the histidyl side chain. Bromophenacyl bromide also is useful; the reaction is preferably performed in 0.1 M sodium cacodylate at pH 6.0. Lysinyl and amino terminal residues are reacted with succinic or other carboxylic acid anhydrides. Derivatization with these agents reverses the charge of the lysinyl residues. Other suitable reagents for derivatizing α-amino-containing residues include imidoesters such as methylpicolinimidate; pyridoxal phosphate; pyridoxal; chloroborohydride; trinitrobenzenesulfonic acid; O-methylisourea; 2,4 pentanedione; and transaminase-catalyzed reaction with glyoxylate. Arginyl residues are modified by reaction with one or several conventional reagents, including phenylglyoxal, 2,3-butanedione, 1,2-cyclohexanedione, and ninhydrin. Such derivatization requires that the reaction be performed in alkaline conditions because of the high pKa of the guanidine functional group. Furthermore, these reagents may react with the groups of lysine as well as the arginine F-amino group. Modification of tyrosyl residues has permits introduction of spectral labels into a peptide. This is accomplished by reaction with aromatic diazonium compounds or tetranitromethane. Most commonly, N-acetylimidizol and tetranitromethane are used to create O-acetyl tyrosyl species and 3-nitro derivatives, respectively. Carboxyl side groups (aspartyl or glutamyl) are selectively modified by reaction with carbodiimides (R′—N—C—N—R′) such as 1-cyclohexyl-3-(2-morpholinyl-(4-ethyl) carbodiimide or 1-ethyl-3-(4-azonia-4,4-dimethylpentyl) carbodiimide. Aspartyl and glutamyl residues are converted to asparaginyl and glutaminyl residues by reaction with ammonium ions. Conversely, glutaminyl and asparaginyl residues may be deamidated to the corresponding glutamyl and aspartyl residues. Deamidation can be performed under mildly acidic conditions. Either form of these residues falls within the scope of this invention. Derivatization with bifunctional agents is useful for cross-linking the peptide to a water-insoluble support matrix or other macromolecular carrier. Commonly used cross-linking agents include 1,1-bis(diazoacetyl)-2-phenylethane, glutaraldehyde, N-hydroxysuccinimide esters, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Other chemical modifications include hydroxylation of proline and lysine, phosphorylation of the hydroxyl groups of seryl or threonyl residues, methylation of the α-amino groups of lysine, arginine, and histidine side chains (Creighton, supra), acetylation of the N-terminal amine, and, in some instances, amidation of the C-terminal carboxyl. Such chemically modified and derivatized moieties may improve the peptide's solubility, absorption, biological half-life, and the like. These changes may eliminate or attenuate undesirable side effects of the proteins in vivo.

It should be appreciated that the invention further encompasses any of the peptides of the invention referred herein, e.g., any polypeptide of any one of SEQ ID NO: 1-6, 17-19, 21, 23, 25-93, 99, 101, 102, 133, and specifically, the linkers of SEQ ID NO: 7, 42-52 and the RBMs of any one of SEQ ID NO: 65-84, any serogates thereof, any salt, base, ester or amide thereof, any enantiomer, stereoisomer or disterioisomer thereof, or any combination or mixture thereof. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. The present invention encompasses any fragment, derivative or analogue of any of the reconstituted RBM polypeptides or any RBDs and spike proteins of the invention, or any of the τ-linkers disclosed by the invention. In certain embodiments, any of the polypeptides of the invention and derivatives thereof can bind the viral receptor as well as neutralizing antibodies (nAb/s), and more importantly, possess the ability to elicit the production of neutralizing antibodies in a subject vaccinated by the polypeptides of the invention. Thus, in some embodiments a “functional” reconstituted RBM polypeptide or any RBDs and spike proteins of the invention is a peptide possessing the ability to elicit the production of neutralizing antibodies, and/or inducing immunity against the infecting SARS-CoV2. In certain embodiments, the reconstituted RBMs or any RBDs and spike proteins of the invention may be fused to additional peptide sequences. The invention further encompasses any fusion protein comprising the reconstituted RBMs or any RBDs and spike proteins of the invention as described herein. More specifically, additional peptide sequences can be added to the polypeptides (reconstituted RBMs or any RBDs and spike proteins) of the invention thereby forming fusion proteins, which act to promote stability, purification, and/or detection. For example, a reporter peptide portion (e.g., green fluorescent protein (GFP), β-galactosidase, His Tag, a detectable domain thereof, or any other immunogenic determinants) can be used. Purification-facilitating peptide sequences include those derived or obtained from maltose binding protein (MBP), glutathione-S-transferase (GST), or thioredoxin (TRX).

It should be appreciated that the invention further provides plurality of the reconstituted RBMs, the RBDs and spike proteins comprising the reconstituted RBMs of the invention as well as any of the RBDs and/or spike proteins that comprise at least one linker that replaces at least in part the anchor loop, or any combinations thereof. It should be note that the plurality of the polypeptides of the invention may comprise either homogenous polypeptide, comprising for example, reconstituted RBM molecules that comprise the same linker, or a plurality of polypeptides that comprise reconstituted RBMs with various τ-linkers, or a plurality of polypeptides that comprise reconstituted RBMs with either identical or various linkers that replace different fragments or portions of the anchor loop, or plurality of polypeptides comprising at least two of the reconstituted RBMs, RBDs and Spike proteins as described by the invention comprising the same linker, or various different linkers. Still further, the plurality of polypeptides may comprise either identical or different polypeptides having identical or different modifications. Still further, the plurality of polypeptides may be provided by the invention either in a solution, or in a dry form or attached to a solid support.

A further aspect of the invention relates to a multimeric and/or multivalent antigen displaying platform comprising at least one reconstituted RBM of a Spike protein of SARS CoV2 or of any fragment thereof, or any fusion protein or conjugate thereof, any polypeptide, RBD or Spike protein comprising the reconstituted RBM, any RBD or Spike protein comprising at least one linker that replaces an anchor loop or any part thereof, or amino acid residue/s thereof, specifically, any of the τ-modified polypeptides of the invention and any combinations thereof. More specifically, the anchor loop replaced herein, may comprise an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein. In some embodiments, the reconstituted RBM used in the multimeric and/or multivalent antigen displaying platform of the invention may comprise at least one linker and at least one fragment of the native RBM. In more specific embodiments, the native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein. Still further, the native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. It should be noted that at least one of the linker/s replaces the anchor loop or any part thereof, or amino acid residue/s thereof, in the reconstituted RBM used by the multimeric and/or multivalent antigen displaying platform of the invention. In yet some further embodiments, the invention further relates to a Polymeric and/or Oligomeric, i.e., any duplicity of two or more copies of the reconstituted RBM or any RBDs and spike proteins in tandem or on separate scaffolds assemble together or associated together as in trimeric spikes etc. or more copies of the RBM in tandem or on separate scaffolds assemble.

In some embodiments, the polypeptide, RBD or Spike protein comprising the reconstituted RBM or linkers used by the multimeric and/or multivalent antigen displaying platform of the invention, is as defined by the invention. Still further, the multimeric and/or multivalent antigen displaying platform may further use any of the RBD or Spike protein comprising at least one linker as defined by the invention. It should be understood that the multimeric and/or multivalent antigen displaying platform of the invention may comprise any combination of reconstituted RBMs, for example, reconstituted RBMs that comprise different linkers, or reconstituted RBMs that comprise the same linker that replace a different fragment of the anchor loop, or any combinations thereof. Still further, the multimeric and/or multivalent antigen displaying platform of the invention may comprise any combination of any of the reconstituted RBMs of the invention, any polypeptide comprising these reconstituted RBMs (e.g., any of the RBDs and/or Spike proteins described by the invention), and/or any of the Spike proteins and/or RBDs that comprise at least one inker that replaces the anchor loop thereof. In some embodiments, the polypeptides, RBDs or Spike proteins comprising the reconstituted RBM or linkers used by the multimeric and/or multivalent antigen displaying platform of the invention may comprise any of the linkers disclosed herein, for example, any of the τ-linkers of any one of SEQ ID NO: 57, or 42 to 52, or any of the reconstituted RBMs disclosed herein, for example, any RBM comprising any one of SEQ ID NO: 65 to 84, and any variants thereof. As indicated above, when referred to variants of RBMs, RBDs or spike proteins, it is meant, any variant that comprises with at least one of the following substitutions, Y489N, Q474R, V483E, L492I, N501Y, K417N, E484K, L452R, Y453F (for RBMs, RBDs and spike protein), and S13I, L18F, T20N, P26S, H69-V70 deletion, D80A, D138Y, Y144/145 deletions, W152C, R190S, D215G, L242/A243/L244 deletion, R246I, A570D, D614G, H655Y, P681H, I692V, A701V, T716I, S982A, T1027I, D1118H, V1176F, M1229I (for spike protein), or any combinations thereof.

In some specific embodiments, the multimeric and/or multivalent antigen displaying platform of the invention may comprise a self-assembling nanostructure.

In some embodiments, the self-assembling nanostructure comprising a plurality of polypeptides, arranged according to at least one symmetry operator. In some further embodiments, the nanostructure comprises a first plurality of antigens, specifically, any of the polypeptides of the invention, RBMs, RBDs and spike proteins disclosed by the invention, each of the first plurality of the antigens has a proximal end and a distal end. In some embodiments, the proximal ends of the antigens are each attached to a member of the first plurality of polypeptides. The multimeric and/or multivalent antigen displaying platform of the invention may comprise any reconstituted RBM (either with the same or with various linkers), RBD, Spike that comprise the same or various linkers as disclosed by the invention (e.g., any one of the τ-linkers of SEQ ID NO: 57, 42-52 and GVP), and any combinations thereof.

In yet some further specific embodiments, the polypeptides of the invention, specifically, any of the reconstituted RBM/s, and/or or any polypeptide comprising the reconstituted RBM, specifically, any of the RBDs and/or Spike proteins (comprising either S1 and/or the S1 and S2 subunits) that comprise the reconstituted RBMs of the invention, or any of the RBDs and/or Spike proteins that comprise at least one τ-linker that replaces the anchor loop or any fragment or part thereof, is incorporated in a multimeric/multivalent antigen displaying platform. In some specific and non-limiting embodiments, the polypeptides of the invention may be comprised or arranged within a self-assembling nanostructure. In yet some further embodiments, such nanostructure may be the nanostructure as defined by King et al., [11]. In more specific embodiments, the reconstituted RBMs, or any of the polypeptides of the invention, specifically any of the RBDs, and Spike proteins disclosed by the invention, is expressed as component A in the self-assembling capsid nanoparticle. Component A is expressed in any host cell, for example, a bacterial host cell such as E. coli and is mixed with Component B. This mixture initiates the formation of a nanomolecular capsid structure in which the Components A for trimers and associate with pentameric Component B. In some specific embodiments, several trimeric Component A can assemble with Component B. It should be noted that about 2, 3, 4, 5, 6, 7, 8, 9, 10, 11, 12, 13, 14, 15, 16, 17, 18, 19, 20, 21, 22, 23, 24, 25, 26, 27, 28, 29, 30 or more trimeric Component A can assemble with Component B. Thus, a polyvalent immunogenic capsid is formed and can be used in some embodiments of the invention. Other presentations of the reconstituted RBM are also envisioned using a variety of scaffolds. Dendrimers presenting multiple copies of any of the RBMs, and/or RBDs, and/or Spike proteins provided by the invention are also encompassed by the invention. Tetrameric RBM is easily produced by incorporating an Avitag sequence or any connecting moiety at the carboxyterminal end of any of the polypeptides provided by the invention, e.g., the reconstituted RBM. Hence each RBM can be C-terminally biotinylated. Mixing biotinylated RBM with Avidin produces stable tetramers.

It should be appreciated that in some embodiments, heteromeric mixes of the RBMs, RBDs and/or Spike proteins of the invention is also encompassed by the invention. For example, variations of functional reconstituted RBMs that contain variations of functional linkers ranging from 3 to 7 amino acids, and/or different or identical linkers that replace the same or various fragments or parts of the anchor loop. Thus, each functional linker may generate slight variations in the RBMs, RBDs, and spike protein/s conformation representing the dynamic conformational variations of the RBM in the virus. Still further, in some embodiments, the production of chimeric capsids is also encompassed by the invention by mixing component B with a variety of Component A units each displaying functional RBMs that differ in their functional linker compositions. It should be further appreciated that the capsid nano structures of the invention may be further modified, e.g., by glycosylation, phosphorylation and the like.

In yet some further embodiments, the RBMs, RBDs and/or Spike proteins of the invention may be presented as a multimeric/multivalent antigen, using the bacteriophage Protein 3 scaffold as shown by the examples, or alternatively, the bacteriophage Protein 8 scaffold, or Proteins 7 or 9 scaffold. More specifically, Example 13 illustrates an effective scaffold for the reconstituted active RBM of the invention that may be used as a bait (as will be described in more detail in connection with other aspects of the invention). More specifically, as discussed by Examples 1-4, in some embodiments, the first, second, third and fourth generation RBMs of the invention is selected from a conformer library displayed on a bacteriophage. In some specific embodiments, such conformer library is displayed on filamentous bacteriophages based on the fth1 phage vector previously described by the inventor (Enshell D et al., Nucleic Acids Res. 2001 May 15; 29(10): e50). The combinatorial linker library is expressed on Protein 3 which exists in five copies. Thus, in accordance with some embodiments of the invention, the reconstituted RBM of the invention is expressed and displayed on Protein 3 scaffold. In some embodiments, the RBM of the invention displayed on Protein 3 scaffold, may be referred to herein as a multimeric version of the RBM, containing a pentamer of the RBM on five copies of the P3 protein. Similarly, any of the RBDs and/or Spike proteins of the invention may be displayed using the P3 protein scaffold. In yet, some further embodiments, the reconstituted RBM of the invention is expressed and displayed on Protein 8 scaffold. In some embodiments, the RBM of the invention displayed on Protein 8 scaffold, may be referred to herein as a polyvalent version of the RBM, as the number or recombinant Protein 8 molecules in any chimeric phage could be greater than 10 and possibly hundreds of copies per phage. As will be discussed in more detail in connection with other aspects of the invention, should be appreciated that any of the RBMs, RBDs and/or Spike proteins and any multimeric/multivalent antigen displaying platform thereof, may be connected directly or indirectly to at least one tag, detectable moiety, affinity moiety or solid support. It should be understood however, that any display vehicle can be used in the multimeric/multivalent antigen displaying platform of the invention, for example, bacteriophage (e.g., M13, fd, f1, T4 and T7), yeast, ribosome, peptide, or any other display systems, or any combinations thereof. Still further, when bacteriophage display systems are used, any phage protein can be used as a scaffold.

A further aspect of the invention relates to a nucleic acid sequence encoding at least one reconstituted RBM of a Spike protein of SARS CoV2 or of any fragment thereof, or any fusion protein thereof, any polypeptide, RBD or Spike protein comprising the reconstituted RBM, any RBD or Spike protein comprising at least one linker that replaces an anchor loop or any part thereof, or amino acid residue/s thereof, or any matrix, nano- or micro-particle thereof. More specifically, the loop comprising an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein, any multimeric and/or multivalent antigen displaying platform thereof, and any combinations thereof. In some embodiments, the RBM encoded by the nucleic acid sequence of the invention comprises at least one linker and at least one fragment of the native RBM. More specifically, the native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein. Still further, the native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein. It should be noted that at least one of said linker/s replaces the anchor loop or any part thereof, or amino acid residue/s thereof, in the reconstituted RBM encoded by the nucleic acid sequence of the invention. In some embodiments, the nucleic acid sequence provided by the invention may encode any of the polypeptide/s, RBD/s or Spike protein/s comprising the reconstituted RBMs of the invention or any variants thereof as disclosed by the invention (e.g., a variant may comprise at least one of the following substitutions, Y489N, Q474R, V483E, L492I, N501Y, K417N, E484K, L452R, Y453F (for RBMs, RBDs and spike protein), and S13I, L18F, T20N, P26S, H69-V70 deletion, D80A, D138Y, Y144/145 deletions, W152C, R190S, D215G, L242/A243/L244 deletion, R246I, A570D, D614G, H655Y, P681H, I692V, A701V, T716I, S982A, T1027I, D1118H, V1176F, M1229I (for spike protein), or any combinations thereof), or any polypeptide/s, RBD/s or Spike protein/s comprising any of the τ-linkers as defined by the invention. Still further, the nucleic acid sequence provided by the invention may encode any of the RBD or spike protein comprising at least one linker that replaces the anchor loop, as defined by the invention. In yet some further embodiments, the invention provides nucleic acid sequences that encode any of the multimeric and/or multivalent antigen displaying platform disclosed by the invention. In some embodiments, the invention provides nucleic acid molecules encoding any polypeptide comprising the linkers of the invention, for example, any of the linkers of SEQ ID NO: 57, 42-52, the GVP linker, and/or an of the RBMs of the invention, specifically, any one of SEQ ID NO: 65-84, or any variants thereof as discussed by the invention.

The invention further encompasses in further aspects thereof any vector, nucleic acid cassette or vehicle or any matrix, nano- or micro-particle thereof, comprising any of the nucleic acid sequences of the invention. Still further, according to the present aspect, the invention encompasses any nucleic acid sequence or molecule, specifically, polynucleotide encoding any of the reconstituted RBM polypeptides or any RBDs and spike proteins described herein, as well as any derivatives, variant and fusion proteins thereof, as well as any expression vector comprising said encoding nucleic acid sequence, or any host cell expressing the same. Such peptides or polypeptides, specifically, the polypeptide, RBD or Spike protein comprising the reconstituted RBM, any RBD or Spike protein comprising at least one τ-linker according to the invention may serve as an antigen or antigenic molecule. The term “nucleic acid,” in its broadest sense, includes any compound and/or substance that comprise a polymer of nucleotides. These polymers are often referred to as polynucleotides. As used herein, the term ‘polynucleotide’ or a ‘nucleic acid sequence’ refers to a polymer of nucleic acids, such as deoxyribonucleic acid (DNA) or ribonucleic acid (RNA), any combination or hybrids thereof, or any combinations or conjugates thereof with peptides. As used herein, ‘nucleic acid’ (also or nucleic acid molecule or nucleotide) refers to any DNA or RNA polynucleotides, oligonucleotides, fragments generated by the polymerase chain reaction (PCR) and fragments generated by any of ligation, scission, endonuclease action, and exonuclease action, either single- or double-stranded. Nucleic acid molecules can be composed of monomers that are naturally-occurring nucleotides (such as DNA and RNA), or analogs of naturally-occurring nucleotides (e.g., alpha-enantiomeric forms of naturally-occurring nucleotides), or modified nucleotides or any combination thereof. Herein this term also encompasses a cDNA, i.e. complementary or copy DNA produced from an RNA template by the action of reverse transcriptase (RNA-dependent DNA polymerase). Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, a-LNA having an a-L-ribo configuration (a diastereomer of LNA), 2′-amino-LNA having a 2′-amino functionalization, and 2′-amino-a-LNA having a 2′-amino functionalization), ethylene nucleic acids (ENA), cyclohexenyl nucleic acids (CeNA) or hybrids or combinations thereof. In another embodiment, the polynucleotides of the present invention which have portions or regions which differ in size and/or chemical modification pattern, chemical modification position, chemical modification percent or chemical modification population and combinations of the foregoing are known as “chimeric polynucleotides.” A “chimera” according to the present invention is an entity having two or more incongruous or heterogeneous parts or regions. As used herein a “part” or “region” of a polynucleotide is defined as any portion of the polynucleotide which is less than the entire length of the polynucleotide. In yet another embodiment, the polynucleotides of the present invention that are circular are known as “circular polynucleotides” or “circP.” As used herein, “circular polynucleotides” or “circP” means a single stranded circular polynucleotide which acts substantially like, and has the properties of, an R A. The term “circular” is also meant to encompass any secondary or tertiary configuration of the circP. In some embodiments, the polynucleotide includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

In this connection an ‘isolated polynucleotide’ is a nucleic acid molecule that is separated from the genome of an organism. For example, a DNA molecule and/or RNA molecule that encodes any of the reconstituted RBM polypeptides or any RBDs and spike proteins of the invention or any derivative, variant, fragment or fusion protein thereof that has been separated from the genomic DNA of a cell is an isolated DNA molecule. Thus, in one embodiment, the polynucleotides of the present invention is/or functions as a messenger RNA (mRNA). As used herein, the term “messenger RNA” (mRNA) refers to any polynucleotide which encodes at least one any of the reconstituted RBM polypeptides or any RBDs and spike proteins of the invention or any derivative, variant and which is capable of being translated to produce the encoded any of the reconstituted RBM polypeptides or any RBDs and spike proteins of the invention or any derivative or variant thereof, in vitro, in vivo, in situ or ex vivo. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

The invention further relates to recombinant DNA constructs comprising the polynucleotides of the invention or variants, homologues or derivatives thereof. The constructs of the invention may further comprise additional elements such as promoters, regulatory and control elements, translation, expression and other signals, operably linked to the nucleic acid sequence of the invention. As used herein, the term “recombinant DNA” or “recombinant gene” refers to a nucleic acid comprising an open reading frame encoding one of the proteins of the invention.

Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. This typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

Accordingly, the term control and regulatory elements includes promoters, terminators and other expression control elements. For instance, any of a wide variety of expression control sequences that control the expression of a DNA sequence when operatively linked to it may be used in these vectors to express DNA sequences encoding any desired protein using the method of this invention. A vector may additionally include appropriate restriction sites, antibiotic resistance or other markers for selection of vector-containing cells. Plasmids are the most commonly used form of vector but other forms of vectors which serve an equivalent function and which are, or become, known in the art are suitable for use herein. The nucleic acid molecule of the invention is used in accordance with some embodiments as a nucleic acid vaccine, and thus, can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with the nucleic acid vaccines (e.g., for transplantation into a subject), hyaluronidase, nanoparticle mimics and combinations thereof. Still further, complexes, micelles, liposomes or particles can be prepared containing these lipidoids and therefore, can result in an effective delivery of the polynucleotide, as judged by the production of an encoded protein, following the injection of a lipidoid formulation via localized and/or systemic routes of administration. Lipidoid complexes of polynucleotides in accordance with the invention can be administered by various means including, but not limited to, intravenous, intramuscular, or subcutaneous routes. Complexes, micelles, liposomes or particles can be prepared containing these lipidoids and/or lipid nano-particles (LNP), and therefore, can result in an effective delivery of the polynucleotide, as judged by the production of an encoded protein, following the injection of a lipidoid formulation via localized and/or systemic routes of administration. Lipidoid complexes of polynucleotides can be administered by various means including, but not limited to, intravenous, intramuscular, or subcutaneous routes.

In vivo delivery of nucleic acids may be affected by many parameters, including, but not limited to, the formulation composition, nature of particle PEGylation, degree of loading, polynucleotide to lipid ratio, and biophysical parameters such as, but not limited to, particle size. As an example, small changes in the anchor chain length of poly(ethylene glycol) (PEG) lipids may result in significant effects on in vivo efficacy. Formulations with the different lipidoids, including, but not limited to penta[3-(1-laurylaminopropionyl)]-triethylenetetramine hydrochloride, CI 2-200 (including derivatives and variants), and MD1, are also encompassed by the present disclosure.

It should be understood that a nucleic acid vaccine as referred to herein, further encompasses any mixture of nucleic acid molecules that encode various polypeptides, specifically, at least 1, 2, 3, 4, 5, 6, 7, 8, 9, 10, 20, 30, 40, 50, 60, 70, 80, 90, 100 or more of the various RBMs, or RBDs or spike proteins comprising the RBMs or any of the linkers of the invention.

Still further, the invention also provides any natural or artificial host cell and any components thereof (e.g., membrane fragments) comprising any of the nucleic acid sequence of the invention or expressing any polypeptide encoded thereby, or any combinations thereof.

A further aspect of the invention relates to a composition comprising an effective amount of at least one polypeptide comprising an amino acid sequence of at least one reconstituted RBM of a viral Spike protein or of any fragment thereof, any RBD or Spike protein comprising such reconstituted RBM. Still further, the composition of the invention may comprise any RBD or Spike protein comprising at least one linker that replaces an anchor loop or any part thereof, or amino acid residue/s thereof. It should be noted that such loop comprises an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. Still further, the composition of the invention may comprise any multimeric and/or multivalent antigen displaying platform thereof as defined by the invention. It should be understood that the composition of the invention may comprise any combinations of any of the RBMs, RBDs, Spike proteins and/or multimeric and/or multivalent antigen displaying platform described by the invention, any derivative, enantiomer, fusion protein, conjugate, polyvalent dendrimer thereof, and any nucleic acid sequence encoding any of these polypeptides, or any matrix, nano- or micro-particle thereof. In some specific embodiments, the reconstituted RBM of the composition of the invention comprises at least one linker and at least one fragment of the native RBM, the native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein. Still further, the native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein. At least one of the linker/s replaces this anchor loop or any part thereof, or amino acid residue/s thereof. In some embodiments, the compositions of the invention may optionally further comprise at least one pharmaceutically acceptable carrier/s, excipient/s, auxiliaries, adjuvant/s, and/or diluent/s.

In some embodiments, the composition of the invention may comprise any of the polypeptide/s, RBD/s or Spike protein/s comprising the reconstituted RBM, or a plurality of various polypeptide/s, RBD/s or Spike protein/s as defined by the invention. Non-limiting embodiments relate to any of the RBMs as denoted by SEQ ID NO: 65-84, and any variants thereof as discussed herein. In yet some further embodiments, the compositions of the invention may comprise any of the RBD/s or Spike protein/s that comprise at least one linker that replaces the anchor loop, as defined by the invention, specifically, an of the τ-linkers disclosed herein. Non-limiting embodiments for the linkers include the linker of SEQ ID NO: 57, the GVP linker, or any of the linkers of SEQ ID NO: 42-52, and variants thereof. Still further, the composition of the invention may comprise any of the multimeric and/or multivalent antigen displaying platform defined by the invention. Still further, the composition of the invention may comprise any of the nucleic acid sequences as defined by the invention. It should be understood that the composition of the invention may comprise any combination of the various polypeptides, RBM/s, RBDs, spike proteins, multimeric and/or multivalent antigen displaying platforms and/or nucleic acid sequences as defined by the invention. It should be understood that the plurality of various RBMs and any polypeptide/s, RBD/s or Spike protein/s comprising these various RBM, may vary either in the linker sequence, the entity of replaced loop residues, and the flanking residues that flank the N′ and C′ terminal fragments A and B, as presented for example by SEQ ID NO: 58-64, and any variants thereof.

The term “pharmaceutical composition” in the context of the invention means that the composition is of a grade and purity suitable for prophylactic or therapeutic administration to human subjects and is present together with at least one of carrier/s, diluent/s, excipient/s, adjuvant/s and/or additive/s that are pharmaceutically acceptable. The pharmaceutical composition may be suitable for any mode of administration whether oral or parenteral, by injection or by topical administration by inhalation, intranasal spray or intraocular drops. More specifically, pulmonary, oral, transmucosal, intestinal or parenteral delivery, including intramuscular, subcutaneous and intramedullary injections as well as rectal, intrathecal, direct intraventricular, intravenous, intraocular injections or any other medically acceptable methods of administration may be considered as appropriate administration mode for the compositions of the invention. Pharmaceutical compositions according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations. It should be understood that in addition to the ingredients particularly mentioned above, the formulations may also include other agents conventional in the art having regard to the type of formulation in question, for example those suitable for oral administration may include flavoring agents. As noted above, any of the compositions of the invention may comprise pharmaceutically acceptable carriers, vehicles, adjuvants, excipients, or diluents. As used herein pharmaceutically acceptable carriers, vehicles, adjuvants, excipients, or diluents, are well-known to those skilled in the art and are readily available to the public. It is preferred that the pharmaceutically acceptable carrier be one which is chemically inert to the active compounds and one which has no detrimental side effects or toxicity under the conditions of use.

The choice of a carrier will be determined in part by the particular active agent, as well as by the particular method used to administer the composition. The carrier can be a solvent or a dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. Each carrier should be both pharmaceutically and physiologically acceptable in the sense of being compatible with the other ingredients and not injurious to the subject. Formulations include those suitable for immersion, oral, parenteral (including subcutaneous, intramuscular, intravenous, intraperitoneal, implantation for slow release and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein. In yet some further embodiments, the composition of the invention may be an immunogenic composition. Thus, in some embodiments, the immunogenic composition of the invention may induce an immune response in a subject. “Immune response” as used herein means the activation of a host's immune system, e.g., that of a mammal, in response to the introduction of antigen. The immune response can be in the form of a cellular or humoral response, or both.

A further aspect of the invention relates to a SARS CoV2 vaccine comprising at least one polypeptide comprising an amino acid sequence of at least one reconstituted RBM of a viral Spike protein or of any fragment thereof and/or any RBD or Spike protein comprising the reconstituted RBM. Still further, in some embodiments, the SARS CoV2 vaccine of the invention may comprise any RBD or Spike protein comprising at least one linker that replaces an anchor loop or any part thereof, or amino acid residue/s thereof. It should be noted that the loop comprising an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. In yet some further embodiments, the SARS CoV2 vaccine of the invention may comprise any multimeric and/or multivalent antigen displaying platform of any of the polypeptides disclosed by the invention, as well as of any derivative, enantiomer, fusion protein, conjugate, polyvalent dendrimer thereof. Still further, the SARS CoV2 vaccine of the invention any combinations of the RBMs, RBDs, spike proteins, multimeric and/or multivalent antigen displaying platforms, and any derivative, enantiomer, fusion protein, conjugate, polyvalent dendrimer thereof, any nucleic acid sequence encoding the same, and any composition thereof. Thus, in some embodiments, the vaccine provide by the invention may be a nucleic acid vaccine, specifically, a DNA and/or RNA vaccine that encodes any of the polypeptides of the invention discussed herein and is expressed in the vaccinated subject. Specifically, any of the nucleic acid vaccines of the present disclosure.

In some embodiments, the reconstituted RBM comprises at least one linker and at least one fragment of the native RBM. More specifically, the native RBM of the vaccine of the invention, comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein. Still further, the native RBM comprises an anchor loop comprising an amino acid sequence starting at any one of the amino acid residues F456, R457 or K458 and ending at any one of the amino acid residues T470, S469 or I468 of the SARS CoV2 Spike protein. It should be noted that the at least one linker/s replaces the anchor loop or any part thereof, or amino acid residue/s thereof. In some embodiments, the vaccine of the invention optionally further comprises at least one pharmaceutically acceptable carrier/s, adjuvant/s, excipient/s, auxiliaries, and/or diluent/s.

In some embodiments, the SARS CoV2 vaccine of the invention is capable of eliciting an immune response directed at SARS CoV2 in a mammalian subject.

In some embodiments, the immune response elicited by the vaccine of the invention may involve at least one of, humoral response and cellular response. Still further, in some embodiments, the vaccine of the invention may elicit an immune response involving at least one of T cell and B cell lymphocytes. In some specific embodiments, the vaccine of the invention is capable of eliciting neutralizing antibody response to SARS CoV2. It should be understood that the vaccines as referred to herein, further encompasses any mixture of plurality of various RBMs, or RBDs or spike proteins comprising the RBMs or any of the linkers of the invention or nucleic acid molecules that encode these various polypeptides. The plurality may comprise various variants, specifically, at least 1, 2, 3, 4, 5, 6, 7, 8, 9, 10, 20, 30, 40, 50, 60, 70, 80, 90, 100 or more of the various RBMs, or RBDs or spike proteins comprising the RBMs or any of the linkers of the invention, specifically, any of the linkers of SEQ ID NO: 57, 42-52 or the GVP linker, and/or any polypeptides comprising the amino acid sequence of any one of SEQ ID NO: 65-84, or any variants and combinations thereof as discussed above.

In some embodiments, the SARS CoV2 vaccine of the invention may comprise any of the polypeptide/s, RBD/s or Spike protein/s comprising the reconstituted RBM, as defined by the invention. In yet some further embodiments, the SARS CoV2 vaccine of the invention may comprise any of the RBD/s or Spike protein/s that comprise at least one linker that replaces the anchor loop, as defined by the invention. Still further, the SARS CoV2 vaccine of the invention may comprise any of the multimeric and/or multivalent antigen displaying platform defined by the invention. Still further, the SARS CoV2 vaccine of the invention may comprise any of the nucleic acid sequences as defined by the invention. It should be understood that the SARS CoV2 vaccine of the invention may comprise any combination of the polypeptides, RBM/s, RBDs, spike proteins, multimeric and/or multivalent antigen displaying platforms and/or nucleic acid sequences as defined by the invention. Provided herein are immunogenic compositions, such as vaccines, comprising at least one of reconstituted RBM polypeptide, specifically, reconstituted SARS-CoV2 RBMs, any fragment, derivative, enantiomer, variant, conjugate and fusion protein thereof, or a combination thereof. The vaccine can be used to protect against SARS CoV2 infection that results is COVID-19, thereby treating, preventing, and/or protecting against SARS-CoV2 associated pathologies. The vaccine can significantly induce an immune response of a subject administered the vaccine, thereby protecting against and treating SARS-CoV2 infections, and related conditions. Still further, when provided prophylactically, the reconstituted RBMs or any RBDs and spike proteins of the invention or any derivative, enantiomer, fusion protein or conjugate thereof, may be provided in advance of the SARS-CoV2 infection such as to patients or subjects who are at risk for being exposed to SARS-CoV2 or who have been newly exposed to SARS-CoV2, such as healthcare workers, blood products, or transplantation tissue, and other individuals who have been exposed to a body fluid (e.g., any mucosal fluid, blood), or to any surface, substance, material, article or device that contains or may contain SARS-CoV2. The prophylactic administration of the reconstituted RBMs or any RBDs and spike proteins of the invention or any derivative, enantiomer, fusion protein or conjugate thereof prevents, ameliorates, or delays SARS-CoV2 infection. In subjects who have been newly exposed to SARS-CoV2 but who have not yet displayed the presence of the virus (as measured by PCR or other assays for detecting the virus) in various biological fluid samples, specifically, mucosal samples (nasal or oral swabs as well as in stool) efficacious treatment with the reconstituted RBMs or any RBDs and spike proteins of the invention or any derivative, enantiomer, fusion protein or conjugate thereof partially or completely inhibits or delays the appearance of the virus or minimizes the level of the virus in the blood or other body fluid of the exposed individual.

The efficacy of the reconstituted RBMs or any RBDs and spike proteins of the invention or any derivative, enantiomer, fusion protein or conjugate thereof, can be assessed in various ways well known to the skilled practitioner. For instance, one of ordinary skill in the art will understand that vaccine of the invention is efficacious in treating or inhibiting SARS-CoV2 infection in a subject by observing that the neutralizing antibodies induced thereby reduce viral load or delays or prevents a further increase in viral load. Viral loads can be measured by methods that are known in the art, for example, using PCR assays to detect the presence of SARS-CoV2 nucleic acid or antibody assays to detect the presence of SARS CoV2 protein in a sample (e.g., blood or another body fluid) from a subject or patient, or by measuring the level of circulating or anti-SARS-CoV2 antibodies in the patient. In some embodiments, the vaccine may induce a humoral immune response in the subject administered the vaccine or the immunogenic composition. In some embodiments, the induced humoral immune response may be specific for the SARS CoV2. The humoral immune response may be induced in the subject administered the vaccine by about 1.5-fold to about 100-fold, about 2-fold to about 90-fold, or about 3-fold to about 80-fold. The humoral immune response can be induced in the subject administered the vaccine by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 20-fold, at least about 30-fold, at least about 40-fold, at least about 50-fold, at least about 60-fold, at least about 70-fold, at least about 80-fold, at least about 90-fold, at least about 100-fold, or more. The humoral immune response induced by the vaccine may include an increased level of neutralizing antibodies associated with the subject administered the vaccine as compared to a subject not administered the vaccine. The neutralizing antibodies may be specific for the SARS CoV2, specifically, for the reconstituted RBMs of the invention. The neutralizing antibodies can provide protection against and/or treatment of SARS CoV2 infection and its associated pathologies in the subject administered the vaccine.

The humoral immune response induced by the vaccine may include an increased level of IgG antibodies associated with the subject administered the vaccine as compared to a subject not administered the vaccine. The level of IgG antibody associated with the subject administered the vaccine may be increased by about 1.5-fold to about 100-fold, about 2-fold to about 50-fold, or about 3-fold to about 25-fold as compared to the subject not administered the vaccine. The level of IgG antibody associated with the subject administered the vaccine can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 20-fold, at least about 30-fold, at least about 40-fold, at least about 50-fold, at least about 60-fold, at least about 70-fold, at least about 80-fold, at least about 90-fold, at least about 100-fold, or more.

In yet some further embodiments, the vaccine can induce a cellular immune response in the vaccinated subject, such response may be specific for the reconstituted RBMs or any RBDs and spike proteins of the invention. The induced cellular immune response may include eliciting a CD8⁺ T cell response, that may according to certain embodiments, include the production of cytokines such as interferon-gamma (IFN-γ), tumor necrosis factor alpha (TNF-alpha), interleukin-2 (IL-2), or any combinations thereof. In yet some further embodiments, the cellular immune response induced by the vaccine can include eliciting a CD4⁺ T cell response. In some embodiments, the CD4⁺ T cells may produce IFN-γ, TNF-α, IL-2, or a combination of IFN-γ and TNF-α. The vaccine may further induce an immune response when administered to different tissues such as the lungs or any airways, mucosal tissues, muscle or skin. The vaccine can further induce an immune response when administered via electroporation, or inhalation, or injection, or subcutaneously, or intramuscularly.

Also provided herein are methods of treating, protecting against, and/or preventing disease in a subject in need thereof by administering the vaccine to the subject.

Administration of the vaccine to the subject may induce or elicit an immune response in the subject. The induced immune response can be used to treat, prevent, and/or protect against disease, for example, pathologies relating to SARS CoV2 infection. The induced immune response provided the subject administered the vaccine resistance to one or more or SARS CoV2 strains. The vaccine dose may range between 0.001 μg to 100 mg active ingredient, specifically, the reconstituted RBMs or any RBDs and spike proteins of the invention/kg body weight/time, and in some embodiments may be 0.01 μg to 100 mg reconstituted RBM or any RBDs and spike proteins/kg body weight/time. The vaccine may be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, 2 months, 3, 4, 5, 6, 7, 8, 9, 10, 11, 12 months, or more. The number of vaccine doses for effective treatment may be 1, 2, 3, 4, 5, 6, 7, 8, 9, 10, or more.

The vaccine may be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, specifically a primate such as a human, as well as other mammals, specifically domestic animals for example, a camel or any other camelids, such as the llama, alpaca, guanaco, and vicuna of South America, a horse, a cow, a pig, a sheep, goat, a cat, a dog, or any laboratory animal, for example any rodent such as a rat, a mouse, a rabbit and the like.

In yet some further embodiments, the subject may be an avian subject, specifically, wild or domestic birds. It should be noted that the vaccine can be administered prophylactically or therapeutically. In prophylactic administration, the vaccines may be administered in an amount sufficient to induce an immune response. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician. The reconstituted RBM polypeptide of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the reconstituted RBM polypeptide. The vaccine can be delivered via a variety of routes. Typical delivery routes include parenteral administration, e.g., intradermal, intramuscular or subcutaneous delivery. Other routes include oral administration, intranasal, intravaginal and mucosal administration (such as intranasal, oral, intratracheal, and ocular). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant. The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine. The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion. The vaccine can be incorporated into liposomes, microspheres or other polymer matrices. Liposomes can consist of phospholipids or other lipids, and can be nontoxic, physiologically acceptable and metabolizable carriers that are relatively simple to make and administer. Vaccine in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively. Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof. Mucosal vaccines may be, for example, liquid dosage forms, such as pharmaceutically acceptable emulsions, solutions, suspensions, syrups, and elixirs. Excipients suitable for such vaccines include, for example, inert diluents commonly used in the art, such as, water, saline, dextrose, glycerol, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol. Excipients also can comprise various wetting, emulsifying, suspending, flavoring (e.g., sweetening), and/or perfuming agents.

Oral mucosal vaccines also may, for example, be tableted or encapsulated for convenient administration. Such capsules or tablets can contain a controlled-release formulation. In the case of capsules, tablets, and pills, the dosage forms also can comprise buffering agents, such as sodium citrate, or magnesium or calcium carbonate or bicarbonate. Tablets and pills additionally can be prepared with enteric coatings. It is contemplated that the vaccine may be administered via the human, camel or avian patient's drinking water and/or food.

“Parenteral administration” that is also contemplated by the invention includes subcutaneous injections, submucosal injections, intravenous injections, intramuscular injections, intrasternal injections, transcutaneous injections, and infusion. Injectable preparations (e.g., sterile injectable aqueous or oleaginous suspensions) can be formulated according to the known art using suitable excipients, such as vehicles, solvents, dispersing, wetting agents, emulsifying agents, and/or suspending agents. These typically include, for example, water, saline, dextrose, glycerol, ethanol, corn oil, cottonseed oil, peanut oil, sesame oil, benzyl alcohol, benzyl alcohol, 1,3-butanediol, Ringer's solution, isotonic sodium chloride solution, bland fixed oils (e.g., synthetic mono- or diglycerides), fatty acids (e.g., oleic acid), dimethyl acetamide, surfactants (e.g., ionic and non-ionic detergents), propylene glycol, and/or polyethylene glycols. Excipients also may include small amounts of other auxiliary substances, such as pH buffering agents.

The vaccine may include one or more excipients that enhance the vaccinated patient's immune response (which may include an antibody response, cellular response, or both), thereby increasing the effectiveness of the vaccine. The adjuvant(s) may be a substance that has a direct (e.g., cytokine or Bacille Calmette-Guerin (“BCG”)) or indirect effect (liposomes) on cells of the patient's immune system. Examples of often suitable adjuvants include oils (e.g., mineral oils), metallic salts (e.g., aluminum hydroxide or aluminum phosphate), bacterial components (e.g., bacterial liposaccharides, Freund's adjuvants, and/or MDP), plant components (e.g., Quil A), and/or one or more substances that have a carrier effect (e.g., bentonite, latex particles, liposomes, and/or Quil A, ISCOM). As noted above, adjuvants also include, for example, CARBIGEN™ and carbopol. It should be recognized that this invention encompasses both vaccines that comprise an adjuvant(s), as well as vaccines that do not comprise any adjuvant.

It is contemplated that the vaccine may be freeze-dried (or otherwise reduced in liquid volume) for storage, and then reconstituted in a liquid before or at the time of administration. Such reconstitution may be achieved using, for example, vaccine-grade water.

SARS-CoV2 has been associated with a viral disorder named COVID-19 which is characterized by numerous symptoms, while the most common symptoms are fever and a dry cough. The third most common symptom is fatigue. Almost 40% of cases suffered from it. ‘Sputum production’ (a thick mucus which is coughed up from the lungs) was experienced by every third person. Sputum is not saliva. It is thick mucus which is coughed up from the lungs. About 18.6% experienced shortness of breath (‘dyspnoea’). Many of the most common symptoms are shared with those of the common flu or cold while SARS-CoV2 infection rarely causes a runny nose. According to the WHO, people infected with SARS-CoV2 generally develop signs and symptoms, including mild respiratory symptoms and fever, on an average of 5-6 days after infection. While the mean incubation period is 5 to 6 days, the WHO adds that the incubation period can vary in a wide range of between 1 to 14 days. Symptoms were categorized as mild, severe, or critical and the research article describes these as follows: Critical cases: Critical cases include patients who suffered respiratory failure, septic shock, and/or multiple organ dysfunction or failure, that may lead to death. Severe cases: This includes patients who suffered from shortness of breath, respiratory frequency ≥30/minute, blood oxygen saturation ≤93%, PaO₂/FiO₂ ratio <300,30 and/or lung infiltrates >50% within 24-48 hours. Mild cases: The majority (81%) of these coronavirus disease cases were mild cases. Mild cases include all patients without pneumonia or cases of mild pneumonia. Diagnosis of SARS-COV2 is performed by RT-PCR analysis of at least two viral genes. For example, two target genes, including open reading frame1ab (ORF1ab) and nucleocapsid protein (N), may be simultaneously amplified and tested during the real-time RT-PCR assay (Xu, Y., Li, X., Zhu, B. et al. Characteristics of pediatric SARS-CoV-2 infection and potential evidence for persistent fecal viral shedding. Nat Med (2020)).

Thus, in yet a further aspect, the invention relates to a method for treating, preventing, inhibiting, reducing, eliminating, protecting or delaying the onset of an infection or an infectious clinical condition caused by SARS CoV2 in a subject in need thereof. More specifically, the method comprising the step of administering to the subject an effective amount of at least one polypeptide comprising an amino acid sequence of at least one reconstituted RBM of a viral Spike protein of SARS CoV2 or of any fragment thereof, and/or any RBD or Spike protein comprising the reconstituted RBM and/or any RBD or Spike protein comprising at least one linker that replaces an anchor loop or any part thereof, or amino acid residue/s thereof. It should be noted that the loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein. The method of the invention may comprise administering the subject any multimeric and/or multivalent antigen displaying platform of any of the RBMs, RBDs and/or Spike proteins of the invention, or any derivative, enantiomer, fusion protein, conjugate, polyvalent dendrimer thereof, any nucleic acid sequence encoding the same, and any combinations thereof, or any matrix, nano- or micro-particle thereof, any compositions thereof or any vaccine thereof. In some embodiments, the reconstituted RBM comprises at least one linker and at least one fragment of the native RBM. Still further, the native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein. More specifically, the native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein. It should be noted that at least one of said linker/s replaces the anchor loop or any part thereof, or amino acid residue/s thereof.

In some embodiments, the subject is further administered with at least one additional anti-SARS CoV2 vaccine and any therapeutic agent. It should be noted that such at least one additional anti-SARS CoV2 vaccine and/or additional therapeutic agent is administered prior to, after and/or simultaneously with administration of the polypeptide comprising an amino acid sequence of at least one reconstituted RBM of a viral Spike protein or of any fragment thereof, any RBD or Spike protein comprising the reconstituted RBM, any RBD or Spike protein comprising at least one linker that replaces an anchor loop or any part thereof, or amino acid residue/s thereof, any derivative, enantiomer, fusion protein, conjugate, polyvalent dendrimer, any multimeric and/or multivalent antigen displaying platform thereof, any nucleic acid molecule encoding the same, or any matrix, nano- or micro-particle thereof any composition thereof, any vaccine thereof. It should be noted that the loop comprising an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein.

In some embodiments, the methods of the invention may use any of the polypeptide/s, RBD/s or Spike protein/s comprising the reconstituted RBM, as defined by the invention. In yet some further embodiments, the methods of the invention may use any of the RBM/s or Spike protein/s that comprise at least one linker that replaces the anchor loop, as defined by the invention. Still further, the methods of the invention may use any of the multimeric and/or multivalent antigen displaying platform defined by the invention. Still further, the methods of the invention may use any of the nucleic acid sequences or any vectors thereof, as defined by the invention. Still further, the methods of the invention may use any of the compositions and/or vaccines as defined by the invention. It should be understood that the method of the invention may use any combination of the polypeptides, RBM/s, RBDs, spike proteins, multimeric and/or multivalent antigen displaying platforms and/or nucleic acid sequences or any matrix, nano- or micro-particle thereof, compositions and/or vaccines as defined by the invention.

In some particular and non-limiting embodiments, the polypeptides, RBM/s, RBDs, spike proteins that may be used in the methods of the invention may comprise any polypeptide or plurality of polypeptides (e.g., RBM/s, RBDs, spike proteins) comprising any of the τ-linkers of the invention, for example, any of the linkers of SEQ ID NO: 57, the GVP linker or any of the linkers of SEQ ID NOS: 42-52, or any variants thereof, specifically, any of the variants disclosed by the invention. Still further, the methods disclosed herein may use any polypeptide or plurality of polypeptides (e.g., RBM/s, RBDs, spike proteins) comprising any of the RBM/s of the invention, specifically, at least one of the RBMs of SEQ ID NO: 65-84, and any variants thereof, as disclosed in connection with other aspects of the preset disclosure. It should be noted that the invention further encompasses methods for treating and preventing infectious disease caused by viral pathogens, specifically, SARS CoV2, that causes COVID-19. The term “treatment” in accordance with disorders associated with infectious conditions may refer to one or more of the following: elimination, reducing or decreasing the intensity or frequency of disorders associated with said infectious condition. The treatment may be undertaken when disorders associated with said infection, incidence is beginning or may be a continuous administration, for example by administration every 1 to 14 days, to prevent or decrease occurrence of infectious condition in an individual prone to said condition. Such individual may be for example a subject having a compromised immune-system, in case of cancer patients undergoing chemotherapy or HIV infected subjects. Thus, the term “treatment” is also meant as prophylactic or ameliorating treatment. The term “prophylaxis” refers to prevention or reduction the risk of occurrence of the biological or medical event, specifically, the occurrence or re occurrence of disorders associated with infectious disease, that is sought to be prevented in a tissue, a system, animal or human by a researcher, veterinarian, medical doctor or other clinician, and the term “prophylactically effective amount” is intended to mean that amount of a pharmaceutical composition that will achieve this goal. Thus, in particular embodiments, the methods of the invention are particularly effective in the prophylaxis, i.e., prevention of conditions associated with infectious viral disease. Thus, subjects administered with the compositions or vaccines of the invention are less likely to experience symptoms associated with said infectious condition that are also less likely to re-occur in a subject who has already experienced them in the past. The term “amelioration” as referred to herein, relates to a decrease in the symptoms, and improvement in a subject's condition brought about by the compositions and methods according to the invention, wherein said improvement may be manifested in the forms of inhibition of pathologic processes associated with any infectious viral disease, a significant reduction in their magnitude, or an improvement in a diseased subject physiological state. The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein. The terms “delay”, “delaying the onset”, “retard” and all variations thereof are intended to encompass the slowing of the progress and/or exacerbation of an infectious disease, specifically, of SARS CoV2 infection and their symptoms slowing their progress, further exacerbation or development, so as to appear later than in the absence of the treatment according to the invention. As used herein, “disease”, “disorder”, “condition” and the like, as they relate to a subject's health, are used interchangeably and have meanings ascribed to each and all of such terms.

The present invention relates to the treatment of subjects, or patients, in need thereof. By “patient” or “subject in need” it is meant any organism who may be affected by the above-mentioned conditions, and to whom the vaccinating and treatment methods herein described is desired, including humans, domestic and non-domestic mammals such as canine and feline subjects, bovine, simian, equine and murine subjects, rodents, domestic or wild birds, aquaculture, fish and exotic aquarium fish. It should be appreciated that the treated subject may be also any reptile or zoo animal and laboratory animals. More specifically, the composition/s and method/s of the invention are intended for mammals or avian subjects. By “mammalian subject” is meant any mammal for which the proposed therapy is desired, including any primate, specifically, human, camelids, bats, equine, canine, and feline subjects, most specifically humans. It should be noted that specifically in cases of non-human subjects, the method of the invention may be performed using administration via injection, drinking water, feed, spraying, oral gavage and directly into the digestive tract of subjects in need thereof. Single or multiple administrations of the compositions or vaccines of the invention are administered depending on the dosage and frequency as required and tolerated by the patient. In any event, the composition and/or vaccine should provide a sufficient quantity of the reconstituted RBMs or any RBDs and spike proteins of the invention to effectively vaccinate and thereby treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient. Still further, the reconstituted RBMs of the invention or any compositions or kits thereof may be applied as a single daily dose or multiple daily doses, preferably, every 1 to 7 days. It is specifically contemplated that such application may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month. The application of the reconstituted RBMs or any RBDs and spike proteins of the invention or any compositions or kits thereof may last up to a day, two days, three days, four days, five days, six days, a week, two weeks, three weeks, four weeks, a month, two months three months or even more. Specifically, application may last from one day to one month. Most specifically, application may last from one day to 7 days. The invention thus provides methods for inhibiting and preventing viral infection and treating an ameliorating any pathologic condition associated therewith, specifically, CoV infections in a subject. It should be appreciated that such method may results in an inhibition, reduction, elimination, attenuation, retardation, decline, prevention or decrease of at least about 5%-99.9999%, about 10%-90%, about 15%-85%, about 20%-80%, about 25%-75%, about 30%-70%, about 35%-65%, about 40%-60% or about 45%-55%, and more specifically may be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%, of the SARS CoV2 infections, or infectious condition associated therewith as discussed above. It should be appreciated that the therapeutic methods and administration modes discussed herein are applicable for any of the therapeutic methods provided by the invention. Specifically, herein after in connection with other aspects of the invention. In more specific embodiments, any of the therapeutic definitions defined herein are applicable for any therapeutic aspects of the invention that use any passive vaccines provided by the invention, and any compounds that prevent or inhibit binding of the virus to its cognate receptor, and any antibody produced and/or isolated by the invention and any composition thereof.

The invention further provides at least one polypeptide, RBD or Spike protein comprising at least one reconstituted RBM as defined by the invention, at least one RBD or spike protein comprising at least one linker as defined by the invention, at least one multimeric and/or multivalent antigen displaying platform as defined by the invention, at least one nucleic acid sequence as defined by the invention or any matrix, nano- or micro-particle thereof, at least one composition as defined by the invention, and at least one vaccine as defined by the invention, for use in a method for treating, preventing, inhibiting, reducing, eliminating, protecting or delaying the onset of an infection or an infectious clinical condition caused by SARS CoV2 in a subject in need thereof.

A further aspect of the invention relates to a method of inducing an immune response against a SARS CoV2 in a subject in need thereof. More specifically, the method comprising the step of administering to the subject an immunogenic effective amount of at least one polypeptide comprising an amino acid sequence of at least one reconstituted RBM of a viral Spike protein of SARS CoV2 or of any fragment thereof, and/or any RBD or Spike protein comprising the reconstituted RBM and/or any RBD or Spike protein comprising at least one linker that replaces an anchor loop or any part thereof, or amino acid residue/s thereof. It should be noted that the loop comprising an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. The method of the invention may comprise administering the subject any multimeric and/or multivalent antigen displaying platform of any of the RBMs, RBDs and/or Spike proteins of the invention, or any derivative, enantiomer, fusion protein, conjugate, polyvalent dendrimer thereof, any nucleic acid sequence encoding the same or any matrix, nano- or micro-particle thereof, and any combinations thereof, any compositions thereof or any vaccine thereof. In some embodiments, the reconstituted RBM comprises at least one linker and at least one fragment of the native RBM. Still further, the native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein. More specifically, the native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein. It should be noted that at least one of said linker/s replaces the anchor loop or any part thereof, or amino acid residue/s thereof.

In some embodiments, the method of the invention may elicit a neutralizing antibody response to SARS CoV2 in said subject. It should be appreciated that in some embodiments, the subject may be further administered with at least one additional anti-SARS CoV2 vaccine. Such at least one additional anti-SARS CoV2 vaccine and/or therapeutic agent may be administered prior to, after and/or simultaneously with administration of the polypeptide comprising an amino acid sequence of at least one reconstituted RBM of a viral Spike protein, or any RBDs and spike proteins of the invention, or of any fragment thereof, any RBD or Spike protein comprising the reconstituted RBM, any derivative, enantiomer, fusion protein, conjugate, polyvalent dendrimer, any multimeric and/or multivalent antigen displaying platform thereof, any composition thereof, any vaccine thereof or any matrix, nano- or micro-particle thereof.

In some embodiments, the methods of the invention may use any of the polypeptide/s, RBD/s or Spike protein/s comprising the reconstituted RBM, as defined by the invention. In yet some further embodiments, the methods of the invention may use any of the RBD/s or Spike protein/s that comprise at least one linker that replaces the anchor loop, as defined by the invention. Still further, the methods of the invention may use any of the multimeric and/or multivalent antigen displaying platform defined by the invention. Still further, the methods of the invention may use any of the nucleic acid sequences as defined by the invention. Still further, the methods of the invention may use any of the compositions and/or vaccines as defined by the invention. It should be understood that the methods of the invention may use any combination of the polypeptides, RBM/s, RBDs, spike proteins, multimeric and/or multivalent antigen displaying platforms and/or nucleic acid sequences, compositions and/or vaccines as defined by the invention. In some particular and non-limiting embodiments, the polypeptides, RBM/s, RBDs, spike proteins that may be used in the methods of the invention may comprise any polypeptide or plurality of polypeptides (e.g., RBM/s, RBDs, spike proteins) comprising any of the τ-linkers of the invention, for example, any of the linkers of SEQ ID NO: 57, the GVP linker or any of the linkers of SEQ ID NOS: 42-52, or any variants thereof, specifically, any of the variants disclosed by the invention. Still further, the methods disclosed herein may use any polypeptide or plurality of polypeptides (e.g., RBM/s, RBDs, spike proteins) comprising any of the RBM/s of the invention, specifically, at least one of the RBMs of SEQ ID NO: 65-84, and any variants thereof, as disclosed in connection with other aspects of the preset invention.

The invention further provides at least one polypeptide, RBD or Spike protein comprising at least one reconstituted RBM as defined by the invention, at least one RBD or Spike protein comprising at least one linker as defined by the invention, at least one multimeric and/or multivalent antigen displaying platform as defined by the invention, at least one nucleic acid sequence as defined by the invention, at least one composition as defined by the invention, and at least one vaccine as defined by the invention, for use in a method of inducing an immune response against a SARS CoV2 in a subject in need thereof.

As disclosed by the examples, a fully functional SARS CoV2 RBM, as well as or any RBDs and spike proteins may be presently reconstituted by the inventors employing phage-display peptide-libraries. This is achieved by generating a vast collection of candidate RBM peptides that present a diversity of conformations. Screening such “Conformer Libraries” with corresponding ligands produced short RBM constructs (ca. 40 amino acids) that can bind both the viral receptor, specifically, the ACE2 receptor, in case of SARS CoV2, and neutralizing mAbs. Wrapp D, et al. [7], discloses a cryoEM that compares the structure of the SARS CoV2 with known structures of SARS CoV. The recent article by Yan et al., [10], discloses structural analysis of the binding interface of the ACE2 with the viral RBM.

Thus, a further aspect of the invention relates to a method for the preparation of a functional reconstituted RBM of a Spike protein of SARS CoV2. In some embodiments, the method comprising the step of:

First (a), screening a conformer library of RBMs of the viral Spike protein with at least one binding molecule. Specifically, the library comprising plurality of combinatorial display vehicles, e.g., bacteriophages, or any other vehicles or platforms for any combinatorial display systems such as yeast display, ribosome-display, or peptide display, each expressing a reconstituted RBM comprising an amino acid sequence of at least one fragment of a native RBM of a Spike protein of the SARS CoV2 and at least one combinatorial linker. The term “vehicle” as used herein is meant any display means, platform or carrier used as a scaffold to enable the expression and display of the reconstituted RBMs to the binding molecules (e.g., antibodies and/or receptor). It should be noted that the native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein. Still further, the native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein. It should be noted that at least one of said linker/s replaces said anchor loop or any part thereof, or amino acid residue/s thereof. The second step of the method of the invention (b), involves identifying and producing reconstituted RBM peptides which bind at least one of the binding molecules. It should be understood that the method as discussed herein further allows the identification of an appropriate linker that can replace the anchor loop of the SARS CoV2 spike protein or a fragment thereof (e.g., RBD or RBM), to produce a functional spike protein, RBD and/or RBM. A linker that supports a functional spike protein that can be recognized by neutralizing antibodies or the receptor ACE2, can be further isolated and used for various polypeptides as discussed above. Examples for such linkers isolated using the methods of the invention include, but are not limited to any of the τ-linkers of the invention, for example, any of the linkers of SEQ ID NO: 57, the GVP linker or any of the linkers of SEQ ID NOS: 42-52, or any variants thereof, specifically, any of the variants disclosed by the invention.

It should be further appreciated that the invention encompasses any reconstituted RBM prepared by the method of the invention. Non-limiting embodiments relate to any RBM/s, of the invention, specifically, at least one of the RBMs of SEQ ID NO: 65-84, and any variants thereof. Specific variants include at least one of the following substitutions Y489N, V483E, Q474R, L489I, N501Y, K417N, E484K, L452R, Y453F, and any combinations thereof.

In some embodiments, the functional reconstituted RBM or any RBDs and spike proteins comprising the reconstituted RBM of the invention is capable of eliciting the production of neutralizing antibodies in an immunized subject.

As indicated above, the reconstituted RBMs are constructed in conformer libraries containing combinatorial linkers. A “peptide library” is a collection of peptides, that may range from 2 to 100, specifically, from 3 to 90, 4 to 80, 5 to 70, 6 to 60, 7 to 50, 8 to 40, 9 to 30, 10 to 20 or 5 to 25 amino acid residues in length. The collection of peptides may be a random collection or it may be rationally designed based on the composition of the proteinaceous material of which the binding surface is a part. The greater the number of different peptides in the library, the better. Preferably, in the case of a random peptide library, it may contain more than 10⁶, 10⁷, 10⁸, 10⁹ or more different peptides. In the peptide library, the peptides may be displayed by any means, such as, for example, peptides displayed on phage, a combinatorial library of synthetic peptides on any solid support, such as beads, plates etc. Phage display libraries of random peptides are well known in the art. More specifically, as shown by the examples, the functional reconstituted RBMs of the invention were isolated by screening of a conformer peptide library, that enables the screening of library expressing genuine fragments of the native RBM inter-connected with a vast collection (millions) of combinatorial linkers, thus generating a “Conformer Library” where each linker enables the RBM fragments to assume a unique three-dimensional conformation. One of the factors contributing to the diversity of a phage-displayed peptide library is the method of library construction. When designing peptide-encoding oligonucleotide DNA to be inserted into the phage genome, the type of genetic code employed is of utmost importance because the observed frequency of amino acids displayed in the library is directly correlated to the number of codons encoding each amino acid. For instance, the standard 64-codon genetic code encodes each of the twenty amino acids and three stop codons with the number of codons per amino acid ranging from one (methionine, tryptophan) to six (leucine, serine, arginine). Amino acids encoded by higher numbers of codons have a greater chance of being incorporated into the library, while amino acids with fewer codons have a lesser chance. This discrepancy results in non-uniform amino acid frequencies and thus, limited peptide diversity. Ideally, a library would contain peptides with even 5% amino acid frequencies (one amino acid out of twenty) per each amino acid position within the peptides. To produce libraries with more even amino acid frequencies, phage-displayed peptide libraries are typically created using a reduced genetic code when designing the insert oligonucleotide DNA encoding the displayed peptides. The oligonucleotides are designed in the ‘NNK’ format, where N represents equal proportions of guanine, cytosine, thymine, and adenine nucleotides, and K represents equal proportions of thymine and guanine nucleotides. This method encodes all twenty amino acids and one stop codon, while smoothing the number of codons per amino acid to one, two, or three. Although the NNK method of library construction provides each amino acid a more equal opportunity of incorporation into the peptide library, the method still imparts some amino acid sequence bias because the amino acids are encoded by varying numbers codons. More specifically, for identifying functional reconstituted RBM polypeptides, conformer libraries used in step (a) of the method of the invention, are screened to identify reconstituted RBMs that contain a sufficient portion of a binding surface, folded and presented in a correct functional structure. In some embodiments, such portion of a binding surface may comprise at least one amino acid residue that participate in binding. By “Functional” as used herein, it is meant that the binding surface is capable of mimicking the native binding surface of the RBD and therefore is capable of binding the receptor as well as several neutralizing antibodies. A “binding surface” as used herein, is that portion of a proteinaceous material that associates with a binding molecule. Thus, to screen the conformer libraries provided and used by the invention, at least one binding molecule is used. The “binding molecule” is any molecule, whether or not proteinaceous, that associates with a binding surface, i.e., binds to the binding surface with specificity dependent on the structure of that surface and with high affinity. The binding molecule-binding surface association is mainly discussed herein from the standpoint of the antibody-antigen and/or a ligand-receptor association, however, it should be noted that such associations may also include a receptor-hormone association, an enzyme-substrate association, or any other protein-protein interaction. It should be however understood that while the binding molecule may itself be a proteinaceous material, such as an antibody, an enzyme, a ligand, a receptor, etc. in some embodiments, it may not be necessarily proteinaceous. Thus, for example, the binding molecule may be a polynucleotide sequence or a sugar molecule, gangliosides, lipids, etc.

To identify functional reconstituted RBM polypeptides, specifically, the RBMs that are capable of eliciting the production of neutralizing antibodies in the immunized subject, thereby inhibiting and preventing viral entry to the target cells of said subject, the library provided by the invention is screened with “binding molecules” as discussed above.

In some embodiments, the binding molecule used by the methods of the invention may be at least one of: (a) the receptor for the SARS CoV2 or any fragments thereof; (b) neutralizing antibodies of convalescent serum of at least one patient recovered from SARS CoV2 infection; (c) antibodies (e.g., monoclonal antibodies, nanobodies, i.e., camelid heavy chain derived antibodies, or fragments thereof, e.g. single chain Fvs or Fabs as further defined by the invention herein after in connection with other aspects of the invention) that neutralize the virus and compete with receptor binding, or any antigen-binding fragments thereof; and (d) and any combinations of (a), (b) and (c).

As noted herein above, the binding molecule used by the method of the invention may be an antibody, specifically, a neutralizing antibody (nAb). A neutralizing antibody capable of disturbing, reducing, decreasing, and eliminating the interaction of the virus with its host cell and thereby prevents and reduces viral entry to the target cell. More specifically, the neutralizing antibody as used herein, leads to an inhibition, reduction, elimination, attenuation, retardation, decline, prevention or decrease of at least about 5%-99.9999%, specifically, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%, of the interaction of the virus with its host cell and thereby prevents and reduces viral entry to the target cell. It should be understood that the definition of the extent of neutralization as discussed herein also apply for neutralizing antibodies used in connection with any other aspect of the invention. As indicated above, antibodies applicable in the present invention are further defined herein after in connection with other aspects of the invention. Non-limiting embodiments for antibodies that can be used by the methods of the invention include, but are not limited to the AO5, 441 and D11 antibodies used by the present examples.

In yet some further embodiments, the binding molecule used for screening the library of the invention, may be the viral receptors, or any fragments thereof. In more specific embodiments, the receptor for the SARS CoV2 is the angiotensin converting enzyme 2 (ACE2). In yet some further embodiments, the ACE2 receptor as used herein may be the human ACE2 receptor. In yet some further embodiments, the amino acid sequence of the human ACE2 receptor is as denoted by GenBank: BAB40370.1. Still further, in some embodiments, the human ACE2 receptor comprises the amino acid sequence as denoted by SEQ ID NO: 23 and encode by the nucleic acid sequence as denoted by SEQ ID NO: 24.

As indicated herein before, studies have shown that SARS-CoV-2 has a higher affinity to human ACE2 than the original SARS virus strain. An atomic-level image of the S protein has been created using cryogenic electron microscopy [7]. However, recently, additional receptors or co-receptors were identified for the SARS-Cov2. Thus, in some embodiments, the following putative receptors or co-receptors may be used as binding molecules for the methods of the invention, specifically, ANPEP (Alanyl Aminopeptidase, Membrane), DPP4 (Dipeptidyl peptidase-4 (DPP4), or adenosine deaminase complexing protein 2 or CD26 (cluster of differentiation 26), ENPEP (glutamyl aminopeptidase), and CD147 (cluster of differentiation 147). More specifically, ACE2 expression levels are rather low in lung Alveolar Type 2 cells (AT2) (4.7-fold lower than the average expression level of all ACE2 expressing cell types), raising the possible existence of co-receptors facilitating SARS-COV2 infection. It is well recognized that ssRNA viruses tend to have multiple receptors. A recent study suggests that the candidate co-receptors may be ANPEP, DPP4 and ENPEP [12]. ANPEP, alanyl aminopeptidase, is a host receptor targeted by porcine epidemic diarrhoea virus, human coronavirus 229E, feline coronavirus, canine coronavirus, transmissible gastroenteritis virus and infectious bronchitis virus. These viruses all belong to Coronaviridae. ENPEP, Glutamyl Aminopeptidase, belongs to the peptidase M1 family which is the mammalian type II integral membrane zinc-containing endopeptidases. ENPEP regulates blood pressure regulation and blood vessel formation through the catabolic pathway of the renin-angiotensin system. The relationship between ENPEP and viral infection is currently unknown. Dipeptidyl peptidase-4 (DPP4), also known as adenosine deaminase complexing protein 2 or CD26 is the receptor of MERS-CoV. It is an enzyme expressed on the surface of most cell types and is associated with immune regulation, signal transduction, and apoptosis. All of the three genes encode peptidase, which are uniquely adopted by coronavirus as their receptors.

Furthermore, it was recently reported that SARS-CoV-2 may invade host cells via a novel route of CD147-spike protein (SP). SP was shown to bind to CD147, a receptor on the host cells, thereby mediating the viral invasion [13]. It was also shown that Meplazumab, a humanized anti-CD147 antibody, could competitively inhibit the binding of SP and CD147 and prevent the viruses from invading host cells. CD147, also known as Basigin or EMMPRIN, is a transmembrane glycoprotein that belongs to the immunoglobulin superfamily, which is involved in tumor development, plasmodium invasion and virus infection. Previous studies showed that CD147 plays a functional role in facilitating SARS-CoV invasion for host cells, and CD147-antagonistic peptide-9 has a high binding rate to HEK293 cells and an inhibitory effect on SARS-CoV. Initial spike protein priming by transmembrane protease, serine 2 (TMPRSS2) is essential for entry of SARS-CoV-2 [8]. After the virus' spike protein has attached to the ACE2 molecule of its target cell, the spike is cut open by another protein on the cell exterior, protease TMPRSS2, exposing a fusion peptide. SARS-CoV-2 produces at least three virulence factors that promote shedding of new virions from host cells and inhibit immune response [5].

Initial spike protein priming by transmembrane protease, serine 2 (TMPRSS2) is essential for entry of SARS-CoV-2 [8]. After the virus' spike protein has attached to the ACE2 molecule of its target cell, the spike is cut open by another protein on the cell exterior, protease TMPRSS2, exposing a fusion peptide. SARS-CoV-2 produces at least three virulence factors that promote shedding of new virions from host cells and inhibit immune response [5].

In yet some further embodiments, the reconstituted RBM prepared by the methods of the invention may comprise at least one linker and at least two fragments of the native RBM, specifically, fragments (A) and (B) as disclosed herein. More specifically, (a) at least one of the at least two fragments, also referred to herein in some embodiments as fragment “A”, may comprise the amino acid sequence of any one of: (i) residues S443 to F456; (ii) residues S443 to R457; (iii) residues S443 to K458; (iv) residues S443 to R454; (v) residues S443 to L455; (vi) residues A435 to F456; (vii) residues A435 to R457; (viii) residues A435 to K458; (ix) residues A435 to R454; (x) residues A435 to L455; (xi) residues W436 to F456; (xii) residues W436 to R457; (xiii) residues W436 to K458; (xiv) residues W436 to R454; (xv) residues W436 to L455; (xvi) residues N437 to F456; (xvii) residues N437 to R457; (xviii) residues N437 to K458; (xix) residues N437 to R454; (xx) residues N437 to L455; (xxi) residues S438 to F456; (xxii) residues S438 to R457; (xxiii) residues S438 to K458; (xxiv) residues S438 to R454; (xxv) residues S438 to L455; (xxvi) residues N439 to F456; (xxvii) residues N439 to R457; (xxviii) residues N439 to K458; (xxix) residues N439 to R454; (xxx) residues N439 to L455; (xxxi) residues N440 to F456; (xxxii) residues N440 to R457; (xxxiii) residues N440 to K458; (xxxiv) residues N440 to R454; (xxxv) residues N440 to L455; (xxxvi) residues L441 to F456; (xxxvii) residues L441 to R457; (xxxviii) residues L441 to K458; (xxxix) residues L441 to R454; (xl) residues L441 to L455; (xli) residues D442 to F456; (xlii) residues D442 to R457; (xliii) residues D442 to K458; (xliv) residues D442 to R454; (xlv) residues D442 to L455; (xlvi) residues K444 to F456; (xlvii) residues K444 to R457; (xlviii) residues K444 to K458; (xlix) residues K444 to R454; (1) residues K444 to L455; (li) residues V445 to F456; (lii) residues V445 to R457; (liii) residues V445 to K458; (liv) residues V445 to R454; (lv) residues V445 to L455; (lvi) residues G446 to F456; (lvii) residues G446 to R457; (lviii) residues G446 to K458; (lix) residues G446 to R454; (lx) residues G446 to L455; (lxi) residues G447 to F456; (lxii) residues G447 to R457; (lxiii) residues G447 to K458; (lxiv) residues G447 to R454; (lxv) residues G447 to L455; (lxvi) residues N448 to F456; (lxvii) residues N448 to R457; (lxviii) residues N448 to K458; (lxix) residues N448 to R454; (lxx) residues N448 to L455; (lxxi) the amino acid sequence of residues of the SARS CoV2 spike protein as defined in any one of (i) to (lxx) with at least one or two flanking amino acid residue/s; (lxxii) any mutant, variant, parts or fragments of the amino acid sequence of residues of the SARS CoV2 spike protein as defined in any one of (i) to (lxx); and (b) at least one of the at least two fragments, also referred to herein in some embodiments as fragment “B”, may comprises the amino acid sequence of any one of: (i) residues Y473 to Y495; (ii) residues Y473 to G496; (iii) residues Y473 to F497; (iv) residues Y473 to Q498; (v) residues Y473 to P499; (vi) residues Y473 to T500; (vii) residues Y473 to N501; (viii) residues Y473 to G502; (ix) residues Y473 to V503; (x) residues Y473 to G504; (xi) residues Y473 to Y505; (xii) residues Y473 to Q506; (xiii) residues Y473 to P507; (xiv) residues Y473 to Y508; (xv) residues Y473 to R509; (xvi) residues Y473 to V510; (xvii) residues I472 to Y495; (xviii) residues I472 to G496; (xix) residues I472 to F497; (xx) residues I472 to Q498; (xxi) residues I472 to P499; (xxii) residues I472 to T500; (xxiii) residues I472 to N501; (xxiv) residues I472 to G502; (xxv) residues I472 to V503; (xxvi) residues I472 to G504; (xxvii) residues I472 to Y505; (xxviii) residues I472 to Q506; (xxix) residues I472 to P507; (xxx) residues I472 to Y508; (xxxi) residues I472 to R509; (xxxii) residues I472 to V510; (xxxiii) residues E471 to Y495; (xxxiv) residues E471 to G496; (xxxv) residues E471 to F497; (xxxvi) residues E471 to Q498; (xxxvii) residues E471 to P499; (xxxviii) residues E471 to T500; (xxxix) residues E471 to N501; (xl) residues E471 to G502; (xli) residues E471 to V503; (xlii) residues E471 to G504; (xliii) residues E471 to Y505; (xliv) residues E471 to Q506; (xlv) residues E471 to P507; (xlvi) residues E471 to Y508; (xlvii) residues E471 to R509; (xlviii) residues E471 to V510; (xlix) residues T470 to Y495; (1) residues T470 to G496; (li) residues T470 to F497; (lii) residues T470 to Q498; (liii) residues T470 to P499; (liv) (iv) residues T470 to T500; (lv) (v) residues T470 to N501; (lvi) residues T470 to G502; (lvii) residues T470 to V503; (lviii) residues T470 to G504; (lix) residues T470 to Y505; (lx) residues T470 to Q506; (lxi) residues T470 to P507; (lxii) residues T470 to Y508; (lxiii) residues T470 to R509; (lxiv) residues T470 to V510; (lxv) residues S469 to Y495; (lxvi) residues S469 to G496; (lxvii) residues S469 to F497; (lxviii) residues S469 to Q498; (lxix) residues S469 to P499; (lxx) residues S469 to T500; (lxxi) residues S469 to N501; (lxxii) residues S469 to G502; (lxxiii) residues S469 to V503; (lxxiv) residues S469 to G504; (lxxv) residues S469 to Y505; (lxxvi) residues S469 to Q506; (lxxvii) residues S469 to P507; (lxxviii) residues S469 to Y508; (lxxix) residues S469 to R509; (lxxx) residues S469 to V510; (lxxxi) residues I468 to Y495; (lxxxii) residues I468 to G496; (lxxxiii) residues I468 to F497; (lxxxiv) residues I468 to Q498; (lxxxv) residues I468 to P499; (lxxxvi) residues I468 to T500; (lxxxvii) residues I468 to N501; (lxxxviii) residues I468 to G502; (lxxxix) residues I468 to V503; (xc) residues I468 to G504; (xci) residues I468 to Y505; (xcii) residues I468 to Q506; (xciii) residues I468 to P507; (xciv) residues I468 to Y508; (xcv) residues I468 to R509; (xcvi) residues I468 to V510; (xcvii) residues Q474 to Y495; (xcviii) residues Q474 to G496; (xcix) residues Q474 to F497; (c) residues Q474 to Q498; (ci) residues Q474 to P499; (cii) residues Q474 to T500; (ciii) residues Q474 to N501; (civ) residues Q474 to G502; (cv) residues Q474 to V503; (cvi) residues Q474 to G504; (cvii) residues Q474 to Y505; (cviii) residues Q474 to Q506; (cix) residues Q474 to P507; (cx) residues Q474 to Y508; (cxi) residues Q474 to R509; (cxii) residues Q474 to V510; (cxiii) residues A475 to Y495; (cxiv) residues A475 to G496; (cxv) residues A475 to F497; (cxvi) residues A475 to Q498; (cxvii) residues A475 to P499; (cxviii) residues A475 to T500; (cxix) residues A475 to N501; (cxx) residues A475 to G502; (cxxi) residues A475 to V503; (cxxii) residues A475 to G504; (cxxiii) residues A475 to Y505; (cxxiv) residues A475 to Q506; (cxxv) residues A475 to P507; (cxxvi) residues A475 to Y508; (cxxvii) residues A475 to R509; (cxxviii) residues A475 to V510; (cxxix) residues G476 to Y495; (cxxx) residues G476 to G496; (cxxxi) residues G476 to F497; (cxxxii) residues G476 to Q498; (cxxxiii) residues G476 to P499; (cxxxiv) residues G476 to T500; (cxxxv) residues G476 to N501; (cxxxvi) residues G476 to G502; (cxxxvii) residues G476 to V503; (cxxxviii) residues G476 to G504; (cxxxix) residues G476 to Y505; (cxl) residues G476 to Q506; (cxli) residues G476 to P507; (cxlii) residues G476 to Y508; (cxliii) residues G476 to R509; (cxliv) residues G476 to V510; (cxlv) the amino acid sequence of residues of the SARS CoV2 Spike protein as defined in any one of (i) to (cxliv) with at least one or two flanking amino acid residue/s; (cxlvi) any variants, mutants, parts or fragments of the amino acid sequence of residues of the SARS CoV2 Spike protein as defined in any one of (i) to (cxliv). It should be understood that in some specific and non-limiting embodiments, the amino acid residues and positions disclosed herein for fragments “A” and “B”, may refer to the spike protein of SARS CoV2. In some embodiments, the spike protein may comprise the amino acid sequence as denoted by SEQ ID NO: 6, or any variants and mutants thereof. In some particular and non-limiting embodiments, variants and/or mutants as used herein, refer to a spike protein, for example, the spike protein comprising the amino acid sequence as denoted by SEQ ID NO: 6, with at least one of the following substitutions Y489N, Q474R, V483E, L492I, N501Y, K417N, E484K, L452R, Y453F, S13I, L18F, T20N, P26S, H69-V70 deletion, D80A, D138Y, Y144/145 deletions, W152C, R190S, D215G, L242/A243/L244 deletion, R246T, A570D, D614G, H655Y, P681H, I692V, A701V, T716I, S982A, T1027I, D1118H, V1176F, M1229I, or any combinations thereof. Non-limiting embodiments for such variants are denoted by SEQ ID NO: 38, 39 and 41. In some embodiments, the reconstituted RBM prepared by the methods of the invention comprises at least one linker and at least two fragments of the native RBM or any variants thereof. More specifically, at least two fragments of the reconstituted RBM used and screened by the methods of the invention comprise the amino acid sequence of any one of:

A first fragment (a), also referred to herein in some embodiments as fragment “A”, that comprise the amino acid sequence of any one of:
(i) residues S443 to F456 of the SARS CoV2 Spike protein; (ii) residues S443 to F456 of the SARS CoV2 Spike protein with at least one to eight flanking amino acid residue/s; or (iii) any variants, mutants, parts or fragments of the amino acid sequence of residues S443 to F456 of the SARS CoV2 Spike protein; and A second fragment (b), also referred to herein in some embodiments as fragment “B”, that comprise the amino acid sequence of any one of:
(i) residues Y473 to P499 of the SARS CoV2 Spike protein;
(ii) residues Y473 to P499 of the SARS CoV2 Spike protein with at least one to eleven flanking amino acid residue/s; or
(iii) any variants, mutants, parts or fragments of the amino acid sequence of residues Y473 to P499 of the SARS CoV2 Spike protein. It should be understood that in some specific and non-limiting embodiments, the amino acid residues and positions disclosed herein for fragments “A” and “B”, may refer to the spike protein of SARS CoV2. In some embodiments, the spike protein may comprise the amino acid sequence as denoted by SEQ ID NO: 6, or any variants and mutants thereof. In some particular and non-limiting embodiments, variants and/or mutants as used herein, refer to a spike protein, for example, the spike protein comprising the amino acid sequence as denoted by SEQ ID NO: 6, with at least one of the following substitutions Y489N, Q474R, V483E, Q474R, L492I, N501Y, E484K, L452R, Y453F or any combinations thereof. Non-limiting embodiments for such variants are denoted by SEQ ID NO: 38, 39 and 41. In some particular and non-limiting embodiments, fragment “A” comprises the S443 to F456 of the SARS CoV2 Spike protein, as denoted by SEQ ID NO: 3, and any variants and mutants thereof, as discussed above. In yet some further embodiments, fragment “B” comprises the Y473 to P499 of the SARS CoV2 Spike protein, as denoted by SEQ ID NO: 4, and any variants and mutants thereof, as discussed above. The at least one linker used for the reconstituted RBM screened by the methods of the invention may be a bridging linker that bridges residue that flank the anchor loop removed by the invention. Such bridging linker, also referred to herein as a τ-linker (tau linker) may bridge an one of the amino acid residues F456, R457, K458, R454 or L455 with any one of the amino acid residues Y473, I472, E471, T470, S469, I468, Q474, A475 or G476 of the SARS CoV2 Spike protein. In some embodiments, the bridging linker may bridge the amino acid residue F456 with T473 of the SARS CoV2 Spike protein. Various embodiments for various residues that are bridged by the linkers of the present disclosure are as specified herein before in connection with other aspects of the invention. In some embodiments, at least one linker that is a linker that replaces the anchor loop or any part thereof, or amino acid residue/s thereof, is a bridging linker. Such bridging linker bridges and the last amino acid residue of the N′ terminal fragment (also referred to herein as the first fragment or as fragment A) of the reconstituted RBM with the first amino acid residue of the C′ terminal fragment (also referred to herein as the second fragment or as fragment B) of the reconstituted RBM. In some embodiments the bridging linker bridges the amino acid residues that flank the replaced anchor loop or any part thereof, or amino acid residue/s thereof. In yet some further embodiments, the linker may comprise any compound bridging the two fragments of the reconstituted RBM. In more embodiments, the linker is an inert linker. In yet some further embodiments, such linker is any inorganic or organic molecule, any small molecule, any peptide (L- as well as D-aa residues), or any combinations thereof.

In some embodiments, the amino acid linker in accordance with the invention is a bridging linker comprising between about 3 to about 7 consecutive amino acid residues. In yet some further embodiments, the sequence of any of said linkers of 3, 4, 5, 6, or 7 amino acid residues, does not exist in the anchor loop of the spike protein of SARS CoV2, specifically, the anchor loop as defined by the invention. In yet some further embodiments, the sequence of any of the linkers of 3, 4, 5, 6, or 7 amino acid residues, does not exist in the anchor loop of a spike protein of any beta Corona Virus.

In yet some further specific embodiments, the at least one combinatorial linker of the reconstituted RBM used and screened by the methods of the invention, is an amino acid linker. In more specific embodiments, such linker is a combinatorial linker comprising any combination of 3, or 4, or 5, or 6, or 7 or more, amino acid residues. Non-limiting embodiments for such linkers are provided by FIG. 16. More specifically, the τ-linkers of the invention may be any of the linkers of SEQ ID NO: 57, the GVP linker or any of the linkers of SEQ ID NOS: 42-52, or any variants thereof, specifically, any of the variants disclosed by the invention.

In the present context, Conformer Libraries express constant compositions and sequences of the RBM per se yet differ in the details of their three-dimensional conformations. This was accomplished by implementing a vast collection of random combinatorial linkers varying in length and composition. The affinity selection of conformers that satisfy the spatial orientation and display of the native contact residues has been proven very efficient. Validity of this method was supported by the fact that three independent conformation-specific probes detected constructs incorporating the same linkers. These three probes represent two very different modes of interaction with an extended surface of the RBM.

For any specific case, the inventors propose to remove/delete the “anchor loop”, “loop” that functions for RBM anchoring, tacking, binding and attaching to the body of RBD core, any parts thereof or any RBM fragment or amino acid residue not involved, participating or essential to receptor interaction and binding, and thus faces in the opposite direction relative to the binding surface that interacts with the receptor and is the target of neutralizing antibodies. As a result of this deletion, a gap is generated that requires a linker. The inventors have presently demonstrated how to design a potential linker, in terms of length and composition, by introducing the concept of combinatorial linkers and conformer libraries, and a method for screening these libraries with concrete reagents that have affinity for RBM, i.e. the receptor and neutralizing antibodies.

More specifically, the selection of the correct linker length and composition is based on a pull down and affinity selection of conformers bearing the correct linker, and thereby reconstitution of a recognizable conformation.

Composition of the successful linkers indicates that they are not random, but rather represent a motif in themselves, thus illustrating the uniqueness of these linkers and the fact that affinity selection not only works but apparently is necessary to discriminate the correct linkers from the vast collection of all other linkers in the conformer library. It should be appreciated that the method of preparation of a functional reconstituted RBM in accordance with this aspect of the invention may further provide a method for identifying at least one linker that can functionally replace the anchor loop in the RBM, the RBD the S1 subunit or the entire spike protein of any coronavirus, specifically the SARS CoV2. Such functional linker may provide an effective approach for improving vaccines based on the spike protein of corona viruses, specifically, the SARS CoV2. The immunogenic peptides conceived by the present invention are applicable for generation of specific vaccines and vaccine-based therapeutic compositions targeting SARS CoV2. A number of methods for producing specific formulation for vaccines are known in the art. A rational systematic approach for the development of vaccine formulations was provided for example by Morefield G. L. in AAPS J. 2011, Vol. 13(2), pp. 191-200.

Thus, in yet another aspect, the invention provides a method for producing SARS CoV2 vaccine comprising reconstituted RBM. More specifically, the method comprising the steps of:

First in step (a), preparing reconstituted functional RBM/s of a Spike protein of SARS CoV2 by a method as defined by the invention and discussed herein above. The next step (b), involves admixing at least one of the reconstituted functional RBM/s of a Spike protein of SARS CoV2 or any derivative or enantiomer thereof, or any fusion protein, conjugate, or polyvalent dendrimer comprising the same with at least one adjuvant/s, carrier/s, excipient/s, auxiliaries, and/or diluent/s. In yet some further embodiments, the reconstituted RBMs of the invention or any derivative, enantiomer, fusion protein or conjugate thereof may be presented in the vaccines of the invention as a polyvalent antigen by incorporation thereof in a polyvalent dendrimer. This embodiment is based on the knowledge in the art that a multiple antigen peptide carrying a multiplicity of epitopes induces superior immune responses compared to responses following immunization with corresponding equal amounts of monovalent epitopes. Thus, in some embodiments, the present invention is intended to broadly encompass antigenic products carrying multiple copies of the reconstituted RBM polypeptides or any RBDs and spike proteins of the present invention an in a multiple antigen peptide system. The present dendritic polymers are antigenic products in which the reconstituted RBM polypeptides are covalently bound to the branches that radiate from a core molecule. These dendritic polymers are characterized by higher concentrations of functional groups per unit of molecular volume than ordinary polymers. Generally, they are based upon two or more identical branches originating from a core molecule having at least two functional groups. The polymers are often referred to as dendritic polymers because their structure may be symbolized as a tree with a core trunk and several branches. Unlike a tree, however, the branches in dendritic polymers are substantially identical. The dendrite system has been termed the “multiple antigen peptide system” (MAPS), which is the commonly used name for a combination antigen/antigen carrier that is composed of two or more, usually identical, antigenic molecules, specifically, the reconstituted RBM polypeptides or any RBDs and spike proteins of the invention covalently attached to a dendritic core which is composed of principal units which are at least bifunctional/difunctional. Each bifunctional unit in a branch provides a base for added growth.

The dendritic core of a multiple antigen peptide system may be composed of lysine molecules. For example, a lysine is attached via peptide bonds through each of its amino groups to two additional lysines. This second generation molecule has four free amino groups each of which can be covalently linked to an additional lysine to form a third generation molecule with eight free amino groups. A peptide may be attached to each of these free groups to form an octavalent multiple peptide antigen (MAP). The process can be repeated to form fourth or even higher generations of molecules. With each generation, the number of free amino groups increases geometrically and can be represented by 2ⁿ, where n is the number of the generation. Alternatively, the second-generation molecule having four free amino groups can be used to form a tetravalent MAP with four peptides covalently linked to the core. Many other molecules, including, e.g., the amino acids Asp and Glu, both of which have two carboxyl groups and one amino group to produce poly Asp or poly Glu with 2n free carboxyl groups, can be used to form the dendritic core of MAPS.

The term “dendritic polymer” is sometimes used herein to define a product of the invention. The term includes carrier molecules which are sufficiently large to be regarded as polymers as well as those which may contain as few as three monomers.

The chemistry for synthesizing dendritic polymers is known and available. With amino acids, the chemistry for blocking functional groups which should not react and then removing the blocking groups when it is desired that the functional groups should react has been described in detail in numerous patents and scientific publications. The dendritic polymers and the entire MAP can be produced on a resin and then removed from the polymer. Ammonia or ethylenediamine may be utilized as the core molecule. In this procedure, the core molecule is reacted with an acrylate ester and the ester groups removed by hydrolysis. The resulting first-generation molecules contain three free carboxyl groups in the case of ammonia and four free carboxyl groups when ethylenediamine is employed. The dendritic polymer may be further extended with ethylenediamine followed by another acrylic ester monomer and repeats the sequence until the desired molecular weight was attained. It is readily apparent to one skilled in the art, that each branch of the dendritic polymer can be lengthened by any of a number of selected procedures. For example, each branch can be extended by multiple reactions with Lys molecules.

Some important features of the dendritic polymer as an immunogenic carrier are that the precise structure is known, there are no “antigenic” contaminants or those that irritate tissue or provoke other undesirable reactions. The precise concentration of the reconstituted RBM polypeptide of the invention is known; and is symmetrically distributed on the carrier; and the carrier can be utilized as a base for more than one reconstituted RBM polypeptide or any RBDs and spike proteins so that multivalent immunogens or vaccines can be produced.

When the MAPS is to be employed to produce a vaccine or immunogenic composition, it is preferred that the core molecule of the dendrimer be a naturally occurring amino acid such as Lys so that it can be properly metabolized. However, non-natural amino acids residues may be also employed. The amino acids used in building the core molecule can be in either the D or L-form. In brief, in manufacturing vaccine products it is important to have a good understanding of what factors can impact the safety, efficacy, and stability of the formulation all along the development path. The main phases in this process are: biophysical characterization of the antigen, evaluation of stabilizers, investigation of antigen interactions with adjuvants, evaluation of product contact materials such as sterile filter membranes, and monitoring stability both in real time and under accelerated conditions. Biophysical Characterization Phase refers to evaluation of the physical characteristics of the antigen, i.e. understanding how parameters such as pH, buffer species, and ionic strength impact the folded state of the antigen as well as the propensity of the antigen to aggregate. Knowing how characteristics of the formulation will impact physical stability of the antigen will aid selection of appropriate excipients during the development process.

Empirical Phase refers to initiation of preformulation studies for the systematic development of a vaccine formulation. A logical place to start, is understanding how the physical stability of the antigen is impacted by changes in pH and temperature. The pH of the formulation can impact both the physical stability of the antigen, such as whether the antigen maintains the appropriate folding and if the antigen will aggregate, as well as the chemical stability of the antigen. The pH can impact the chemical degradation rate of many mechanisms of degradation such as hydrolysis, oxidation, and deamidation. The Empirical Phase Diagram offers a convenient way to display how the physical stability of an antigen is impacted with changes in pH and temperature. Generally, in this approach, characterization data are taken from various spectroscopic techniques such as second derivative UV/Vis, intrinsic fluorescence, extrinsic fluorescence, and circular dichroism are combined and transformed into data vectors to construct the empirical phase diagram. In addition to pH evaluation, the Empirical Phase Diagram approach can be utilized to determine the impact of other variables on antigen stability like buffer type and concentration, ionic strength, and impact of product contact material.

Evaluation of Stabilizers refers to optimization of formulation parameters such as pH, ionic strength, and buffer species may not prove to be enough to stabilize an antigen for the typically desired 3-year shelf life of vaccine products. In this case stabilizing excipients need to be investigated for incorporation into the vaccine formulation. Evaluation of antigen stabilizers typically begins with investigation of generally regarded as safe (GRAS) excipients. By utilizing GRAS excipients, development may proceed more rapidly as regulatory concerns regarding safety of the formulation excipients will be lower. Since at the early stage of development the primary mechanism of antigen degradation may not be known it is important to evaluate excipients from various classes of stabilizers. Excipient screening such as monitoring of optical density or extrinsic fluorescence can be performed in a 96 well or more format to allow high-throughput screening of many excipients and excipient combinations at one time.

Correlation of Real-time and Accelerated Stability, refer to analysis of the stability of a formulation under extreme environmental conditions such as high temperatures. Correlation of accelerated stability with real-time data is valuable to support activities such as expiration dating and assessment of the impact of temperature excursions during shipment and storage of the vaccine for clinical trials. When initiating stability studies it is important to understand potential mechanisms of antigen can degradation. In general, physical instability is associated with loss of protein structure and aggregation while common forms of chemical degradation are oxidation and deamidation. In addition to high temperature excursions, it is useful to determine the impact of other factors on formulation stability, such as exposure to environmental stresses such as cycles of freezing and thawing, extended exposure to light, and contact with various storage container materials. Adjuvants or Adjuvantation refers to enhancement of antigen immunogenicity. A side effect of vaccine antigens becoming more pure as purification technology has advanced is a reduction in the immunogenicity of the antigen. To retain antigen immunogenicity with more highly purified antigens, adjuvants can be incorporated into the vaccine formulation. Adjuvants interact with the immune system through various mechanisms thereby enhancing the immune response. Currently, the most utilized adjuvants in licensed products are aluminum salts and squalene-based oil-in-water emulsions. Aluminum-containing adjuvants, including aluminum hydroxide (AlO(OH) and aluminum phosphate (Al(OH)ₓ(PO₄)_{y} adjuvants, have a long history of use and an excellent safety profile. Sterile Filtration refers to prevention of microbial contamination of vaccines is an important part of producing a safe vaccine formulation. As vaccines are administered to infants, children, and adults who are generally healthy at the time of injection there is a high level of safety that must be ensured when manufacturing the vaccine product. Typically, this can be achieved through aseptic processing and sterile filtration of the vaccine formulation. However, formulations with aluminum-containing adjuvants cannot be sterilized by filtration due to the particle size of the adjuvant being greater than 0.2 μm. Materials used to prepare vaccines with aluminum-containing adjuvants must be sterilized prior to formulation and handled aseptically during the formulation and filling process. Sterile filter membranes are produced with various materials, typical membranes used in vaccine production are cellulose acetate, polyethersulfone and polyvinylidene fluoride.

The functional reconstituted RBMs provided by the methods of the invention may be used as a vaccines, as described herein. Importantly, in addition to uses thereof as effective vaccines, the reconstituted RBMs provided by the methods of the invention may be used as effective baits for isolating any molecules or compounds that bind the SARS CoV2 spike protein and thereby reduces, eliminates, prevents and inhibits binding of the virus to the cognate receptor, specifically, the ACE2 receptor. Such inhibitory molecules or compounds may be antibodies, specifically, neutralizing antibodies, as well as any small molecules, aptamers and the like. Screening candidate compounds and/or antibodies (either using antibody libraries, serum and antibody producing lymphocytes) may be effectively performed using the reconstituted RBM of the invention, either in a soluble form thereof, or displayed in the conformer library provided by the invention. More specifically, Example 13 presents the conformer library displayed on a bacteriophage as an effective scaffold for RBM that may be used as a bait. In some specific embodiments, such conformer library is displayed on filamentous bacteriophages based on the fth1 phage vector. The combinatorial linker library is expressed on Protein 3 which exists in five copies. Thus, in accordance with some embodiments of the invention, the reconstituted RBM of the invention is expressed and displayed on Protein 3 scaffold. Still further, in some embodiments, the reconstituted RBM of the invention may be further connected directly or indirectly to at least one tag, detectable moiety or affinity moiety. In some embodiments such tag, detectable moiety or affinity moiety may be any affinity molecule or moiety disclosed by the invention, for example, at least one biotinylation site, histidine tag, Flag, HA, myc and the like. In some embodiments, the RBM of the invention may be associated to biotin. Thus, in some embodiments, the invention provides a biotinylated phage expressing the functional RBM. In yet some further embodiments, such biotinylated phage expressing the functional RBM of the invention can used as a bait. More specifically, in a previous study (Smelyanski L et al., Virology Journal 2011, 8:495), the inventors described the introduction of a biotinylation site (“Avi-Tag”) in anyone of the structural proteins of the filamentous phage, proteins 3, 7, 8 and 9. In each case, the phage can be biotinylated in vivo and thus released from the bacterium with a biotin molecule. In yet some alternative embodiments, the Avi-Tag can be biotinylated in vitro by reacting the phage with the natural biotinylating enzyme, biotin holoenzyme synthetase the product of the BirA gene in E. coli. Thus, in some particular and non-limiting embodiments, the invention provides a RBM expressing phage. More specifically, the RBM being expressed on Protein 3, which is also biotinylated on Protein 7, 8 and/or 9. In some embodiments, the RBM expressing phage is biotinylated on Protein 8. As discussed by Example 13, this may be achieved by either expressing the “Avi-Tag” using the recombinant Protein 8 gene in the vector or using an extraneous Protein 8 gene. This configuration could be very easily used without the need to clone the functional RBM into a new vector or scaffold system. The successfully cloned ACE2 binding RBM expressed on Protein 3 could be easily then biotinylated using the extraneous Avi-Tag containing protein 8. In yet some further embodiments, the biotinylated phage could then be immobilized on any Streptavidin or Avidin or anti-Avidin antibody containing surface or chromatographic medium such as sepharose 4b, or magnetic bead system. The immobilized RBM expressing phage could then be used directly to affinity purify RBM specific antibodies from convalescent serum, used to affinity select memory B-cells for single cell antibody cloning, or used to affinity select any compound (e.g., small molecule, an aptamer) that may bind the RBM thereby inhibiting and/or preventing binding of the virus to the cognate receptor.

It should be however appreciated that the invention further encompasses the use of any detectable moiety, affinity moiety or tag for any of the polypeptides disclosed by the invention, specifically, any of the reconstituted RBMs, or any RBDs or spike protein comprising the reconstituted RBMs of the invention, or any RBDs or spike proteins comprising a linker that replaces the anchor loop thereof, specifically, the τ-linker of the invention. In some further embodiments, the detectable moiety, affinity moiety or tag associated directly or indirectly with the reconstituted functional RBM of the invention, may refer to any chemical moiety that can be used to provide a detectable signal, and or attachment or affinity to a solid support, and that can be attached to an encoding nucleic acid sequence or protein via a covalent bond or noncovalent interaction (e.g., through ionic or hydrogen bonding, or via immobilization, adsorption, or the like). Labels generally provide signals detectable by at least one of fluorescence, chemiluminescence, radioactivity, colorimetry, mass spectrometry, X-ray diffraction or absorption, magnetism, enzymatic activity, electrochemical active compounds, or the like. In some specific embodiments, the detectable moiety, affinity moiety or tag useful by the invention may be at least one of conductive, electrochemical, fluorescent, chemiluminescent, enzymatic, radioactive, magnetic, metal, and colorimetric label, or any combinations thereof. Examples of labels useful in connection with the invention, include, but are not limited to at least one of haptens, enzymes, enzyme substrates, coenzymes, enzyme inhibitors, fluorophores, quenchers, chromophores, magnetic particles or beads, redox sensitive moieties (e.g., electrochemically active moieties), luminescent markers, radioisotopes (including radionucleotides), conductive materials, or electrochemical materials that in some embodiments may be suitable for electrochemical detection, specifically, nano- and micro-sized materials, such as gold nanoparticles (GNPs), latex, carbon nanotubes (CNTs), graphene (GR), magnetic particles (MBs), quantum dots (QDs) and conductive polymers, biobarcodes and members of binding pairs. More specific examples include at least one of fluorescein, phycobiliprotein, tetraethyl rhodamine, and beta-galactosidase. Binding pairs may include biotin/Strepavidin, biotin/avidin, biotin/neutravidin, biotin/captavidin, GST/glutathione, maltose binding protein/maltose, calmodulin binding protein/calmodulin, enzyme-enzyme substrate, receptor-ligand binding pairs, and analogs and mutants of the binding pairs. It should be appreciated that the use of tags for labeling directly or indirectly the reconstituted RBM of the invention, or any scaffold displaying such RBM is also encompassed by the invention. Non-limiting examples for such tag may include His-tag, Flag, HA, myc and the like. It should be further appreciated that the detectable moieties disclosed herein are applicable for any aspect of the invention. The invention further encompasses the use of any of the labeled or tagged reconstituted RBMs, or any RBDs or spike protein comprising the reconstituted RBMs of the invention, or any RBDs or spike proteins comprising a linker that replaces the anchor loop thereof, specifically, the τ-linker of the invention, in any of the aspects of the invention, specifically, in any of the methods for selecting antibodies or compounds that bind the Spike protein of SARS CoV2 and prevents binding of the spike protein to the cognate receptor. Particularly for methods for identifying neutralizing antibodies, as well as for the diagnostic methods and kits disclosed by the invention herein after.

Thus, a further aspect of the invention relates to a method for the preparation, affinity selection and/or isolation of neutralizing antibodies that neutralize the SARS CoV2 and compete with receptor binding. More specifically, the method comprising the steps of:

The first step (a), involves contacting a serum or lymphocytes of at least one donor with an effective amount of at least one reconstituted RBM of the Spike protein of SARS CoV2, associated directly or indirectly to a solid support and/or a detectable moiety. Alternatively, or in addition, the contacting step may be performed using any fragment of the reconstituted RBM, any RBD or Spike protein comprising the reconstituted RBM associated directly or indirectly to a solid support and/or a detectable moiety may be used. In yet some further embodiments, any RBD or Spike protein comprising at least one linker that replaces an anchor loop or any part thereof, or amino acid residue/s thereof, associated or attached directly or indirectly to a solid support and/or a detectable moiety may be used for this contacting step. In this connection, it should be noted that in some embodiments, the loop replaced either completely or partly by the linkers in the RBD or Spike protein used in these embodiments, comprises an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. Still further, it should be appreciated that any multimeric and/or multivalent antigen displaying platform of any of the RBMs, RBDs and/or spike proteins discussed above, any derivative, enantiomer, fusion protein, conjugate, polyvalent dendrimer thereof, or and any combinations thereof, associated directly or indirectly to a solid support and/or a detectable moiety, may be used for this step of contacting. The next step of the methods of the invention (b), involves recovering the antibodies or at least one lymphocyte bound to the reconstituted RBM, or to any RBDs and/or Spike proteins comprising the reconstituted RBM, to any RBDs and/or spike proteins discussed above, any multimeric and/or multivalent antigen displaying platform any derivative, enantiomer, fusion protein, conjugate, polyvalent dendrimer thereof, used in step (a). In some specific and non-limiting embodiments, the serum or lymphocytes may be obtained from a mammalian subject. In yet some further embodiments, the mammalian subject may be a human subject. Still further, in some embodiments the serum or lymphocytes may be obtained from a patient recovered from SARS CoV2 infection. In yet some further embodiments, the serum or lymphocytes may be obtained from at least one individual, or a population of individuals previously exposed to a viral infection. In some further embodiments, the at least one individual, or a population of individuals previously exposed to an infection with at least one corona virus. In yet some further embodiments, the serum or lymphocytes may be obtained from a at least one individual, or a population of individuals previously exposed, and specifically recovered from at least one infection with a corona virus such as SARS CoV and MERS CoV. Still further, the serum or lymphocytes may be obtained from a healthy individual that was not exposed to a viral infection with any corona virus.

In more specific embodiments, the methods of the invention are particularly suitable for the production of human monoclonal neutralizing antibodies that neutralize the SARS CoV2. More specifically, such methods comprise the steps of: In a first step (a), contacting lymphocytes of at least one donor with an effective amount of at least one reconstituted RBM of the Spike protein of SARS CoV2, associated directly or indirectly to a solid support and/or a detectable moiety. Alternatively, or in addition, the contacting step may be performed using any fragment of the reconstituted RBM, any RBD or Spike protein comprising the reconstituted RBM associated directly or indirectly to a solid support and/or a detectable moiety may be used. In yet some further embodiments, any RBD or Spike protein comprising at least one linker that replaces an anchor loop or any part thereof, or amino acid residue/s thereof, associated or attached directly or indirectly to a solid support and/or a detectable moiety may be used for this contacting step. In this connection, it should be noted that in some embodiments, the loop replaced either completely or partly by the linkers in the RBD or Spike protein used in these embodiments, comprises an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. Still further, it should be appreciated that any multimeric and/or multivalent antigen displaying platform of any of the RBMs, RBDs and/or spike proteins discussed above, any derivative, enantiomer, fusion protein, conjugate, polyvalent dendrimer thereof, and/or any combinations thereof, associated directly or indirectly to a solid support and/or a detectable moiety, may be used for this step of contacting.

The next step (b), involves selection and single cell cloning of antibody producing lymphocyte bound to the reconstituted RBM, or to any RBDs and/or Spike proteins comprising the reconstituted RBM, to any RBDs and/or spike proteins discussed above, any multimeric and/or multivalent antigen displaying platform any derivative, enantiomer, fusion protein, conjugate, polyvalent dendrimer thereof, used in step (a).

In the next step (c), cloning the nucleic acid sequence encoding the variable regions or any segments thereof of at least one of the heavy and the light chains of an antibody produced by the selected cells, is performed. In some embodiments the lymphocytes used by the invention are lymphocytes obtained from a donor or a group of donors including at least one patient recovered from SARS CoV2 infection.

The methods of the invention require contacting lymphocyte/s of at least one donor, for example at least one subject recovered from SARS CoV2 infection with the polypeptides of the invention, specifically, the RBMs, RBDs, and spike proteins/disclosed by the invention.

It should be noted that a lymphocyte, as used herein specifically refer in some embodiments of the invention to antibody producing lymphocytes. In yet some further embodiments, the antibody producing lymphocyte is a B cell.

In more specific embodiments, the B cells used by the methods of the invention may be memory B cells. In yet some further embodiments, the invention provides methods for the production of human polyclonal neutralizing antibodies that neutralize the SARS CoV2. In more specific embodiments, the method comprising the steps of:

First in step (a), contacting serum of at least one donor or any immunoglobulin fraction thereof, with an effective amount of at least one reconstituted RBM of the Spike protein of SARS CoV2, associated directly or indirectly to a solid support and/or a detectable moiety. Alternatively, or in addition, the contacting step may be performed using any fragment of the reconstituted RBM, any RBD or Spike protein comprising the reconstituted RBM associated directly or indirectly to a solid support and/or a detectable moiety may be used. In yet some further embodiments, any RBD or Spike protein comprising at least one linker that replaces an anchor loop or any part thereof, or amino acid residue/s thereof, associated or attached directly or indirectly to a solid support and/or a detectable moiety may be used for this contacting step. In this connection, it should be noted that in some embodiments, the loop replaced either completely or partly by the linkers in the RBD or Spike protein used in these embodiments, comprises an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. Still further, it should be appreciated that any multimeric and/or multivalent antigen displaying platform of any of the RBMs, RBDs and/or spike proteins discussed above, any derivative, enantiomer, fusion protein, conjugate, polyvalent dendrimer thereof, or and any combinations thereof, associated directly or indirectly to a solid support and/or a detectable moiety, may be used for this step of contacting.

In the next step (b), recovering the antibodies bound to the reconstituted RBM, or to any RBDs and/or Spike proteins comprising the reconstituted RBM, to any RBDs and/or spike proteins discussed above, any multimeric and/or multivalent antigen displaying platform any derivative, enantiomer, fusion protein, conjugate, polyvalent dendrimer thereof, immobilized to the solid support and/or detectable moiety, used in step (a).

In some embodiments the serum used by the invention are lymphocytes obtained from a donor or a group of donors including at least one patient recovered from SARS CoV2 infection.

The invention provides in a further aspect thereof, a therapeutic passive vaccine comprising neutralizing antibodies that neutralize the SARS CoV2, prepared by any of the methods of the invention, specifically, as discussed above.

A further aspect of the invention relates to a method for the preparation of neutralizing antibodies directed at the Spike protein of SARS CoV2. In some embodiments, the method comprising the step of:

First in step (a), contacting at least one lymphocyte or serum of an immunized non-human animal, with an effective amount of at least one reconstituted RBM of the Spike protein of SARS CoV2, associated directly or indirectly to a solid support and/or a detectable moiety. Alternatively, or in addition, the contacting step may be performed using any fragment of the reconstituted RBM, any RBD or Spike protein comprising the reconstituted RBM associated directly or indirectly to a solid support and/or a detectable moiety. In yet some further embodiments, any RBD or Spike protein comprising at least one linker that replaces an anchor loop or any part thereof, or amino acid residue/s thereof, or any SARS CoV2 virus comprising at least one linker that replaces the anchor loop of the Spike protein, associated or attached directly or indirectly to a solid support and/or a detectable moiety, may be used for this contacting step. In this connection, it should be noted that in some embodiments, the loop replaced either completely or partly by the linkers in the RBD or Spike protein used in these embodiments, comprises an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. Still further, it should be appreciated that any multimeric and/or multivalent antigen displaying platform of any of the RBMs, RBDs and/or spike proteins discussed above, any derivative, enantiomer, fusion protein, conjugate, polyvalent dendrimer thereof, or and any combinations thereof, associated directly or indirectly to a solid support and/or a detectable moiety, may be used for this step of contacting with serum or lymphocytes.

The next step (b), involves recovering the antibodies or lymphocyte/s bound to the at least one reconstituted RBM, or to any RBDs and/or Spike proteins comprising the reconstituted RBM, to any RBDs and/or spike proteins discussed above, any multimeric and/or multivalent antigen displaying platform any derivative, enantiomer, fusion protein, conjugate, polyvalent dendrimer thereof, immobilized to the solid support and/or detectable moiety, used in step (a) thereby obtaining a passive vaccine comprising neutralizing antibodies that neutralize SARS CoV2. It should be noted that the non-human animal is immunized with an effective amount of the reconstituted RBM, RBD, or Spike protein comprising such reconstituted RBM. The non-human animal may be immunized with the RBD or Spike protein comprising at least one linker replacing the anchor loop or any part thereof, or amino acid residue/s thereof. Still further, the non-human animal may be immunized with any multimeric and/or multivalent antigen displaying platform thereof, any nucleic acid sequence encoding any of the RBMs, RBDs and/or Spike proteins as described by the invention, or any combinations thereof, any derivative, enantiomer, fusion protein, conjugate, polyvalent dendrimer thereof, or any SARS CoV2 virus comprising at least one linker that replaces the anchor loop of the Spike protein. In yet some further embodiments, the non-human animal may be immunized with at least one attenuated or killed SARS CoV2 virus or any variant or mutant thereof, and any composition or vaccine thereof. It should be noted that the reconstituted RBM used by the methods of the invention comprises at least one linker and at least one fragment of the native RBM. More specifically, the native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein. Still further, in some embodiments, the native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein. It should be noted that at least one of the linker/s replaces the anchor loop or any part thereof, or amino acid residue/s thereof and any RBM fragment or amino acid residue/s thereof.

In some embodiments, the polypeptide, RBD or Spike protein comprising at least one reconstituted RBM used by the methods are as defined by the invention, the at least one RBD or Spike protein comprising at least one linker used by the methods, are as defined by the invention, the at least one multimeric and/or multivalent antigen displaying platform used by the methods may be as defined by the invention, the at least one nucleic acid sequence used by the methods discussed herein, are as defined by the invention, the at least one composition as defined by the invention, and at least one vaccine used for the methods described herein, are as defined by the invention.

In some embodiments, the non-human mammal used for production of antibodies by the invention may be any small mammal (e.g., Rabbit, mouse rat and other rodents), any large mammal (e.g., goat, horse, or any non-human primate), or any avian subject.

In some specific embodiments, the methods of the invention may be specifically applicable for the production of monoclonal neutralizing antibodies. More specifically, the methods of the invention may comprise the steps of:

First (a), contacting lymphocytes of the immunized non-human animal with at least one reconstituted RBM of the Spike protein of SARS CoV2, associated directly or indirectly to a solid support and/or a detectable moiety. Alternatively, or in addition, the contacting step may be performed using any fragment of the reconstituted RBM, any RBD or Spike protein comprising the reconstituted RBM associated directly or indirectly to a solid support and/or a detectable moiety. In yet some further embodiments, any RBD or Spike protein comprising at least one linker that replaces an anchor loop or any part thereof, or amino acid residue/s thereof, associated or attached directly or indirectly to a solid support and/or a detectable moiety may be used for this contacting step. In this connection, it should be noted that in some embodiments, the loop replaced either completely or partly by the linkers in the RBD or Spike protein used in these embodiments, comprises an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. Still further, it should be appreciated that any multimeric and/or multivalent antigen displaying platform of any of the RBMs, RBDs and/or spike proteins discussed above, any derivative, enantiomer, fusion protein, conjugate, polyvalent dendrimer thereof, or and any combinations thereof, associated directly or indirectly to a solid support and/or a detectable moiety, may be used for this step of contacting with lymphocytes of the immunized non-human animal. The next step (b) involves selection and single cell cloning of antibody producing lymphocyte bound to the reconstituted RBM, or to any RBDs and/or Spike proteins comprising the reconstituted RBM, to any RBDs and/or spike proteins discussed above, any multimeric and/or multivalent antigen displaying platform any derivative, enantiomer, fusion protein, conjugate, polyvalent dendrimer thereof, immobilized to the solid support and/or detectable moiety, used in step (a). Step (c) involves cloning the nucleic acid sequence encoding the variable regions of at least one of the heavy and light chains of an antibody produced by these cells. It should be noted that a lymphocyte, as used herein specifically refer in some embodiments of the invention to antibody producing lymphocytes. In yet some further embodiments, the antibody producing lymphocyte is a B cell. In more specific embodiments, the B cells used by the methods of the invention may be memory B cells.

In yet some further embodiments, the methods of the invention may be applicable for the production of polyclonal neutralizing antibodies. More specifically, in some embodiments, the method may comprise the steps of:

First (a), contacting serum or immunoglobulin fraction thereof, of the immunized non-human animal with at least one reconstituted RBM of the Spike protein of SARS CoV2, associated directly or indirectly to a solid support and/or a detectable moiety. Alternatively, or in addition, the contacting step may be performed using any fragment of the reconstituted RBM, any RBD or Spike protein comprising the reconstituted RBM associated directly or indirectly to a solid support and/or a detectable moiety. In yet some further embodiments, any RBD or Spike protein comprising at least one linker that replaces an anchor loop or any part thereof, or amino acid residue/s thereof, associated or attached directly or indirectly to a solid support and/or a detectable moiety may be used for this contacting step. In this connection, it should be noted that in some embodiments, the loop replaced either completely or partly by the linkers in the RBD or Spike protein used in these embodiments, comprises an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. Still further, it should be appreciated that any multimeric and/or multivalent antigen displaying platform of any of the RBMs, RBDs and/or spike proteins discussed above, any derivative, enantiomer, fusion protein, conjugate, polyvalent dendrimer thereof, or and any combinations thereof, associated directly or indirectly to a solid support and/or a detectable moiety, may be used for this step of contacting with the serum of the immunized non-human animal. The next step (b), involves recovering the antibodies bound to reconstituted RBM, or to any RBDs and/or Spike proteins comprising the reconstituted RBM, to any RBDs and/or spike proteins discussed above, any multimeric and/or multivalent antigen displaying platform any derivative, enantiomer, fusion protein, conjugate, polyvalent dendrimer thereof, immobilized to the solid support and/or detectable moiety.

In some embodiments, the methods of the invention as described herein, may be used for the preparation of a therapeutic passive vaccine comprising neutralizing antibodies that neutralize the SARS CoV2. As discussed herein above, the neutralizing antibodies of the therapeutic vaccine, may be any of the polyclonal or the monoclonal neutralizing antibodies produced by any of the methods discussed above, or any composition or vehicle thereof.

In yet a further aspect thereof, the invention provides a therapeutic passive vaccine comprising neutralizing antibodies that neutralize the SARS CoV2. In more specific embodiments, such vaccine is prepared by the methods of the invention as described herein. Therapeutic vaccine according to some embodiments may comprise a therapeutically effective amount of the antibodies of the invention, for treating a subject infected with SARS CoV2. It should be understood that in some embodiments, the therapeutic vaccine of the invention may comprise in addition to the neutralizing antibodies also traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles. It should be further appreciated that the vaccine of the invention may be formulated for administration via localized and/or systemic routes, for example intravenous, intramuscular, or subcutaneous routes.

A further aspect of the invention relates to a method of screening for a compound that inhibits binding of at least one Spike protein of a SARS CoV2 virus to the cognate receptor in a target cell. In some embodiments, the method comprising the step of:

First in step (a), contacting at least one candidate compound or a plurality of candidate compounds with an effective amount of at least one reconstituted SARS CoV2 RBM, any RBD or Spike protein comprising the reconstituted RBM. In yet some further embodiments, the candidate compound may be contacted with any RBD or Spike protein comprising at least one linker that replaces an anchor loop or any part thereof, or amino acid residue/s thereof. More specifically, the loop replaced, at least in part, may comprise in some embodiments, an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein. Still further, the candidate compound/s may be contacted with any multimeric and/or multivalent antigen displaying platform of the RBMs, RBDs and/or Spike proteins of the invention, and any combinations thereof, any derivative, enantiomer, fusion protein, conjugate, polyvalent dendrimer thereof, associated directly or indirectly to a solid support and/or a detectable moiety; and The next step (b) involves recovering the candidate compound bound to the reconstituted RBM immobilized to the solid support and/or detectable moiety, or to any RBDs and/or Spike proteins comprising the reconstituted RBM, to any RBDs and/or spike proteins discussed above, any multimeric and/or multivalent antigen displaying platform any derivative, enantiomer, fusion protein, conjugate, polyvalent dendrimer thereof, used in step (a), thereby obtaining a compound that binds the Spike protein of a SARS CoV2 and inhibits binding of the virus to the cognate receptor.

In some embodiments, the reconstituted RBM used by the methods of the invention may comprise at least one linker and at least one fragment of the native RBM. More specifically, the native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein. Still further, the native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. The at least one of said linker/s replaces the anchor loop or any part thereof, or amino acid residue/s thereof.

In some embodiments, the candidate compound may be at least one of an antibody, an aptamer, a small molecule, a peptide and a nucleic acid molecule and any combinations thereof.

In yet some further embodiments, the candidate compound that inhibits binding of at least one Spike protein of a SARS CoV2 virus to the cognate receptor is an antibody. According to such embodiments, the plurality of the candidate antibody compounds is comprised in a phage display antibody library. Thus, according to these embodiments, the at least one reconstituted RBM, or any of the RBDs and Spike proteins described by the invention may be used for screening any antibody phage display library.

In yet some further embodiments the at least one reconstituted RBM, or any of the RBDs and Spike proteins described by the invention may be used for screening any small molecule, or aptamers or any peptide library.

A further aspect of the invention relates to a compound that inhibits binding of at least one Spike protein of a SARS CoV2 virus to the cognate receptor in a target cell. In some embodiments, the compound is prepared by any of the methods as defined by the invention.

Another aspect of the invention relates to a method for treating, inhibiting, reducing, eliminating, protecting or delaying the onset of COVID-19 in a subject in need thereof. More specifically, the method of the invention may comprise the step of administering to the subject an effective amount of a therapeutic passive vaccine comprising neutralizing antibodies that neutralize the SARS CoV2, or of a compound that inhibits binding of at least one Spike protein of a SARS CoV2 virus to the cognate receptor in a target cell. In some embodiments, any of the passive vaccines described by the invention, as well as any of the compounds disclosed herein, may be used by the therapeutic methods of the invention. In some embodiments, the therapeutic vaccine used by the methods of the invention may comprise a therapeutically effective amount of the polyclonal neutralizing antibodies, or the monoclonal neutralizing antibodies produced by any of the methods discussed above, or any combinations or composition, or vehicle thereof.

In yet some embodiments, the treated subject may be further administered with at least one additional anti-SARS CoV2 vaccine and/or with any other therapeutic compound. It should be noted that such at least one additional anti-SARS CoV2 vaccine and/or therapeutic compound may be administered prior to, after and/or simultaneously with administration of the passive vaccines provided by the invention or any of the vaccine or polypeptides disclosed by the invention.

A further aspect of the invention relates to an effective amount of the therapeutic passive vaccine comprising neutralizing antibodies that neutralize the SARS CoV2, or of a compound that inhibits binding of at least one Spike protein of a SARS CoV2 virus to the cognate receptor in a target cell, for use in a method for treating, preventing, inhibiting, reducing, eliminating, protecting or delaying the onset of COVID-19 in a subject in need thereof. In some embodiments, any of the passive vaccines and/or the compounds of the invention as described herein, may be used in accordance with the invention.

The invention provides in a further aspect thereof, an antibody that specifically recognizes and binds at least one polypeptide comprising an amino acid sequence of at least one reconstituted RBM of a viral Spike protein or of any fragment thereof, any RBD or Spike protein comprising the reconstituted RBM. In yet some further embodiments, the antibody provided by the invention may be an antibody that specifically recognizes and binds any RBD or Spike protein comprising at least one linker that replaces an anchor loop or any part thereof, or amino acid residue/s thereof. In some embodiments, the loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein. In yet some further embodiments, the antibody of the invention may recognize and bind any multimeric and/or multivalent antigen displaying platform of any of the RBMs, RBDs and Spike proteins, and any combinations thereof, any derivative, enantiomer, fusion protein, conjugate, polyvalent dendrimer thereof. Still further, in some embodiments, the reconstituted RBMs recognized by antibodies of the invention comprise at least one linker and at least one fragment of the native RBM. The native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein. Still further, the native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. It should be noted that at least one linker/s replaces this anchor loop or any part thereof, or amino acid residue/s thereof.

In some embodiments, the antibody of the invention is a neutralizing antibody that inhibits binding of at least one Spike protein of a SARS CoV2 virus to the cognate receptor in a target cell.

In yet some further embodiments, the antibody of the invention is any antibody prepared by any of the methods disclosed by the invention.

The term ‘antibody’ as meant herein encompasses the whole antibodies as well as any antigen binding fragment (i.e., ‘antigen-binding portion’) or single chain thereof. An ‘antibody’ refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region (abbreviated herein as CH). Each light chain is comprised of a light chain variable region (abbreviated herein as VL) and a light chain constant region (abbreviated herein as CL). The VH and VL regions can be further subdivided into regions of hypervariability, termed ‘complementarity determining regions’ (CDRs), interspersed with regions that are more conserved, termed “framework regions” (FRs). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A typical antibody is composed of two immunoglobulin (Ig) heavy chains and two Ig light chains. In humans, antibodies are encoded by three independent gene loci, namely the immunoglobulin heavy locus (IgH) on chromosome 14, containing the gene segments for the immunoglobulin heavy chain, the immunoglobulin kappa (κ) locus (IgK) on chromosome 2, containing the gene segments for part of the immunoglobulin light chain and the immunoglobulin lambda (λ) locus (IgL) on chromosome 22, containing the gene segments for the immunoglobulin light chain. The antibody and BCR heavy chains comprises 51 Variable (V) gene segments, 27 Diversity (D) gene segments, 6 Joining (J) gene segments. The antibody and BCR light chains comprise 40 Vκ, 31 Vλ, 5 Jκ, 4 Jλ gene segments. The term ‘antigen-binding portion’ or ‘antigen binding domain’ of an antibody, as used herein, refers to one or more fragments of an antibody that retain the ability to specifically bind to an antigen. It has been shown that the antigen-binding function of an antibody can be performed by fragments of a full-length antibody. Still further, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc. Non-limiting examples of binding fragments encompassed within the term ‘antigen-binding portion’ of an antibody include (i) a Fab fragment, a monovalent fragment consisting of the VL, VH, CL and Cm domains; (ii) a F(ab′)2 fragment, a bivalent fragment comprising two Fab fragments linked by a disulfide bridge at the hinge region; (iii) a Fd fragment consisting of the VH and Cm domains; (iv) a Fv fragment consisting of the VL and VH domains of a single arm of an antibody, (v) a dAb fragment, which consists of a VH domain; (vi) single-chain Fv (scFv) molecules; (vii) an isolated complementarity determining region (CDR), (viii) a combination of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules. Such single chain antibodies are also intended to be encompassed within the term “antigen-binding portion” of an antibody; and (ix) minimal recognition units consisting of the amino acid residues that mimic the hypervariable region of an antibody (e.g., an isolated complementarity determining region (CDR)). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein. A further example is binding-domain immunoglobulin fusion proteins comprising (i) a binding domain polypeptide that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The binding domain polypeptide can be a heavy chain variable region or a light chain variable region. These antibody fragments are obtained using conventional techniques known to those with skill in the art, and the fragments are screened for utility in the same manner as are intact antibodies.

More specifically, an ‘antibody fragment’ is a portion of an antibody such as F(ab′)2, F(ab)2, Fab′, Fab, and the like. Regardless of structure, an antibody fragment binds with the same antigen that is recognized by the intact antibody. For example, an anti-(polypeptide according to the present invention) monoclonal antibody fragment binds an epitope of a polypeptide according to the present invention. The term ‘antibody fragment’ also includes a synthetic or a genetically engineered polypeptide that binds to a specific antigen, such as polypeptides consisting of the light chain variable region, ‘Fv’ fragments consisting of the variable regions of the heavy and light chains, recombinant single chain polypeptide molecules in which light and heavy variable regions are connected by a peptide linker (‘scFv proteins’), and minimal recognition units consisting of the amino acid residues that mimic the hypervariable region. Still further, an antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain. The antibody suitable for the invention may also be a bi-specific antibody (such as Bi-specific T-cell engagers-BiTEs) or a tri-specific antibody. The antibody suitable for the invention may also be a variable new antigen receptor antibody (V-NAR). VNARs are a class of small, immunoglobulin-like molecules from the shark immune system. Humanized versions of VNARs could be used. Still further, in some embodiments, the antibodies disclosed by the invention may be camelid single-domain antibodies (sdAbs), also known as heavy chain-only antibodies (HCAbs) or V_{H}Hs, and widely known as nanobodies. The term ‘epitope’ means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. Methods for preparing antibodies are known to the art. See, for example, Harlow & Lane (1988) Antibodies: a Laboratory Manual, Cold Spring Harbor Lab., Cold Spring Harbor, NY). Monoclonal antibodies may be prepared from a single B cell line taken from the spleen or lymph nodes of immunized animals, in particular rats or mice, by fusion with immortalized B cells under conditions which favor the growth of hybrid cells. The technique of generating monoclonal antibodies is described in many articles and textbooks, such as the above-noted Chapter 2 of Current Protocols in Immunology. Spleen or lymph node cells of these animals may be used in the same way as spleen or lymph node cells of protein-immunized animals, for the generation of monoclonal antibodies as described in Chapter 2 therein. The techniques used in generating monoclonal antibodies are further described in by Kohler and Milstein, Nature 256; 495-497, (1975), and in U.S. Pat. No. 4,376,110. Antibodies that are isolated from organisms other than humans, such as mice, rats, rabbits, cows, goat, horse, can be made more human-like through chimerization or humanization. It should be understood that the various antibodies and antigen-fragments thereof as defined herein, are applicable for any antibody (e.g., neutralizing antibodies used in the screening methods, diagnostic methods and kits or in the therapeutic vaccines) in any of the aspects of the invention.

A further aspect of the invention relates to a diagnostic method for the detection of SARS CoV2 infection in a mammalian subject. The diagnostic methods of the invention may comprise the steps of: First (a), contacting at least one biological sample of the subject with at least one of (i) at least one reconstituted RBM or any RBD or Spike protein comprising the reconstituted RBM associated directly or indirectly to a solid support and/or a detectable moiety; with (ii) antibodies specific for the reconstituted RBM associated directly or indirectly to a solid support and/or a detectable moiety; or with (iii) any RBM binding molecule (e.g., aptamers, peptides) associated directly or indirectly to a solid support and/or a detectable moiety. It should be noted that option (i) discussed above, further encompasses the use of any of the RBMs, RBDs or Spike proteins or any multimeric and/or multivalent antigen displaying platform thereof, as disclosed by the invention. In some non-limiting embodiments, the methods disclosed herein may use any polypeptide or plurality of polypeptides (e.g., RBM/s, RBDs, spike proteins) comprising any of the RBM/s of the invention, specifically, at least one of the RBMs of SEQ ID NO: 65-84, and any variants thereof, as disclosed in connection with other aspects.

In some embodiments, the sample is contacted with any of the elements described in (i), (ii) or (iii), and the unbound material of the sample may be washed by one or more washing step/s.

The next step (b), involves determining that the subject is infected with SARS CoV2, if the detectable moiety is detected in the sample. In some embodiments, the reconstituted RBM used by the diagnostic method of the invention may comprise at least one linker and at least one fragment of the native RBM. More specifically, the native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein. In yet some further embodiments, the native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. At least one of said linker/s replaces the anchor loop or any part thereof, or amino acid residue/s thereof and any RBM fragment or amino acid residue/s thereof.

Still further, the diagnostic methods of the invention may use any of the polypeptides, RBM/s, RBDs or Spike protein comprising the reconstituted RBM as defined by the invention. In yet some further embodiments, any of the RBMs or Spike proteins that comprise at least one linker described by the invention may be used by the diagnostic methods. In yet some further embodiments, the diagnostic methods may use any of the multimeric and/or multivalent antigen displaying platforms defined by the invention. In yet some further embodiments, the diagnostic method of the invention may use at least one antibody, specifically an antibody recognizing any of the reconstituted RMBs of the invention. In some specific embodiments, the antibody specific for the reconstituted RBM may be any one of: (a) human antibodies prepared by any of the methods defined by the invention; (b) antibodies prepared by any of the methods disclosed by the invention involving immunized non-human animal; and (c) antibodies or RBM binding compounds prepared by screening a phage display antibody library with the reconstituted RBM or any RBD or Spike protein comprising the reconstituted RBM, as defined by the invention.

It should be understood that when the RBMs, RBDs and/or Spike proteins of the invention are used as detecting molecules in the diagnostic methods and kits of the invention, any antibody found in the potential patient's sample, e.g., serum sample, indicates that the subject was exposed to SARS-CoV2 and therefore its immune system produced antibodies detectable by the methods and kits of the invention. In yet some alternative and/or additional embodiments, antibodies that recognize the RBMs, RBDs and/or Spike proteins of the invention are used as detecting molecules, or any other compound that specifically recognizes and binds the RBMs, RBDs and/or Spike proteins of the invention, may be used as a detecting molecules. According to such embodiments, thee detecting molecule, specifically the antibodies and any other binding molecule, can recognize and bind the viral SARS CoV2 protein in a virus that exists in the biological samples of the diagnosed subject. Still further, in some embodiments, the detecting molecules used by the diagnostic methods and kits of the invention may be connected to a solid support and/or detectable moiety. Thus, in some embodiments, where the detecting molecule (antibody, binding molecule or the RBM polypeptide of the invention) is attached to a solid support, the sample is contacted with the detecting molecule, the unbound material is washed and the virus or antibodies in the sample are detected using a second detecting molecule bound to a detectable moiety. In yet some alternative embodiments, the sample or any fractions thereof may be immobilized to a solid support, and contacted with the detecting molecule that is bound directly or indirectly to a detectable moiety. Detecting a detectable signal indicates the presence of the anti-SARS CoV2 antibodies or the virus in the sample.

A further aspect of the invention provide a diagnostic kit comprising at least one of:

One element (a), may be at least one reconstituted RBM or any RBD or Spike protein comprising the reconstituted RBM, any RBD or Spike protein comprising at least one linker that replaces an anchor loop or any part thereof, or amino acid residue/s thereof. The loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein. Alternatively, and/or additionally, any multimeric and/or multivalent antigen displaying platform of any of the RBMs, RBDs or Spike proteins and any combinations thereof, associated directly or indirectly to a solid support and/or a detectable moiety, may be used in the kits of the invention. The kit of the invention may alternatively or additionally comprise (b), antibodies specific for said reconstituted RBM associated directly or indirectly to a solid support and/or a detectable moiety. Still further, in some embodiments, the kit of the invention may comprise (c) at least one reconstituted RBM binding molecule associated directly or indirectly to a solid support and/or a detectable moiety. In some embodiments, the kit of the invention may comprise any of the polypeptides, RBDs or Spike proteins comprising at least one reconstituted RBM as defined by the invention, at least one RBD or spike protein comprising at least one linker as defined by the invention, at least one multimeric and/or multivalent antigen displaying platform as defined by the invention. Non-limiting embodiments for RBM/s, RBDs, spike proteins comprising any of the RBM/s or linkers of the invention, include the RBMs of SEQ ID NO: 65-84, and any variants thereof, as disclosed in connection with other aspects.

In some embodiments, the kits of the invention may comprise any of the antibodies disclosed by the invention. More specifically, any antibodies specific for the reconstituted RBM, for example, any one of: (a) any of the human antibodies prepared by any of the methods of the invention; (b) any antibodies prepared using non-human animals as defined by any of the methods of the invention; (c) any antibodies or RBM binding compounds prepared by screening a phage display antibody library with the reconstituted RBM or any RBD or Spike protein comprising the reconstituted RBM, as defined by the screening methods of the invention.

In some embodiments, the invention provides the use of any of the kits of the invention as disclosed herein, in a method for the detection of SARS CoV2 infection in a mammalian subject.

In a further aspect thereof, the invention further provides an effective method for improving epitope-based vaccine that comprise at least one Spike protein of SARS CoV2, or any fragments or parts thereof. The method of the invention comprises the step of replacing the anchor loop of the Spike protein of SARS CoV2, or any fragments or parts thereof with a linker. In some embodiment, the anchor loop of the RBM comprise an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 spike protein. In yet some further embodiments, the linker is an amino acid linker comprising 3 to 7 amino acid residues.

The invention further provides in some aspects thereof, at least one linker that replaces an anchor loop or any part thereof, or amino acid residue/s thereof, of a SARS CoV2 Spike protein. More specifically, the loop may comprise an amino acid sequence starting at any one of the amino acid residues F456, R457, K458, R454 or L455 and ending at any one of the amino acid residues Y473, I472, E471, T470, S469, I468, Q474, A475 or G476 of the SARS CoV2 Spike protein. It should be noted that the linker of the invention bridges the amino acid residues that flank the replaced anchor loop or any part thereof, or amino acid residue/s thereof, thereby forming a functional RBM, RBD and/or Spike protein or any functional fragments thereof. In some embodiments, at least one linker that is a linker that replaces the anchor loop, is a bridging linker. Such bridging linker bridges and the last amino acid residue of the N′ terminal fragment (also referred to herein as the first fragment or as fragment A) of the reconstituted RBM with the first amino acid residue of the C′ terminal fragment (also referred to herein as the second fragment or as fragment B) of the reconstituted RBM. In some embodiments the bridging linker bridges the amino acid residues that flank the replaced anchor loop or any part thereof, or amino acid residue/s thereof. In yet some further embodiments, the linker may comprise any compound bridging the two fragments of the reconstituted RBM. In more embodiments, the linker is an inert linker. In yet some further embodiments, such linker is any inorganic or organic molecule, any small molecule, any peptide (L- as well as D-aa residues), or any combinations thereof. In some embodiments, the linker of the invention is designated herein as the “τ-linker” or “T-linker” (tau-linker). In some embodiments, the bridging linker may bridge the amino acid residue F456 with T473. In some embodiments, the bridging linker may bridge the amino acid residue F456 with I472. In some embodiments, the bridging linker may bridge the amino acid residue F456 with E471. In some embodiments, the bridging linker may bridge the amino acid residue F456 with T470. In some alternative embodiments, the bridging linker may bridge the amino acid residue F456 with S469. In some further embodiments, the bridging linker may bridge the amino acid residue F456 with I468. In some embodiments, the bridging linker may bridge the amino acid residue F456 with Q474. In some embodiments, the bridging linker may bridge the amino acid residue F456 with A475. In some embodiments, the bridging linker may bridge the amino acid residue F456 with G476. In some embodiments, the bridging linker may bridge the amino acid residue R457 with Y473. In some embodiments, the bridging linker may bridge the amino acid residue R4576 with I472. In some embodiments, the bridging linker may bridge the amino acid residue R457 with E471. Still further, in some embodiments, the bridging linker may bridge the amino acid residue R457 with T470. In some alternative embodiments, the bridging linker may bridge the amino acid residue R457 with S469. In some further embodiments, the bridging linker may bridge the amino acid residue R457 with I468. In some embodiments, the bridging linker may bridge the amino acid residue R457 with Q474. In some embodiments, the bridging linker may bridge the amino acid residue R457 with A475. In some embodiments, the bridging linker may bridge the amino acid residue R457 with G476. In some embodiments, the bridging linker may bridge the amino acid residue K458 with Y473. In some embodiments, the bridging linker may bridge the amino acid residue K458 with I472. In some embodiments, the bridging linker may bridge the amino acid residue K458 with E471. In some embodiments, the bridging linker may bridge the amino acid residue K458 with T470. In some alternative embodiments, the bridging linker may bridge the amino acid residue K458 with S469. In some further embodiments, the bridging linker may bridge the amino acid residue K458 with I468. In some embodiments, the bridging linker may bridge the amino acid residue K458 with Q474. In some embodiments, the bridging linker may bridge the amino acid residue K458 with A475. In some embodiments, the bridging linker may bridge the amino acid residue K458 with G476. In some embodiments, the bridging linker may bridge the amino acid residue R454 with Y473. In some embodiments, the bridging linker may bridge the amino acid residue R454 with I472. In some embodiments, the bridging linker may bridge the amino acid residue R454 with E471. In some embodiments, the bridging linker may bridge the amino acid residue R454 with T470. In some alternative embodiments, the bridging linker may bridge the amino acid residue R454 with S469. In some further embodiments, the bridging linker may bridge the amino acid residue R454 with I468. In some embodiments, the bridging linker may bridge the amino acid residue R454 with Q474. In some embodiments, the bridging linker may bridge the amino acid residue R454 with A475. In some embodiments, the bridging linker may bridge the amino acid residue R454 with G476. In some embodiments, the bridging linker may bridge the amino acid residue L455 with Y473. In some embodiments, the bridging linker may bridge the amino acid residue L455 with I472. In some embodiments, the bridging linker may bridge the amino acid residue L455 with E471. In some embodiments, the bridging linker may bridge the amino acid residue L455 with T470. In some alternative embodiments, the bridging linker may bridge the amino acid residue L455 with 5469. In some further embodiments, the bridging linker may bridge the amino acid residue L455 with I468. In some embodiments, the bridging linker may bridge the amino acid residue L455 with Q474. In some embodiments, the bridging linker may bridge the amino acid residue L455 with A475. In some embodiments, the bridging linker may bridge the amino acid residue L455 with G476.

In some embodiments, the linker is an amino acid linker comprising about 3 to 7 amino acid residues. In some embodiments, the amino acid linker in accordance with the invention is a bridging linker comprising between about 3 to about 7 consecutive amino acid residues. In yet some further embodiments, the sequence of any of said linkers of 3, 4, 5, 6, or 7 amino acid residues, does not exist in the anchor loop of the spike protein of SARS CoV2, specifically, the anchor loop as defined by the invention. In yet some further embodiments, the sequence of any of the linkers of 3, 4, 5, 6, or 7 amino acid residues, does not exist in the anchor loop of a spike protein of any beta Corona Virus. In yet some further embodiments, the linker of the invention must differ from the replaced native sequence in at least one amino acid residue, and specifically, two, three, four, five, six, seven or more residues. In yet some further embodiments, the linker used to replace the native sequences (e.g., the loop or any fragments thereof), differs from the native replaced sequence, such that any polypeptide e.g., the reconstituted RBM, RBD, and/or spike protein, that comprise said at least one linker cannot be considered as a natural product. In some embodiments, the τ-linkers of the invention may be any of the linkers of SEQ ID NO: 57, the GVP linker or any of the linkers of SEQ ID NOS: 42-52, or any variants thereof, specifically, any of the variants disclosed by the invention. The main goal of the present invention is to provide means to combat Coronaviruses (CoVs) infections, specifically SARS-CoV2 infections and to prevent spread of such infections via other animals to humans and/or to domestic animals as well as to prevent human to human infections. As shown by the present invention, replacing the anchor loop with a linker screened from a combinatorial library, enables the RBM, the RBD and even the entire Spike protein, to assume a physiological conformation. It should be noted that the “anchor loop”, as used herein, is a segment of the RBM, that provide attachment, fastening or fixation of the RBM to the core of the RBD, via several hydrogen bonds. The invention therefore provides an effective approach for production of functional RBMs, RBDs and spike proteins of any corona virus, simply by replacing the anchor loop with a specific linker, screened and evaluated for receptor and neutralizing antibody binding.

Thus, the invention further provides in an additional aspect thereof, an RBD of a Corona virus (CoV) comprising the native RBD of the spike protein of at least one CoV or any fragments thereof and at least one linker, wherein at least one of said linker/s replaces an anchor loop of said Spike protein or any part thereof, or amino acid residue/s thereof, said anchor loop comprises between 5 to 20 amino acid residues and forms 2 to 5 hydrogen bonds to the core of the RBD, wherein the amino acid sequence of said loop starts approximately 10 amino acid residues before the first hydrogen bond and ends approximately 10 amino acid residues after the last hydrogen bond to the core of the RBD, said core comprise about five beta strands, wherein at least one of said linker is a bridging linker. It should be understood that the term “approximately”, as used herein throughout the following aspects of the invention, refers to about 1, 2, 3, 4, 5, 6, 7, 8, 9, 10, 11, 12, 13, 14, 15 or more residues. Specifically, in some embodiments, approximately 10 amino acid residues, is meant about 10+/−1 or 2 residues, more specifically, about 8, 9, 10, 11, or 12 residues.

A further aspect of the invention relates to a Spike protein of a CoV, comprising the native Spike protein of at least one CoV or any fragment thereof and at least one linker. At least one of the linker/s replaces an anchor loop of the Spike protein or any part thereof, or amino acid residue/s thereof. The anchor loop comprises between 5 to 20 amino acid residues and forms 2 to 5 hydrogen bonds to the core of the RBD of said Spike protein. More specifically, the amino acid sequence of said loop starts approximately 10 amino acid residues (e.g., 10+/−1 or 2 residues) before the first hydrogen bond and ends approximately 10 amino acid residues (e.g., 10+/−1 or 2 residues) after the last hydrogen bond to the core of the RBD. The core comprises about five beta strands, wherein said at least one linker is a bridging linker. It should be noted that the spike protein as provided by the invention, encompasses in accordance with some embodiments, the S1 subunit of the spike protein and at least one linker that replaces the anchor loop as specified herein above. In yet some further embodiments, the spike protein provided herein comprises the S1 subunit and the S2 subunit and at least one linker that replaces the anchor loop, specifically the anchor loop residing at the S1 subunit. Thus, in some embodiments, the invention provides the entire spike protein that comprises at least one linker that replaces the anchor loop as specified by the invention.

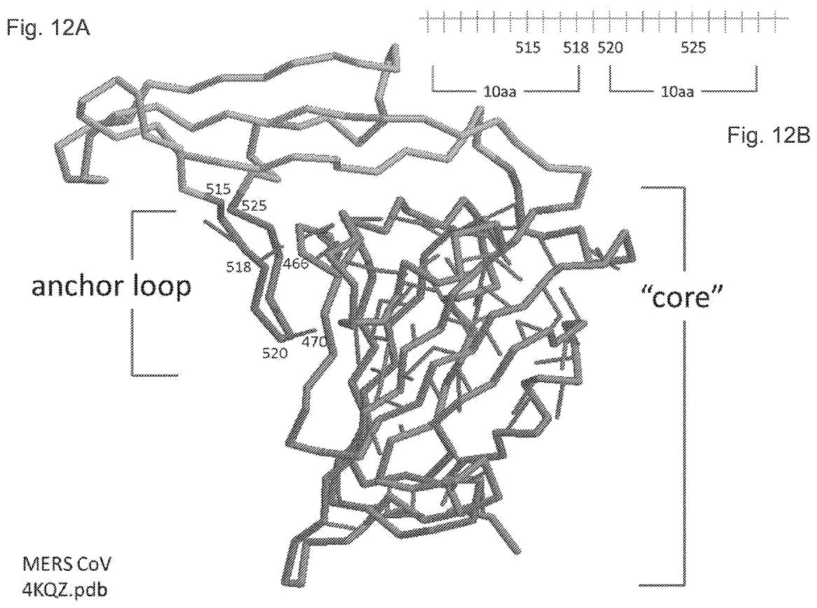

A Corona virus or any variant or mutant thereof, comprising at least one Spike protein that comprises at least one linker, wherein at least one of said linker/s replaces an anchor loop of the Spike protein or any part thereof, or amino acid residue/s thereof of said Spike protein. The anchor loop comprises between 5 to 20 amino acid residues and forms 2 to 5 hydrogen bonds to the core of the RBD of said Spike protein. The amino acid sequence of said loop starts approximately 10 amino acid residues before the first hydrogen bond and ends approximately 10 amino acid residues after the last hydrogen bond to the core of the RBD, said core comprises about five beta strands. In some embodiments, the at least one linker is a bridging linker. In some embodiments the bridging linker bridges the amino acid residues that flank the replaced anchor loop or any part thereof, or amino acid residue/s thereof. The invention further encompasses any attenuated or killed CoV as defined herein. As demonstrated by FIGS. 11-13, the Receptor Binding Domain of beta-coronaviruses consists of a secondary structure extension, that is also referred to herein as an excursion, that initiates from the 4^{th} beta strand of the core of the Receptor binding domain and ends just before the 5^{th} beta strand of the core. More specifically, in some embodiments, the “core” of the RBD of the spike protein of the CoV, contains 5 beta strands. In some specific embodiments, 4 strands precede the RBM excursion and the 5^{th} strand initiating after the excursion. Whereas the RBM forms contact with the virus receptor, e.g. ACE2 for SARS CoV and SARS CoV2, there exists a short loop stemming from the RBM excursion that does not contact the receptor. Rather this loop serves to tack the RBM excursion to the core via a number of hydrogen bonds, hence this loop is referred to as the anchor loop. Thus, in accordance with some embodiments of the invention, the RBM structure is defined herein as the secondary structure extension, or the extended excursion following beta strand 4 of the core of the RBD and ending just before beta strand 5 of the RBD core. Each RBM excursion is held in place via an anchor loop that tacks the RBM excursion to the surface of the core via a number of hydrogen bonds. The dimensions of the anchor loop can be defined as a loop stemming from the RBM excursion of typically 10-16 amino acids in length and no more than 20-25 residues. In yet some further embodiments, the anchor loop is further characterized by the fact that it forms 2, 3, 4 or 5, but in some embodiments, no more than 5 hydrogen bonds with the surface of the RBD core. Specifically, 2 or 3 hydrogen bonds. The residues that contribute to these hydrogen bonds are contained within the anchor loop. In some embodiments, the first residue of the anchor loop that participates in the first hydrogen bond is located within 10 residues of the beginning of the loop. In yet some further embodiments, the last residue of the anchor loop that participates in the last hydrogen bond is located within 10 residues from the end of the loop. Thus, the anchor loop is located about 10 amino acids before the first hydrogen bond to the core, specifically, about 1, 2, 3, 4, 5, 6, 7, 8, 9, or 10 amino acid residues (e.g., 10+/−1 or 2 residues) before the amino acid reside that participate in the first hydrogen bond, and about 10 amino acids after the last hydrogen bond to the core, specifically, about 1, 2, 3, 4, 5, 6, 7, 8, 9, or 10 amino acid residues (e.g., 10+/−1 or 2 residues) after the amino acid reside that participate in the last hydrogen bond to the core.

In some embodiments the bridging linker bridges the amino acid residues that flank the replaced anchor loop or any part thereof, or amino acid residue/s thereof. In yet some further embodiments, the linker may comprise any compound bridging the amino acid residues that flank the replaced anchor loop or any part thereof, or amino acid residue/s thereof. In more embodiments, the linker is an inert linker. In yet some further embodiments, such linker is any inorganic or organic molecule, any small molecule, any peptide (L- as well as D-aa residues), or any combinations thereof. In yet some further embodiments, the at least one linker is an amino acid linker comprising 3 to 7 amino acid residues. In some embodiments, the linkers applicable in the present aspects may be any of the τ-linkers of the invention, for example, any of the linkers of SEQ ID NO: 57, the GVP linker or any of the linkers of SEQ ID NOS: 42-52, or any variants thereof, specifically, any of the variants disclosed by the invention. It should be noted that the RBMs, RBDs, Spike proteins and the Corona viruses provided by the various aspects of the invention as discussed herein above, are relevant for any known or yet unknown corona virus. CoVs are common in humans and usually cause mild to moderate upper-respiratory tract illnesses. There are four main sub-groupings of coronaviruses, known as alpha, beta, gamma, and delta. More specifically, Coronaviruses are species in the genera of virus belonging to one of two subfamilies Coronavirinae and Torovirinae in the family Coronaviridae, in the order Nidovirales. Herein this term refers to the entire family of Coronavirinae (in the order Nidovirales). Coronaviruses are enveloped viruses with a positive-sense single-stranded RNA genome and with a nucleocapsid of helical symmetry. The genomic size of coronaviruses ranges from approximately 26 to 32 kilobases, the largest for an RNA virus. The name “coronavirus” is derived from the Latin corona, meaning crown or halo, and refers to the characteristic appearance of virions under electron microscopy (E.M.) with a fringe of large surface projections creating an image reminiscent of a crown. This morphology is created by the viral spike (S) peplomers, which are proteins that populate the surface of the virus and determine host tropism. There are many CoVs that naturally infect animals, the majority of these usually infect only one animal species or, at most, a small number of closely related species, but not humans. CoV strains that are particular subject of the present invention, due to their extreme virulence and hazard in humans, are those that have been transmitted from animals to humans, specifically the SARS-COV2. Thus, the term Coronaviruses (designated herein CoVs) for the purposes of the present invention encompasses four main sub-groupings of coronaviruses, known as Alpha, Beta, Gamma, and Delta. More specifically, under this term is meant the enveloped viruses with a positive-sense RNA genome (ssRNA+) and with a nucleocapsid of helical symmetry; and also large RNA viruses with the genomic size of ranges from approximately 26 to 32 kilobases; and further viruses with the characteristic morphology of large, bulbous surface projections under electron microscopy, which is created by the viral spike (S) peplomers, i.e. viral surface proteins determining host tropism and immunogenicity.

The present invention in particular concerns the novel coronaviruses that have emerged in humans in 2019: SARS-COV2 which is associated with high contagion and relatively high mortality rates. There are currently no clinically approved vaccines or antiviral drugs available for this infection; thus, the development of effective therapeutic and preventive strategies that can be readily applied to new emergent strains of SARS-COV2 is a research priority. However, the approach and method provided by the present invention are applicable for any coronavirus. In more specific embodiments, the invention may be applicable for any beta corona virus, and any various mutants and variants thereof, specifically, any of the variants disclosed by the invention. Betacoronaviruses (0-CoVs or Beta-CoVs) are one of four genera of coronaviruses of the subfamily Orthocoronavirinae in the family Coronaviridae, of the order Nidovirales. They are enveloped, positive-sense, single-stranded RNA viruses of zoonotic origin. The coronavirus genera are each composed of varying viral lineages with the betacoronavirus genus containing four such lineages. In older literature, this genus is also known as group 2 coronaviruses. Still further, in some embodiments, the seven coronaviruses known to-date as infecting humans are: alpha coronaviruses 229E and NL63, and beta coronaviruses OC43, HKU1, SARS-CoV and SARS-CoV2, and MERS-CoV (the coronavirus that causes Middle East Respiratory Syndrome, or MERS). The SARS-CoV and SARS-CoV2 are a lineage B beta Coronavirus and the MERS-CoV is a lineage C beta Coronavirus. The Beta-CoVs of the greatest clinical importance concerning humans are OC43 and HKU1 of the A lineage, SARS-CoV and SARS-CoV-2 of the B lineage, and MERS-CoV of the C lineage. Coronaviruses have a large genome size that ranges from 26 to 32 kilobases. The overall structure of β-CoV genome is similar to that of other CoVs, with an ORF1ab replicase polyprotein (rep, pp1ab) preceding other elements. This polyprotein is cleaved into many nonstructural proteins. Within the genus Betacoronavirus (Group 2 CoV), four lineages (A, B, C, and D) are commonly recognized. The four lineages have also been named using Greek letters or numerically. A further subgenus is Hibecovirus including Bat Hp-betacoronavirus Zhejiang 2013. The ectodomain of all CoV spike proteins share the same organization in two domains: a N-terminal domain named S1 that is responsible for receptor binding and a C-terminal S2 domain responsible for fusion. The S₁ subunit of the betacoronavirus spike proteins displays a multidomain architecture and is structurally organized in four distinct domains A-D of which domains A and B may serve as a RBD. The core structure of domain A displays a galectin-like β-sandwich fold, whereas domain B contains a structurally conserved core subdomain of antiparallel β-sheets. Importantly, domain B is decorated with an extended loop on the viral membrane-distal side. This loop may differ greatly in size and structure between virus species of the betacoronavirus genus and is therefore also referred to as hypervariable region (HVR). Compared to the S₂ subunit, the S₁ subunit displays low level of sequence conversation among species of different CoV genera. In the specific case of the SARS CoV2, a defined receptor-binding domain on S mediates the attachment of the virus to its cellular receptor, angiotensin-converting enzyme 2 (ACE2). Some CoVs, specifically the members of Beta CoV subgroup A, also have a shorter spike-like protein called hemagglutinin esterase (HE).

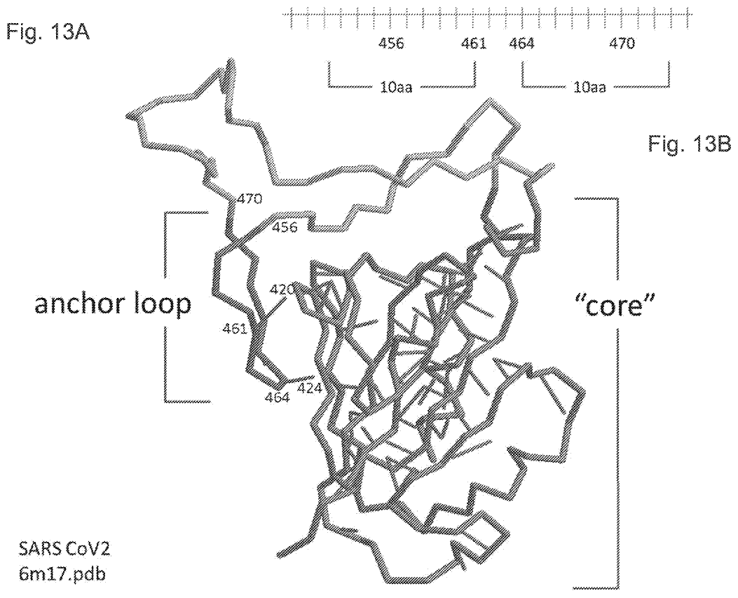

Another example is the spike glycoprotein (S) of MERS-CoV that targets the cellular receptor dipeptidyl peptidase 4 (DPP4). Interaction between the spike protein and DPP4 is mediated by a RBD on the viral spike. It has been reported that MERS-CoV RBD consists of a core and a receptor-binding subdomain (which is also referred to herein as a “receptor binding motif”, RBM). RBM may be identified by any method known in the art based on the interaction formed between the spike protein and a host cell. Particular RBD encompassed by the present disclosure are of the spike proteins of SARS CoV2, MERS CoV and SARS CoV, the amino acid sequences of which are denoted by the amino acid sequences SEQ ID NO: 6, SEQ ID NO: 18 and SEQ ID NO: 17, respectively, or any variants and mutants thereof. Of particular interest are the variants of the spike protein of the SARS CoV2, as denoted by SEQ ID NO: 6. Non-limiting examples for such variants may include any of the variants disclosed by the invention, for example, any variants with at least one of the following substitutions Y489N, Q474R, V483E, L492I, N501Y, K417N, E484K, L452R, Y453F, S13I, L18F, T20N, P26S, H69-V70 deletion, D80A, D138Y, Y144/145 deletions, W152C, R190S, D215G, L242/A243/L244 deletion, R246I, A570D, D614G, H655Y, P681H, I692V, A701V, T716I, S982A, T1027I, D1118H, V1176F, M1229I or any combinations thereof. The other human CoVs are believed to cause a significant percentage of all common colds in human adults (primarily in the winter and early spring seasons). In certain individuals CoVs may further be a direct or indirect cause of pneumonia, i.e. direct viral pneumonia or a secondary bacterial pneumonia. In some embodiments, the RBDs, Spike proteins and viruses provided by the invention may be any one of the SARS CoV2 as defined herein before, as well as the MERS CoV, and the SARS CoV. In some specific embodiments, the Coronavirus may be Middle East Respiratory Syndrome coronavirus (MERS CoV). FIG. 12 shows the RBD structure, and specifically the anchor lop that may be replaced in the MERS CoV, for providing the RBD or spike protein in accordance with the invention. Thus, in some embodiments, the invention provides an RBD and a spike protein of MERS CoV, having a linker that replaces the anchor loop. In some embodiments the anchor loop comprises the amino acid sequence starting at any one of residues 509, 510, 511, 512, 513, 514, 515, 516 or 517 and ending at any one of residues 521, 522, 523, 524, 525, 526, 527, 528 or 529 of the spike protein of MERS CoV, specifically, as denoted by SEQ ID NO: 18. In some embodiments, the anchor loop of SARS CoV comprises residues 515 to 525 of the spike protein of MERS CoV, specifically, as denoted by SEQ ID NO: 18.

The term MERS CoV as known in the art refers to a lineage C beta coronavirus (+RNA 30 kb) whose primary natural reservoir resides in bats that infect domesticated camels as opportunistic hosts, which go on to infect humans. MERS-CoV genomes are phylogenetically classified into two clades, clade A and B. The earliest cases of MERS were of clade A clusters (EMC/2012 and Jordan-N3/2012), and new cases are genetically distinct (clade B). MERS-CoV is distinct from SARS-CoV and distinct from the common-cold coronavirus and known endemic human betacoronaviruses HCoV-OC43 and HCoV-HKU1. Until 23 May 2013, MERS-CoV had frequently been referred to as a SARS-like virus, or simply the novel coronavirus, and early it was referred to colloquially as the “Saudi SARS”. Over 1,600 cases of MERS have been reported by 2015 and the case fatality rate is >30%. 182 genomes have been sequenced by 2015 (94 from humans and 88 from dromedary camels). All sequences are >99% similar. The genomes can be divided into two clades—A and B—with the majority of cases being cased by clade B. Human and camel strains are intermixed suggesting multiple transmission events. Like other coronaviruses, the MERS-CoV virion utilizes a large surface spike (S) glycoprotein for interaction with and entry into the target cell. The S glycoprotein consists of a globular S1 domain at the N-terminal region, followed by membrane-proximal S2 domain, a transmembrane domain and an intracellular domain. Determinants of cellular tropism and interaction with the target cell are within the S 1 domain, while mediators of membrane fusion have been identified within the S2 domain. Through co-purification with the MERS-CoV SI domain, dipeptidyl peptidase 4 (DPP4) was identified as cellular receptor for MERS-CoV. DDP4 is expressed on the surface of several cell types, including those found in human airways, and possesses ectopeptidase activity, although this enzymatic function does not appear to be essential for viral entry.

FIG. 11 shows the RBD structure, and specifically the anchor loop that may be replaced in the SARS CoV, for providing the RBD or spike protein in accordance with the invention. Thus, in some embodiments, the invention provides an RBD and a spike protein of SARS CoV, having a linker that replaces the anchor loop. In some embodiments the anchor loop comprises the amino acid sequence starting at any one of residues 439, 440, 441, 442, 443, 444, 445, or 446 and ending at any one of residues 455, 456, 457, 458, 49, 460, 461, 462, 463 of the spike protein of SARS CoV, specifically, as denoted by SEQ ID NO: 17. In some embodiments, the anchor loop of SARS CoV comprises residues 444 to 457 of the spike protein of SARS CoV, specifically, as denoted by SEQ ID NO: 17. Non-binding examples of linkers useful in the present invention in connection with MERS CoV may include linkers comprising the amino acid sequence of GFDEQ, PYNYH, SQAPL, LHLP and VVAT, as denoted by SEQ ID NO: 25, 26, 27, 28 and 29, respectively, as well as NNK₃, NNK₂ and NNK₁ linkers such as VYS, RQ, SY, FS, C Y, CF, LG, FQS, SIR, A, QLT, IRK, LQ, PT, RTK, RLT and LP. It should be noted that amino acid residues are represented herein in their acceptable one-letter code, and in the sequence listing amino acid sequences of 4 amino acid residues or mole are presented in their three-letter code.

In yet some further embodiments, In yet further specific embodiments, the linkers used for the reconstituted RBM of the polypeptide of the invention may comprise the amino acid sequence of Xaa¹₍ₙ₎-Xaa²-Xaa³-Xaa⁴-Xaa₍ₙ₎ as denoted by SEQ ID NO: 30 or any fragment thereof. More specifically, Xaa¹ may be any amino acid and n is zero or an integer of from 1 to 5, and wherein: Xaa² may be a Gly, Glu, Leu or Thr; Xaa³, may be Glu or Asp; and Xaa⁴, may be Glu, Asn, Pro, Met, Val or Asp. Still further, the linker applicable for the reconstituted RBM of the polypeptide of the invention may comprise the amino acid sequence of Xaa¹-Xaa²-Xaa³ or any fragment thereof. It should be noted that Xaa may be any amino acid. In some embodiments, X¹, may be a Gly, Glu or Thr; X², may be Glu; and X³, may be Glu, Asn, Pro, Met, Val or Asp.

In some specific embodiments, the linker may comprise the amino acid sequence of any one of: Gly-Glu-Met (GEM) and Glu-Glu-Pro (GGP). In yet some further embodiments, the linker may comprise the amino acid sequence of Xaa¹-Xaa²-Xaa³-Xaa⁴ as denoted by SEQ ID NO: 31 or any fragment thereof, wherein Xaa is any amino acid, and wherein:

X¹, is a Gly or Leu; X², is Glu or Asp; and X³, is Pro, Val or Gly; and X⁴, is Gly, Met, Asn, Asp or Leu. In some specific embodiments, linker/s comprising the amino acid sequence of any one of: Gly-Asp-Pro-Met, as denoted by SEQ ID NO: 32, Gly-Asp-Pro-Asn as denoted by SEQ ID NO: 33, Gly-Glu-Val-Asp as denoted by SEQ ID NO: 34 and Gly-Glu-Pro-Leu, as denoted by SEQ ID NO: 35, may be also applicable for the SARS CoV RBDs and spike proteins of the invention.

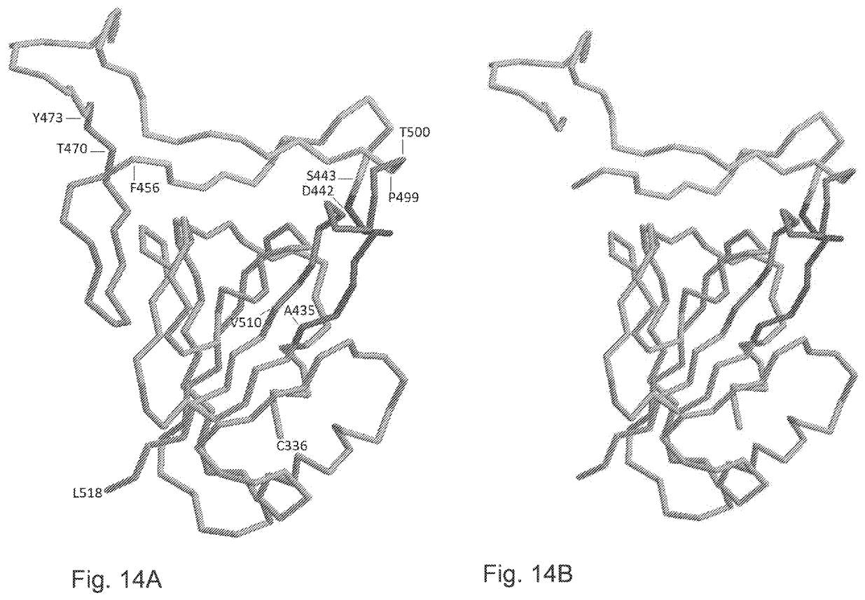

FIG. 13 shows the RBD structure, and specifically the anchor loop that may be replaced in the SARS CoV2, for providing the RBD or spike protein in accordance with the invention. Thus, in some embodiments, the invention provides an RBD and a spike protein of SARS CoV2, having a linker that replaces the anchor loop. In some embodiments the anchor loop comprises the amino acid sequence starting at any one of residues F456, R457, K458, R454 or L455 and ending at any one of residues Y473, I472, E471, T470, S469, I468, Q474, A475 or G476 of the spike protein of SARS CoV2, specifically, as denoted by SEQ ID NO: 6, and variants thereof. In some embodiments, the anchor loop of SARS CoV2 comprises residues 457 to 472 of the spike protein of SARS CoV, specifically, as denoted by SEQ ID NO: 5. Non-binding examples of linkers useful in the present invention in connection with SARS CoV2 may include any of the τ-linkers of the invention comprising the amino acid sequence of any of SEQ ID NO: 57, the GVP linker or any of the linkers of SEQ ID NOS: 42-52, or any variants thereof, specifically, any of the variants disclosed by the invention.

A further aspect of the invention relates to a multimeric and/or multivalent antigen displaying platform comprising at least one RBD or Spike protein of at least one CoV comprising at least one linker that replaces an anchor loop of said Spike protein or any part thereof, or amino acid residue/s thereof, or any fusion protein or conjugate thereof. The anchor loop comprises between 5 to 20 amino acid residues and forms 2 to 5 hydrogen bonds to the core of the RBD of said Spike protein. The amino acid sequence of the loop starts approximately 10 amino acid residues before the first hydrogen bond and ends approximately 10 amino acid residues after the last hydrogen bond to the core of the RBD. The core comprises about five beta strands, and the at least one linker is a bridging linker.

Another aspect of the invention relates to a nucleic acid sequence encoding at least one RBD or at least one Spike protein of at least one CoV comprising at least one linker that replaces an anchor loop of said Spike protein or any part thereof, or amino acid residue/s thereof, or any fusion protein thereof, multimeric and/or multivalent antigen displaying platform thereof or any combinations thereof or any matrix, nano- or micro-particle thereof. More specifically, the anchor loop comprises between 5 to 20 amino acid residues and forms 2 to 5 hydrogen bonds to the core of the RBD of said Spike protein, wherein the amino acid sequence of the loop starts approximately 10 amino acid residues before the first hydrogen bond and ends approximately 10 amino acid residues after the last hydrogen bond to the core of the RBD. The core comprises about five beta strands. It should be noted that the at least one linker is a bridging linker. In some embodiments, such linker may be any of the τ-linkers disclosed by the invention.

In yet a further aspect, the invention relates to a composition comprising an effective amount of at least one RBD or at least one Spike protein of at least one CoV comprising at least one linker that replaces an anchor loop of said Spike protein or any part thereof, or amino acid residue/s thereof, or any fusion protein, multimeric and/or multivalent antigen displaying platform thereof, any derivative, enantiomer, fusion protein, conjugate, polyvalent dendrimer thereof, any nucleic acid sequence encoding the same, and any combinations thereof. The anchor loop comprises between 5 to 20 amino acid residues and forms 2 to 5 hydrogen bonds to the core of the RBD of the Spike protein. The amino acid sequence of the loop starts approximately 10 amino acid residues before the first hydrogen bond and ends approximately 10 amino acid residues after the last hydrogen bond to the core of the RBD. The core comprises about five beta strands. More specifically, the at least one linker is a bridging linker. In some embodiments, such linker may be any of the τ-linkers disclosed by the invention. In some embodiments, the composition optionally further comprises at least one pharmaceutically acceptable carrier/s, adjuvant/s, excipient/s, auxiliaries, and/or diluent/s.

A further aspect of the invention relates to a CoV vaccine comprising at least east one RBD or at least one Spike protein of at least one CoV comprising at least one linker that replaces an anchor loop of said Spike protein or any part thereof, or amino acid residue/s thereof, or any fusion protein, multimeric and/or multivalent antigen displaying platform thereof, any derivative, enantiomer, fusion protein, conjugate, polyvalent dendrimer thereof, any nucleic acid sequence encoding the same, and any combinations thereof. In some embodiments the anchor loop comprises between 5 to 20 amino acid residues and forms 2 to 5 hydrogen bonds to the core of the RBD of said Spike protein. The amino acid sequence of the loop starts approximately 10 amino acid residues before the first hydrogen bond and ends approximately 10 amino acid residues after the last hydrogen bond to the core of the RBD. The core comprises about five beta strands, and the at least one linker is a bridging linker. In some embodiments, such linker may be any of the τ-linkers disclosed by the invention. In some embodiments the vaccine optionally further comprises at least one pharmaceutically acceptable carrier/s, excipient/s, adjuvant/s, auxiliaries, and/or diluent/s.

A further aspect of the invention relates to a method for treating, preventing, inhibiting, reducing, eliminating, protecting or delaying the onset of an infection or an infectious clinical condition caused by at least one CoV in a subject in need thereof. The method comprising the step of administering to the subject an effective amount of at least one RBD or at least one Spike protein of at least one CoV comprising at least one linker that replaces an anchor loop of said Spike protein or any part thereof, or amino acid residue/s thereof, or any fusion protein, multimeric and/or multivalent antigen displaying platform thereof, any derivative, enantiomer, fusion protein, conjugate, polyvalent dendrimer thereof, any nucleic acid sequence encoding the same or any matrix, nano- or micro-particle thereof, and any combinations thereof, or any composition or vaccine thereof. It should be noted that the anchor loop comprises between 5 to 20 amino acid residues and forms 2 to 5 hydrogen bonds to the core of the RBD of said Spike protein. More specifically, the amino acid sequence of the loop starts approximately 10 amino acid residues before the first hydrogen bond and ends approximately 10 amino acid residues after the last hydrogen bond to the core of the RBD, said core comprises about five beta strands, and wherein said at least one linker is a bridging linker. In some embodiments, such linker may be any of the τ-linkers disclosed by the invention.

In some embodiments, the subject is further administered with at least one additional anti CoV vaccine and/or therapeutic agent. It should be noted that the at least one additional anti-CoV vaccine and/or therapeutic agent is administered prior to, after and/or simultaneously with administration of the at least one RBD or at least one Spike protein of at least one CoV comprising at least one linker that replaces an anchor loop of said Spike protein or any part thereof, or amino acid residue/s thereof, or any fusion protein, multimeric and/or multivalent antigen displaying platform thereof, any derivative, enantiomer, fusion protein, conjugate, polyvalent dendrimer thereof, any nucleic acid sequence encoding the same or any matrix, nano- or micro-particle thereof, and any combinations thereof, or any composition or vaccine thereof. In some embodiments, the anchor loop comprises between 5 to 20 amino acid residues and forms 2 to 5 hydrogen bonds to the core of the RBD of said Spike protein. The amino acid sequence of the loop starts approximately 10 amino acid residues before the first hydrogen bond and ends approximately 10 amino acid residues after the last hydrogen bond to the core of the RBD. The core comprises about five beta strands, and the at least one linker is a bridging linker.

Another aspect of the invention relates to a method of inducing an immune response against at least one CoV in a subject in need thereof, the method comprising administering to the subject an immunogenic effective amount of at least one RBD or at least one Spike protein of at least one CoV comprising at least one linker that replaces an anchor loop of said Spike protein or any part thereof, or amino acid residue/s thereof, or any fusion protein, multimeric and/or multivalent antigen displaying platform thereof, any derivative, enantiomer, fusion protein, conjugate, polyvalent dendrimer thereof, any nucleic acid sequence encoding the same or any matrix, nano- or micro-particle thereof, and any combinations thereof, or any composition or vaccine thereof. In some embodiments, the anchor loop comprises between 5 to 20 amino acid residues and forms 2 to 5 hydrogen bonds to the core of the RBD of said Spike protein, wherein the amino acid sequence of said loop starts approximately 10 amino acid residues before the first hydrogen bond and ends approximately 10 amino acid residues after the last hydrogen bond to the core of the RBD. Specifically, the core comprises about five beta strands, and the at least one linker is a bridging linker.

In yet another aspect, the invention provides a method for the preparation, affinity selection and/or isolation of neutralizing antibodies that neutralize at least one CoV and compete with receptor binding of said CoV, the method comprising the steps of:
(a) contacting a serum or lymphocytes of at least one donor with an effective amount of at least one RBD or at least one Spike protein of said CoV, comprising at least one linker that replaces an anchor loop of said Spike protein or any part thereof, or amino acid residue/s thereof, any multimeric and/or multivalent antigen displaying platform thereof, any derivative, enantiomer, fusion protein, conjugate, polyvalent dendrimer thereof, and any combinations thereof, wherein said anchor loop comprises between 5 to 20 amino acid residues and forms 2 to 5 hydrogen bonds to the core of the RBD of said Spike protein. The amino acid sequence of the loop starts approximately 10 amino acid residues before the first hydrogen bond and ends approximately 10 amino acid residues after the last hydrogen bond to the core of the RBD. The core comprises about five beta strands, and wherein said at least one linker is a bridging linker; and

The next step (b), involves recovering the antibodies or at least one lymphocyte bound to the at least one RBD or at least one Spike protein of said CoV. In some embodiments, the serum and/or lymphocytes are obtained from at least one patient recovered from a CoV infection.

A further aspect of the invention relates to a therapeutic passive vaccine comprising neutralizing antibodies that neutralize at least one CoV. In some embodiments, such neutralizing antibodies may be any of the polyclonal and/or monoclonal antibodies prepared by the methods of the invention as disclosed herein before. In some embodiments, the vaccine is prepared by the method according the invention.

Another aspect of the invention relates to a method for the preparation of neutralizing antibodies directed at the Spike protein of at least one CoV, the method comprising the step of:

First (a), contacting at least one lymphocyte or serum of at least one immunized non-human animal, with an effective amount of at least one RBD or at least one Spike protein of the CoV comprising at least one linker that replaces an anchor loop of the Spike protein or any part thereof, or amino acid residue/s thereof, any multimeric and/or multivalent antigen displaying platform thereof, any derivative, enantiomer, fusion protein, conjugate, polyvalent dendrimer thereof, or any CoV comprising at least one linker that replaces the anchor loop of the Spike protein, or any combinations thereof. Next in step (b), recovering the antibodies or lymphocytes bound to said at least one RBD or at least one Spike protein; thereby obtaining neutralizing antibodies that neutralize said CoV. The non-human animal is immunized with an effective amount of said at least one RBD or Spike protein comprising at least one linker replacing the anchor loop or any part thereof, or amino acid residue/s thereof, any multimeric and/or multivalent antigen displaying platform thereof, any nucleic acid sequence encoding the same, any combinations thereof, any derivative, enantiomer, fusion protein, conjugate, polyvalent dendrimer thereof, or any CoV comprising at least one linker that replaces the anchor loop of the Spike protein, at least one attenuated or killed CoV or any variant or mutant thereof, and any composition or vaccine thereof. The anchor loop comprises between 5 to 20 amino acid residues and forms 2 to 5 hydrogen bonds to the core of the RBD of said Spike protein. in some embodiments, the amino acid sequence of the loop starts approximately 10 amino acid residues before the first hydrogen bond and ends approximately 10 amino acid residues after the last hydrogen bond to the core of the RBD, said core comprises about five beta strands, and wherein said at least one linker is a bridging linker.

In yet a further aspect, the invention relates to a therapeutic passive vaccine comprising neutralizing antibodies that neutralize at least one CoV. In some embodiments, such neutralizing antibodies may be any of the polyclonal and/or monoclonal antibodies prepared by the methods of the invention as disclosed herein before. The vaccine is prepared by the method according the invention.

A further aspect of the invention relates to a method of screening for a compound that inhibits binding of at least one Spike protein of at least one CoV to the cognate receptor in a target cell, the method comprising the steps of:

First (a), contacting at least one candidate compound or a plurality of candidate compounds with an effective amount of at least one RBD or Spike protein of the CoV comprising at least one linker that replaces an anchor loop or any part thereof, or amino acid residue/s thereof, or any multimeric and/or multivalent antigen displaying platform thereof, and any combinations thereof, any derivative, enantiomer, fusion protein, conjugate, polyvalent dendrimer thereof, associated directly or indirectly to a solid support and/or a detectable moiety. In some embodiments, the anchor loop comprises between 5 to 20 amino acid residues and forms 2 to 5 hydrogen bonds to the core of the RBD of said Spike protein. The amino acid sequence of said loop starts approximately 10 amino acid residues before the first hydrogen bond and ends approximately 10 amino acid residues after the last hydrogen bond to the core of the RBD, said core comprises about five beta strands, and wherein said at least one linker is a bridging linker; and in step (b), recovering the candidate compound bound to the at least one RBD or Spike protein immobilized to said solid support and/or a detectable moiety, thereby obtaining a compound that binds the Spike protein of said CoV and inhibits binding of the virus to said cognate receptor.

A further aspect of the invention relates to a compound that inhibits binding of at least one Spike protein of at least one CoV to the cognate receptor in a target cell. In some embodiments, the compound is prepared by the method as defined by the invention.

Another aspect of the invention relates to a method for treating, inhibiting, reducing, eliminating, protecting or delaying the onset of at least one pathological condition caused by and/or associated with at least one CoV infection in a subject in need thereof. More specifically, the method comprising the step of administering to said subject an effective amount of a therapeutic passive vaccine comprising neutralizing antibodies that neutralize said CoV, or of a compound that inhibits binding of at least one Spike protein of said CoV to the cognate receptor in a target cell. In some embodiments, the passive vaccine is as defined by the invention, and wherein the compound as defined by the invention.

The invention further provides an effective amount of the therapeutic passive vaccine comprising neutralizing antibodies that neutralize at least one CoV or of a compound that inhibits binding of at least one Spike protein of said CoV to the cognate receptor in a target cell, for use in a method for treating, preventing, inhibiting, reducing, eliminating, protecting or delaying the onset of at least one pathological condition caused by and/or associated with at least one CoV infection in a subject in need thereof. The passive vaccine is as defined by the invention, and wherein the compound as defined herein.

The invention further provides a diagnostic kit comprising at least one of:
(a) at least one RBD or Spike protein of at least one CoV comprising at least one linker that replaces an anchor loop or any part thereof, or amino acid residue/s thereof, or any multimeric and/or multivalent antigen displaying platform thereof, and any combinations thereof, any derivative, enantiomer, fusion protein, conjugate, polyvalent dendrimer thereof, associated directly or indirectly to a solid support and/or a detectable moiety. More specifically, the anchor loop comprises between 5 to 20 amino acid residues and forms 2 to 5 hydrogen bonds to the core of the RBD of said Spike protein. The amino acid sequence of said loop starts approximately 10 amino acid residues before the first hydrogen bond and ends approximately 10 amino acid residues after the last hydrogen bond to the core of the RBD, the core comprises about five beta strands, and wherein said at least one linker is a bridging linker;
(b) antibodies specific for the at least one RBD or Spike protein of at least one CoV associated directly or indirectly to a solid support and/or a detectable moiety.

A further aspect of the invention relates to a diagnostic method for the detection of at least one CoV infection in a mammalian subject, the method comprising the steps of: First step (a), involves contacting at least one biological sample of the subject with at least one of (i) at least one RBD or Spike protein of at least one CoV comprising at least one linker that replaces an anchor loop or any part thereof, or amino acid residue/s thereof, associated directly or indirectly to a solid support and/or a detectable moiety; with (ii) antibodies specific for the RBD or Spike protein associated directly or indirectly to a solid support and/or a detectable moiety; or with (iii) any RBD or Spike protein binding molecule associated directly or indirectly to a solid support and/or a detectable moiety.

The next step (b), involves determining that the subject is infected with CoV if the detectable moiety is detected in the sample.

In some embodiments the anchor loop comprises between 5 to 20 amino acid residues and forms 2 to 5 hydrogen bonds to the core of the RBD of said Spike protein. The amino acid sequence of said loop starts approximately 10 amino acid residues before the first hydrogen bond and ends approximately 10 amino acid residues after the last hydrogen bond to the core of the RBD, said core comprises about five beta strands, and wherein said at least one linker is a bridging linker.

In a further aspect thereof, the invention further provides an effective method for improving epitope based vaccine that comprise at least one Spike protein of CoV, or any fragments or parts thereof. The method of the invention comprises the step of replacing the anchor loop of the Spike protein of said CoV, or any fragments or parts thereof with a linker. In some embodiment, the anchor loop comprises between 5 to 20 amino acid residues and forms 2 to 5 hydrogen bonds to the core of the RBD of said Spike protein, wherein the amino acid sequence of said loop starts approximately 10 amino acid residues before the first hydrogen bond and ends approximately 10 amino acid residues after the last hydrogen bond to the core of the RBD, said core comprises about five beta strands, and wherein said at least one linker is a bridging linker.

The invention further provides at least one linker that replaces an anchor loop or any part thereof, or amino acid residue/s thereof, of a CoV Spike protein. In some embodiments, the anchor loop comprises between 5 to 20 amino acid residues and forms 2 to 5 hydrogen bonds to the core of the RBD of said Spike protein, wherein the amino acid sequence of said loop starts approximately 10 amino acid residues before the first hydrogen bond and ends approximately 10 amino acid residues after the last hydrogen bond to the core of the RBD, said core comprises about five beta strands, and wherein said at least one linker is a bridging linker, thereby forming a functional RBM, RBD and/or Spike protein or any functional fragments thereof. Non-limiting embodiments for the linkers useful in the present aspects are any of the linkers disclosed by the invention, any of the τ-linkers of the invention, for example, any of the linkers of SEQ ID NO: 57, the GVP linker or any of the linkers of SEQ ID NOS: 42-52, or any variants thereof, specifically, any of the variants disclosed by the invention, as well as any of the linkers disclosed by the invention for SARS CoV and MERS, that also include the linkers of SEQ ID NO: 25-25 and any variants thereof.

It should be appreciated that all he definitions and various embodiments disclosed by the present disclosure, are applicable for any of the aspects of the invention, even if not specifically indicated. For example, any of the linkers defined by the invention are applicable for all aspects of the invention. Similarly, the various reconstituted RBMs, RBDs and spike proteins disclosed and defined by the invention are also applicable for all aspects of the invention. Still further, definitions of various general terms, for example, compositions, vaccines, kits, vehicles, and methods, are applicable for any of the aspects of the invention.

All definitions, as defined and used herein, should be understood to control over dictionary definitions, definitions in documents incorporated by reference, and/or ordinary meanings of the defined terms. The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof. The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

Antibodies and Recombinant Receptor

Neutralizing monoclonal antibodies, AO5, 441 and D11 were kindly provided by Dr. Kayvon Modjarrad—Walter Reed Army Institute of Research.

Human ACEII receptor was kindly provided by Dr. Neil King University of Washington Seattle WA.

Construction of SARS CoV2-RBM Conformer Library, for First Generation Reconstituted RBMs:

Reconstitution of RBMs is based on the ability to display segments of the natural RBM connected via combinatorial linkers thus, yielding a diversity of conformers. These are expressed and displayed on Protein 3 of filamentous bacteriophages (5 copies per phage). Thus, virus specific combinatorial phage-display conformer libraries are produced. For this, the previously reported fth-1 vector (D. Enshell-Seijffers, et al., Nucleic acids research 29, e50-e50 (2001)10.1093/nar/29.10.e50)), is modified to contain BstX1 cloning sites at the 5′ end of the protein 3 gene and is used for the construction of the SARS CoV2-RBM conformer libraries.

A gBlock of double stranded DNA sequences corresponding to the SARS CoV2-RBM, containing fth1 BstXI overlapping sequences on both 5′ and 3′ ends is purchased from Integrated DNA Technology (IDT, Israel). This is then used as a PCR-template to generate two segments corresponding to:
Segment A, amino acid residues S443-F456 (SEQ ID NO: 3) of the SARS CoV2-RBM using sense Oligo #1 as denoted by SEQ ID NO: 8 annealed against antisense oligo #2 as denoted by SEQ ID NO: 9.
Segment B, a series of linkers of 3, 4, 5, 6 and 7 random amino acids in length followed by residues T470-N501 (SEQ ID NO: 90) of the SARS CoV2-RBM (using the sense Primer #3, #4, #5, #6, #7; and anti-sense Primer #8, as denoted by SEQ ID NO: 10, 11, 12, 13, 14 and 15 respectively), sense primer #9 as dented by SEQ ID NO: 16 and antisense primer #8 were for construction of loop less RBM.

#1:
(SEQ ID NO: 8)
5′TTCTATTCTCACTCCGCTCCAGAGCTCTGGTCTAAGGTCGGAGGCAA
TTACAATTATTTATATCGCCTTTTC 3′,
#2:
(SEQ ID NO: 9)
5′GAAAAGGCGATATAAATAATTGTAATTGCCTCCGACCTTAGACCAGA
GCTCTGGAGCGGAGTGAGAATAGAA 3′,
#3:
(SEQ ID NO: 10)
5′AATTATTTATATCGCCTTTTCNNKNNKNNKACCGAAATCTACCAAGC
AGGG 3′,
#4:
(SEQ ID NO: 11)
5′AATTATTTATATCGCCTTTTCNNKNNKNNKNNKACCGAAATCTACCA
AGCAGGG 3′,
#5:
(SEQ ID NO: 12)
5′AATTATTTATATCGCCTTTTCNNKNNKNNKNNKNNKACCGAAATCTA
CCAAGCAGGG 3′,
#6:
(SEQ ID NO: 13)
5′AATTATTTATATCGCCTTTTCNNKNNKNNKNNKNNKNNKACCGAAAT
CTACCAAGCAGGG 3′,
#7:
(SEQ ID NO: 14)
5′AATTATTTATATCGCCTTTTCNNKNNKNNKNNKNNKNNKNNKACCGA
AATCTACCAAGCAGGG 3′,
#8:
(SEQ ID NO: 15)
5′TAAACAACTTTCAACAGTTTCCCAGACG 3′,
#9:
(SEQ ID NO: 16)
5′AATTATTTATATCGCCTTTTCACCGAAATCTACCAAGCAGGG3′,

All PCR products were cleaned using AMPure beads (Beckman Coulter, Indianapolis, IN, USA) and cloned into BstX1 precut vector by Gibson assembly reactions. Thus, the following constructs were generated:
full length RBM for 1^{st} generation reconstituted RBMs (residues 443-501 (SEQ ID NO: 40) including the “anchor loop” (residues R457-S469, SEQ ID NO: 37)
“loopless” RBM of 1^{st} generation, in which residues F456 and T470 are connected directly with no amino acids in place of the deleted loop
5 random linkers of 3, 4, 5, 6 and 7 amino acids respectively, in place of the deleted “anchor loop”.

Ethanol-purified Gibson reaction products were used to electroporate E. coli ER2738 electrocompetent cells (cat. no. 60522-1, Lucigen, Middleton, WI, USA) and clones were isolated and confirmed for correct sequence by standard Sanger's sequencing. Bacteria were cultured (shaking at 225 rpm, 37° C., overnight), and phages were precipitated from culture media using polyethelyne glycol 6000/NaCl and resuspended in Tris-buffered saline (50 mM Tris-HCl pH 7.5, 150 mM NaCl (TBS)).

Screening of the Conformer Libraries:

Screening the conformer library can be performed using anyone of three potential probes:
Human ACE-2, the viral receptor.
Convalescent sera or IgG fractions from recovered COVID-19 patients
Neutralizing monoclonal antibodies that compete for ACE-2 binding to the RBM

The following protocol provides the details for screening the library against ACE-2. It is understood that the same protocol can be used when replacing the ACE-2 with either convalescent sera or neutralizing mAbs, e.g., AO5, 441 and D11.

Bio-Panning of SARS-COV2 RBM Linker Phage-Display Library Against ACE2-hFc

Materials:
Dynabeads Protein G, 10004D, Invitrogen (Rhenium).
0.5 ml plastic tubes.
Magnetic stand (Promega).
Blocking solution: sterilized Tris-buffered saline (TBS), containing 3% BSA, filtered through a 0.22 μm filter.
Wash Buffer: Tris-buffered saline (TBS) containing 0.5% Tween-20.
Elution Buffer pH2.2: 0.1M glycine, pH 2.2 with 1 mg/ml BSA, filtered through a 0.22 μm filter.
Neutralization Buffer: 1M Tris-HCl; pH 9.1, autoclaved.
Media: Lysogeny broth (LB); 2XYT; Terrific broth (TB).

1^{st} Round:

Day 0
Prepare a 2 ml DH5αF+ starter in LB medium to be used for amplification and titration.

Day 1
1. ACE2-Fc/library mix: mix 2 ug (2.5 ul from a 0.8 mg/ml stock) of ACE2-hFc and 1×10¹⁰ phages (10 μl) from the COVID-19 RBM linker phage-display library.
Complete the volume to 100 μl (87.5 ul) with blocking solution (TBS+3% BSA). Incubate the tube on a rotating shaker for 1 h at room temperature.
2. Magnetic beads: Re-suspend Dynabeads—vortex well >30 sec.
Add 30 μl of Invitrogen Protein G Magnetic Dynabeads Beads into each tube.
Incubate the tube for 30 min at room temperature on a rotating shaker.
3. Place the tube into a magnetic stand for 2 min to collect the beads against the wall of the tube. Discard the supernatant (˜130 μl) using a pipettor.
4. Wash: Remove the tube from the magnetic stand and add 200 μl of Wash Buffer (TBS+0.5% Tween20) to the tube. Pipette gently 3 times, and then place the tube into a magnetic stand to collect the beads against the wall of the tube. Discard the supernatant. Repeat step 4 (Wash) for a total of 3 times.
5. Remove the tube from the magnetic stand, re-suspend the beads-ACE2-phage complex in 100 μl Wash buffer and transfer the whole volume to a clean tube (this is recommended to avoid co-elution of proteins bound to the tube wall). Place the tubes in a magnetic stand and take out the Wash buffer.
6. Elution: Add 105 μl of Elution Buffer pH2.2 to the tube, pipette up and down and incubate the tube on a shaker at room temperature for 10 min.
7. Neutralization: Place the tube on the magnetic stand in order to separate the beads and transfer 100 μl of the supernatant containing eluted phages to a new Eppendorf tube containing 19 ul neutralization Buffer pH 9.1 and vortex.
8. Repeat steps 6 and 7. Combine the 2 eluates to one tube—elution 1 (Elu1).
9. Titrate the 1^{st} eluate (optional).
10. Prepare a 2 ml DH5αF+ starter in LB medium for the following day.

Amplification:

Day 1
1. Prepare 5 ml 2XYT medium in a 50 ml tube and add 100 μl of the DHF5αF+ starter. Incubate at 37° C., 225 rpm until the concentration of the bacteria reaches about 1.5 OD600 and above (typically takes 4-5 hours).
2. Pili—Reduce the speed of shaking to around 60 rpm for 30 min at 37° C., to allow the bacterial pili to regenerate.
3. Infection—Add 150p of the eluate from the first (or previous) panning to the bacteria and incubate for 30 min at 60 rpm at 37° C. Gently swirl the contents of the flask every 10 min so to disrupt bacterial aggregates, making sure not to damage the bacterial pili.
4. Phenotypic expression—Transfer the infected bacteria culture (5 ml) into 100 ml of 2XYT medium in 0.5 L flasks. Incubate at 37° C., 225 rpm for 1 hour.
5. Growth—Add tetracycline (20 μg/ml final concentration, 1:1,000 from our stock) to the flasks and continue to incubate them at 370 C, 225 for 18-20 hours.

Day 2
6. Phage extraction—Transfer bacteria into sterile 250 ml centrifugation bottles and pellet the bacteria by centrifugation at 8000 rpm for 20 min.
7. PEG precipitation—Transfer the sup (100 ml) to sterile 250 ml centrifugation bottles and add 0.4 volumes (40 ml) of PEG/NaCl. Mix thoroughly by inverting at least 30 times. Place bottles at 4° C. overnight to allow precipitation of the phages.
8. Prepare a 2 ml DH5a F+ starter in LB medium—as in step 0. This will allow you to titrate your Amplificate (optional).

Day 3
9. Centrifuge bottles to pellet the phages, 45 min, 8,000 rpm, 4° C.
10. Discard supernatant, spin-down the pellet for 5 min at 6000 rpm and remove excess fluid by aspiration or with a pipette.
11. Re-suspend pellet in 2 ml of sterile TBS and incubate at 37° C., 225 rpm for 30 min to allow complete resuspension.
12. Transfer the re-suspended phages to new a 2 ml tube and centrifuge 5 min at maximal speed to precipitate debris.
13. Transfer the phages to a clean tube marked as Amplificate 1 (Amp1).

2^{nd} Round
14. ACE2-Fc/Amp1 mix: mix 2 ug (2.5 ul from a 0.8 mg/ml stock) of ACE2-hFc and 10 μl from Amp1. Complete the volume to 100 μl (87.5 ul) with blocking solution (TBS+3% BSA). Incubate the tube on a rotating shaker for 1 h at room temperature.
11. Magnetic beads: Vortex (>30 sec) the Dynabeads until complete resuspension in a vial or tube. Add 30 μl of Invitrogen Protein G Magnetic Beads into each tube that contains ACE2+Amp1 phages. Incubate the tube for 30 minutes at room temperature on a rotator.
15. Place the tube into a magnetic stand for 2 min to collect the beads against the wall of the tube. Discard the supernatant (˜130 μl) using a pipettor.
16. Wash: Remove the tube from the magnetic stand and add 200 μl of Wash Buffer (TBS+0.5% Tween20) to the tube. Pipette gently 3 times, and then place the tube into a magnetic stand to collect the beads against the wall of the tube. Discard the supernatant. Repeat step 16 (Wash) to a total of 3 times.
17. Remove the tube from the magnetic stand, re-suspend the beads-ACE2-phage complex in 100 μl Wash buffer and transfer the beads suspension to a clean tube (this is recommended to avoid co-elution of proteins bound to the tube wall). Place the tubes in a magnetic stand and take out the Wash buffer.
18. Elution: Add 105 μl of Elution Buffer pH2.2 to the tube, pipette up and down and incubate the tube on a shaker for 10 min.
19. Neutralization: Place the tube on the magnetic stand in order to separate the beads and transfer 100 μl of the supernatant containing the eluted phages to a new 1.5 ml Eppendorf tube with 19 μl neutralization Buffer pH 9.1, vortex.
20. Repeat steps 18 and 19. Combine the 2 eluates to one tube—elution 2 (Elu2).
21. Titrate Elu2 (optional).
22. Prepare a 2 ml DH5αF+ starter in LB medium for the following day.

Continue to a Second Amplification and a 3^{rd} Round of Bio-Panning as has been Done in Round 2.

Picking of Positive Clones:
1. Dilute 200 μl of bacteria (from the starter) in 5 ml of TB medium complete with 10% Potassium Phosphate buffer (PB) in a 50 ml tube. Incubate at 37° C., 225 rpm until the concentration of the bacteria reaches about 1.5 OD₆₀₀ and above (this typically takes 4-5 hours).
2. Reduce the speed of shaking to around 60 rpm for 30 min to allow the bacterial pili to regenerate (maintain 37° C.). In parallel warm LB-tet agar plates.
3. Prepare 3 Eppendorf tubes with 30 μl bacteria from previous step (swirl the test tube gently to mix the bacteria). Add phages from the eluate being screened to the bacteria so to have 500 phages in tube 1 (tubes 2 and 3 should have 2 times more and 2 times less than 500 to try and obtain plates that have colonies comfortably dispersed for picking—this step is empirical).
4. Let stand for 15 min at room temperature for infection of the bacteria to take place. Make sure not to agitate the bacteria at this stage in order not to destroy their pili.
5. Complete the volume with LB medium to a final volume of 200 μl, incubate for 20 min at room temperature for phenotypic expression. Plate on LB-tet agar plates to produce hundreds of tet-resistant colonies. Incubate over night at 37° C.
6. The next day fill the wells of a U-bottom sterile 96-well plate with 200 μl of TB medium complete with 10% Potassium Phosphate buffer (PB) containing 20 μg/ml tetracycline (1:1000 from our stock) thus, 45 ml TB+5 ml PB+50 μl tet. Mark the plates thoroughly since these will be your “master plates”.
7. “Pick” colonies from the LB-tet agar plates by stabbing single colonies using sterile toothpicks and inoculate the wells of the plate by dipping the contaminated-tip of the toothpick into the media. Make sure your plates contain an empty well without any colony, a positive control colony and a negative control colony should you have them.
8. Seal plates with parafilm and secure them in a humidified box to reduce the amount of evaporation. Shake overnight (about 18 hours) at 160 rpm, 37° C. thus, producing mini-cultures of phage clones.

Identify Positive Phages—“Dot-Blots”
9. Centrifuge the mini-cultures plates at 4,000 rpm for 30 min at room temperature. A tight bacterial pellet should be formed. Using a multichannel pipettor transfer sterilely (avoiding the bacterial pellet, by tilting the U bottom plate and the multi-channel) 125 μl/well of the supernatant (containing the phages) to a flat-bottom 96-well plate already containing 50 μl/well of PEG/NaCl, and mix by pipetting 10 times.
11. Incubate the plates at 4° C. for at least 2 hours to allow the phages to precipitate. The original plates containing the bacterial pellets are the “master plates” and should be sealed with parafilm and stored at 4° C. The “shelf-life” of “master plates”, stored in this manner, is no more than a few weeks. Another consideration is that for some phage systems deletions can often occur in the phage genomes and time, therefore, may be a factor. Thus, you want to complete the analyses and establish long term stocks of important phages/bacteria as soon as possible.
12. Centrifuge the flat-bottom plates at 4,000 rpm for 40 min at room temperature. To remove the bulk of the fluid, invert the plate into the sink. Remove the residual fluid by slapping the plate face down on several layers of paper-towels. Then re-suspend the pellet in 110 μl TBS.
13. As phages are difficult to re-suspend, shake the plates on a plate shaker at 600 rpm for 30 min.
14. Prepare dot-blots by applying the re-suspended phages from each well to nitrocellulose membrane filters using a vacuum manifold system. First flush each well of the assembled manifold/filter with 100 μl of TBS. Then apply 100 μl of the phages under vacuum. Finally wash the phages on the filter with an additional 100 μl of TBS. Mark the positions of the wells so you can identify and correlate them later on to the corresponding wells of your “master plate”.
15. Block the membranes by rocking the membranes for 1 hour at room temperature in Blocking solution (5% skim milk, in TBS). The volume of the solution should be enough to completely cover the filter so to ensure that is does not stick to the bottom of the container (about 0.3-0.5 ml/cm² of filter).
16. Add antibody/ACE2 to a final concentration of 2 μg/ml in Blocking and incubate the filter at 4° C., with gentle rocking overnight or for 4 hours at room temperature.
17. Discard the incubation solution and wash the membrane for 30 minutes, changing the TBS every 5 minutes.
18. Add secondary antibody conjugated to HRP (goat-anti-human-HRP) diluted 1:5000 in Blocking solution and incubate the membrane for 45 min at most at room temperature, with gentle rocking.
17. Wash the membrane for 30 minutes, changing the TBS every 5 minutes.
18. The positive signals can be detected by ECL immuno-detection.

Small Scale Purification of Phages for Validation of Positive Clones
1. Prepare 2 ml starters of the bacteria containing the phages identified as positives in step 18, in TB medium complete with 10% Potassium Phosphate buffer (PB) containing 20 μg/ml tetracycline (1:1000 from our stock). Pick the colonies from the “master plates”. Do not forget to include positive and negative control clones.
2. The next day, transfer the cultures to 2 ml Eppendorf tubes and centrifuge them at maximal speed, 30 min, at room temperature. Keep the starter tubes for future growth of validated clones.
3. Transfer 1.5 ml of the supernatant to 2 ml Eppendorf tubes with 600 μl of PEG-NaCl prepared in advance (and kept on ice) for each phage. Vortex well. Dry the pellets and freeze them for future use for mini-preps.
4. Incubate the phages in PEG-NaCl for 1 hour on ice at 4° C. to precipitate the phages.
5. Centrifuge at maximal speed for 30 min, at 4° C. Discard supernatant and spin-down for another 5 min at maximal speed.
6. Aspirate the supernatant using vacuum, do not disturb the pellet.
7. Add 200 ul of sterile TBS to each tube, vortex well and let re-suspend on a shaker for 15 min, at room temperature.
8. Spin-down for 5 min at maximal speed to remove debris, transfer supernatant to a fresh tube.
9. Keep phages at 4° C. until application to Dot Blot.
10. For validation in Dot Blot use 100 ul of the phages for incubation with protein/antibody of choice and another 100 ul of the phages for a membrane that will be incubated without protein/antibody—just with the secondary antibody to insure specificity. Otherwise follow the same protocol as for the screening of picked clones.

Enzyme-Linked Immunosorbent Assay:

ELISA plate wells are coated overnight with 0.5-1.0 μg/well of Ab or ACE2 receptor in TBS (100 microliters of TBS solutions of 5-10 micrograms per ml). Next, the plates are washed with TBS, blocked with 5% skim milk in TBS and incubated with 1010 phages/well. Wells are washed with TBS and incubated with polyclonal rabbit anti-M13 antibody. Next, wells are washed and incubated with HRP-conjugated goat anti-rabbit antibody (1:5000, Jackson Immuno Research Lab, Inc, West Grove, PA, USA). Following an additional round of washing, wells are reacted with the TMB/E ELISA substrate (Merck Millipore, Billerica, MA, USA). Absorbance is measured at 650 nm using a micro-plate reader (Bio Tek, Winooski, VT, USA). All measurements are made in duplicate and experiments are repeated at least three times.

### Example 1

Reconstitution of the SARS CoV2 RBM, First Generation

Due to the recent emergence of the SARS CoV2 in China, a design was proposed for the immediate reconstitution of its RBM.

The SARS CoV2 spike protein is typical of beta coronaviruses consisting of some 1200 amino acids and two domains, the S1 domain which contains the NTD (N-terminal domain) and the CTD (C-terminal domain) and the S2 domain which contains the fusion hydrophobic helices. The S1 domain, like that of SARS CoV of 2003 and MERS CoV of 2012 epidemics, contains the Receptor Binding Domain (RBD) which harbors the Receptor Binding Motif (RBM).

Table 1 provides a comparison between the sequences of the landmark structures of the Spike proteins of SARS-CoV, MERS-CoV and SARS-Cov2.

TABLE 1
Comparison of landmark structures of the Spike proteins of SARS-COV,
MERS-CoV and SARS-CoV2.

| | SARS | MERS | SARS |
|---|---|---|---|
| | CoV | CoV | CoV2 |
| | (SEQ ID | (SEQ ID | (SEQ ID |
| | NO: 17) | NO: 18) | NO: 6) |
| PDB | 2gvh | 4kqz | 6m17 |
| β1 | 339-346 | 398-405 | 352-360 |
| β2 | 362-368 | 421-427 | 375-381 |
| β3 | 382-388 | 439-447 | 394-402 |
| β4 | 417-425 | 475-483 | 431-438 |
| β5 | 493-502 | 567-579 | 507-518 |
| RBM | 430-487 | 489-565 | 443-499 |
| loop | 444-456 | 515-525 | 457-572 |

The amino acid residues of the Spike protein of SARS-CoV, MERS-CoV and SARS-CoV2 indicated in this Table correspond to amino acid residues denoted by SEQ ID NO: 6, 17 and 18 respectively.

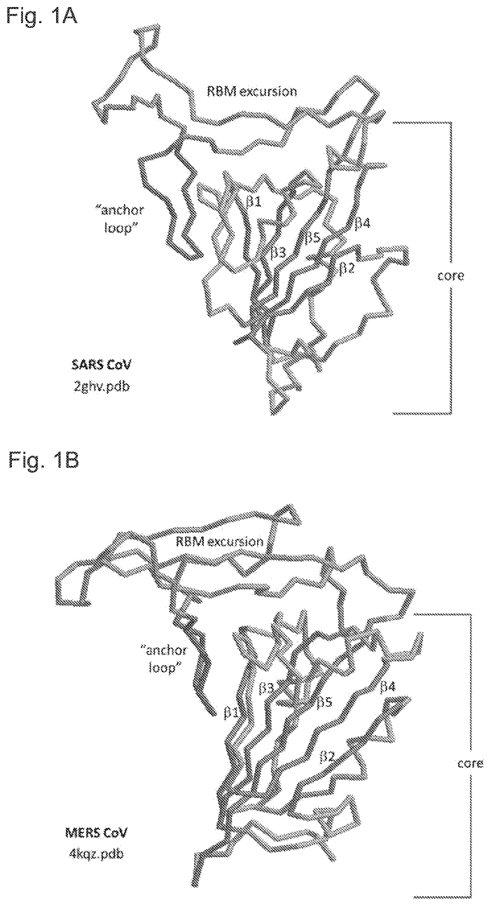

There is a high level of genomic homology between the SARS-CoV and SARS CoV2 (about 88%). The availability of the SARS CoV2 genome has allowed us to model its RBD. As is shown in FIG. 1C, the proposed RBD contains all the following structural hallmarks: the 5 beta-strands of the core, the RBM excursion, and the “anchor loop”. Hence, RBM reconstitution was performed by identifying 4 amino acid residues: the beginning of the excursion, the end of the excursion and the two residues flanking the “anchor loop”. Critical examination of the SARS CoV2 model suggests the construction of the RBM conformer library as follows:

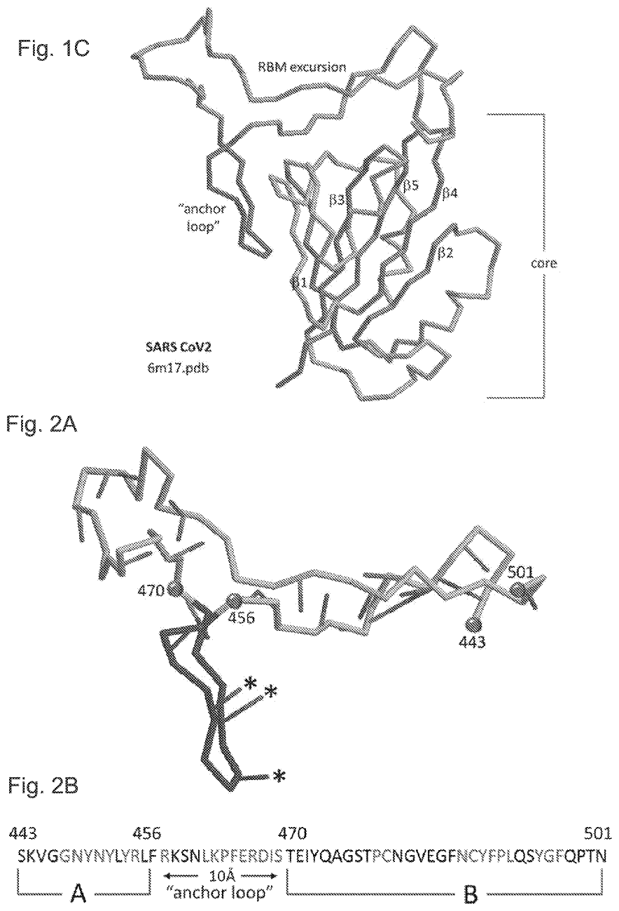

The SARS CoV2 RBM sequence, as designated by SEQ ID NO: 40 (corresponding to residues S443 to N501, 59 amino acids) is cloned into the phage protein 3 in two segments, A: S443-F456, as denoted by SEQ ID NO: 3; and B: T470 to N501 as denoted by SEQ ID NO: 90, connected via a series of 5 combinatorial linkers ranging from 3 to 7 amino acids in length, thus replacing the “anchor loop” (13 residues R457 to S469, as denoted by SEQ ID NO: 37, FIG. 2B). For the first generation of reconstituted RBM, removal of the 13 residues, creates a gap of 10 Å between the two flanking residues 456 and 470. The linkers bridge the 10 Å distance between F456 and T470, thus removing the loop that, as in SARS, is tacked via three hydrogen bonds to the core (asterisks in FIG. 2A). In order to select RBM candidates for further development, the proposed library must be screened for functional conformers. First a screening against ACE2, the receptor for SARS-CoV2 [6] (or SARS-CoV neutralizing mAbs) is performed.

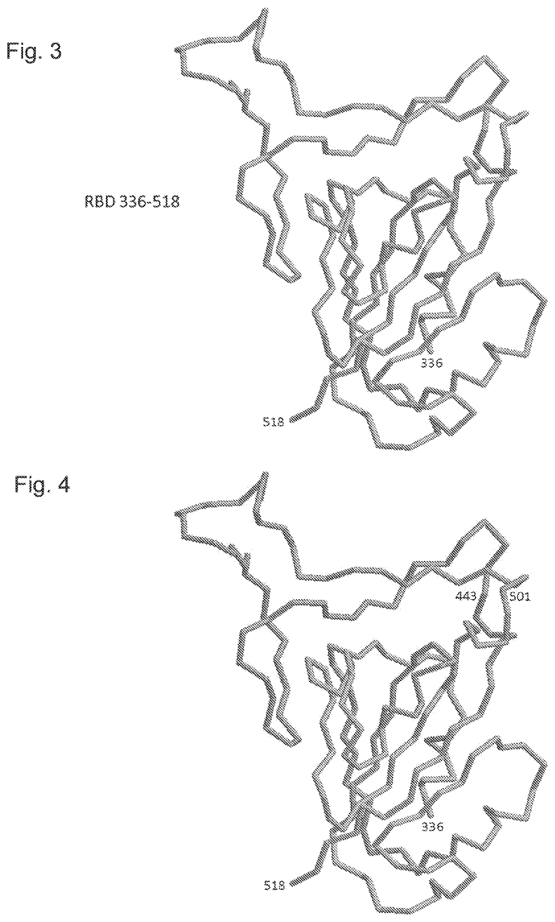

According to the structural basis for the recognition of the SARS-CoV-2 by full-length human ACE2 [10], in order to design the reconstituted RBM, the following elements of SARS-COV2 were identified: As illustrated in FIG. 3, the SARS CoV2 RBD corresponds to residues 336-518 (SEQ ID NO: 1) based on the cryo-EM structure 6m17 PDB [10].

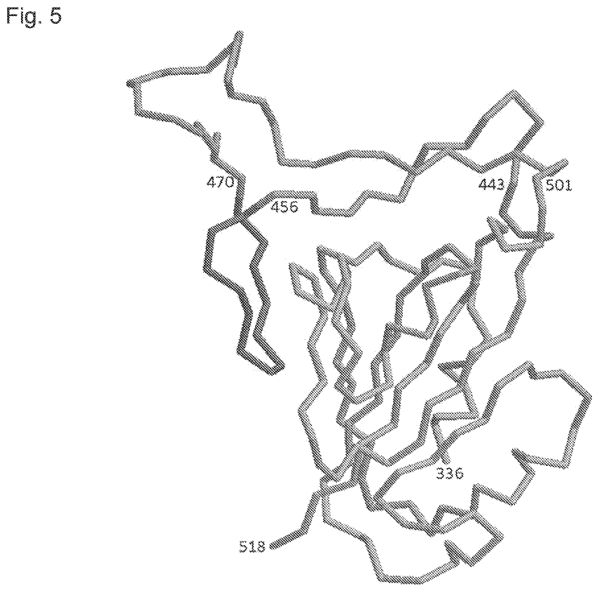

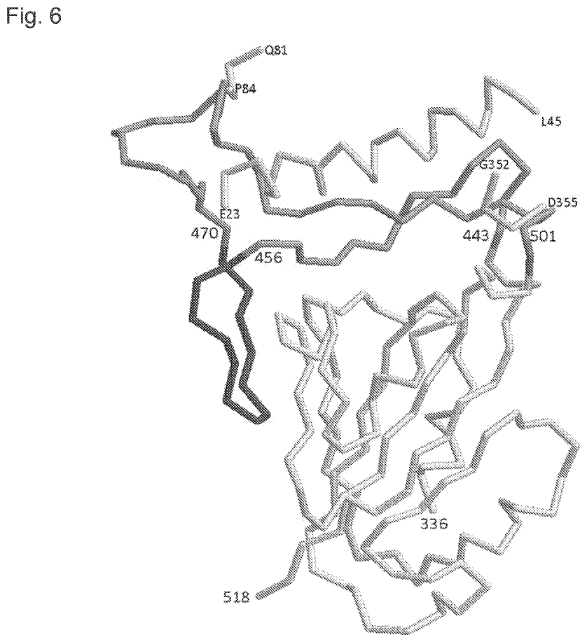

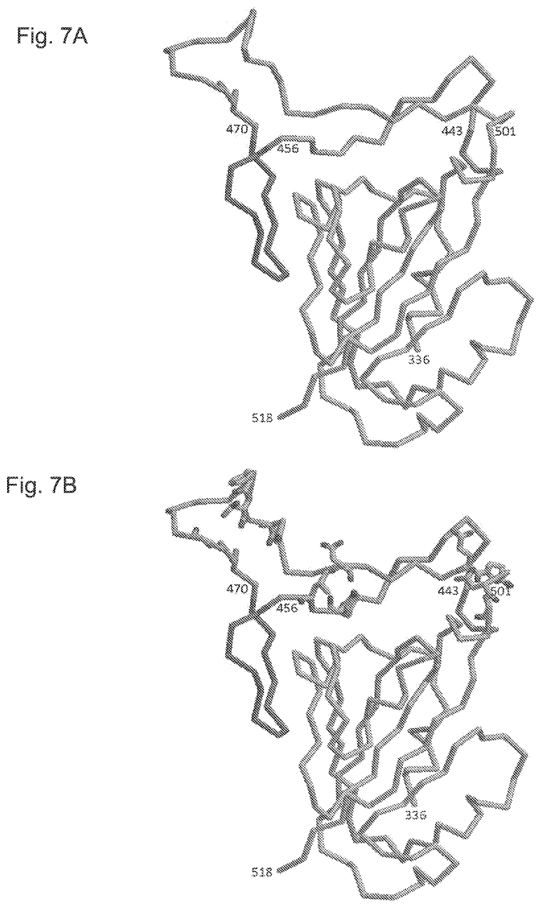

In addition and as shown in FIG. 4, the RBM excursion—residues 443-501 (SEQ ID NO: 40) into the SARS CoV2 RBD residues 336-518 (SEQ ID NO: 1) are highlighted in orange. FIG. 5 shows the anchor loop (residues 457-469 (SEQ ID NO: 37)), used for the first generation highlighted in blue. FIG. 6 illustrates how three strands of ACE2 interact with the RBM, as well as the extended alpha-helix (residues E23 to L45) that contacts multiple residues of the RBM.

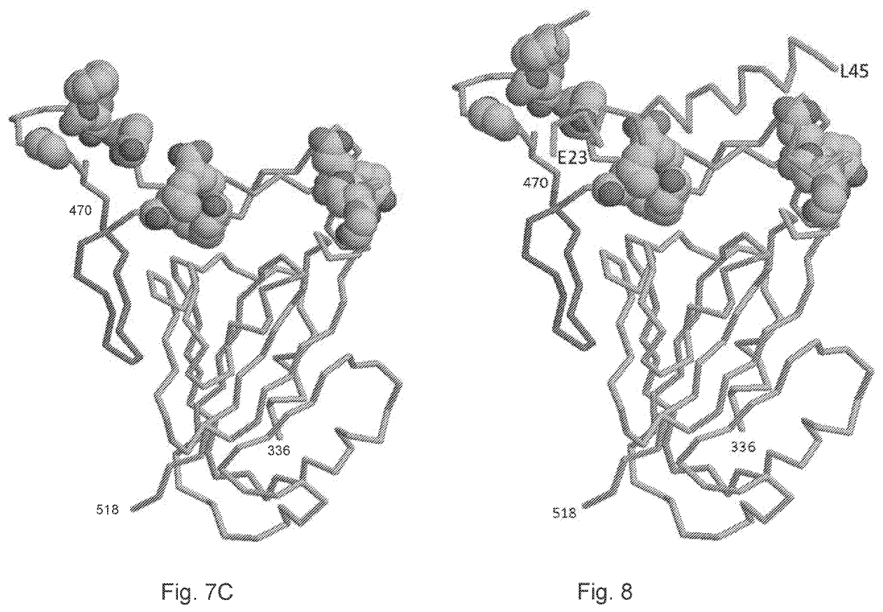

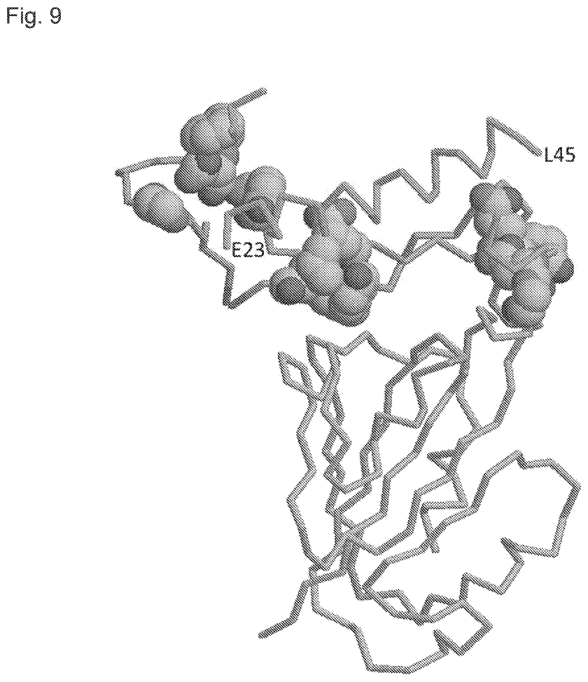

The exact contact residues in the RBM are indicated as grey backbone (FIG. 7A), sticks (FIG. 7B) and spacefill (FIG. 7C). FIG. 8 provides a schematic representation of the contact residues of the RBM together with the three strands of ACE2 shown in green.

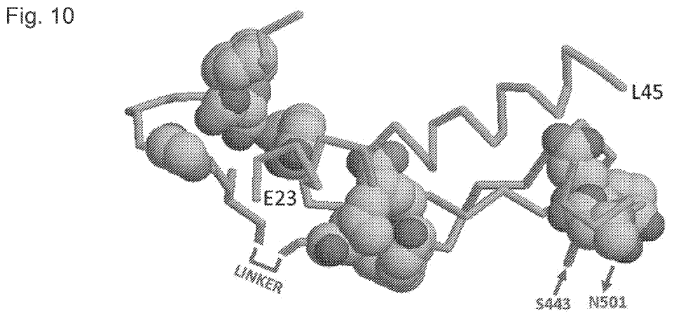

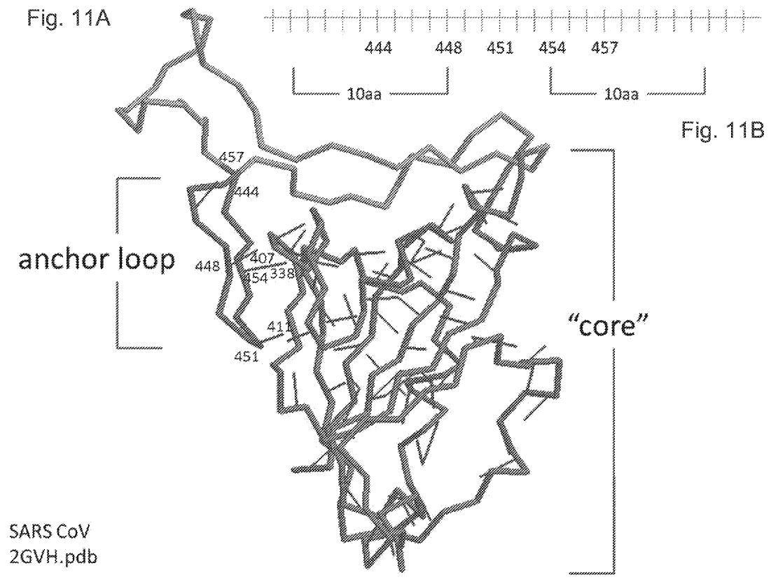

A first step in the RBM reconstitution consists of removal of the anchor loop as illustrated in FIG. 9. Finally FIG. 10 indicates the design of the reconstituted RBM showing the contact residues, the orientation of the ACE2 segments and the position of the combinatorial linker bridging between residues 456 and 470 thus replacing the anchor loop (residues 457 to 469, SEQ ID NO: 37). Furthermore, concerning the exact definition of the anchor loop, the Receptor Binding Domain of beta-coronaviruses consists of an excursion that initiates from the 4th beta strand of the core of the Receptor binding domain and ends just before the 5th beta strand of the core, as illustrated in FIG. 11A-11B for SARS-CoV, FIG. 12A-12B for MERS-CoV and in FIG. 13A-13B for SARS-CoV2. Whereas the RBM forms contact with the virus receptor, e.g., ACE2 for SARS CoV and SARS CoV2, there exists a short loop stemming from the RBM excursion that does not contact the receptor. Rather this loop serves to tack the excursion to the core via a number of hydrogen bonds, hence this loop is referred to as the anchor loop. Thus, the RBM structure can be easily identified and defined as the excursion following beta strand 4 of the core of the RBD and ending just before beta strand 5 of the RBD core. Each RBM excursion is held in place via an anchor loop that tacks the excursion to the surface of the core via a number of hydrogen bonds. The dimensions of the anchor loop can be defined as a loop stemming from the RBM excursion of typically 10-16 amino acids in length and no more than 20-25 residues. The anchor loop is further characterized by the fact that it forms 2-3 and no more than 5 hydrogen bonds with the surface of the RBD core. The residues that contribute to these hydrogen bonds are contained within the anchor loop, the first of which is within 10 residues of the beginning of the loop and the last of which is within 10 residues from the end of the loop. In the construction of the conformer library and consequently the reconstituted RBM this anchor loop is replaced by a linker that enables the remainder of the RBM to assume a physiological conformation.

The RBD of SARS CoV (FIG. 11A), MERS CoV (FIG. 12A) and SARS CoV2 (FIG. 13A) are shown. The core of each RBD (pink) contains the 5 hallmark beta strands. The RBM features (grey) contain the anchor loop (blue) that forms hydrogen bonds with the core. The contributing members of each hydrogen bond is indicated as well as the beginning and end of each of the anchor loops. The “rulers” schematically represented for SARS-CoV, MERS-CoV and SARS-CoV2 in FIG. 11B, FIG. 12B and FIG. 13B respectively, indicate the distance from the first hydrogen bond which is less than 10 residues from the first residues of the anchor loop. Similarly, the distance from the last hydrogen bond to the last residues of the loop is also less than 10 residues.

An alternative to screening using the ACE-2 receptor or neutralizing antibodies is the use of convalescent sera from recovered patients. Over the numerous cases of 2019-CoV infection reported, the vast majority has fully recovered. The sera of those who have recovered contain antibodies that have efficiently cleared the infection. Therefore, these sera are used to screen the SARS CoV2 conformer library and select for functional RBMs of this new emerging threat.

### Example 2

Construction of the Second-Generation Combinatorial RBM Library

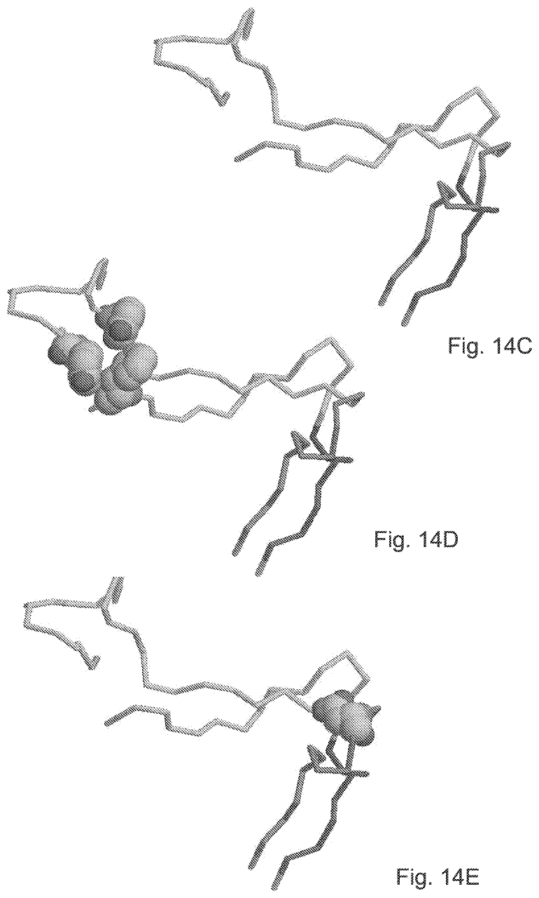

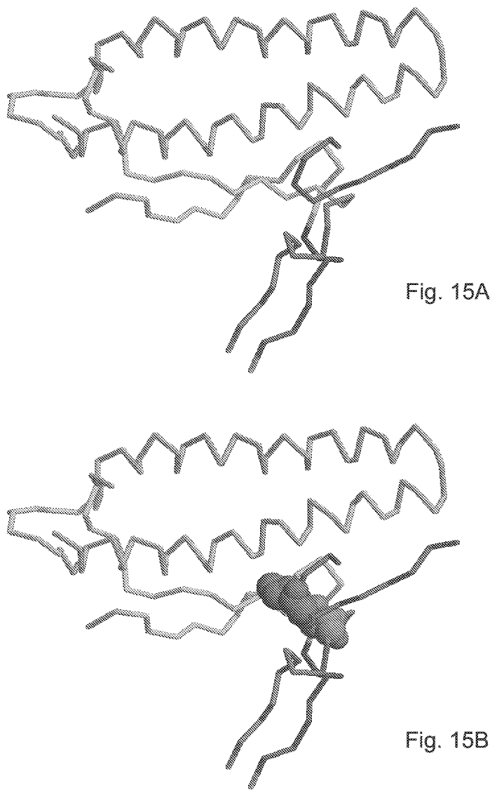

As shown by FIGS. 14-15, the RBM is an extension of the RBD which binds the viral corresponding receptor ACE2. As shown by these figures, the loop can start from residue 457 to 472 (SEQ ID NO: 5), and the RBM can be defined from residue 443 to 499, SEQ ID NO: 2.

The inventors therefore prepared second and third generation RBM libraries. The second generation combinatorial RBM library includes residues S443 through P499 (SEQ ID NO: 2) of the spike protein. The Anchor Loop (residues 457 through 472, SEQ ID NO: 5) is replaced with a bridging linker 1-5 random amino acids in length. The construct is cloned between the two BstX1 sites of the recombinant Protein 3 of the filamentous bacteriophage fd. Note that the BstX1 site is: 5′ . . . CCA NNNNN^{↓}N TGG . . . 3′ (SEQ ID NO: 91) generating a 3′ overhang.

Each of the two sites have different overhangs, thus ensuring the correct orientation of the insertion of the constructs. Moreover, the CCA sequence of the second site produces a Proline residue. Hence the actual insert is designed to extends to Q498 and the required P499 is added de facto due to the nature of the BstX1 5′ CCA sequence. The general scheme of the second-generation library (SEQ ID NO: 92) is:

FYSHSAPELWSKVGGNYNYLYRLF-Linker NNK(1-5)-YQAGSTPCNGVEGFNCYFPLQSYGFQPDVWETVESC
BstXI Fragment A Linker Fragment B BstXI

Where the BstX1 amino acid equivalents are highlighted in italics and the combinatorial linkers in bold. Residues S443 through F456 (SEQ ID NO: 3) are in underlined bold and Y473 through Q498 (SEQ ID NO: 93) are underlined. The black flanking residues are derived from Protein 3 of the vector.

For each of the 5 libraries (corresponding to NNK 1 through 5) two oligonucleotides were used. The first oligonucleotide, “Sense”, contained 30 nucleotides overlapping with the fth1 vector at the N′ BstXI site, followed by fragment A of the RBM sequence, S443-F456 (SKVGGNYNYLYRLF, SEQ ID NO: 3) and a linker segment comprised of 1-5 NNK codons (Table 2). The linker segment was followed by the first 21 nucleotides of fragment B of the RBM, Y473-P479 (YQAGSDP, SEQ ID NO: 122).

TABLE 2
Sense oligonucleotides for the construction of the 2^{nd} generation RBM libraries. Linkers
1-5 are denoted by SEQ ID NOs: 94-98.

| | Name | Sequence 5′-3′ |
|---|---|---|
| 1 | 1)NNK | TTCTATTCTCACTCCGCTCCAGAGCTCTGGAGCAAAGTTGGTGGAAACTATAATTATCTGTATCGGCTGTTTNNKTATCAAGC |
| | | TGTTCTACACCG |
| 2 | 2)NNK | TTCTATTCTCACTCCGCTCCAGAGCTCTGGAGCAAAGTTGGTGGAAACTATAATTATCTGTATCGGCTGTTTNNKNNKTATCA |
| | | AGCTGGTTCTACACCG |
| 3 | 3)NNK | TTCTATTCTCACTCCGCTCCAGAGCTCTGGAGCAAAGTTGGTGGAAACTATAATTATCTGTATCGGCTGTTTNNKNNKNNKTA |
| | | TCAAGCTGGTTCTACACCG |
| 4 | 4)NNK | TTCTATTCTCACTCCGCTCCAGAGCTCTGGAGCAAAGTTGGTGGAAACTATAATTATCTGTATCGGCTGTTTNNKNNKNNKNN |
| | | KTATCAAGCTGGTTCTACACCG |
| 5 | 5)NNK | TTCTATTCTCACTCCGCTCCAGAGCTCTGGAGCAAAGTTGGTGGAAACTATAATTATCTGTATCGGCTGTTTNNKNNKNNKNN |
| | | KNNKTATCAAGCTGGTTCTACACCG |

The second oligonucleotide, “Anti sense”, is complementary to the 3′ end of the Sense oligonucleotide (21 bases of fragment B) followed by residues P479-0498 (PCNGVEGFNCYFPLQSYGFQ, SEQ ID NO: 99) of fragment B of the RBM and additional 30 nucleotides of the fth1 vector that include the 3′ BstXI site:

Anti-sense oligonucleotide:
SEQ ID NO: 100
5′-ACAACTTTCAACAGTTTCCCAGACGTCTGGTTGAAAACCGTAACTT
TGTAATGGAAAATAACAGTTAAATCCCTCGACTCCATTGCACGGTGTAG
AACCAGCTTGATA-3′,.

The same Anti Sense oligonucleotide was used for the construction of all 5 libraries. It is noted that P499 of the RBM is actually provided by the Proline of the 3′ BstXI site.

Preparation of the libraries was done by annealing the Sense and Anti sense oligonucleotides. The annealed product was extended using Klenow fragment (3′→5′ exo −) (M0212 L, NEB) to ‘fill-in’ the complementary strand and cleaned by ethanolic precipitation. The inserts were cloned into BstXI digested fth1 vector using Gibson assembly reaction (A46628, Invitrogen). After calibration of the efficacy of the Gibson reaction in DH5alphaF-competent cells (C29871, NEB), five Gibson reactions for each library were used to electroporate TG1 cells (60502, Lucigen). After 1 hr of phenotypic expression, the bacterial cultures were tittered in order to estimate the actual complexity of the libraries (Table 3). The variation of each library was confirmed by sequencing 10-20 randomly picked colonies from the titration plate.

TABLE 3
Calculated complexities of the 2^{nd} generation RBM libraries.

| First-generation | Measured |
|---|---|
| RBM library | complexity |
| 1 × NNK | 7 × 10⁵ |
| 2 × NNK | 4.8 × 10⁵ |
| 3 × NNK | 6 × 10⁶ |
| 4 × NNK | 4 × 10⁶ |
| 5 × NNK | 4 × 10⁶ |

Each library was further amplified by growing the bacterial culture after electroporation in 500 ml LB with 20 μg/ml of tetracycline overnight at 37° C., 225 rpm. Next, the culture was centrifuged for 20 min, 8000 rpm at room temperature to pellet the bacteria. The phage-containing supernatant was transferred to cold polyethylene-glycol/NaCl (33% PEG-6000, 3.3M NaCl) and incubated at 4° C. overnight to precipitate the phages. Following centrifugation of 45 min, 8000 rpm at 4° C., the precipitated phages were resuspended in sterile TBS and tittered by plaque assay.

### Example 3

Construction of the Extended-Combinatorial RBM Library, Third Generation

The second-generation library described in Example 2 represents the RBM sequence 443-499 SEQ ID NO: 2, in which the Anchor loop has been swapped for a series of combinatorial linkers from 1-5 residues long.

Here, in Example 3 the construction of a series of 88 different extended combinatorial libraries is describe in detail. These are fundamentally the same in being inclusive of the second-generation library but differ in the N-terminal and C-terminal residues that initiate and terminate the libraries. The initiation residues are selected from anyone of the first 8 residues preceding residue S443, i.e., from residue 435 through 442 (AWNSNNLD, SEQ ID NO: 85). The termination can end at anyone of the 11 residues from residue 500 through 510 (TNGVGYQPYRV, SEQ ID NO:86). Hence a total of 88 possible combinations of initiations and terminations are covered in the Extended library.

General scheme for the Extended Library (SEQ ID NO: 133):
FYSHSA PELW AWNSNNLD SKVGGNYNYLYRLF NNK1-5 YWAGSTPCNGVEGFNCYFPLQSYGFQP TNGVGYQPYRV PDVW ETVESC
BstXI N′ Extension Fragment A Linker Fragment B C′ Extension BstXI

Where the BstX1 amino acid equivalents are highlighted in italics and the combinatorial linkers in bold. The 8 initiating residues and 11 terminating residues are underlined.

The construction of these 3^{rd} generation extended RBM libraries was based on the Klenow fragments generated for the first-generation RBM libraries. These were used as templates for PCR with new sets of forward and reverse oligonucleotides designed to add one amino acid (of the original sequence of the RBM) at a time at the beginning and the end of the RBM (S443-Q498, SEQ ID NO: 101).

For this, a set of 8 Sense oligonucleotides was designed, so that each one adds an additional residue to Fragment A of the RBM starting from A435-D442 (AWNSNNLD, SEQ ID NO: 85). Each oligonucleotide starts with 30 nucleotides that are complementary to the 5′ BstXI site of the fth1 vector (Table 4). A set of 11 Anti-sense oligonucleotides was designed in order to extend Fragment B of the RBM from P499-V510 (PTNGVGYQPYRV, SEQ ID NO: 102). Each Anti-sense oligonucleotide contains 30 nucleotides that are complementary to the 3′ BstXI site of the fth1 vector (Table 5).

TABLE 4
Sense oligonucleotides for the construction of the 3^{rd} generation extended RBM libraries.
The oligos 1-8 sequences are denoted by SEQ ID NOs: 103-110, respectively.

| | Name | Sequence 5′-3′ |
|---|---|---|
| 1 | 442F_RBM | TTCTATTCTCACTCCGCTCCAGAGCTCTGGgatAGCAAAGTTGGTGGA |
| 2 | 441F_RBM | TTCTATTCTCACTCCGCTCCAGAGCTCTGGctggatAGCAAAGTTGGTGGA |
| 3 | 440F_RBM | TTCTATTCTCACTCCGCTCCAGAGCTCTGGaacctggatAGCAAAGTTGGTGGA |
| 4 | 439F_RBM | TTCTATTCTCACTCCGCTCCAGAGCTCTGGaataacctggatAGCAAAGTTGGTGGA |
| 5 | 438F_RBM | TTCTATTCTCACTCCGCTCCAGAGCTCTGGagcaataacctggatAGCAAAGTTGGTGGA |
| 6 | 437F_RBM | TTCTATTCTCACTCCGCTCCAGAGCTCTGGaatagcaataacctggatAGCAAAGTTGGTGGA |
| 7 | 436F_RBM | TTCTATTCTCACTCCGCTCCAGAGCTCTGGtggaatagcaataacctggatAGCAAAGTTGGTGGA |
| 8 | 435F_RBM | TTCTATTCTCACTCCGCTCCAGAGCTCTGGgcttggaatagcaataacctggatAGCAAAGTTGGTGGA |

TABLE 5
Anti-sense oligonucleotides for the construction of the 3^{rd} generation extended RBM
libraries. The oligos 1-11 sequences are denoted by SEQ ID NOs: 111-121, respectively.

| | Name | Sequence 5′-3′ |
|---|---|---|
| 1 | 500R_RBM | ACAACTTTCAACAGTTTCCCAGACGTCTGGtgtTGGTTGAAAACCGTA |
| 2 | 501R_RBM | ACAACTTTCAACAGTTTCCCAGACGTCTGGgtttgtTGGTTGAAAACCGTA |
| 3 | 502R_RBM | ACAACTTTCAACAGTTTCCCAGACGTCTGGaccgtttgtTGGTTGAAAACCGTA |
| 4 | 503R_RBM | ACAACTTTCAACAGTTTCCCAGACGTCTGGaacaccgtttgtTGGTTGAAAACCGTA |
| 5 | 504R_RBM | ACAACTTTCAACAGTTTCCCAGACGTCTGGtccaacaccgtttgtTGGTTGAAAACCGTA |
| 6 | 505R_RBM | ACAACTTTCAACAGTTTCCCAGACGTCTGGatatccaacaccgtttgtTGGTTGAAAACCGTA |
| 7 | 506R_RBM | ACAACTTTCAACAGTTTCCCAGACGTCTGGttgatatccaacaccgtttgtTGGTTGAAAACCGTA |
| 8 | 507R_RBM | ACAACTTTCAACAGTTTCCCAGACGTCTGGtggttgatatccaacaccgtttgtTGGTTGAAAACCGTA |
| 9 | 508R_RBM | ACAACTTTCAACAGTTTCCCAGACGTCTGGatatggttgatatccaacaccgtttgtTGGTTGAAAACCGTA |
| 10 | 509R_RBM | ACAACTTTCAACAGTTTCCCAGACGTCTGGccgatatggttgatatccaacaccgtttgtTGGTTGAAAACCGTA |
| 11 | 510R_RBM | ACAACTTTCAACAGTTTCCCAGACGTCTGGgacccgatatggttgatatccaacaccgtttgtTGGTTGAAAACCGTA |

Mixes of the 8 Sense oligonucleotides and 11 Anti-sense oligonucleotides were prepared individually. In order to ensure the amplification of all extended combinations, 11 PCR reactions of the mix of the Sense oligonucleotides with each one of the Anti-sense oligonucleotides were performed by using the Klenow fragment of each library separately as template and vice versa. The PCR products for each library were consolidated and cloned into BstXI digested fth1 vector using Gibson assembly reaction. After calibration of the efficacy of the Gibson reaction in DH5alphaF-competent cells, Gibson reactions for each library were used to electroporate TG1 E. coli cells. After 1 hr of phenotypic expression, the bacterial cultures were tittered in order to estimate the actual complexity of the libraries (Table 6). The variation of each library was confirmed by sequencing 10-20 randomly picked colonies from the titration plate.

TABLE 6
Calculated complexities of the 3^{rd} generation RBM libraries.

| Extended RBM | Measured |
|---|---|
| library | complexity |
| 1 × NNK | 1.6 × 10⁵ |
| 2 × NNK | 1.2 × 10⁵ |
| 3 × NNK | 2.4 × 10⁶ |
| 4 × NNK | 3.5 × 10⁵ |
| 5 × NNK | 3 × 10⁵ |

Each library was further amplified by growing the bacterial culture after electroporation in 500 ml LB with 20 μg/ml of tetracycline overnight at 37° C., 225 rpm. Next, the culture was centrifuged for 20 min, 8000 rpm at room temperature to pellet the bacteria. The phage-containing supernatant was transferred to cold polyethylene-glycol/NaCl (33% PEG-6000, 3.3M NaCl) and incubated at 4° C. overnight to precipitate phages. Following centrifugation of 45 min, 8000 rpm at 4° C., the precipitated phages were resuspended in sterile TBS and tittered by plaque assay.

was constructed in the same fashion using the sense oligos and a new set of anti-sense oligos in which the codon for N 501 was modified to produce Y501 as is given in the Table.

Similarly, a fourth generation extended RBM libraries were constructed in which the Asparagine at the 501 position was replaced with Tyrosine. The generation of the in the same fashion using the sense oligos and a new set of anti-sense oligos in which the codon for N 501 was modified to produce Y501 as is given in the Table 7.

TABLE 7
Anti-sense oligonucleotides for the construction of the 3^{rd} generation extended RBM
libraries with the N501Y substitution. The oligos sequences are denoted by SEQ ID NOs:
123-132, respectively.

| Name | 5-3 |
|---|---|
| Y501R_RBM | ACAACTTTCAACAGTTTCCCAGACGTCTGGgtAtgtTGGTTGAAAACCGTA |
| Y502R_RBM | ACAACTTTCAACAGTTTCCCAGACGTCTGGaccgtAtgtTGGTTGAAAACCGTA |
| Y503R_RBM | ACAACTTTCAACAGTTTCCCAGACGTCTGGaacaccgtAtgtTGGTTGAAAACCGTA |
| Y504R_RBM | ACAACTTTCAACAGTTTCCCAGACGTCTGGtccaacaccgtAtgtTGGTTGAAAACCGTA |
| Y505R_RBM | ACAACTTTCAACAGTTTCCCAGACGTCTGGatatccaacaccgtAtgtTGGTTGAAAACCGTA |
| Y506R_RBM | ACAACTTTCAACAGTTTCCCAGACGTCTGGttgatatccaacaccgtAtgtTGGTTGAAAACCGTA |
| Y507R_RBM | ACAACTTTCAACAGTTTCCCAGACGTCTGGtggttgatatccaacaccgtAtgtTGGTTGAAAACCGTA |
| Y508R_RBM | ACAACTTTCAACAGTTTCCCAGACGTCTGGatatggttgatatccaacaccgtAtgtTGGTTGAAAACCGTA |
| Y509R_RBM | ACAACTTTCAACAGTTTCCCAGACGTCTGGccgatatggttgatatccaacaccgtAtgtTGGTTGAAAACCGTA |
| Y510R_RBM | ACAACTTTCAACAGTTTCCCAGACGTCTGGgacccgatatggttgatatccaacaccgtAtgtTGGTTGAAAACCGTA |

### Example 4

Bio-Panning of 2^{rd} and 3^{rd} Generation RBM Libraries Against a Panel of Monoclonal Neutralizing Anti-RBM Antibodies or Human ACE2-Fc

The general concept and protocol of bio-panning is described in detail in (Freund N T, Enshell-Seijffers D, Gershoni J M: Phage display selection, analysis, and prediction of B cell epitopes. Curr Protoc Immunol 2009, 86:9.8.1-9.8.30). Specifically, for bio-panning of the second-generation RBM library and the 3^{rd} generation Extended RBM library, 2 μg of each mAb or human ACE2-Fc were mixed with approximately 1×10¹⁰ phages (10-30 μl) from the library of choice (or a mixture of libraries) and completed to 100 μl with blocking solution (sterilized Tris-buffered saline, TBS, containing 3% BSA, filtered through 0.22 μm filter). Next, the mix was incubated on a rotator for 1 hr at room temperature. Magnetic protein G beads (Dynabeads 10004D, Invitrogen) were mixed until complete resuspension and 50 μl of the beads were added to the mAb/human ACE2-Fc-phage mix for 30 min on a rotator at room temperature. In order to pull down mAbs or human ACE2-Fc and the associated phages, tubes containing the mAb/human ACE2-Fc-phages mix were placed in a magnetic stand (Promega) and supernatant was discarded. The tubes were removed from the magnetic stand, resuspended in wash buffer (sterilized Tris-buffered saline, TBS, containing 0.5% Tween-20, filtered through 0.22 μm filter) and placed back in the magnetic stand to discard the supernatant. This washing step was repeated to a total of three times. After washing, the beads were resuspended in 100p of wash buffer and transferred to a new tube in order to avoid co-elution of non-specific proteins. The tubes were placed in the magnetic stand and supernatant was removed. For elution of phages bound to target mAbs or human ACE2-Fc, elution buffer (100 mM HCl-Glycine, 1 mg/ml BSA, pH2.2, filtered through 0.22 μm filter) was added to the beads for 10 min at room temperature, tubes were placed back on the magnetic stand, supernatant was collected and immediately neutralized (1M Tris-HCl—NaOH, pH9.1). The elution step was repeated to a total of two times. The two eluates were unified after neutralization and phages were tittered by plaque assay. To amplify the phages of the 1^{st} eluate, E. coli DH5αF′ were grown to approximately 1.5 OD₆₀₀ in 5 ml of YTX2 at 225 rpm, 37° C. Next, the speed of shaking was reduced to 60 rpm for 30 min in order to allow the reconstitution of bacterial pili. 150 ul of eluate 1 was added to the bacteria, incubated for 30 min at 60 rpm, 37° C. and then transferred to a flask containing 100 ml of 2XYT and incubated for 1 hr, 225 rpm at 37° C. for phenotypic expression. Subsequently, 20 μg/ml of tetracycline was added to the bacterial culture which was then grown for 20 hrs at 225 rpm, 37° C. To extract the phages, the bacterial culture was transferred to sterile 250 ml centrifugation bottles and centrifuged for 20 min, 8000 rpm at room temperature to pellet the bacteria. The supernatant was transferred to new 250 ml centrifugation bottles containing 0.4 volumes of cold Polyethylene-glycol/NaCl (33% PEG-6000, 3.3M NaCl), thoroughly mixed and incubated over night at 4° C. to precipitate the phages. In order to pellet the phages, bottles were centrifuged for 45 min, 8000 rp, at 4° C., supernatant was discarded and after a short spin down, the phages were resuspended in 2 ml of sterile TBS for 30 min, 225 rpm, 37° C. The phages were centrifuged for 5 min, 14000 rpm at room temperature to pellet debris and supernatant was transferred to a new tube. Typically, two to six rounds of amplification were carried out in each bio-panning using 10-30 μl of the amplified phages.

In order to confirm mAb or human ACE2-Fc binding to the affinity-selected phages, E. coli DH5αF′ were infected with the eluted phages and the bacteria were plated on LB with 20 μg/ml of tetracycline. Single colonies were picked and grown as mini-cultures in U-bottom 96-well cell culture plates (Corning Inc. Life Sciences, Tewksbury, MA). The plates were centrifuged for 45 min, 4000 rpm at room temperature to pellet the bacteria and supernatants were transferred to 96-well flat bottom plates (Greiner Bio-One GmbH, Germany) containing polyethylene-glycol/NaCl solution to precipitate phages followed by another centrifugation step to pellet the phages. Phages were resuspended in 100 μl of TBS and used in confirmatory dot blot analyses.

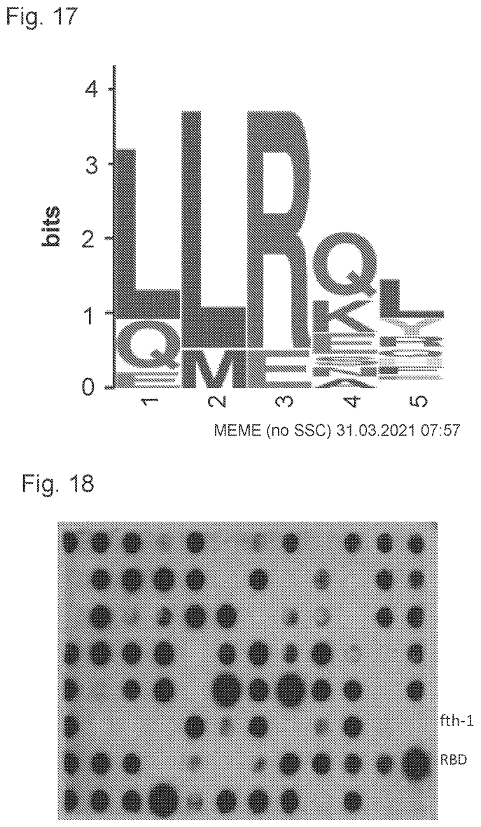

For dot blot analyses of potential binders, phages were applied to nitrocellulose membrane filters using a vacuum manifold and quenched using 5% skim milk in TBS for 1 hr at room temperature. FIG. 18 shows a representative dot blot analysis. Next, the filters were incubated with mAbs or human ACE2-Fc overnight at 4° C. at a concentration of 2.5 μg/ml in 5% skim milk/TBS. After washing 5 times, for a total of 30 min with TBS containing 0.05% Tween-20, the filters were incubated with HRP-conjugated antibodies at 1:5000 dilution (0.2 μg/ml) (Jackson, West Grove, PA) for 45 min at room temperature in 5% skim milk/TBS. Next, the filters were washed 5 times with TBS/0.05% Tween-20 and signals were developed using the enhanced chemo-luminescence (ECL) reaction (Rhenium, Israel).

To further validate the binding of the phages to their respective targets, the phages were grown in 100 ml 2×YT for 20 hrs, 225 rpm at 37° C. and extracted as described above.

FIG. 16 illustrates the results of the extended third generation library and the resulting reconstituted RBMs (also denoted by SEQ ID NO: 66-84). As shown by the figure, combinatorial several linkers were also revealed (SEQ ID NO: 42-52). FIG. 17 shows a schematic illustration of the linkers found. As shown by the figure, although thus far the number of functional linkers is limited, an obvious motif is apparent. Hence aliphatic methyls are preferred in the first two positions of the motif and Leucine is most prevalent. The third position is charged where arginine is most common. Positions 4 and 5 are more variable where polar residues dominate position 4 and position 5 is more hydrophobic.

### Example 5

Selection of RBM Variants Via Biased Random Mutagenesis

Once a functional ACE2-binding reconstituted RBM is produced it is further used to produce a vast panel of genetically modified RBMs via Biased Random Mutagenesis (BRM, Ophir et al., Protein Engineering vol. 8 no. 2 pp. 143-146, 1995). This is accomplished by constructing a synthetic oligonucleotide corresponding to the RBM sequence using contaminated phosphoramidite stock solutions. Thus, for example the stock of G phosporamidite contains 97 percent G and 1 percent of A, T and C respectively. The same is for the stock of A which is laced with 1 percent G, T and C respectively. Using such contaminated stocks will ensure that at each position of the oligonucleotide there will be a 97 percent chance for the correct intended base. However, there is also a chance of 3 percent error. Constructing the oligonucleotide corresponding to the RBM using such contaminated stock solutions will produce a library of RBM variants in which each of the original amino acids has a possible chance for variation. The degree of variation can be modified and regulated by increasing or decreasing the degree of contamination in the stock solutions. Thus, a library of RBM variants is produced with a strong bias for the correct residue at each position but with a chance of variation as well. The BRM-RBM library will then be screened against ACE2 at various stringencies. Thus, for example, the library is screened at elevated temperature, or at ever decreasing ACE2 concentrations, or subjecting the affinity selected RBM candidates to more rigorous washing conditions, etc. The result of this procedure will be the selection of RBM variants with improved and more robust affinity for ACE2. This will reveal the structure of most efficiently adapted RBM for ACE2 binding and thus will assist in the prediction of future mutations of SARS CoV2 that may acquire preferred structures that have optimized ACE2 binding activities.

### Example 6

Spike Containing Reconstituted RBM

The spike protein and stabilized spike proteins are proposed vaccine modalities for SARS CoV2. Here swapping the native RBM with the reconstituted RBM is performed. It should be noted that each of the reconstituted RBMs can be used herein, for example, each of SEQ ID NOs. 66-84.

This is expected to be beneficial and to improve dramatically the immunogenicity of this critical neutralizing determinant of the spike protein. The rationale for this is that by replacing the anchor loop with the selected linker to give RBM flexibility and render improved immunogenicity compared to the native RBM which is hydrogen bonded to the core of the RBD via the anchor loop which was removed. Thus, a proposed modality for vaccine immunogen is the Spike protein in which the native RBM is replaced with the reconstituted version in which the anchor loop is replaced with a functional linker.

### Example 7

RBD Containing Reconstituted RBM

As in the previous examples, the native RBM is replaced with the reconstituted RBM in the RBD of the SARS CoV2 (residues ca. 336-518). Here also, it is expected anticipate that the more flexible expression of RBM in the context of the RBD is a preferred vaccine modality for elicitation of RBM targeted antibodies.

### Example 8

Production of an Active Prophylactic Vaccine Against SARS-Cov2

The reconstituted RBM or Spike protein or any fragment thereof or RBD comprising said reconstituted RBM, is expressed and used as a vaccine immunogen to elicit protective antibodies in vaccinated subjects. More specifically, the reconstituted RBM or any polypeptide comprising the reconstituted RBM, is incorporated in a multimeric/multivalent antigen displaying platform, for example, a self-assembling nanostructure (e.g., the platform described by Dr. Neil King of University of Washington Seattle [11]. The reconstituted RBMs are expressed as Component A in the self-assembling capsid nanoparticle. Component A is expressed in E. coli and is mixed with Component B. This forms a nanomolecular capsid structure in which the Components A for trimers and associate with pentameric Component B. Some 20 trimeric Component A can assemble with Component B. Thus, a polyvalent immunogenic capsid is formed and can be used as a preferred embodiment of the invention. Other presentations of the reconstituted RBM are also envisioned using a variety of scaffolds. Dendrimers presenting multiple copies of the RBM are also encompassed by the invention. Tetrameric RBM is easily produced by incorporating an Avitag sequence at the RBM carboxy-terminal end. Hence each RBM can be C-terminally biotinylated. Mixing biotinylated RBM with Avidin produces stable tetramers.

Heteromeric mixes of the RBM is also possible. Screening the conformer library produces variations of functional RBM that contain variations of functional linkers ranging from 3 to 7 amino acids. Thus, each functional linker may generate slight variations in the RBM conformation representing the dynamic conformational variations of the RBM in the virus. In producing the nano-particle self-assembling capsid of Neil King, one can produce chimeric capsids by mixing component B with a variety of Component A units each displaying functional RBMs that differ in their functional linker compositions.

Adding glycosylation sites to either Component A or B can increase the solubility of the ultimate capsid if it is expressed in a mammalian cell line such as Vero cells or 293T cells.

### Example 9

Passive Therapeutic Vaccine

The RBM immunogen is used to elicit specific antibodies in rabbits or other small animals or non-human primates. The sera are collected and immunoglobulins are purified and used to counteract SARS CoV2 in critically ill patients as a therapeutic passive vaccine. The immunoglobulin fractions are further purified by affinity selection of RBM-specific antibodies by passing them over an immobilized RBM column and elution of the RBM-specific fraction.

### Example 10

Affinity Purification of RBM-Specific Antibodies

As in the Passive therapeutic vaccine application, affinity purification of RBM specific antibodies are purified from convalescent sera of individuals that have cleared the SARS CoV2 and recovered. More specifically, the immunoglobulin fractions of the collected convalescent sera are further purified by affinity selection of RBM-specific antibodies by passing them over an immobilized RBM column and elution of the RBM-specific fraction. The affinity-purified human RBM specific mAbs are used to treat the critically ill patients.

### Example 11

Purification of Human Anti-RBM Antibodies

PBMCs of recovered SARS CoV2 patients are collected and used to isolate memory B-cells using standard protocols for single cell cloning of select B-cells. RBM specific B-cells are selected and enriched by FACscan or other means in which the reconstituted RBM is used as bait. The RBM selected B-cells are further developed to produce and clone out the specific heavy chains and light chains to produce RBM specific human monoclonal antibodies. These can in turn be used therapeutically to treat critical patients or used in solid phase immuno-assays for diagnosis.

### Example 12

Purification of Anti-RBM Antibodies from Phage Display Antibody Libraries

Phage display antibody libraries are screened against reconstituted RBM of SARS CoV2. The affinity selected phages are then used to produce RBM specific antibodies. The Heavy Chain Fv and Light Chain Fv are then cloned onto a human IgG1 Fc scaffold to produce full length RBM specific IgG1. These can in turn be used therapeutically to treat critical patients or used in solid phase immuno-assays for diagnosis.

### Example 13

Use of the RBM as Bait for the Affinity Selection of RBM Specific Antibodies

The RBM is selected from a conformer library displayed on filamentous bacteriophages based on the fth1 phage vector previously described by the inventor (Enshell D et al., Nucleic Acids Res. 2001 May 15; 29(10): e50). The combinatorial linker library is expressed on Protein 3 which exists in five copies. Thus, in accordance with some embodiments of the invention, the reconstituted RBM of the invention expressed on Protein 3, may be referred to herein as a multimeric version of the RBM. Still further, to ensure that one can use the phage as bait a biotinylated phage expressing a desired peptide, for example, the functional RBM of the invention, was next produced. More specifically, in a previous study (Smelyanski L et al., Virology Journal 2011, 8:495), the inventors described the introduction of a biotinylation site (“Avi-Tag”) in anyone of the structural proteins of the filamentous phage, proteins 3, 7, 8 and 9. In each case, the phage can be biotinylated in vivo and thus released from the bacterium with a biotin molecule. Alternatively, the Avi-Tag can be biotinylated in vitro by reacting the phage with the natural biotinylating enzyme, biotin holoenzyme synthetase the product of the BirA gene in E. coli. Thus, a RBM expressing phage can be produced, the RBM being expressed on Protein 3, which is also biotinylated on Protein 7, 8 and/or 9. The preferred embodiment would be to biotinylated on Protein 8. This is achieved by either expressing the “Avi-Tag” using the recombinant Protein 8 gene in the vector, or using an extraneous Protein 8 gene as described in Smelyanski et al. This configuration could be very easily used without the need to clone the functional RBM into a new vector or scaffold system. The successfully cloned ACE2 binding RBM expressed on Protein 3 could be easily then biotinylated using the extraneous Avi-Tag containing protein 8. The biotinylated phage could then be immobilized on any Streptavidin or Avidin or anti-Avidin antibody containing surface or chromatographic medium such as sepharose 4b, or magnetic bead system. The immobilized RBM expressing phage could then be used directly to affinity purify RBM specific antibodies from convalescent serum, used to affinity select memory B-cells for single cell antibody cloning, or used to affinity select any compound (e.g., small molecule) that may bind the RBM thereby inhibiting and/or preventing binding of the virus to the cognate receptor.

### Example 14

Diagnostic Platforms

The RBM is used directly in solid phase immunoassays as immobilized target antigen. This has the added advantage of simply using spike protein as the presence of anti-RBM antibodies indicates a more positive prognosis.

### Example 15

Clinical Experiments

The following experiment are conducted in order to evaluate the efficacy of the reconstituted RBMs as potential vaccine immunogens. Small animal models, mice and rabbits can be used to first test the humoral response towards the reconstituted RBMs. The RBMs are produced as protein-fusions with Maltose Binding Protein (MBP) as described in the past [2]. Three experimental arms with 5 animals in each are used.
A. Vaccination with MBP-RBM
B. Vaccination with MBP
C. Vaccination with adjuvant alone.

Each of the animals in A receives an initial immunization of 10-50 microgram of MBP-RBM in 100 microliters aluminum hydroxide salt solution as adjuvant. The injections are delivered subcutaneous or intra-muscular. The animals are then boosted after 3-4 weeks and then repeated with a second boost 3-4 weeks later (total 3 injections, immunization and two boosts). Blood is drawn one just prior the first and second boosts and 2 weeks after the second boost. A serum sample of each animal prior the initial immunization is kept as “pre-immune” control.

The animals in groups B and C follow the same immunization scheme as in A but using either MBP alone or just adjuvant solution, respectively.

The serum samples are then test in ELISA tests against SARS CoV2 spike protein to evaluate the degree of RBM specific antibodies in group A as compared to the response towards MBP alone and the negative control of adjuvant alone. The sera can also be tested against the phage displayed RBM.

In addition, the sera are tested against SARS CoV2 virus like particles or infectious virus as well. Finally, the sera are mixed with infectious virus and to evaluate the neutralization activity in an infectivity assay using cells in culture.

These experiments measure the level of antibody response towards the RBM and its potential use in the elicitation of neutralizing antibodies.

### Example 16

Experiments to Evaluate the Use of RBM as a Universal Boost

Currently there are at least 6 different SARS CoV2 vaccines being used in humans: Moderna and Pfizer mRNA vaccines, J&J adeno 26 vaccine, Gamaleya Sputnik V using both adeno 5 and adeno 26 and AstraZeneca CHADOX based vaccine as well as killed inactivated vaccines produced and used in China (e.g., SinoVax). All these vaccines elicit protective immunity against the intact full-length Spike protein (ca. 1200 amino acids). Obviously, the most potent active component of the elicited response are the neutralizing antibodies that target the RBM of the virus. Thus far it is still not known how robust or durable the vaccine response is, how long it will last, what is the kinetics of the waning of the serum antibody titer and how will this effect level of protection against infection and development of disease.

In the event that the protective efficacy of the vaccine diminishes over time, there may be the need for additional boosts. The reconstituted RBM can serve as a universal boost irrespective of which vaccine was administered originally. An RBM based boost would be advantageous in that it would enhance and strengthen the neutralizing antibodies preferentially and not boost B-cells that target otherwise irrelevant epitopes of the Spike. Hence, clinical trials for selective boost efficacy can be conducted as follows:

Human subjects are recruited under informed consent who are at least 6 months after completion of their vaccination protocol. Preferably, participants will be enrolled representing a variety of different vaccines (Moderna, Pfizer, J&J etc). The participants are divided into three groups: A—boosted with RBM, B—boosted with the corresponding original vaccine modality, boosted with mock solution containing no immunogen (spike, virus or RBM). Subjects are injected intramuscular as with the original protocol or with RBM at effective concentration (to be determined in preliminary Phase 1 trial using escalating doses ranging from 10 to 200 μg, specifically, 30 μg or 100 μg.

Serum samples are taken before boosts and 3 weeks after the boost. The level of neutralizing activity is measured in each arm and for each subject. Moreover, the sera are tested for IgOme profiling and the level of RBM specific antibodies will be measured for each sample.

1-50. (canceled)

51. A polypeptide comprising an amino acid sequence of at least one reconstituted Receptor Binding Motif (RBM) of a Spike protein of the Severe Acute Respiratory Syndrome coronavirus 2 (SARS CoV2) or of any fragment thereof, wherein said reconstituted RBM comprises at least one linker and at least one fragment of the native RBM or of any variant or mutant thereof, said native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510, of the SARS CoV2 spike protein, wherein said native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, and wherein said at least one of said linker/s replaces said anchor loop or any part thereof or amino acid residue/s thereof and any RBM fragment or amino acid residue/s thereof.

52. The polypeptide according to claim 51, wherein said SARS CoV2 spike protein comprises the amino acid sequence as denoted by SEQ ID NO: 6, or any variants, mutants, derivatives and homologs thereof, optionally, wherein said native RBM comprises an amino acid sequence of any one of: (a) residues S443 to P499 of the SARS CoV2 Spike protein; (b) residues S443 to P499 of the SARS CoV2 Spike protein with at least one to eleven flanking amino acid residue/s; and (c) any variant, mutant, parts or fragments of the amino acid sequence of residues S443 to P499 of the SARS CoV2 Spike protein; and wherein said anchor loop of said native RBM comprises the amino acid sequence of any one of (d) residues R457 to I472 of the SARS CoV2 Spike protein; (e) residues R457 to I472 of the SARS CoV2 Spike protein with at least one or two flanking amino acid residue/s; and (f) any variant, mutant, parts or fragments of the amino acid sequence of residues R457 to I472 of the SARS CoV2 Spike protein.

53. The polypeptide according to claim 51, wherein said reconstituted RBM comprises at least one linker and at least two fragments of the native RBM or of any variant or mutant thereof, wherein said at least two fragments comprise:
(a) the amino acid sequence of any one of:
(i) residues S443 to F456 of the SARS CoV2 Spike protein;
(ii) residues S443 to F456 of the SARS CoV2 Spike protein with at least one to eight flanking amino acid residue/s; or
(iii) any variant, mutant, parts or fragments of the amino acid sequence of residues S443 to F456 of the SARS CoV2 Spike protein; and
(b) the amino acid sequence of any one of:
(i) residues Y473 to P499 of the SARS CoV2 Spike protein;
(ii) residues Y473 to P499 of the SARS CoV2 Spike protein with at least one to eleven flanking amino acid residue/s; or
(iii) any variant, mutant, parts or fragments of the amino acid sequence of residues Y473 to P499 of the SARS CoV2 Spike protein;
optionally, wherein at least one of:
(I) wherein said at least one linker is a bridging linker that bridges residue 456 with residue 473 of the SARS CoV2 Spike protein;
(II) wherein said at least one linker is an amino acid linker comprising 3 to 7 amino acid residues; and
(III) wherein said at least one linker comprises the amino acid sequence of Xaa1-Xaa2-Xaa3-Xaa(n) as denoted by SEQ ID NO: 57 or any fragment thereof, wherein Xaa is any amino acid, wherein n is zero or an integer of from 1 to 4, and wherein: Xaa 1 is a Leu, Gln, Glu or Gly; Xaa2 is Leu, Met or Val; and Xaa3 is Arg, Glu or Pro.

54. The polypeptide according to claim 51, wherein said polypeptide is the Receptor Binding Domain (RBD) of said Spike protein of SARS CoV2, or the Spike protein of SARS CoV2, wherein said RBD comprises the reconstituted RBM comprising at least two fragments of the native RBM and at least one linker, and wherein said Spike protein comprises the reconstituted RBM comprising at least two fragments of the native RBM and at least one linker.

55. A Receptor Binding Motif (RBM) of SARS CoV2 comprising the native Receptor Binding Domain (RBD) of the spike protein of SARS CoV2 or any fragments thereof and at least one linker, or a Spike protein of SARS CoV2, comprising the native Spike protein of SARS CoV2 or any fragment thereof and at least one linker, wherein at least one of said linker replaces an anchor loop or any part thereof or amino acid residue/s thereof, said loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, wherein at least one of said linker is a bridging linker.

56. A multimeric and/or multivalent antigen displaying platform comprising at least one polypeptide comprising at least one reconstituted RBM of a Spike protein of SARS CoV2 according to claim 51, or of any variant, mutant or fragment thereof, or any fusion protein or conjugate thereof, any RBD or Spike protein comprising said reconstituted RBM, any RBD or Spike protein comprising at least one linker that replaces an anchor loop or any part thereof or amino acid residue/s thereof, said loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, and any combinations thereof.

57. A nucleic acid sequence encoding at least one polypeptide comprising at least one reconstituted RBM of a Spike protein of SARS CoV2 according to claim 51, or of any fragment thereof, or any fusion protein thereof, any RBD or Spike protein comprising said reconstituted RBM, any RBD or Spike protein comprising at least one linker that replaces an anchor loop or any part thereof or amino acid residue/s thereof, said loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, any multimeric and/or multivalent antigen displaying platform thereof, and any combinations thereof, or any matrix, nano- or micro-particle thereof.

58. A composition or a SARS CoV2 vaccine comprising an effective amount of at least one polypeptide comprising an amino acid sequence of at least one reconstituted RBM of a viral Spike protein of SARS CoV2, according to claim 51, or of any fragment thereof, any RBD or Spike protein comprising said reconstituted RBM, any RBD or Spike protein comprising at least one linker that replaces an anchor loop or any part thereof or amino acid residue/s thereof, said loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, any multimeric and/or multivalent antigen displaying platform thereof, and any combinations thereof, any derivative, enantiomer, fusion protein, conjugate, polyvalent dendrimer thereof, and any nucleic acid sequence encoding the same or any matrix, nano- or micro-particle thereof said composition or vaccine optionally further comprises at least one pharmaceutically acceptable carrier/s, excipient/s, adjuvant/s, auxiliaries, and/or diluent/s.

59. A method for treating, preventing, inhibiting, reducing, eliminating, protecting or delaying the onset of an infection or an infectious clinical condition caused by SARS CoV2, and/or for inducing an immune response against a SARS CoV2, in a subject in need thereof, the method comprising the step of administering to said subject an effective amount of at least one polypeptide comprising an amino acid sequence of at least one reconstituted Receptor Binding Motif (RBM) of a viral Spike protein of SARS CoV2 or of any fragment thereof, any Receptor Binding Domain (RBD) or Spike protein comprising said reconstituted RBM, any RBD or Spike protein comprising at least one linker that replaces an anchor loop or any part thereof or amino acid residue/s thereof, said loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, any multimeric and/or multivalent antigen displaying platform thereof, any derivative, enantiomer, fusion protein, conjugate, polyvalent dendrimer thereof, any nucleic acid sequence encoding the same or any matrix, nano- or micro-particle thereof, any combinations thereof, any compositions thereof and any vaccine thereof; wherein said reconstituted RBM comprises at least one linker and at least one fragment of the native RBM or of any variants and mutants thereof, said native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein, wherein said native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, and wherein said at least one of said linker/s replaces said anchor loop or any part thereof or amino acid residue/s thereof, optionally, at least one of:
(I) wherein said polypeptide, RBD or Spike protein comprising said reconstituted RBM, is a polypeptide comprising an amino acid sequence of at least one reconstituted Receptor Binding Motif (RBM) of a Spike protein of the Severe Acute Respiratory Syndrome coronavirus 2 (SARS CoV2) or of any fragment thereof, wherein said reconstituted RBM comprises at least one linker and at least one fragment of the native RBM or of any variant or mutant thereof, said native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510, of the SARS CoV2 spike protein, wherein said native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, and wherein said at least one of said linker/s replaces said anchor loop or any part thereof or amino acid residue/s thereof and any RBM fragment or amino acid residue/s thereof; and
(II) wherein said method is for eliciting a neutralizing antibody response to SARS CoV2 in said subject.

60. A method for the preparation of a polypeptide comprising a functional reconstituted RBM of a Spike protein of SARS CoV2, according to claim 51, the method comprising the step of:
(a) screening a conformer library of RBMs of said viral Spike protein with at least one binding molecule, said library comprising plurality of combinatorial display vehicles, each expressing a reconstituted RBM comprising an amino acid sequence of at least one fragment of a native RBM of a Spike protein of said SARS CoV2, or any variant or mutant thereof, and at least one combinatorial linker, said native RBM comprises an amino acid sequence starting at any one of the amino acid residues S443, A435, W436, N437, S438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein, wherein said native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, and wherein said at least one of said linker/s replaces said anchor loop or any part thereof or amino acid residue/s thereof;
(b) identifying and producing reconstituted RBM peptides which bind at least one of said binding molecules; optionally, wherein at least one of:
(I) wherein said binding molecule is at least one of: (i) the receptor for said SARS CoV2 or any fragments thereof; (ii) neutralizing antibodies of convalescent serum of at least one patient recovered from SARS CoV2 infection; (iii) antibodies that neutralize the virus and compete with receptor binding, and any antigen-binding fragments thereof; and (iv) and any combinations of (i), (ii) and (iii); and
(II) wherein said method is for producing SARS CoV2 vaccine comprising reconstituted RBM, the method further comprising the steps of admixing at least one of said reconstituted functional RBM/s of a Spike protein of SARS CoV2 or any derivative or enantiomer thereof, or any fusion protein, conjugate, or polyvalent dendrimer comprising the same with at least one adjuvant/s, carrier/s, excipient/s, auxiliaries, and/or diluent/s.

61. A method for the preparation, affinity selection and/or isolation of neutralizing antibodies that neutralize the SARS CoV2 and compete with receptor binding, the method comprising the steps of:
(a) contacting a serum or lymphocytes of at least one donor with an effective amount of a polypeptide comprising at least one reconstituted RBM of the Spike protein of SARS CoV2 according to claim 51, associated directly or indirectly to a solid support and/or a detectable moiety, or any fragment of said reconstituted RBM, any RBD or Spike protein comprising said reconstituted RBM, any RBD or Spike protein comprising at least one linker that replaces an anchor loop or any part thereof or amino acid residue/s thereof, said loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, any multimeric and/or multivalent antigen displaying platform thereof, any derivative, enantiomer, fusion protein, conjugate, polyvalent dendrimer thereof, and any combinations thereof; and
(b) recovering the antibodies or at least one lymphocyte bound to said reconstituted RBM; Wherein said reconstituted RBM comprises at least one linker and at least one fragment of the native RBM or of any variant or mutant thereof, said native RBM comprises an amino acid sequence starting at any one of the amino acid residues S443, A435, W436, N437, 5438, N439, N440, L441, D442, K444, V445, G446, G447 or N448, and ending at any one of the amino acid residues P499, Y495, G496, F497, Q498, T500, N501, G502, V503, G504, Y505, Q506, P507, Y508, R509 or V510 of the SARS CoV2 Spike protein, wherein said native RBM comprises an anchor loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, and wherein said at least one of said linker/s replaces said anchor loop or any part thereof or amino acid residue/s thereof and any RBM fragment or amino acid residue/s thereof;
optionally, wherein one of:
(I) the method is for the production of human monoclonal neutralizing antibodies that neutralize the SARS CoV2, the method comprising the steps of:
(i) contacting lymphocytes of at least one donor with an effective amount of said reconstituted CoV2 RBM, any RBD or Spike protein comprising said reconstituted RBM, any RBD or Spike protein comprising at least one linker that replaces an anchor loop or any part thereof or amino acid residue/s thereof, said loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, any multimeric and/or multivalent antigen displaying platform thereof, and any combinations thereof, any derivative, enantiomer, fusion protein, conjugate, polyvalent dendrimer thereof, associated directly or indirectly to a detectable moiety and/or any solid support;
(ii) selection and single cell cloning of antibody producing lymphocyte bound to said reconstituted RBM; and
(iii) cloning the nucleic acid sequence encoding the variable regions or any segments thereof of at least one of the heavy and the light chains of an antibody produced by said cells; or
(II) the method is for the production of human polyclonal neutralizing antibodies that neutralize the SARS CoV2, the method comprising the steps of:
(i) contacting serum of at least one donor or any immunoglobulin fraction thereof, with an effective amount of said reconstituted SARS CoV2 RBM, any RBD or Spike protein comprising said reconstituted RBM, any RBD or Spike protein comprising at least one linker that replaces an anchor loop or any part thereof or amino acid residue/s thereof, said loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, any multimeric and/or multivalent antigen displaying platform thereof, and any combinations thereof, any derivative, enantiomer, fusion protein, conjugate, polyvalent dendrimer thereof, associated directly or indirectly to a solid support and/or a detectable moiety;
(ii) recovering the antibodies bound to said reconstituted RBM immobilized to said solid support.

62. A method for the preparation of neutralizing antibodies directed at the Spike protein of SARS CoV2, the method comprising the step of:
(a) contacting at least one lymphocyte or serum of an immunized non-human animal, with an effective amount of at least one polypeptide comprising at least one reconstituted RBM of a Spike protein of SARS CoV2 according to claim 51, wherein said reconstituted RBM is associated directly or indirectly to a solid support and/or a detectable moiety, or any fragment of said reconstituted RBM, any RBD or Spike protein comprising said reconstituted RBM, any RBD or Spike protein comprising at least one linker that replaces an anchor loop or any part thereof or amino acid residue/s thereof, said loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, any multimeric and/or multivalent antigen displaying platform thereof, and any combinations thereof, any derivative, enantiomer, fusion protein, conjugate, polyvalent dendrimer thereof, or any SARS CoV2 virus comprising at least one linker that replaces the anchor loop of the Spike protein; and
(b) recovering the antibodies or lymphocytes bound to said reconstituted RBM; thereby obtaining neutralizing antibodies that neutralize said SARS CoV2;
wherein said non-human animal is immunized with an effective amount of said reconstituted RBM, RBD or Spike protein comprising said RBM, said RBD or Spike protein comprising at least one linker replacing the anchor loop or any part thereof or amino acid residue/s thereof, any multimeric and/or multivalent antigen displaying platform thereof, any nucleic acid sequence encoding the same or any matrix, nano- or micro-particle thereof, any combinations thereof, any derivative, enantiomer, fusion protein, conjugate, polyvalent dendrimer thereof, or any SARS CoV2 virus comprising at least one linker that replaces the anchor loop of the Spike protein, at least one attenuated or killed SARS CoV2 virus or any variant or mutant thereof, and any composition or vaccine thereof; optionally, wherein one of:
(III) the method is for the production of monoclonal neutralizing antibodies, the method comprising the steps of:
(i) contacting lymphocytes of said immunized non-human animal with said reconstituted SARS CoV2 RBM, any RBD or Spike protein comprising said reconstituted RBM, any RBD or Spike protein comprising at least one linker that replaces an anchor loop or any part or amino acid residue/s thereof, said loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, any multimeric and/or multivalent antigen displaying platform thereof, and any combinations thereof, any derivative, enantiomer, fusion protein, conjugate, polyvalent dendrimer thereof, associated directly or indirectly to a detectable moiety and/or any solid support;
(ii) selection and single cell cloning of antibody producing lymphocyte bound to said reconstituted RBM; and
(iii) cloning the nucleic acid sequence encoding at least one of the variable regions of the heavy and light chains of an antibody produced by said cells; or
(IV) the method is for the production of polyclonal neutralizing antibodies, the method comprising the steps of:
(i) contacting serum or immunoglobulin fraction thereof, of said immunized non-human animal with an effective amount of said reconstituted SARS CoV2 RBM, any RBD or Spike protein comprising said reconstituted RBM, any RBD or Spike protein comprising at least one linker that replaces an anchor loop or any part or amino acid residue/s thereof, said loop comprising an amino acid sequence starting at any one of the amino acid residues F456, R457, K458, R454 or L455 and ending at any one of the amino acid residues Y473, I472, E471, T470, S469, I468, Q474, A475 or G476 of said SARS CoV2 Spike protein, any multimeric and/or multivalent antigen displaying platform thereof, and any combinations thereof, any derivative, enantiomer, fusion protein, conjugate, polyvalent dendrimer thereof, associated directly or indirectly to a solid support and/or a detectable moiety;
(ii) recovering the antibodies bound to said reconstituted RBM immobilized to said solid support.

63. A method for treating, inhibiting, reducing, eliminating, protecting or delaying the onset of COVID-19 in a subject in need thereof, the method comprising the step of administering to said subject an effective amount of a therapeutic passive vaccine comprising neutralizing antibodies prepared by the method of claim 61.

64. A diagnostic method for the detection of SARS CoV2 infection in a mammalian subject comprising the steps of:
(a) contacting at least one biological sample of said subject with at least one of:
(i) at least one polypeptide comprising at least one reconstituted RBM according to claim 51, or any RBD or Spike protein comprising said reconstituted RBM associated directly or indirectly to a solid support and/or a detectable moiety; with
(ii) antibodies specific for said reconstituted RBM associated directly or indirectly to a solid support and/or a detectable moiety; or with
(iii) any RBM binding molecule associated directly or indirectly to a solid support and/or a detectable moiety; and
(b) determining that said subject is infected with SARS CoV2 if said detectable moiety is detected in said sample.

65. A Receptor Binding Domain (RBD) of a Coronavirus (CoV) comprising the native RBD of the spike protein of at least one CoV or any fragments thereof and at least one linker, a Spike protein of a CoV, comprising the native Spike protein of at least one CoV or any fragment thereof and at least one linker, or a multimeric and/or multivalent antigen displaying platform comprising said at least one RBD or said Spike protein of at least one CoV comprising at least one linker, wherein at least one of said linker/s replaces an anchor loop of said Spike protein or any part or amino acid residue/s thereof, said anchor loop comprises between 5 to 20 amino acid residues and forms 2 to 5 hydrogen bonds to the core of the RBD, wherein the amino acid sequence of said loop starts approximately 10 amino acid residues before the first hydrogen bond and ends approximately 10 amino acid residues after the last hydrogen bond to the core of the RBD, said core comprise about five beta strands, wherein at least one of said linker is a bridging linker.

66. A nucleic acid sequence encoding the at least one RBD or at least one Spike protein of at least one CoV according to claim 65.

67. A composition or a CoV vaccine comprising an effective amount of at least one RBD or at least one Spike protein of at least one CoV, or a multimeric and/or multivalent antigen displaying platform thereof, according to claim 65, said composition optionally further comprises at least one pharmaceutically acceptable carrier/s, excipient/s, auxiliaries, and/or diluent/s.

68. A method for treating, preventing, inhibiting, reducing, eliminating, protecting or delaying the onset of an infection or an infectious clinical condition caused by at least one CoV, and/or for inducing an immune response against at least one CoV, in a subject in need thereof, the method comprising the step of administering to said subject an effective amount of at least one RBD or at least one Spike protein of at least one CoV, or a multimeric and/or multivalent antigen displaying platform thereof, according to claim 65, or any fusion protein, multimeric and/or multivalent antigen displaying platform thereof, any derivative, enantiomer, fusion protein, conjugate, polyvalent dendrimer thereof, any nucleic acid sequence encoding the same or any matrix, nano- or micro-particle thereof, and any combinations thereof, or any composition or vaccine thereof.

69. A method for the preparation, affinity selection and/or isolation of neutralizing antibodies that neutralize at least one CoV and compete with receptor binding of said CoV, the method comprising the steps of:
(a) contacting a serum or lymphocytes of at least one donor with an effective amount of at least one RBD or at least one Spike protein of said CoV, according to claim 65, or any multimeric and/or multivalent antigen displaying platform thereof, any derivative, enantiomer, fusion protein, conjugate, polyvalent dendrimer thereof, and any combinations thereof; and
(b) recovering the antibodies or at least one lymphocyte bound to said at least one RBD or at least one Spike protein of said CoV.

70. A method for the preparation of neutralizing antibodies directed at the Spike protein of at least one CoV, the method comprising the step of:
(a) contacting at least one lymphocyte or serum of at least one immunized non-human animal, with an effective amount of at least one RBD or at least one Spike protein of said CoV according to claim 65, or any multimeric and/or multivalent antigen displaying platform thereof, any derivative, enantiomer, fusion protein, conjugate, polyvalent dendrimer thereof, or any CoV comprising at least one linker that replaces the anchor loop of the Spike protein, or any combinations thereof; and
(b) recovering the antibodies or lymphocytes bound to said at least one RBD or at least one Spike protein; thereby obtaining neutralizing antibodies that neutralize said CoV;
wherein said non-human animal is immunized with an effective amount of said at least one RBD or Spike protein comprising at least one linker replacing the anchor loop or any part or amino acid residue/s thereof, any multimeric and/or multivalent antigen displaying platform thereof, any nucleic acid sequence encoding the same or any matrix, nano- or micro-particle thereof, any combinations thereof, any derivative, enantiomer, fusion protein, conjugate, polyvalent dendrimer thereof, or any CoV comprising at least one linker that replaces the anchor loop of the Spike protein, at least one attenuated or killed CoV or any variant or mutant thereof, and any composition or vaccine thereof; and wherein said anchor loop comprises between 5 to 20 amino acid residues and forms 2 to 5 hydrogen bonds to the core of the RBD of said Spike protein, wherein the amino acid sequence of said loop starts approximately 10 amino acid residues before the first hydrogen bond and ends approximately 10 amino acid residues after the last hydrogen bond to the core of the RBD, said core comprises about five beta strands, and wherein said at least one linker is a bridging linker.

71. A method for treating, inhibiting, reducing, eliminating, protecting or delaying the onset of COVID-19 in a subject in need thereof, said method comprising the step of administering to said subject an effective amount of a therapeutic passive vaccine comprising neutralizing antibodies prepared by the method of claim 62.
